(19) United States
(12) Patent Application Publication
(10) Pub. No.: US 2011/0207113 A1
(43) Pub. Date: Aug. 25, 2011

(54) PROTEIN STRUCTURE AND METHOD OF USING PROTEIN STRUCTURE

(75) Inventors: Hans Hebert, Lidingo (SE); Caroline Jegerschold, Stockholm (SE); Per-Johan Jakobsson, Kungsangen (SE); Ralf Morgenstern, Stockholm (SE)

(73) Assignee: NOVASAID AB, Stockholm (SE)

(21) Appl. No.: 12/812,760

(22) PCT Filed: Jan. 23, 2009
(86) PCT No.: PCT/EP09/50787

§ 371 Date: Jul. 14, 2010

(30) Foreign Application Priority Data
Jan. 24, 2008 (SE) 0800169-5

(51) Int. Cl. C12Q 1/533; C12N 9/90; G06G 7/48

(52) U.S. Cl. 435/4; 435/233; 703/11

(57) ABSTRACT

Methods for building an atomic model of a protein molecule that include: (a) identifying a protein molecule with at least 20% sequence identity to Microsomal Prostaglandin E Synthase 1 (MPGES1) and (b) utilizing the atomic coordinates of MPGES1 to obtain an atomic model of the identified protein molecule are provided. Methods for determining a drug candidate compound that interacts with members of the MAPEG family, in particular MPGES1, are also provided.

### FIELD OF THE INVENTION

The present invention relates to a three dimensional structure of microsomal prostaglandin E synthase 1 (MPGES1) and methods for building an atomic model of a protein molecule using MPGES1, and methods for identifying a drug candidate compound that interacts with MPGES1.

Prostaglandins (PG) are bioactive lipids produced from arachidonic acid via the action of cyclooxygenases and terminal PG synthases. Microsomal prostaglandin E synthase 1 (MPGES1) constitutes an inducible, glutathione dependent, integral membrane protein that catalyzes the isomerisation of cyclooxygenase derived PGH₂ into PGE₂. MPGES1 has been implicated in a number of human diseases or pathological conditions such as rheumatoid arthritis, fever and pain and is therefore regarded as a primary target for the development of novel anti-inflammatory drugs.

Microsomal prostaglandin E synthase 1 (MPGES1) is the key enzyme in pathology related production of prostaglandin (PG) E₂ from cyclooxygenase (Cox) derived PGH₂ (P. J. Jakobsson, S. Thoren, R. Morgenstern, B. Samuelsson, Proc. Natl. Acad. Sci. U.S.A. 96, 7220 (1999)). The protein is a member of the MAPEG protein family including 5-lipoxygenase activating protein (FLAP), leukotriene C₄ synthase (LTC4S), microsomal glutathione transferase (MGST)1, MGST2 and MGST3 (P. J. Jakobsson, R. Morgenstern, J. Mancini, A. Ford-Hutchinson, B. Persson, Prot. Sci. 8, 689 (1999), and A. Bresell et al., FEBS J. 272, 1688 (2005)). MPGES1 is the most efficient PGES known and catalyzes the oxidoreduction of prostaglandin endoperoxide H₂ into PGE₂ with an apparent k_{cat}/Km of 310 mM⁻¹ s⁻¹. The enzyme equally well catalyses the oxidoreduction of endocannabinoids into prostaglandin glycerol esters (K. R. Kozak et al., J Biol. Chem. 277, 4487 (2002)) and PGG₂ into 15-hydroperoxy-PGE₂ (S. Thorén et al., J. Biol. Chem. 278, 22199 (2003)). In addition, the enzyme confers low glutathione transferase and glutathione dependent peroxidase activities (S. Thorén et al., J. Biol. Chem. 278, 22199 (2003)).

### SUMMARY OF THE INVENTION

The inventors have solved the three-dimensional structure of the microsomal prostaglandin E synthase 1 (MPGES1). This will aid research aimed at identifying novel compounds able to modulate the activity of MPGES1. Due to the role of MPGES1 in inflammation and detoxification, such compounds could be used in treating inflammatory diseases, rheumathoid arthritis, fever and pain. Accordingly, the present invention provides methods for building an atomic model of a protein molecule. The methods comprise: (a) identifying a protein molecule with at least 20%, and more specifically at least |30|_{[0.1]}%, sequence identity with microsomal prostaglandin E synthase 1 (MPGES1) and (b) utilizing the atomic coordinates of MPGES1 to obtain an atomic model of the identified protein molecule.

The present invention also provides a method for determining a drug candidate compound that interacts with microsomal prostaglandin E synthase 1 (MPGES1), said method comprising: (a) identifying a drug candidate compound that interacts with MPGES1 and (b) analyzing the interaction of the drug candidate compound with MPGES1 or related proteins.

### BRIEF DESCRIPTION OF THE FIGURES

The following description may be more fully understood in view of the drawings, in which:

FIG. 1

Overall structure of MPGES1. The trimer subunits are shown in ribbon representation. The upper figure is a top view from the face corresponding to the cytoplasm when the protein is positioned in its natural membrane environment. In the side view, with the cytoplasmic side up (cytosol), the transmembrane helices of one subunit have been labelled at their C-terminal ends (TM1, TM2, TM3 and TM4).

FIG. 2

Binding of GSH. (A) Top view of the MPGES1 trimer from the cytoplasmic side with the omit map contoured at 2.5 σ showing the positions of GSH molecules. (B) The U-shaped GSH molecule in ball and stick representation with the omit map density as seen from the side at the interface between transmembrane helices 1 and 4 from two different subunits. (C) Side chains involved in formation of the binding pocket for GSH.

FIG. 3

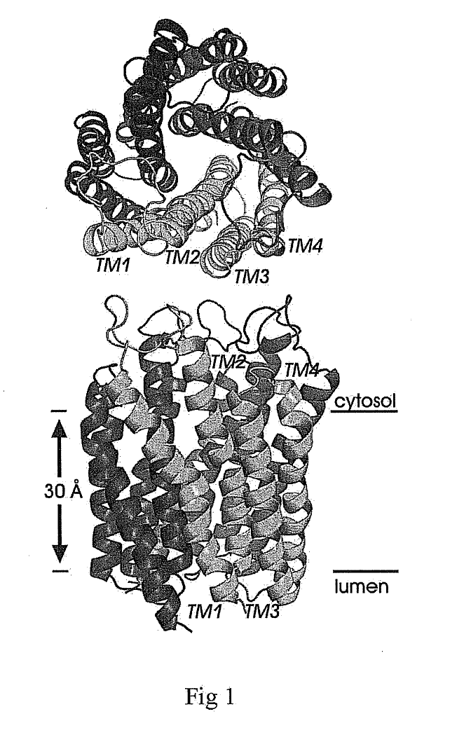

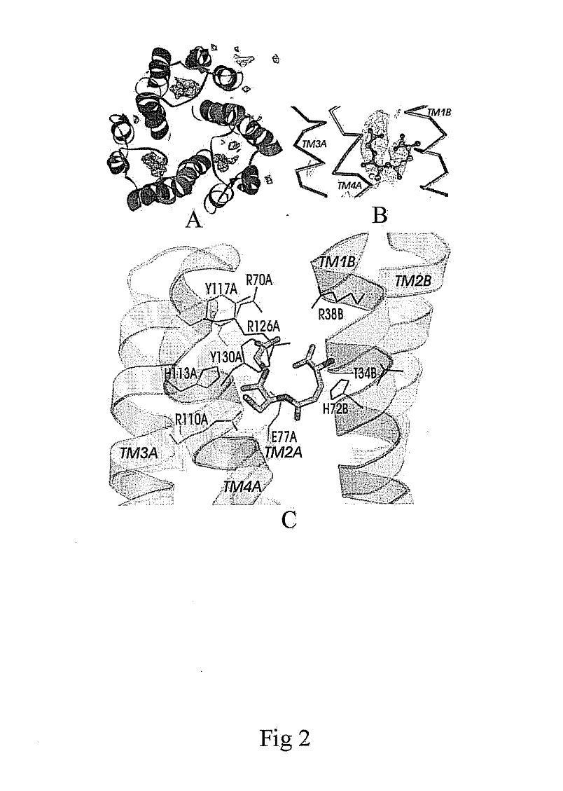

Conformational change allowing PGH₂ access and schematic chemical mechanism for MPGES1. (A) Left: Surface representation of the MPGES1 trimer structure as determined from the 2-D crystals showing a closed conformation. The three subunits are coloured in dark grey, grey and light grey respectively. Right: Open conformation based on homology modelling from the structure of LTC4S (protein data bank identificator (pdb id) 2UUH, www.pdb.org) exposes the GSH molecules. Inset: Enlargement of the cleft between subunits where PGH2 is expected to bind. GSH is shown in light grey and the PGH2 molecule fitted according to the molecular ruler concept. The surface rendering shows electrostatic potential of the open homology model. Dark grey represents positively or negatively charged and light grey non-polar surface. (B) Left: The largest difference between the closed and open conformation is a bending of the cytoplasmic half of TM1 about a hinge fixed by the Lys26/Asp75 salt bridge (arrow). Middle: The closed conformation with Ser127, Tyr130, Thr131 and Gln134 on TM4 pointing towards residues Ile32, Gln34 and Leu39 in TM1 in the next subunit. Pointing inwards binding to the GSH are Arg126 from TM4 and Tyr28 form TM1. Right: The open conformation model with the same residues and GSH shown. (C) Suggested chemical mechanism for the MPGES1 catalysed PGE₂ synthesis involves an attack of the GSH thiolate on the O(9) of the endoperoxide bridge followed by proton donation to O(11) via Arg126. Arg126 then abstracts a proton from C(9) where a carbonyl forms as the oxygen sulfur bond is broken. The leaving GSH thiolate is stabilised by Arg126.

FIG. 4

Reaction scheme for the formation of PGE2 catalysed by cyclooxygenase and mpges1.

FIG. 5

Packing arrangement in the 2-D crystals of MPGES1. (A) Top view of the electron diffraction potential map from the lumenal side of the membrane with four trimeric protein model backbones depicted within the p22₁2₁ two-sided plane group unit cell. The polar interaction of Arg150 and the backbone carboxyl of Ala112 from a neighbouring trimer (indicated with a star) is forming a tight connection along the b-axis.

FIG. 6

View along the plane of the membrane showing the arrangement of the tilted trimers. Arginines at positions 150 and 40 from two trimers in opposite orientations form contacts stabilizing the structure along the a-axis of the crystal.

FIG. 7

2Fo-Fc map contoured at 1.2 Å as seen from the side at the interface between two subunits.

FIG. 8

Sequence alignment of the six human MAPEG members based on the known structures. The positions of the four helices in MPGES1 are indicated. Every ten residue is underlined in each protein. Conserved residues are colored in light gray.

FIG. 9

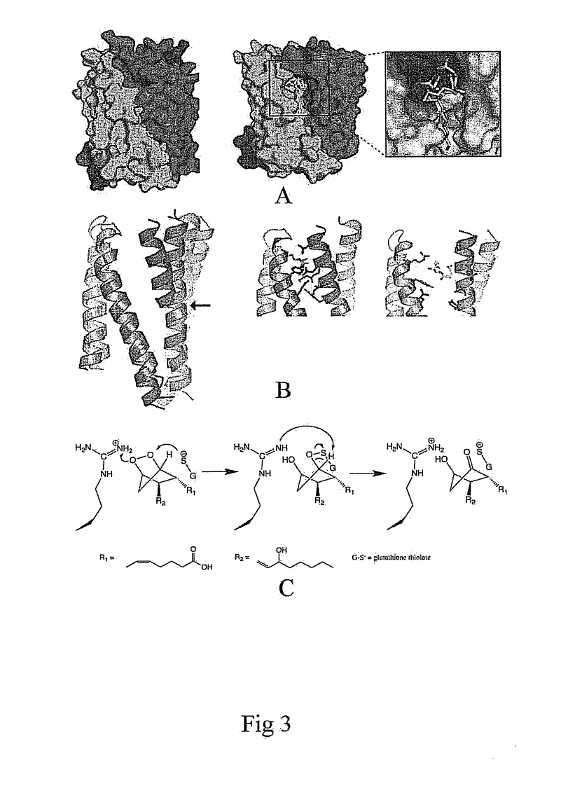

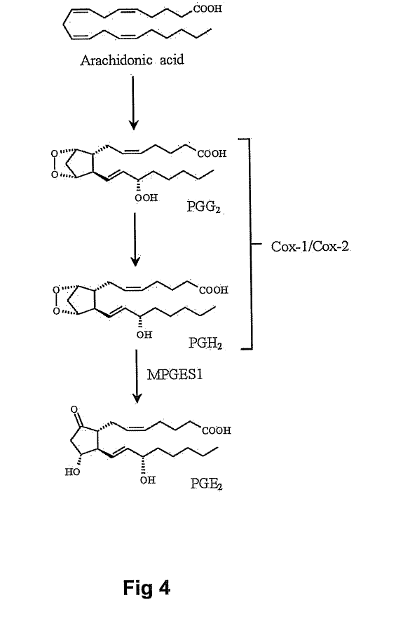

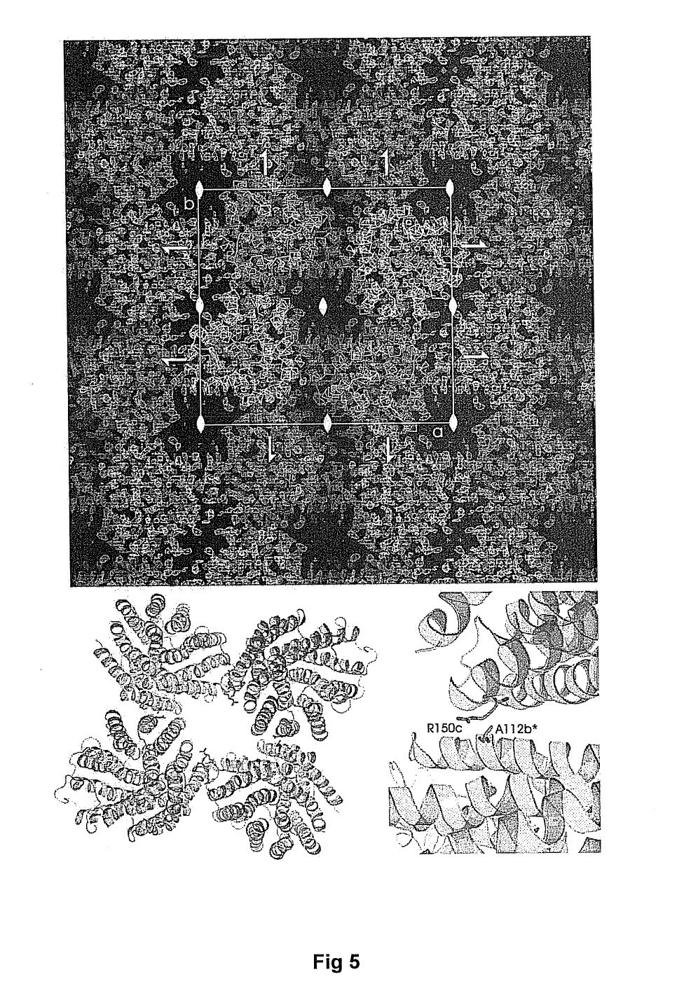

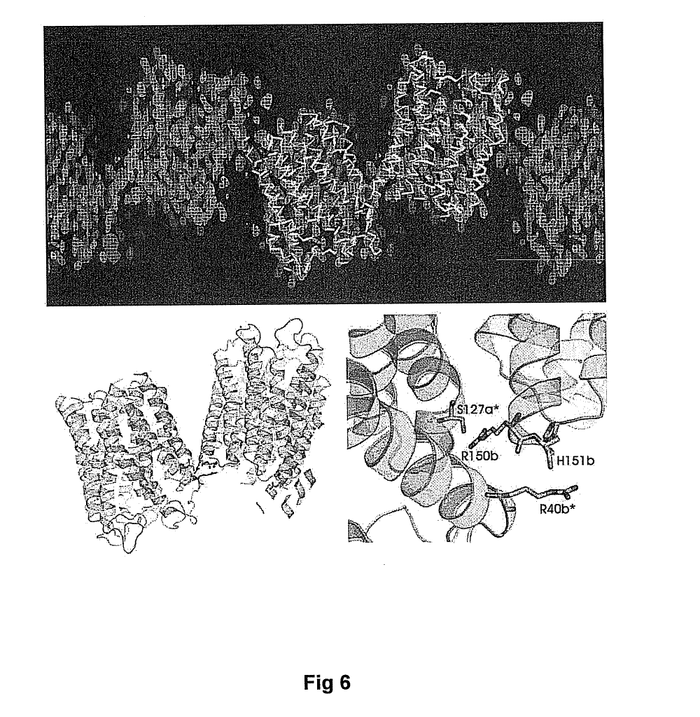

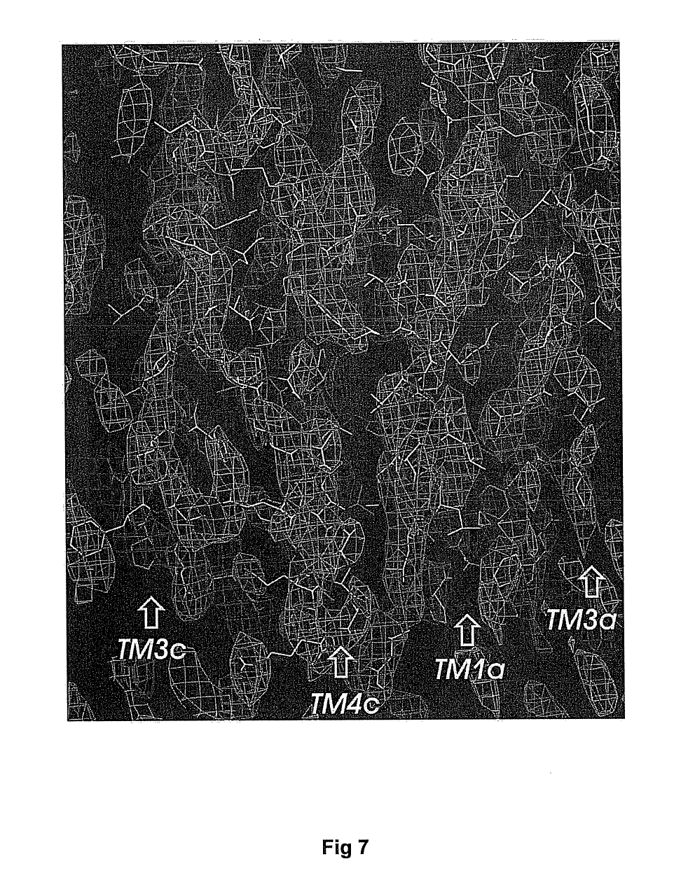

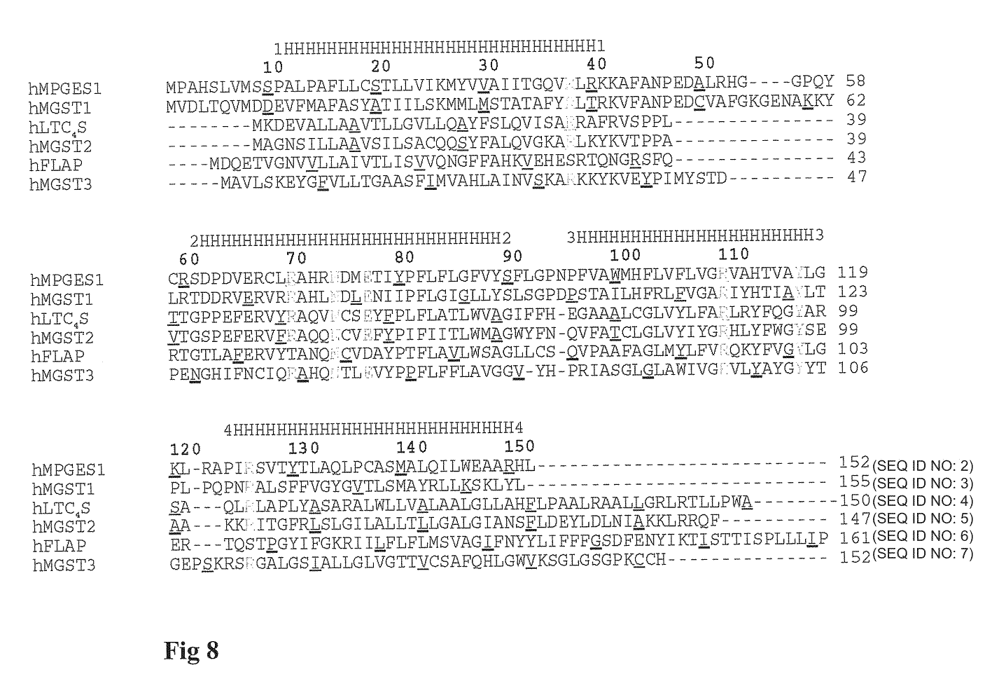

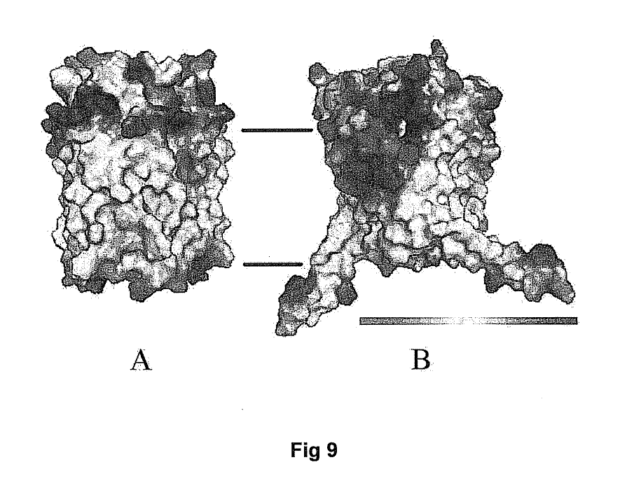

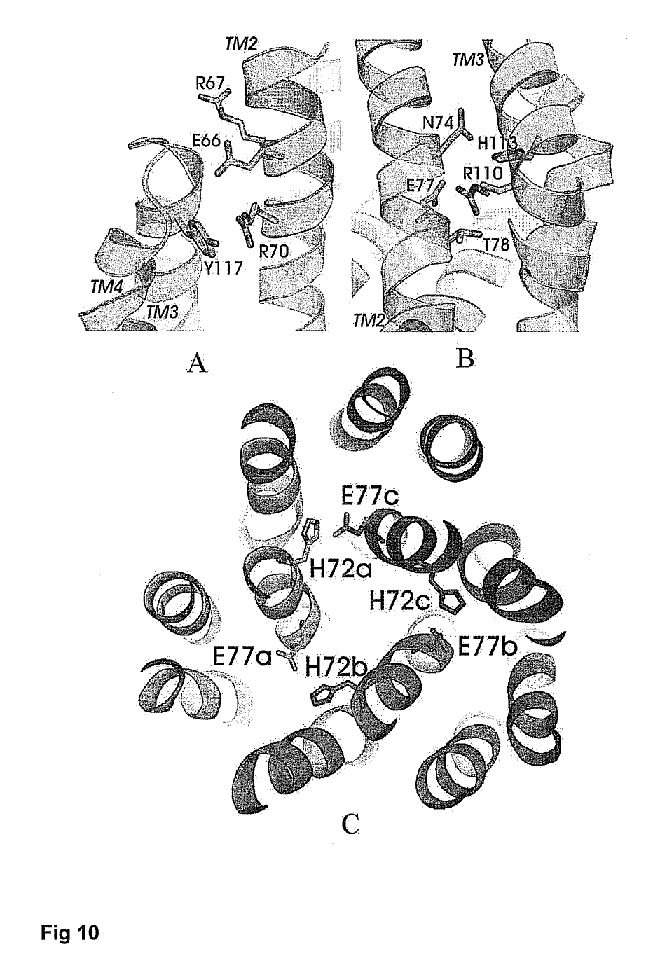

Electronstatic potentials of (A) MPGES1 and (B) LTC4S (pdb id 2UUH). The viewing angle is the same as in FIGS. 3A, B with the cytosolic side up. The horizontal bars indicate the position of the interface between the phospholipids headgroups and non-polar part of the membrane. Dark grey represents positively och negatively charged and white non-polar surface.

FIG. 10

Intra- and intersubunit interactions in MPGES1. (A) Stabilization of the cytoplasmic face of transmembrane helices 2 and 3. (B) Interactions between the same helices close to the GSH binding site. (C) Stabilization of the trimer through the His72-Glu77 ion pair.

### DETAILED DESCRIPTION OF THE INVENTION

The atomic structure of a key enzyme of the MAPEG superfamily, MPGES1 (Microsomal Prostaglandin E Synthase 1), provides a tool which enables the determination of the specificity and function of MPGES1 and related proteins which are potential targets for treatment of common diseases such as rheumatoid arthritis and asthma.

The structure of MPGES1 is an attractive target for developing drugs that could stabilise the closed form and thereby efficiently inhibit catalysis. In addition, the data indicate that LTC4S could also exist in a closed conformation that was not experimentally accessible as the detergent used in crystallisation behaved as a substrate analogue (22, 23). The model according to the present invention may thus be useful in efficient LTC4S inhibitor development as well. The position of the hydrophobic substrate in LTC4S and the similar location of GSH in MPGES1/LTC4S indicate that PGH2 binds to the cleft between TM 1 and TM4. A tentative binding mode has been modelled using the molecular ruler concept that places the omega-end of PGH₂ towards the middle of the bilayer, lets the endoperoxide reside in reasonable proximity to GSH and allows the carboxylate to reside in the hydrophilic protein water interface (FIG. 3C). This tentative model suggests reasonable hydrogen bonding and polar interactions allowing the hydrocarbon part to be bound along the cleft without steric clashes, although this should preferably be confirmed by the crystal structure of MPGES1 with a ligand.

A method for building an atomic model of a protein molecule comprising: (a) identifying a protein molecule with at least 20% sequence identity with Microsomal Prostaglandin E Synthase 1 (MPGES1) and (b) utilizing the atomic coordinates of MPGES1 to obtain an atomic model of the identified protein molecule.

In one embodiment, the protein molecule is a Membrane Associated Protein in Eicosanoid and Glutathione Metabolism (MAPEG) protein molecule.

The atomic model may comprise a homology model, wherein the homology model may be obtained by a modelling software program.

The atomic model may comprise an experimental model, wherein the experimental model is obtained by molecular replacement.

In one embodiment, said method may further comprise (c) identifying a drug candidate compound that interacts with the identified protein molecule, wherein the atomic structure of the identified protein molecule is used to identify a drug candidate compound.

In a further embodiment, said method further comprises (d) analyzing the interaction of the drug candidate compound with the identified protein molecule.

In a further embodiment, said method further comprises the analysis of the interaction of the drug candidate compound with the active site of the identified protein molecule by way of a docking-program.

The detailed interaction of the drug candidate compound with the identified protein molecule may be obtained using molecular replacement.

In yet another embodiment, the method further comprises (c) identifying a drug candidate compound that interacts with the identified protein molecule by (1) contacting the drug candidate compound with the identified protein molecule and (2) measuring for a change in the expression or activity of the identified protein molecule.

In yet another embodiment, the invention provides a method wherein a drug candidate compound that decreases the expression or activity of the identified protein molecule indicates that the drug candidate compound is an inhibitor of the identified protein molecule. A drug candidate compound that increases the expression or activity of the identified protein molecule indicates that the drug candidate compound is a promoter of the identified protein molecule.

The invention also provides a method according to the above, wherein at least one catalytic position of the identified protein molecule is mutated prior to identifying a drug candidate compound that interacts with the identified protein molecule.

The invention also provides a method wherein the mutation comprises a substitution or deletion of at least one amino acid.

The invention also provides a method for determining a drug candidate compound that interacts with Microsomal Prostaglandin E Synthase 1 (MPGES1), said method comprising: (a) identifying a drug candidate compound that interacts with MPGES1 and (b) analyzing the interaction of the drug candidate compound with MPGES1 .

The invention also provides a method according to the preceding paragraph, wherein the interaction of the drug candidate compound with the active site of the MPGES1 is analyzed with a docking-program.

The invention also provides a method according to the preceding paragraph, wherein the structure of the interaction of the drug candidate compound with MPGES1 is obtained using molecular replacement.

The invention also provides a method according to the preceding paragraph, wherein the drug candidate compound is identified by using the atomic structure of MPGES1 to design a drug candidate compound.

The invention also provides a method according to the preceding paragraph, wherein the drug candidate compound is identified by (a) contacting the drug candidate compound with MPGES1; and (b) measuring for a change in the expression or activity of the protein molecule.

The invention also provides a method according to the preceding paragraph, wherein a drug candidate compound that decreases the expression or activity of MPGES1 indicates that the drug candidate compound is an inhibitor of MPGES1.

The invention also provides a method according to the preceding paragraph, wherein a drug candidate compound that increases the expression or activity of MPGES1 indicates that the drug candidate compound is a promoter of MPGES1.

The invention also provides a method according to the preceding paragraph, wherein at least one catalytic position of MPGES1 is mutated prior to identifying a drug candidate compound that interacts with MPGES1 .

The invention also provides a method according to the preceding paragraph, wherein the mutation comprises a substitution of at least one amino acid.

The invention also provides a method according to the preceding paragraph, wherein the mutation comprise a deletion of at least one amino acid.

The skilled person will appreciate the various means in which a drug candidate compound interacts with a protein molecule. In one embodiment, a drug candidate compound that decreases the expression or activity of the protein molecule indicates that the compound is an inhibitor of the protein molecule. In another embodiment, a compound that increases the expression or activity of the protein molecule indicates that the compound is a promoter of the protein molecule. One skilled in the art will appreciate the various methods for contacting the drug candidate compound with a protein molecule, any of which may be employed herein. One skilled in the art will also appreciate the various methods for measuring a change in the expression or activity of the protein, any of which may be employed herein.

The interaction of the drug candidate compound with the identified protein molecule may be analyzed. In one embodiment, the interaction of the drug candidate compound with the active site of the identified protein molecule is analyzed with a docking-program. In addition, the structure of the interaction of the candidate compound with the protein molecule may be obtained using molecular replacement.

Furthermore, the skilled person will appreciate that in order to comprehensively determine a suitable drug candidate compound that interacts with the protein molecule at least one catalytic position of the protein molecule may be mutated prior to contacting the drug candidate compound with the protein molecule. In one embodiment, the mutation comprises a substitution of at least one amino acid. In another embodiment, the mutation comprises a deletion of at least one amino acid.

### EXAMPLES

### Example 1

Specimen preparation. Microsomal prostaglandin E synthase 1 (MPGES1) containing a His₆-tag at the N-terminus was overexpressed in E. coli and purified as published earlier (Thorén et al., J. Biol. Chem. 278, 22199 (2003)). The plasmid pSP19T7LT containing wild-type MPGES1 with an N-terminal His₆-tag (Thorén et al., J. Biol. Chem. 278, 22199 (2003)) was subjected to site-directed mutagenesis using GeneTailor™ Site-Directed Mutagenesis System (Invitrogen) and specific mutation primers. The following sequence (SEQ ID NO:1) was used for subsequent experiments (N-term to C-term):

### HHHHHHMPAH SLVMSSPALP AFLLCSTLLV IKMYVVAIIT GQVRLRKKAF ANPEDALRHG GPQYCRSDPD VERCLRAHRN DMETIYPFLF LGFVYSFLGP NPFVAWMHFL VFLVGRVAHT VAYLGKLRAP IRSVTYTLAQ LPCASMALQI LWEAARHL

Mutant protein was expressed in BL21 Star™ (DE) E. coli cells growing at 37° C. in terrific broth. Subsequently, we isolated the membrane fractions by differential centrifugation and quantified the amount of mutant MPGES1 by comparing the signals with known amounts of purified MPGES1 in Western Blot analysis. For activity measurements, we used accordingly 400 ng mutant or wild type MPGES1 in the assay described in (S. Thoren, P. J. Jakobsson, Eur. J. Biochem. 267, 6428 (2000)).

In the final preparation the protein at a concentration of 1 mg/ml was solubilized with 1% Triton X-100 in a buffer containing 10-50 mM NaPᵢ, 50 mM NaCl, 20% glycerol, 1 mM GSH, and 0.1% EDTA.

The crystals of MPGES1 were grown in the presence of GSH. MPGES1 having a His₆-tag at the N-terminus and in a buffer containing 1% Triton X-100, 100 mM NaPᵢ, 50 mM NaCl, 10% glycerol, 1 mM GSH, and 0.1% EDTA was mixed with bovine liver lecithin solubilized in 1% Triton X-100. The protein concentration was typically 0.5 mg/ml, the total volume 100 μl and the molar lipid-to-protein ratio was 9. The mixture was placed in dialysis tubes with a molecular weight cut off pore size of 12-14 kD and closed at both ends. The tubes were put in 1000 ml containers filled with the same buffer as above but without Triton X-100 and with 20% glycerol. The dialysis was left for one to two weeks at room temperature. 2 μl aliquots of the crystalline suspension were put on carbon coated electron microscope grids and stabilized with 7% trehalose prior to freezing in liquid nitrogen.

The quality of the reconstituted crystals are evaluated by electron microscopy of negatively stained specimens. Excellent crystal preparations are selected for cryo-electron microscopy.

### Example 2

### Data Collection

For electron microscopy, grids were prepared with the inverted technique (T. Hirai, K. Murata, K. Mitsuoka, Y, Kimura, Y. Fujiyoshi, J. Electron Microsc. 48, 653 (1999)) using conventional 400 mesh grids, thin carbon layers, typically 7% trehalose and 2 μl of the crystallization suspension. Grids were placed in liquid nitrogen cooled Gatan 626 or 914 cryo holders before insertion into a JEOL 2100F electron microscope equipped with a TemCam-F415 4k*4k CCD camera (Tietz Video and Image Processing Systems GmbH).

Electron diffraction patterns were recorded. For tilted diffraction patterns pairwise acquisitions were always made in such a way that the first tilted recording was followed by a 0° pattern which was used entirely for classification and quality assessment.

### Example 3

Background corrected amplitudes were extracted using the MRC software (R. A. Crowther, R. Henderson, J. M. Smith, J. Struct. Biol. 116, 9 (1996)). A total number of 100 diffraction patterns constituting a homogenous set as judged initially by the untilted recordings were subjected to merging using DIFFMERG and subsequently LATLINE was used for lattice line fitting. Tilt-angles and CTFs accurately determined during the first processing round are used for reprocessing all good images in a second round of unbending using the MAKETRAN program procedure. Final image amplitudes and phase values are extracted following tilted CTF and beam tilt correction. Diffraction amplitudes collected on the three different CCD cameras are processed using the MRC electron diffraction software. Image phases and electron diffraction amplitudes are merged using the LATLINE program from the MRC suite.

### Example 4

The trimeric model of microsomal glutathione transferase 1 (MGST1) (P. J. Holm et al., J. Mol. Biol. 360, 934 (2006)) was used for a molecular replacement search since the sequence identity is high (approx 40%) and previous studies have shown that MPGES1 also forms a trimer in 2-D crystals (S. Thorén et al., J. Biol. Chem. 278, 22199 (2003)). One of the top score solutions from AMoRe (J. Navaza, Acta Crystallogr. A 50, 157 (1994)) was unique since it did not present severe clashes between trimers and it fitted a low resolution projection map. Model building in O (T. A. Jones, J. Y. Zou, S. W. Cowan, M. Kjeldgaard, Acta Crystallogr. A 47, 110 (1991)) started from a polyalanine model of the protein at the determined position and orientation. Numerous rounds of rigid body and restrained refinement using tight geometry restraints and medium non-crystallographic symmetry in REFMAC5 in combination with geometry idealisation and manual rebuilding in O resulted in a final structure of the MPGES1 trimer where all residues except the His-tags and the first ten N-terminal residues were included. At the present resolution the difference between using electron- or X-ray form factors are negligible, thus X-ray values were used throughout the refinement. 2Fₒ-F_{c} and Fₒ-F_{c} maps were continuously generated to evaluate the accuracy and quality of the refined model. Reliable R_{free} values were obtained by using a final fraction of 4.64% of the observed structure factor amplitudes. PROCHECK (R. A. Laskowski, M. W. MacArthur, D. S. Moss, J. M. Thornton, J. Appl. Cryst. 26, 283 (1993) was used to examine the molecular geometry. For analysis and illustrations we used PYMOL (W. L. DeLano, The PyMOL Molecular Graphics System on World Wide Web http://www.pymol.org (2002)) and CHIMERA (F. Pettersen et al., J. Comput. Chem. 25, 1605 (2004)).

TABLE 1
Data collection and refinement statistics
Two-dimensional crystal parameters

| Two-sided plane-group | p22₁2₁ |
|---|---|
| Cell dimensions | |
| a, b, c (Å) | 93.2, 84.6, 100.0* |
| γ (°) | 90.0 |

Amplitude determination from electron diffraction

| | |
|---|---|
| No. of diffraction pattern | 100 |
| Maximum tilt angle (°) | 62.0 |
| Resolution in and normal to membrane plane (Å)^{†} | 3.5/4.2 |
| No. of observed/used amplitudes | 43035/6185 |
| Fourier space sampled (%) | 58.8 |
| F/σ, overall/4.0-3.5 Å | 4.51/3.51 |
| R_{Friedel} (%) | 14.4 |
| R_{merge} (%) | 46.1 |

Crystallographic refinement

| | |
|---|---|
| Resolution (Å) | 10.0-3.5 |
| No. of reflections | 6185 |
| No. of atoms, protein/substrate | 3396/60 |
| R_{work} (%) | 35.2 |
| R_{Free} (%) | 39.4 |
| R.m.s. deviations | |
| Bond lengths (Å) | 0.012 |
| Bond angles (°) | 1.612 |
| Ramachandran plot distribution (%) | 79.6/17.5/2.7/0.3 |

*Assumed for sampling along z*. For lattice line adaption a thickness of 65 Å was used.
^{†}From calculation of point spread function

### Example 5

Sequence alignment was performed with ClustalW (J. D. Thompson, D. G. Higgins, T. J. Gibson, Nucleic Acids Res. 22, 4673 (1994)). followed by structural based manual adjustments. Modeller (A. Sali, T. L. Blundell, J. Mol. Biol. 234, 779 (1993). 25, 1605 (2004)). was used to calculate homology models.

TABLE 2
Site-directed mutagenesis for MPGES1*

| Mutation | Fraction of wild-type activity (%)^{†} |
|---|---|
| E66A | 53 |
| R67A | 0 |
| R70A | 58 |
| R70S | 68 |
| R70S^{‡} | full activity |
| H72A | 32 |
| E77A | 0 |
| R110A | 0 |
| R110S | 0 |
| R110S^{‡} | lost activity |
| Y117A | 1 |
| Y117F | full activity |
| R70A-Y117A | 7 |
| Y130″ | 15 |

*Residues identified to be involved in stabilisation of the protein and in GSH binding.
^{†}Activities were measured from isolated E. coli membranes (n = 2). Values represent the percentage of the mean value from wt MPGES1 in isolated E. coli membranes (n = 6).
^{‡}From supplementary ref 13.
″From supplementary ref 14.

TABLE 3
Atomic coordinates

| | | |
|---|---|---|
| HEADER | — | XX-XXX-XX xxxx |
| COMPND | — | |

| | | | |
|---|---|---|---|
| REMARK | 3 | | |
| REMARK | 3 | REFINEMENT. | |
| REMARK | 3 | PROGRAM | : REFMAC 5.2.0005 |
| REMARK | 3 | AUTHORS | : MURSHUDOV, VAGIN, DODSON |
| REMARK | 3 | | |

| REMARK | 3 | REFINEMENT TARGET : MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 3.48 |
| REMARK | 3 | RESOLUTION RANGE LOW | (ANGSTROMS) | : 10.00 |
| REMARK | 3 | DATA CUTOFF | (SIGMA(F)) | : NONE |

| | | | | |
|---|---|---|---|---|
| REMARK | 3 | COMPLETENESS FOR RANGE | (%) | : 58.32 |
| REMARK | 3 | NUMBER OF REFLECTIONS | | : 5627 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.35428 |
| REMARK | 3 | R VALUE | (WORKING SET) | : 0.35233 |

| REMARK | 3 | FREE R VALUE | | : 0.39442 |
|---|---|---|---|---|
| REMARK | 3 | FREE R VALUE TEST SET SIZE | (%) | : 4.6 |

| | | | |
|---|---|---|---|
| REMARK | 3 | FREE R VALUE TEST SET COUNT | : 274 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 20 |
| REMARK | 3 | BIN RESOLUTION RANGE HIGH | | : 3.480 |
| REMARK | 3 | BIN RESOLUTION RANGE LOW | | : 3.559 |
| REMARK | 3 | REFLECTION IN BIN | (WORKING SET) | : 240 |
| REMARK | 3 | BIN COMPLETENESS | (WORKING + TEST) (%) | : 36.05 |
| REMARK | 3 | BIN R VALUE | (WORKING SET) | : 0.184 |

| | | | |
|---|---|---|---|
| REMARK | 3 | BIN FREE R VALUE SET COUNT | : 8 |
| REMARK | 3 | BIN FREE R VALUE | : 0.449 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 3456 |
| REMARK | 3 | | |

| | | | | |
|---|---|---|---|---|
| REMARK | 3 | B VALUES. | | |
| REMARK | 3 | FROM WILSON PLOT | (A**2) | : NULL |
| REMARK | 3 | MEAN B VALUE | (OVERALL, A**2) | : 3.016 |

| REMARK | 3 | OVERALL ANISOTROPIC B VALUE. | |
|---|---|---|---|
| REMARK | 3 | B11 (A**2) : | −1.01 |
| REMARK | 3 | B22 (A**2) : | −1.03 |
| REMARK | 3 | B33 (A**2) : | 2.04 |
| REMARK | 3 | B12 (A**2) : | 0.00 |
| REMARK | 3 | B13 (A**2) : | 0.00 |
| REMARK | 3 | B23 (A**2) : | 0.00 |
| REMARK | 3 | | |

| | | | | |
|---|---|---|---|---|
| REMARK | 3 | ESTIMATED OVERALL COORDINATE ERROR. | | |
| REMARK | 3 | ESU BASED ON R VALUE | (A) | : NULL |
| REMARK | 3 | ESU BASED ON FREE R VALUE | (A) | : 1.308 |
| REMARK | 3 | ESU BASED ON MAXIMUM LIKELIHOOD | (A) | : 0.548 |
| REMARK | 3 | ESU FOR B VALUES BASED ON MAXIMUM LIKELIHOOD | (A**2) | : 30.138 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.355 |
| REMARK | 3 | CORRELATION COEFFICIENT FO-FC FREE | : 0.266 |
| REMARK | 3 | | |

| | | | | | |
|---|---|---|---|---|---|
| REMARK | 3 | RMS DEVIATIONS FROM IDEAL VALUES | COUNT | RMS | WEIGHT |

| | | | | | | |
|---|---|---|---|---|---|---|
| REMARK | 3 | BOND LENGTHS REFINED ATOMS | (A): | 3552; | 0.012; | 0.022 |

| | | | | | | |
|---|---|---|---|---|---|---|
| REMARK | 3 | BOND ANGLES REFINED ATOMS | (DEGREES): | 4827; | 1.612; | 1.978 |
| REMARK | 3 | TORSION ANGLES, PERIOD 1 | (DEGREES): | 423; | 8.735; | 5.000 |
| REMARK | 3 | TORSION ANGLES, PERIOD 2 | (DEGREES): | 144; | 29.062; | 20.833 |
| REMARK | 3 | TORSION ANGLES, PERIOD 3 | (DEGREES): | 555; | 22.947; | 15.000 |
| REMARK | 3 | TORSION ANGLES, PERIOD 4 | (DEGREES): | 33; | 21.929; | 15.000 |

| | | | | | | |
|---|---|---|---|---|---|---|
| REMARK | 3 | CHIRAL-CENTER RESTRAINTS | (A**3): | 543; | 0.116; | 0.200 |
| REMARK | 3 | GENERAL PLANES REFINED ATOMS | (A): | 2676; | 0.005; | 0.020 |
| REMARK | 3 | NON-BONDED CONTACTS REFINED ATOMS | (A): | 2727; | 0.338; | 0.200 |
| REMARK | 3 | NON-BONDED TORSION REFINED ATOMS | (A): | 2334; | 0.316; | 0.200 |
| REMARK | 3 | H-BOND (X . . . Y) REFINED ATOMS | (A): | 180; | 0.256; | 0.200 |
| REMARK | 3 | SYMMETRY VDW REFINED ATOMS | (A): | 46; | 0.357; | 0.200 |
| REMARK | 3 | SYMMETRY H-BOND REFINED ATOMS | (A): | 2; | 0.524; | 0.200 |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |
| REMARK | 3 | | | | |
| REMARK | 3 | NCS RESTRAINTS STATISTICS | | | |
| REMARK | 3 | NUMBER OF DIFFERENT NCS GROUPS : 1 | | | |
| REMARK | 3 | | | | |

| | | | |
|---|---|---|---|
| REMARK | 3 | NCS GROUP NUMBER | : 1 |
| REMARK | 3 | CHAIN NAMES | : A B C |

| REMARK | 3 | NUMBER OF COMPONENTS NCS GROUP: 1 | | | | | | |
|---|---|---|---|---|---|---|---|---|
| REMARK | 3 | COMPONENT | C | SSSEQI | TO | C | SSSEQI | CODE |
| REMARK | 3 | 1 | A | 11 | | A | 152 | 4 |
| REMARK | 3 | 1 | B | 11 | | B | 152 | 4 |
| REMARK | 3 | 1 | C | 11 | | C | 152 | 4 |

| | | | | | |
|---|---|---|---|---|---|
| REMARK | 3 | GROUP CHAIN | COUNT | RMS | WEIGHT |

| | | | | | | | |
|---|---|---|---|---|---|---|---|
| REMARK | 3 | MEDIUM POSITIONAL 1 | A | (A): | 1132 ; | 0.59 ; | 0.50 |
| REMARK | 3 | MEDIUM POSITIONAL 1 | B | (A): | 1132 ; | 0.59 ; | 0.50 |
| REMARK | 3 | MEDIUM POSITIONAL 1 | C | (A): | 1132 ; | 0.59 ; | 0.50 |
| REMARK | 3 | | | | | | |
| REMARK | 3 | | | | | | |
| REMARK | 3 | TLS DETAILS | | | | | |

| REMARK | 3 | NUMBER OF TLS GROUPS : NULL | |
|---|---|---|---|
| REMARK | 3 | | |
| REMARK | 3 | | |
| REMARK | 3 | BULK SOLVENT MODELLING. | |
| REMARK | 3 | METHOD USED : MASK | |
| REMARK | 3 | PARAMETERS FOR MASK CALCULATION | |
| REMARK | 3 | VDW PROBE RADIUS | : 1.20 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | | | | | | | |
|---|---|---|---|---|---|---|---|
| CRYST1 | 93.200 | 84.600 | 100.000 | 90.00 | 90.00 | 90.00 | P 21 21 2 |

| | | | | |
|---|---|---|---|---|
| SCALE1 | 0.010730 | 0.000000 | 0.000000 | 0.00000 |
| SCALE2 | 0.000000 | 0.011820 | 0.000000 | 0.00000 |
| SCALE3 | 0.000000 | 0.000000 | 0.010000 | 0.00000 |

| | | | | | | | | | | | |
|---|---|---|---|---|---|---|---|---|---|---|---|
| ATOM | 1 | N | PRO | A | 11 | 81.110 | −33.743 | −18.260 | 1.00 | 3.02 | N |
| ATOM | 2 | CA | PRO | A | 11 | 82.128 | −33.275 | −17.327 | 1.00 | 3.02 | C |
| ATOM | 3 | CB | PRO | A | 11 | 82.762 | −32.094 | −18.067 | 1.00 | 3.02 | C |
| ATOM | 4 | CG | PRO | A | 11 | 82.457 | −32.339 | −19.510 | 1.00 | 3.02 | C |
| ATOM | 5 | CD | PRO | A | 11 | 81.095 | −32.942 | −19.496 | 1.00 | 3.02 | C |
| ATOM | 6 | C | PRO | A | 11 | 81.501 | −32.808 | −16.024 | 1.00 | 3.02 | C |
| ATOM | 7 | O | PRO | A | 11 | 82.132 | −32.897 | −14.967 | 1.00 | 3.02 | O |
| ATOM | 8 | N | ALA | A | 12 | 80.261 | −32.329 | −16.115 | 1.00 | 3.02 | N |
| ATOM | 9 | CA | ALA | A | 12 | 79.523 | −31.806 | −14.969 | 1.00 | 3.02 | C |
| ATOM | 10 | CB | ALA | A | 12 | 78.281 | −31.050 | −15.442 | 1.00 | 3.02 | C |
| ATOM | 11 | C | ALA | A | 12 | 79.153 | −32.906 | −13.962 | 1.00 | 3.02 | C |
| ATOM | 12 | O | ALA | A | 12 | 79.927 | −33.173 | −13.032 | 1.00 | 3.02 | O |
| ATOM | 13 | N | LEU | A | 13 | 77.990 | −33.544 | −14.166 | 1.00 | 3.02 | N |
| ATOM | 14 | CA | LEU | A | 13 | 77.452 | −34.600 | −13.267 | 1.00 | 3.02 | C |
| ATOM | 15 | CB | LEU | A | 13 | 76.389 | −35.475 | −13.966 | 1.00 | 3.02 | C |
| ATOM | 16 | CG | LEU | A | 13 | 75.140 | −34.794 | −14.530 | 1.00 | 3.02 | C |
| ATOM | 17 | CD1 | LEU | A | 13 | 74.388 | −35.736 | −15.455 | 1.00 | 3.02 | C |
| ATOM | 18 | CD2 | LEU | A | 13 | 74.242 | −34.304 | −13.396 | 1.00 | 3.02 | C |
| ATOM | 19 | C | LEU | A | 13 | 78.512 | −35.501 | −12.633 | 1.00 | 3.02 | C |
| ATOM | 20 | O | LEU | A | 13 | 78.386 | −35.858 | −11.459 | 1.00 | 3.02 | O |
| ATOM | 21 | N | PRO | A | 14 | 79.543 | −35.886 | −13.405 | 1.00 | 3.02 | N |
| ATOM | 22 | CA | PRO | A | 14 | 80.521 | −36.724 | −12.724 | 1.00 | 3.02 | C |
| ATOM | 23 | CB | PRO | A | 14 | 81.399 | −37.275 | −13.859 | 1.00 | 3.02 | C |
| ATOM | 24 | CG | PRO | A | 14 | 81.108 | −36.442 | −15.057 | 1.00 | 3.02 | C |
| ATOM | 25 | CD | PRO | A | 14 | 79.857 | −35.669 | −14.832 | 1.00 | 3.02 | C |
| ATOM | 26 | C | PRO | A | 14 | 81.331 | −36.021 | −11.618 | 1.00 | 3.02 | C |
| ATOM | 27 | O | PRO | A | 14 | 81.430 | −36.564 | −10.511 | 1.00 | 3.02 | O |
| ATOM | 28 | N | ALA | A | 15 | 81.883 | −34.836 | −11.888 | 1.00 | 3.02 | N |
| ATOM | 29 | CA | ALA | A | 15 | 82.621 | −34.085 | −10.851 | 1.00 | 3.02 | C |
| ATOM | 30 | CB | ALA | A | 15 | 83.000 | −32.689 | −11.348 | 1.00 | 3.02 | C |
| ATOM | 31 | C | ALA | A | 15 | 81.853 | −34.008 | −9.512 | 1.00 | 3.02 | C |
| ATOM | 32 | O | ALA | A | 15 | 82.441 | −33.725 | −8.461 | 1.00 | 3.02 | O |
| ATOM | 33 | N | PHE | A | 16 | 80.546 | −34.295 | −9.576 | 1.00 | 3.02 | N |
| ATOM | 34 | CA | PHE | A | 16 | 79.635 | −34.279 | −8.418 | 1.00 | 3.02 | C |
| ATOM | 35 | CB | PHE | A | 16 | 78.270 | −33.713 | −8.815 | 1.00 | 3.02 | C |
| ATOM | 36 | CG | PHE | A | 16 | 78.354 | −32.339 | −9.341 | 1.00 | 3.02 | C |
| ATOM | 37 | CD1 | PHE | A | 16 | 78.478 | −32.117 | −10.706 | 1.00 | 3.02 | C |
| ATOM | 38 | CE1 | PHE | A | 16 | 78.603 | −30.824 | −11.218 | 1.00 | 3.02 | C |
| ATOM | 39 | CZ | PHE | A | 16 | 78.625 | −29.743 | −10.348 | 1.00 | 3.02 | C |
| ATOM | 40 | CE2 | PHE | A | 16 | 78.521 | −29.958 | −8.964 | 1.00 | 3.02 | C |
| ATOM | 41 | CD2 | PHE | A | 16 | 78.388 | −31.253 | −8.473 | 1.00 | 3.02 | C |
| ATOM | 42 | C | PHE | A | 16 | 79.445 | −35.629 | −7.755 | 1.00 | 3.02 | C |
| ATOM | 43 | O | PHE | A | 16 | 79.127 | −35.706 | −6.566 | 1.00 | 3.02 | O |
| ATOM | 44 | N | LEU | A | 17 | 79.619 | −36.694 | −8.519 | 1.00 | 3.02 | N |
| ATOM | 45 | CA | LEU | A | 17 | 79.376 | −37.998 | −7.969 | 1.00 | 3.02 | C |
| ATOM | 46 | CB | LEU | A | 17 | 79.764 | −39.109 | −8.960 | 1.00 | 3.02 | C |
| ATOM | 47 | CG | LEU | A | 17 | 79.109 | −39.093 | −10.347 | 1.00 | 3.02 | C |
| ATOM | 48 | CD1 | LEU | A | 17 | 79.482 | −40.288 | −11.173 | 1.00 | 3.02 | C |
| ATOM | 49 | CD2 | LEU | A | 17 | 77.618 | −38.999 | −10.219 | 1.00 | 3.02 | C |
| ATOM | 50 | C | LEU | A | 17 | 80.157 | −38.094 | −6.673 | 1.00 | 3.02 | C |
| ATOM | 51 | O | LEU | A | 17 | 79.568 | −38.283 | −5.611 | 1.00 | 3.02 | O |
| ATOM | 52 | N | LEU | A | 18 | 81.470 | −37.889 | −6.765 | 1.00 | 3.02 | N |
| ATOM | 53 | CA | LEU | A | 18 | 82.397 | −38.165 | −5.664 | 1.00 | 3.02 | C |
| ATOM | 54 | CB | LEU | A | 18 | 83.855 | −38.124 | −6.138 | 1.00 | 3.02 | C |
| ATOM | 55 | CG | LEU | A | 18 | 84.208 | −38.751 | −7.494 | 1.00 | 3.02 | C |
| ATOM | 56 | CD1 | LEU | A | 18 | 85.722 | −38.947 | −7.546 | 1.00 | 3.02 | C |
| ATOM | 57 | CD2 | LEU | A | 18 | 83.436 | −40.080 | −7.775 | 1.00 | 3.02 | C |
| ATOM | 58 | C | LEU | A | 18 | 82.192 | −37.228 | −4.494 | 1.00 | 3.02 | C |
| ATOM | 59 | O | LEU | A | 18 | 82.677 | −37.479 | −3.402 | 1.00 | 3.02 | O |
| ATOM | 60 | N | CYS | A | 19 | 81.449 | −36.156 | −4.731 | 1.00 | 3.02 | N |
| ATOM | 61 | CA | CYS | A | 19 | 81.100 | −35.220 | −3.680 | 1.00 | 3.02 | C |
| ATOM | 62 | CB | CYS | A | 19 | 80.482 | −33.960 | −4.284 | 1.00 | 3.02 | C |
| ATOM | 63 | SG | CYS | A | 19 | 81.323 | −33.390 | −5.804 | 1.00 | 3.02 | S |
| ATOM | 64 | C | CYS | A | 19 | 80.154 | −35.898 | −2.689 | 1.00 | 3.02 | C |
| ATOM | 65 | O | CYS | A | 19 | 80.125 | −35.564 | −1.513 | 1.00 | 3.02 | O |
| ATOM | 66 | N | SER | A | 20 | 79.387 | −36.866 | −3.169 | 1.00 | 3.02 | N |
| ATOM | 67 | CA | SER | A | 20 | 78.608 | −37.697 | −2.284 | 1.00 | 3.02 | C |
| ATOM | 68 | CB | SER | A | 20 | 77.595 | −38.512 | −3.084 | 1.00 | 3.02 | C |
| ATOM | 69 | OG | SEA | A | 20 | 78.233 | −39.346 | −4.040 | 1.00 | 3.02 | O |
| ATOM | 70 | C | SER | A | 20 | 79.564 | −38.595 | −1.490 | 1.00 | 3.02 | C |
| ATOM | 71 | O | SER | A | 20 | 79.783 | −38.339 | −0.298 | 1.00 | 3.02 | O |
| ATOM | 72 | N | THR | A | 21 | 80.163 | −39.588 | −2.180 | 1.00 | 3.02 | N |
| ATOM | 73 | CA | THR | A | 21 | 81.026 | −40.641 | −1.595 | 1.00 | 3.02 | C |
| ATOM | 74 | CB | THR | A | 21 | 81.896 | −41.381 | −2.653 | 1.00 | 3.02 | C |
| ATOM | 75 | OG1 | THR | A | 21 | 81.424 | −41.115 | −3.974 | 1.00 | 3.02 | O |
| ATOM | 76 | CG2 | THR | A | 21 | 81.902 | −42.883 | −2.403 | 1.00 | 3.02 | C |
| ATOM | 77 | C | THR | A | 21 | 82.020 | −40.116 | −0.587 | 1.00 | 3.02 | C |
| ATOM | 78 | O | THR | A | 21 | 82.150 | −40.656 | 0.507 | 1.00 | 3.02 | O |
| ATOM | 79 | N | LEU | A | 22 | 82.723 | −39.060 | −0.972 | 1.00 | 3.02 | N |
| ATOM | 80 | CA | LEU | A | 22 | 83.873 | −38.579 | −0.218 | 1.00 | 3.02 | C |
| ATOM | 81 | CB | LEU | A | 22 | 84.819 | −37.771 | −1.127 | 1.00 | 3.02 | C |
| ATOM | 82 | CG | LEU | A | 22 | 85.344 | −38.465 | −2.391 | 1.00 | 3.02 | C |
| ATOM | 83 | CD1 | LEU | A | 22 | 86.154 | −37.519 | −3.250 | 1.00 | 3.02 | C |
| ATOM | 84 | CD2 | LEU | A | 22 | 86.136 | −39.724 | −2.025 | 1.00 | 3.02 | C |
| ATOM | 85 | C | LEU | A | 22 | 83.478 | −37.761 | 1.001 | 1.00 | 3.02 | C |
| ATOM | 86 | O | LEU | A | 22 | 84.099 | −37.872 | 2.062 | 1.00 | 3.02 | O |
| ATOM | 87 | N | LEU | A | 23 | 82.443 | −36.944 | 0.850 | 1.00 | 3.02 | N |
| ATOM | 88 | CA | LEU | A | 23 | 82.033 | −36.040 | 1.921 | 1.00 | 3.02 | C |
| ATOM | 89 | CB | LEU | A | 23 | 81.493 | −34.722 | 1.356 | 1.00 | 3.02 | C |
| ATOM | 90 | CG | LEU | A | 23 | 82.441 | −33.928 | 0.436 | 1.00 | 3.02 | C |
| ATOM | 91 | CD1 | LEU | A | 23 | 81.643 | −33.072 | −0.520 | 1.00 | 3.02 | C |
| ATOM | 92 | CD2 | LEU | A | 23 | 83.418 | −33.068 | 1.213 | 1.00 | 3.02 | C |
| ATOM | 93 | C | LEU | A | 23 | 81.048 | −36.689 | 2.888 | 1.00 | 3.02 | C |
| ATOM | 94 | O | LEU | A | 23 | 80.784 | −36.142 | 3.964 | 1.00 | 3.02 | O |
| ATOM | 95 | N | VAL | A | 24 | 80.518 | −37.852 | 2.496 | 1.00 | 3.02 | N |
| ATOM | 96 | CA | VAL | A | 24 | 79.877 | −38.783 | 3.422 | 1.00 | 3.02 | C |
| ATOM | 97 | CB | VAL | A | 24 | 79.238 | −39.953 | 2.673 | 1.00 | 3.02 | C |
| ATOM | 98 | CG1 | VAL | A | 24 | 78.866 | −41.107 | 3.628 | 1.00 | 3.02 | C |
| ATOM | 99 | CG2 | VAL | A | 24 | 78.038 | −39.463 | 1.921 | 1.00 | 3.02 | C |
| ATOM | 100 | C | VAL | A | 24 | 80.930 | −39.302 | 4.406 | 1.00 | 3.02 | C |
| ATOM | 101 | O | VAL | A | 24 | 80.839 | −39.027 | 5.602 | 1.00 | 3.02 | O |
| ATOM | 102 | N | ILE | A | 25 | 81.930 | −40.033 | 3.898 | 1.00 | 3.02 | N |
| ATOM | 103 | CA | ILE | A | 25 | 83.093 | −40.428 | 4.689 | 1.00 | 3.02 | C |
| ATOM | 104 | CB | ILE | A | 25 | 84.325 | −40.741 | 3.797 | 1.00 | 3.02 | C |
| ATOM | 105 | CG1 | ILE | A | 25 | 84.037 | −41.910 | 2.840 | 1.00 | 3.02 | C |
| ATOM | 106 | CD1 | ILE | A | 25 | 85.207 | −42.239 | 1.891 | 1.00 | 3.02 | C |
| ATOM | 107 | CG2 | ILE | A | 25 | 85.579 | −41.039 | 4.658 | 1.00 | 3.02 | C |
| ATOM | 108 | C | ILE | A | 25 | 83.445 | −39.282 | 5.618 | 1.00 | 3.02 | C |
| ATOM | 109 | O | ILE | A | 25 | 83.270 | −39.383 | 6.830 | 1.00 | 3.02 | O |
| ATOM | 110 | N | LYS | A | 26 | 83.905 | −38.187 | 5.017 | 1.00 | 3.02 | N |
| ATOM | 111 | CA | LYS | A | 26 | 84.256 | −36.959 | 5.735 | 1.00 | 3.02 | C |
| ATOM | 112 | CB | LYS | A | 26 | 84.429 | −35.778 | 4.762 | 1.00 | 3.02 | C |
| ATOM | 113 | CG | LYS | A | 26 | 84.993 | −34.534 | 5.418 | 1.00 | 3.02 | C |
| ATOM | 114 | CD | LYS | A | 26 | 86.328 | −34.862 | 6.061 | 1.00 | 3.02 | C |
| ATOM | 115 | CE | LYS | A | 26 | 86.667 | −33.913 | 7.193 | 1.00 | 3.02 | C |
| ATOM | 116 | NZ | LYS | A | 26 | 87.878 | −34.377 | 7.916 | 1.00 | 3.02 | N |
| ATOM | 117 | C | LYS | A | 26 | 83.270 | −36.597 | 6.858 | 1.00 | 3.02 | C |
| ATOM | 118 | O | LYS | A | 26 | 83.663 | −36.548 | 8.031 | 1.00 | 3.02 | O |
| ATOM | 119 | N | MET | A | 27 | 82.007 | −36.346 | 6.510 | 1.00 | 3.02 | N |
| ATOM | 120 | CA | MET | A | 27 | 81.020 | −36.100 | 7.544 | 1.00 | 3.02 | C |
| ATOM | 121 | CB | MET | A | 27 | 79.915 | −35.146 | 7.084 | 1.00 | 3.02 | C |
| ATOM | 122 | CG | MET | A | 27 | 80.059 | −33.728 | 7.595 | 1.00 | 3.02 | C |
| ATOM | 123 | SD | MET | A | 27 | 78.832 | −33.203 | 8.832 | 1.00 | 3.02 | S |
| ATOM | 124 | CE | MET | A | 27 | 79.395 | −34.086 | 10.306 | 1.00 | 3.02 | C |
| ATOM | 125 | C | MET | A | 27 | 80.475 | −37.413 | 8.104 | 1.00 | 3.02 | C |
| ATOM | 126 | O | MET | A | 27 | 81.037 | −37.946 | 9.070 | 1.00 | 3.02 | O |
| ATOM | 127 | N | TYR | A | 28 | 79.435 | −37.972 | 7.486 | 1.00 | 3.02 | N |
| ATOM | 128 | CA | TYR | A | 28 | 78.678 | −39.012 | 8.171 | 1.00 | 3.02 | C |
| ATOM | 129 | CB | TYR | A | 28 | 77.247 | −39.095 | 7.647 | 1.00 | 3.02 | C |
| ATOM | 130 | CG | TYR | A | 28 | 76.240 | −39.061 | 8.773 | 1.00 | 3.02 | C |
| ATOM | 131 | CD1 | TYR | A | 28 | 75.576 | −37.888 | 9.091 | 1.00 | 3.02 | C |
| ATOM | 132 | CE1 | TYR | A | 28 | 74.657 | −37.840 | 10.116 | 1.00 | 3.02 | C |
| ATOM | 133 | CZ | TYR | A | 28 | 74.403 | −38.971 | 10.854 | 1.00 | 3.02 | C |
| ATOM | 134 | OH | TYR | A | 28 | 73.488 | −38.897 | 11.870 | 1.00 | 3.02 | O |
| ATOM | 135 | CE2 | TYR | A | 28 | 75.056 | −40.152 | 10.578 | 1.00 | 3.02 | C |
| ATOM | 136 | CD2 | TYR | A | 28 | 75.970 | −40.193 | 9.540 | 1.00 | 3.02 | C |
| ATOM | 137 | C | TYR | A | 28 | 79.323 | −40.414 | 8.408 | 1.00 | 3.02 | C |
| ATOM | 138 | O | TYR | A | 28 | 78.722 | −41.230 | 9.129 | 1.00 | 3.02 | O |
| ATOM | 139 | N | VAL | A | 29 | 80.508 | −40.735 | 7.872 | 1.00 | 3.02 | N |
| ATOM | 140 | CA | VAL | A | 29 | 81.169 | −41.949 | 8.403 | 1.00 | 3.02 | C |
| ATOM | 141 | CB | VAL | A | 29 | 81.932 | −42.794 | 7.335 | 1.00 | 3.02 | C |
| ATOM | 142 | CG1 | VAL | A | 29 | 83.367 | −43.124 | 7.779 | 1.00 | 3.02 | C |
| ATOM | 143 | CG2 | VAL | A | 29 | 81.157 | −44.077 | 7.037 | 1.00 | 3.02 | C |
| ATOM | 144 | C | VAL | A | 29 | 82.000 | −41.643 | 9.650 | 1.00 | 3.02 | C |
| ATOM | 145 | O | VAL | A | 29 | 82.158 | −42.504 | 10.526 | 1.00 | 3.02 | O |
| ATOM | 146 | N | VAL | A | 30 | 82.505 | −40.405 | 9.710 | 1.00 | 3.02 | N |
| ATOM | 147 | CA | VAL | A | 30 | 83.177 | −39.846 | 10.899 | 1.00 | 3.02 | C |
| ATOM | 148 | CB | VAL | A | 30 | 84.032 | −38.576 | 10.566 | 1.00 | 3.02 | C |
| ATOM | 149 | CG1 | VAL | A | 30 | 84.643 | −37.992 | 11.819 | 1.00 | 3.02 | C |
| ATOM | 150 | CG2 | VAL | A | 30 | 85.154 | −38.909 | 9.580 | 1.00 | 3.02 | C |
| ATOM | 151 | C | VAL | A | 30 | 82.120 | −39.536 | 11.965 | 1.00 | 3.02 | C |
| ATOM | 152 | O | VAL | A | 30 | 82.441 | −39.392 | 13.144 | 1.00 | 3.02 | O |
| ATOM | 153 | N | ALA | A | 31 | 80.863 | −39.446 | 11.525 | 1.00 | 3.02 | N |
| ATOM | 154 | CA | ALA | A | 31 | 79.707 | −39.417 | 12.412 | 1.00 | 3.02 | C |
| ATOM | 155 | CB | ALA | A | 31 | 78.488 | −38.929 | 11.654 | 1.00 | 3.02 | C |
| ATOM | 156 | C | ALA | A | 31 | 79.445 | −40.804 | 13.012 | 1.00 | 3.02 | C |
| ATOM | 157 | O | ALA | A | 31 | 79.255 | −40.941 | 14.221 | 1.00 | 3.02 | O |
| ATOM | 158 | N | ILE | A | 32 | 79.457 | −41.824 | 12.157 | 1.00 | 3.02 | N |
| ATOM | 159 | CA | ILE | A | 32 | 79.176 | −43.204 | 12.564 | 1.00 | 3.02 | C |
| ATOM | 160 | CB | ILE | A | 32 | 78.661 | −44.075 | 11.344 | 1.00 | 3.02 | C |
| ATOM | 161 | CG1 | ILE | A | 32 | 77.355 | −43.492 | 10.778 | 1.00 | 3.02 | C |
| ATOM | 162 | CD1 | ILE | A | 32 | 77.015 | −43.880 | 9.361 | 1.00 | 3.02 | C |
| ATOM | 163 | CG2 | ILE | A | 32 | 78.486 | −45.566 | 11.722 | 1.00 | 3.02 | C |
| ATOM | 164 | C | ILE | A | 32 | 80.348 | −43.862 | 13.335 | 1.00 | 3.02 | C |
| ATOM | 165 | O | ILE | A | 32 | 80.119 | −44.666 | 14.233 | 1.00 | 3.02 | O |
| ATOM | 166 | N | ILE | A | 33 | 81.593 | −43.520 | 13.010 | 1.00 | 3.02 | N |
| ATOM | 167 | CA | ILE | A | 33 | 82.728 | −44.026 | 13.809 | 1.00 | 3.02 | C |
| ATOM | 168 | CB | ILE | A | 33 | 84.097 | −43.999 | 13.037 | 1.00 | 3.02 | C |
| ATOM | 169 | CG1 | ILE | A | 33 | 84.063 | −44.988 | 11.867 | 1.00 | 3.02 | C |
| ATOM | 170 | CD1 | ILE | A | 33 | 85.230 | −44.884 | 10.908 | 1.00 | 3.02 | C |
| ATOM | 171 | CG2 | ILE | A | 33 | 85.259 | −44.364 | 13.967 | 1.00 | 3.02 | C |
| ATOM | 172 | C | ILE | A | 33 | 82.803 | −43.374 | 15.212 | 1.00 | 3.02 | C |
| ATOM | 173 | O | ILE | A | 33 | 82.533 | −44.041 | 16.216 | 1.00 | 3.02 | O |
| ATOM | 174 | N | THR | A | 34 | 83.143 | −42.083 | 15.268 | 1.00 | 3.02 | N |
| ATOM | 175 | CA | THR | A | 34 | 83.163 | −41.323 | 16.529 | 1.00 | 3.02 | C |
| ATOM | 176 | CB | THR | A | 34 | 82.905 | −39.782 | 16.323 | 1.00 | 3.02 | C |
| ATOM | 177 | OG1 | THR | A | 34 | 81.781 | −39.573 | 15.452 | 1.00 | 3.02 | O |
| ATOM | 178 | CG2 | THR | A | 34 | 84.148 | −39.059 | 15.770 | 1.00 | 3.02 | C |
| ATOM | 179 | C | THR | A | 34 | 82.141 | −41.834 | 17.548 | 1.00 | 3.02 | C |
| ATOM | 180 | O | THR | A | 34 | 82.519 | −42.460 | 18.550 | 1.00 | 3.02 | O |
| ATOM | 181 | N | GLY | A | 35 | 80.857 | −41.578 | 17.262 | 1.00 | 3.02 | N |
| ATOM | 182 | CA | GLY | A | 35 | 79.739 | −41.859 | 18.168 | 1.00 | 3.02 | C |
| ATOM | 183 | C | GLY | A | 35 | 79.522 | −43.315 | 18.509 | 1.00 | 3.02 | C |
| ATOM | 184 | O | GLY | A | 35 | 79.231 | −43.649 | 19.659 | 1.00 | 3.02 | O |
| ATOM | 185 | N | GLN | A | 36 | 79.654 | −44.183 | 17.507 | 1.00 | 3.02 | N |
| ATOM | 186 | CA | GLN | A | 36 | 79.610 | −45.625 | 17.734 | 1.00 | 3.02 | C |
| ATOM | 187 | CB | GLN | A | 36 | 79.654 | −46.413 | 16.424 | 1.00 | 3.02 | C |
| ATOM | 188 | CG | GLN | A | 36 | 78.456 | −46.204 | 15.502 | 1.00 | 3.02 | C |
| ATOM | 189 | CD | GLN | A | 36 | 77.265 | −47.103 | 15.820 | 1.00 | 3.02 | C |
| ATOM | 190 | OE1 | GLN | A | 36 | 77.324 | −47.954 | 16.711 | 1.00 | 3.02 | O |
| ATOM | 191 | NE2 | GLN | A | 36 | 76.166 | −46.906 | 15.090 | 1.00 | 3.02 | N |
| ATOM | 192 | C | GLN | A | 36 | 80.759 | −46.049 | 18.645 | 1.00 | 3.02 | C |
| ATOM | 193 | O | GLN | A | 36 | 80.532 | −46.840 | 19.567 | 1.00 | 3.02 | O |
| ATOM | 194 | N | VAL | A | 37 | 81.974 | −45.527 | 18.401 | 1.00 | 3.02 | N |
| ATOM | 195 | CA | VAL | A | 37 | 83.131 | −45.858 | 19.257 | 1.00 | 3.02 | C |
| ATOM | 196 | CB | VAL | A | 37 | 84.523 | −45.362 | 18.706 | 1.00 | 3.02 | C |
| ATOM | 197 | CG1 | VAL | A | 37 | 85.644 | −45.627 | 19.718 | 1.00 | 3.02 | C |
| ATOM | 198 | CG2 | VAL | A | 37 | 84.886 | −46.035 | 17.386 | 1.00 | 3.02 | C |
| ATOM | 199 | C | VAL | A | 37 | 82.874 | −45.322 | 20.665 | 1.00 | 3.02 | C |
| ATOM | 200 | O | VAL | A | 37 | 83.300 | −45.943 | 21.654 | 1.00 | 3.02 | O |
| ATOM | 201 | N | ARG | A | 38 | 82.156 | −44.193 | 20.736 | 1.00 | 3.02 | N |
| ATOM | 202 | CA | ARG | A | 38 | 81.822 | −43.539 | 22.010 | 1.00 | 3.02 | C |
| ATOM | 203 | CB | ARG | A | 38 | 81.342 | −42.087 | 21.808 | 1.00 | 3.02 | C |
| ATOM | 204 | CG | ARG | A | 38 | 81.147 | −41.310 | 23.119 | 1.00 | 3.02 | C |
| ATOM | 205 | CD | ARG | A | 38 | 80.618 | −39.900 | 22.891 | 1.00 | 3.02 | C |
| ATOM | 206 | NE | ARG | A | 38 | 79.822 | −39.426 | 24.030 | 1.00 | 3.02 | N |
| ATOM | 207 | CZ | ARG | A | 38 | 79.024 | −38.356 | 24.028 | 1.00 | 3.02 | C |
| ATOM | 208 | NH1 | ARG | A | 38 | 78.882 | −37.599 | 22.943 | 1.00 | 3.02 | N |
| ATOM | 209 | NH2 | ARG | A | 38 | 78.359 | −38.051 | 25.129 | 1.00 | 3.02 | N |
| ATOM | 210 | C | ARG | A | 38 | 80.811 | −44.327 | 22.849 | 1.00 | 3.02 | C |
| ATOM | 211 | O | ARG | A | 38 | 81.053 | −44.575 | 24.033 | 1.00 | 3.02 | O |
| ATOM | 212 | N | LEU | A | 39 | 79.700 | −44.746 | 22.246 | 1.00 | 3.02 | N |
| ATOM | 213 | CA | LEU | A | 39 | 78.642 | −45.425 | 23.005 | 1.00 | 3.02 | C |
| ATOM | 214 | CB | LEU | A | 39 | 77.328 | −45.495 | 22.201 | 1.00 | 3.02 | C |
| ATOM | 215 | CG | LEU | A | 39 | 76.400 | −44.272 | 22.416 | 1.00 | 3.02 | C |
| ATOM | 216 | CD1 | LEU | A | 39 | 75.799 | −43.682 | 21.119 | 1.00 | 3.02 | C |
| ATOM | 217 | CD2 | LEU | A | 39 | 75.308 | −44.564 | 23.457 | 1.00 | 3.02 | C |
| ATOM | 218 | C | LEU | A | 39 | 79.069 | −46.785 | 23.565 | 1.00 | 3.02 | C |
| ATOM | 219 | O | LEU | A | 39 | 78.391 | −47.348 | 24.430 | 1.00 | 3.02 | O |
| ATOM | 220 | N | ARG | A | 40 | 80.203 | −47.296 | 23.087 | 1.00 | 3.02 | N |
| ATOM | 221 | CA | ARG | A | 40 | 80.771 | −48.548 | 23.587 | 1.00 | 3.02 | C |
| ATOM | 222 | CB | ARG | A | 40 | 81.502 | −49.304 | 22.471 | 1.00 | 3.02 | C |
| ATOM | 223 | CG | ARG | A | 40 | 82.024 | −50.690 | 22.893 | 1.00 | 3.02 | C |
| ATOM | 224 | CD | ARG | A | 40 | 81.238 | −51.815 | 22.258 | 1.00 | 3.02 | C |
| ATOM | 225 | NE | ARG | A | 40 | 80.994 | −52.911 | 23.184 | 1.00 | 3.02 | N |
| ATOM | 226 | CZ | ARG | A | 40 | 79.886 | −53.649 | 23.173 | 1.00 | 3.02 | C |
| ATOM | 227 | NH1 | ARG | A | 40 | 78.921 | −53.406 | 22.292 | 1.00 | 3.02 | N |
| ATOM | 228 | NH2 | ARG | A | 40 | 79.727 | −54.625 | 24.052 | 1.00 | 3.02 | N |
| ATOM | 229 | C | ARG | A | 40 | 81.737 | −48.284 | 24.735 | 1.00 | 3.02 | C |
| ATOM | 230 | O | ARG | A | 40 | 81.821 | −49.077 | 25.684 | 1.00 | 3.02 | O |
| ATOM | 231 | N | LYS | A | 41 | 82.466 | −47.171 | 24.627 | 1.00 | 3.02 | N |
| ATOM | 232 | CA | LYS | A | 41 | 83.501 | −46.790 | 25.591 | 1.00 | 3.02 | C |
| ATOM | 233 | CB | LYS | A | 41 | 84.535 | −45.890 | 24.922 | 1.00 | 3.02 | C |
| ATOM | 234 | CG | LYS | A | 41 | 85.429 | −46.611 | 23.932 | 1.00 | 3.02 | C |
| ATOM | 235 | CD | LYS | A | 41 | 86.690 | −45.797 | 23.695 | 1.00 | 3.02 | C |
| ATOM | 236 | CE | LYS | A | 41 | 87.718 | −46.526 | 22.850 | 1.00 | 3.02 | C |
| ATOM | 237 | NZ | LYS | A | 41 | 88.902 | −45.648 | 22.696 | 1.00 | 3.02 | N |
| ATOM | 238 | C | LYS | A | 41 | 82.898 | −46.085 | 26.797 | 1.00 | 3.02 | C |
| ATOM | 239 | O | LYS | A | 41 | 83.600 | −45.762 | 27.769 | 1.00 | 3.02 | O |
| ATOM | 240 | N | LYS | A | 42 | 81.586 | −45.877 | 26.718 | 1.00 | 3.02 | N |
| ATOM | 241 | CA | LYS | A | 42 | 80.822 | −45.098 | 27.683 | 1.00 | 3.02 | C |
| ATOM | 242 | CB | LYS | A | 42 | 79.329 | −45.447 | 27.585 | 1.00 | 3.02 | C |
| ATOM | 243 | CG | LYS | A | 42 | 78.420 | −44.233 | 27.358 | 1.00 | 3.02 | C |
| ATOM | 244 | CD | LYS | A | 42 | 78.945 | −43.379 | 26.202 | 1.00 | 3.02 | C |
| ATOM | 245 | CE | LYS | A | 42 | 77.958 | −42.312 | 25.756 | 1.00 | 3.02 | C |
| ATOM | 246 | NZ | LYS | A | 42 | 77.610 | −41.317 | 26.816 | 1.00 | 3.02 | N |
| ATOM | 247 | C | LYS | A | 42 | 81.310 | −45.194 | 29.125 | 1.00 | 3.02 | C |
| ATOM | 248 | O | LYS | A | 42 | 81.089 | −46.200 | 29.805 | 1.00 | 3.02 | O |
| ATOM | 249 | N | ALA | A | 43 | 82.008 | −44.142 | 29.552 | 1.00 | 3.02 | N |
| ATOM | 250 | CA | ALA | A | 43 | 82.286 | −43.879 | 30.962 | 1.00 | 3.02 | C |
| ATOM | 251 | CB | ALA | A | 43 | 83.626 | −43.134 | 31.127 | 1.00 | 3.02 | C |
| ATOM | 252 | C | ALA | A | 43 | 81.111 | −43.068 | 31.538 | 1.00 | 3.02 | C |
| ATOM | 253 | O | ALA | A | 43 | 81.100 | −42.696 | 32.713 | 1.00 | 3.02 | O |
| ATOM | 254 | N | PHE | A | 44 | 80.108 | −42.825 | 30.698 | 1.00 | 3.02 | N |
| ATOM | 255 | CA | PHE | A | 44 | 78.905 | −42.114 | 31.116 | 1.00 | 3.02 | C |
| ATOM | 256 | CB | PHE | A | 44 | 78.140 | −41.554 | 29.912 | 1.00 | 3.02 | C |
| ATOM | 257 | CG | PHE | A | 44 | 78.780 | −40.327 | 29.314 | 1.00 | 3.02 | C |
| ATOM | 258 | CD1 | PHE | A | 44 | 80.177 | −40.261 | 29.142 | 1.00 | 3.02 | C |
| ATOM | 259 | CE1 | PHE | A | 44 | 80.773 | −39.143 | 28.593 | 1.00 | 3.02 | C |
| ATOM | 260 | CZ | PHE | A | 44 | 79.989 | −38.056 | 28.196 | 1.00 | 3.02 | C |
| ATOM | 261 | CE2 | PHE | A | 44 | 78.605 | −38.100 | 28.358 | 1.00 | 3.02 | C |
| ATOM | 262 | CD2 | PHE | A | 44 | 77.999 | −39.241 | 28.916 | 1.00 | 3.02 | C |
| ATOM | 263 | C | PHE | A | 44 | 78.011 | −42.939 | 32.029 | 1.00 | 3.02 | C |
| ATOM | 264 | O | PHE | A | 44 | 78.168 | −44.166 | 32.126 | 1.00 | 3.02 | O |
| ATOM | 265 | N | ALA | A | 45 | 77.051 | −42.250 | 32.651 | 1.00 | 3.02 | N |
| ATOM | 266 | CA | ALA | A | 45 | 76.499 | −42.613 | 33.958 | 1.00 | 3.02 | C |
| ATOM | 267 | CB | ALA | A | 45 | 76.084 | −44.082 | 34.024 | 1.00 | 3.02 | C |
| ATOM | 268 | C | ALA | A | 45 | 77.602 | −42.268 | 34.971 | 1.00 | 3.02 | C |
| ATOM | 269 | O | ALA | A | 45 | 77.665 | −42.824 | 36.082 | 1.00 | 3.02 | O |
| ATOM | 270 | N | ASN | A | 46 | 78.448 | −41.314 | 34.541 | 1.00 | 3.02 | N |
| ATOM | 271 | CA | ASN | A | 46 | 79.608 | −40.766 | 35.274 | 1.00 | 3.02 | C |
| ATOM | 272 | CB | ASN | A | 46 | 80.095 | −39.448 | 34.634 | 1.00 | 3.02 | C |
| ATOM | 273 | CG | ASN | A | 46 | 80.305 | −39.549 | 33.135 | 1.00 | 3.02 | C |
| ATOM | 274 | OD1 | ASN | A | 46 | 79.648 | −38.862 | 32.351 | 1.00 | 3.02 | O |
| ATOM | 275 | ND2 | ASN | A | 46 | 81.231 | −40.389 | 32.732 | 1.00 | 3.02 | N |
| ATOM | 276 | C | ASN | A | 46 | 79.275 | −40.471 | 36.734 | 1.00 | 3.02 | C |
| ATOM | 277 | O | ASN | A | 46 | 78.169 | −39.966 | 37.012 | 1.00 | 3.02 | O |
| ATOM | 278 | N | PRO | A | 47 | 80.264 | −40.666 | 37.650 | 1.00 | 3.02 | N |
| ATOM | 279 | CA | PRO | A | 47 | 80.180 | −40.813 | 39.106 | 1.00 | 3.02 | C |
| ATOM | 280 | CB | PRO | A | 47 | 81.177 | −39.748 | 39.590 | 1.00 | 3.02 | C |
| ATOM | 281 | CG | PRO | A | 47 | 82.199 | −39.622 | 38.407 | 1.00 | 3.02 | C |
| ATOM | 282 | CD | PRO | A | 47 | 81.681 | −40.534 | 37.280 | 1.00 | 3.02 | C |
| ATOM | 283 | C | PRO | A | 47 | 78.775 | −40.591 | 39.661 | 1.00 | 3.02 | C |
| ATOM | 284 | O | PRO | A | 47 | 78.574 | −39.776 | 40.565 | 1.00 | 3.02 | O |
| ATOM | 285 | N | GLU | A | 48 | 77.829 | −41.358 | 39.112 | 1.00 | 3.02 | N |
| ATOM | 286 | CA | GLU | A | 48 | 76.396 | −41.163 | 39.294 | 1.00 | 3.02 | C |
| ATOM | 287 | CB | GLU | A | 48 | 75.974 | −41.428 | 40.739 | 1.00 | 3.02 | C |
| ATOM | 288 | CG | GLU | A | 48 | 74.512 | −41.766 | 40.898 | 1.00 | 3.02 | C |
| ATOM | 289 | CD | GLU | A | 48 | 74.172 | −42.163 | 42.314 | 1.00 | 3.02 | C |
| ATOM | 290 | OE1 | GLU | A | 48 | 75.113 | −42.527 | 43.058 | 1.00 | 3.02 | O |
| ATOM | 291 | OE2 | GLU | A | 48 | 72.968 | −42.115 | 42.681 | 1.00 | 3.02 | O |
| ATOM | 292 | C | GLU | A | 48 | 76.006 | −39.756 | 38.834 | 1.00 | 3.02 | C |
| ATOM | 293 | O | GLU | A | 48 | 76.314 | −38.743 | 39.497 | 1.00 | 3.02 | O |
| ATOM | 294 | N | ASP | A | 49 | 75.345 | −39.704 | 37.681 | 1.00 | 3.02 | N |
| ATOM | 295 | CA | ASP | A | 49 | 74.897 | −38.447 | 37.091 | 1.00 | 3.02 | C |
| ATOM | 296 | CB | ASP | A | 49 | 76.020 | −37.871 | 36.204 | 1.00 | 3.02 | C |
| ATOM | 297 | CG | ASP | A | 49 | 76.404 | −36.444 | 36.590 | 1.00 | 3.02 | C |
| ATOM | 298 | OD1 | ASP | A | 49 | 75.634 | −35.811 | 37.355 | 1.00 | 3.02 | O |
| ATOM | 299 | OD2 | ASP | A | 49 | 77.465 | −35.950 | 36.128 | 1.00 | 3.02 | O |
| ATOM | 300 | C | ASP | A | 49 | 73.639 | −38.671 | 36.251 | 1.00 | 3.02 | C |
| ATOM | 301 | O | ASP | A | 49 | 73.314 | −39.801 | 35.862 | 1.00 | 3.02 | O |
| ATOM | 302 | N | ALA | A | 50 | 72.915 | −37.585 | 36.011 | 1.00 | 3.02 | N |
| ATOM | 303 | CA | ALA | A | 50 | 72.167 | −37.437 | 34.780 | 1.00 | 3.02 | C |
| ATOM | 304 | CB | ALA | A | 50 | 71.022 | −36.457 | 34.961 | 1.00 | 3.02 | C |
| ATOM | 305 | C | ALA | A | 50 | 73.277 | −36.817 | 33.937 | 1.00 | 3.02 | C |
| ATOM | 306 | O | ALA | A | 50 | 74.166 | −36.154 | 34.503 | 1.00 | 3.02 | O |
| ATOM | 307 | N | LEU | A | 51 | 73.288 | −37.036 | 32.624 | 1.00 | 3.02 | N |
| ATOM | 308 | CA | LEU | A | 51 | 74.431 | −36.533 | 31.851 | 1.00 | 3.02 | C |
| ATOM | 309 | CB | LEU | A | 51 | 74.710 | −37.361 | 30.610 | 1.00 | 3.02 | C |
| ATOM | 310 | CG | LEU | A | 51 | 73.531 | −37.858 | 29.816 | 1.00 | 3.02 | C |
| ATOM | 311 | CD1 | LEU | A | 51 | 73.391 | −37.019 | 28.568 | 1.00 | 3.02 | C |
| ATOM | 312 | CD2 | LEU | A | 51 | 73.825 | −39.304 | 29.492 | 1.00 | 3.02 | C |
| ATOM | 313 | C | LEU | A | 51 | 74.371 | −35.039 | 31.554 | 1.00 | 3.02 | C |
| ATOM | 314 | O | LEU | A | 51 | 73.642 | −34.321 | 32.224 | 1.00 | 3.02 | O |
| ATOM | 315 | N | ARG | A | 52 | 75.128 | −34.602 | 30.545 | 1.00 | 3.02 | N |
| ATOM | 316 | CA | ARG | A | 52 | 75.583 | −33.202 | 30.354 | 1.00 | 3.02 | C |
| ATOM | 317 | CB | ARG | A | 52 | 74.459 | −32.128 | 30.332 | 1.00 | 3.02 | C |
| ATOM | 318 | CG | ARG | A | 52 | 73.691 | −31.859 | 31.627 | 1.00 | 3.02 | C |
| ATOM | 319 | CD | ARG | A | 52 | 72.261 | −32.417 | 31.538 | 1.00 | 3.02 | C |
| ATOM | 320 | NE | ARG | A | 52 | 71.694 | −32.897 | 32.812 | 1.00 | 3.02 | N |
| ATOM | 321 | CZ | ARG | A | 52 | 71.744 | −32.262 | 33.990 | 1.00 | 3.02 | C |
| ATOM | 322 | NH1 | ARG | A | 52 | 72.328 | −31.072 | 34.101 | 1.00 | 3.02 | N |
| ATOM | 323 | NH2 | ARG | A | 52 | 71.184 | −32.817 | 35.064 | 1.00 | 3.02 | N |
| ATOM | 324 | C | ARG | A | 52 | 76.724 | −32.893 | 31.326 | 1.00 | 3.02 | C |
| ATOM | 325 | O | ARG | A | 52 | 77.022 | −33.709 | 32.203 | 1.00 | 3.02 | O |
| ATOM | 326 | N | HIS | A | 53 | 77.375 | −31.746 | 31.143 | 1.00 | 3.02 | N |
| ATOM | 327 | CA | HIS | A | 53 | 78.651 | −31.429 | 31.801 | 1.00 | 3.02 | C |
| ATOM | 328 | CB | HIS | A | 53 | 78.551 | −31.282 | 33.340 | 1.00 | 3.02 | C |
| ATOM | 329 | CG | HIS | A | 53 | 77.301 | −31.846 | 33.949 | 1.00 | 3.02 | C |
| ATOM | 330 | ND1 | HIS | A | 53 | 76.080 | −31.207 | 33.873 | 1.00 | 3.02 | N |
| ATOM | 331 | CE1 | HIS | A | 53 | 75.167 | −31.922 | 34.503 | 1.00 | 3.02 | C |
| ATOM | 332 | NE2 | HIS | A | 53 | 75.751 | −33.000 | 34.991 | 1.00 | 3.02 | N |
| ATOM | 333 | CD2 | HIS | A | 53 | 77.089 | −32.972 | 34.670 | 1.00 | 3.02 | C |
| ATOM | 334 | C | HIS | A | 53 | 79.755 | −32.407 | 31.433 | 1.00 | 3.02 | C |
| ATOM | 335 | O | HIS | A | 53 | 79.820 | −33.502 | 31.983 | 1.00 | 3.02 | O |
| ATOM | 336 | N | GLY | A | 54 | 80.630 | −32.005 | 30.512 | 1.00 | 3.02 | N |
| ATOM | 337 | CA | GLY | A | 54 | 81.769 | −32.840 | 30.093 | 1.00 | 3.02 | C |
| ATOM | 338 | C | GLY | A | 54 | 83.115 | −32.451 | 30.707 | 1.00 | 3.02 | C |
| ATOM | 339 | O | GLY | A | 54 | 83.803 | −31.540 | 30.212 | 1.00 | 3.02 | O |
| ATOM | 340 | N | GLY | A | 55 | 83.494 | −33.150 | 31.781 | 1.00 | 3.02 | N |
| ATOM | 341 | CA | GLY | A | 55 | 84.763 | −32.887 | 32.468 | 1.00 | 3.02 | C |
| ATOM | 342 | C | GLY | A | 55 | 84.866 | −33.300 | 33.934 | 1.00 | 3.02 | C |
| ATOM | 343 | O | GLY | A | 55 | 85.195 | −32.456 | 34.791 | 1.00 | 3.02 | O |
| ATOM | 344 | N | PRO | A | 56 | 84.600 | −34.596 | 34.239 | 1.00 | 3.02 | N |
| ATOM | 345 | CA | PRO | A | 56 | 84.825 | −35.142 | 35.578 | 1.00 | 3.02 | C |
| ATOM | 346 | CB | PRO | A | 56 | 83.781 | −36.248 | 35.672 | 1.00 | 3.02 | C |
| ATOM | 347 | CG | PRO | A | 56 | 83.683 | −36.756 | 34.293 | 1.00 | 3.02 | C |
| ATOM | 348 | CD | PRO | A | 56 | 84.049 | −35.624 | 33.343 | 1.00 | 3.02 | C |
| ATOM | 349 | C | PRO | A | 56 | 86.234 | −35.723 | 35.700 | 1.00 | 3.02 | C |
| ATOM | 350 | O | PRO | A | 56 | 87.059 | −35.185 | 36.447 | 1.00 | 3.02 | O |
| ATOM | 351 | N | GLN | A | 57 | 86.499 | −36.809 | 34.975 | 1.00 | 3.02 | N |
| ATOM | 352 | CA | GLN | A | 57 | 87.854 | −37.314 | 34.795 | 1.00 | 3.02 | C |
| ATOM | 353 | CB | GLN | A | 57 | 87.880 | −38.854 | 34.763 | 1.00 | 3.02 | C |
| ATOM | 354 | CG | GLN | A | 57 | 87.461 | −39.518 | 36.088 | 1.00 | 3.02 | C |
| ATOM | 355 | CD | GLN | A | 57 | 88.197 | −40.821 | 36.405 | 1.00 | 3.02 | C |
| ATOM | 356 | OE1 | GLN | A | 57 | 88.908 | −41.371 | 35.572 | 1.00 | 3.02 | O |
| ATOM | 357 | NE2 | GLN | A | 57 | 88.015 | −41.317 | 37.624 | 1.00 | 3.02 | N |
| ATOM | 358 | C | GLN | A | 57 | 88.374 | −36.728 | 33.487 | 1.00 | 3.02 | C |
| ATOM | 359 | O | GLN | A | 57 | 87.572 | −36.298 | 32.643 | 1.00 | 3.02 | O |
| ATOM | 360 | N | TYR | A | 58 | 89.701 | −36.679 | 33.326 | 1.00 | 3.02 | N |
| ATOM | 361 | CA | TYR | A | 58 | 90.277 | −36.329 | 32.030 | 1.00 | 3.02 | C |
| ATOM | 362 | CB | TYR | A | 58 | 91.811 | −36.255 | 32.072 | 1.00 | 3.02 | C |
| ATOM | 363 | CG | TYR | A | 58 | 92.519 | −37.572 | 32.302 | 1.00 | 3.02 | C |
| ATOM | 364 | CD1 | TYR | A | 58 | 93.061 | −38.297 | 31.231 | 1.00 | 3.02 | C |
| ATOM | 365 | CE1 | TYR | A | 58 | 93.724 | −39.522 | 31.445 | 1.00 | 3.02 | C |
| ATOM | 366 | CZ | TYR | A | 58 | 93.849 | −40.029 | 32.743 | 1.00 | 3.02 | C |
| ATOM | 367 | OH | TYR | A | 58 | 94.501 | −41.225 | 32.965 | 1.00 | 3.02 | O |
| ATOM | 368 | CE2 | TYR | A | 58 | 93.319 | −39.328 | 33.821 | 1.00 | 3.02 | C |
| ATOM | 369 | CD2 | TYR | A | 58 | 92.656 | −38.099 | 33.595 | 1.00 | 3.02 | C |
| ATOM | 370 | C | TYR | A | 58 | 89.764 | −37.359 | 31.021 | 1.00 | 3.02 | C |
| ATOM | 371 | O | TYR | A | 58 | 88.998 | −37.007 | 30.113 | 1.00 | 3.02 | O |
| ATOM | 372 | N | CYS | A | 59 | 90.119 | −38.631 | 31.250 | 1.00 | 3.02 | N |
| ATOM | 373 | CA | CYS | A | 59 | 89.744 | −39.759 | 30.393 | 1.00 | 3.02 | C |
| ATOM | 374 | CB | CYS | A | 59 | 89.791 | −41.078 | 31.180 | 1.00 | 3.02 | C |
| ATOM | 375 | SG | CYS | A | 59 | 91.262 | −42.085 | 30.915 | 1.00 | 3.02 | S |
| ATOM | 376 | C | CYS | A | 59 | 88.380 | −39.576 | 29.740 | 1.00 | 3.02 | C |
| ATOM | 377 | O | CYS | A | 59 | 88.292 | −39.293 | 28.547 | 1.00 | 3.02 | O |
| ATOM | 378 | N | ARG | A | 60 | 87.324 | −39.721 | 30.525 | 1.00 | 3.02 | N |
| ATOM | 379 | CA | ARG | A | 60 | 85.976 | −39.606 | 29.993 | 1.00 | 3.02 | C |
| ATOM | 380 | CB | ARG | A | 60 | 84.976 | −39.267 | 31.111 | 1.00 | 3.02 | C |
| ATOM | 381 | CG | ARG | A | 60 | 83.563 | −39.046 | 30.630 | 1.00 | 3.02 | C |
| ATOM | 382 | CD | ARG | A | 60 | 82.716 | −38.409 | 31.701 | 1.00 | 3.02 | C |
| ATOM | 383 | NE | ARG | A | 60 | 81.838 | −37.380 | 31.133 | 1.00 | 3.02 | N |
| ATOM | 384 | CZ | ARG | A | 60 | 81.082 | −36.506 | 31.812 | 1.00 | 3.02 | C |
| ATOM | 385 | NH1 | ARG | A | 60 | 81.019 | −36.514 | 33.139 | 1.00 | 3.02 | N |
| ATOM | 386 | NH2 | ARG | A | 60 | 80.354 | −35.629 | 31.138 | 1.00 | 3.02 | N |
| ATOM | 387 | C | ARG | A | 60 | 85.923 | −38.565 | 28.867 | 1.00 | 3.02 | C |
| ATOM | 388 | O | ARG | A | 60 | 85.980 | −38.921 | 27.671 | 1.00 | 3.02 | O |
| ATOM | 389 | N | SER | A | 61 | 85.869 | −37.289 | 29.272 | 1.00 | 3.02 | N |
| ATOM | 390 | CA | SER | A | 61 | 85.463 | −36.171 | 28.404 | 1.00 | 3.02 | C |
| ATOM | 391 | CB | SER | A | 61 | 84.422 | −35.320 | 29.134 | 1.00 | 3.02 | C |
| ATOM | 392 | OG | SER | A | 61 | 84.917 | −34.912 | 30.399 | 1.00 | 3.02 | O |
| ATOM | 393 | C | SER | A | 61 | 86.585 | −35.272 | 27.842 | 1.00 | 3.02 | C |
| ATOM | 394 | O | SER | A | 61 | 86.440 | −34.743 | 26.737 | 1.00 | 3.02 | O |
| ATOM | 395 | N | ASP | A | 62 | 87.681 | −35.092 | 28.586 | 1.00 | 3.02 | N |
| ATOM | 396 | CA | ASP | A | 62 | 88.749 | −34.168 | 28.154 | 1.00 | 3.02 | C |
| ATOM | 397 | CB | ASP | A | 62 | 89.941 | −34.133 | 29.129 | 1.00 | 3.02 | C |
| ATOM | 398 | CG | ASP | A | 62 | 90.068 | −32.790 | 29.876 | 1.00 | 3.02 | C |
| ATOM | 399 | OD1 | ASP | A | 62 | 89.104 | −32.329 | 30.527 | 1.00 | 3.02 | O |
| ATOM | 400 | OD2 | ASP | A | 62 | 91.158 | −32.181 | 29.822 | 1.00 | 3.02 | O |
| ATOM | 401 | C | ASP | A | 62 | 89.182 | −34.288 | 26.669 | 1.00 | 3.02 | C |
| ATOM | 402 | O | ASP | A | 62 | 89.051 | −33.299 | 25.943 | 1.00 | 3.02 | O |
| ATOM | 403 | N | PRO | A | 63 | 89.629 | −35.496 | 26.199 | 1.00 | 3.02 | N |
| ATOM | 404 | CA | PRO | A | 63 | 90.042 | −35.592 | 24.790 | 1.00 | 3.02 | C |
| ATOM | 405 | CB | PRO | A | 63 | 91.023 | −36.759 | 24.805 | 1.00 | 3.02 | C |
| ATOM | 406 | CG | PRO | A | 63 | 90.501 | −37.681 | 25.858 | 1.00 | 3.02 | C |
| ATOM | 407 | CD | PRO | A | 63 | 89.749 | −36.815 | 26.868 | 1.00 | 3.02 | C |
| ATOM | 408 | C | PRO | A | 63 | 88.873 | −35.926 | 23.857 | 1.00 | 3.02 | C |
| ATOM | 409 | O | PRO | A | 63 | 88.995 | −35.797 | 22.637 | 1.00 | 3.02 | O |
| ATOM | 410 | N | ASP | A | 64 | 87.753 | −36.337 | 24.452 | 1.00 | 3.02 | N |
| ATOM | 411 | CA | ASP | A | 64 | 86.587 | −36.823 | 23.731 | 1.00 | 3.02 | C |
| ATOM | 412 | CB | ASP | A | 64 | 85.882 | −37.943 | 24.550 | 1.00 | 3.02 | C |
| ATOM | 413 | CG | ASP | A | 64 | 84.422 | −38.240 | 24.107 | 1.00 | 3.02 | C |
| ATOM | 414 | OD1 | ASP | A | 64 | 83.927 | −37.720 | 23.090 | 1.00 | 3.02 | O |
| ATOM | 415 | OD2 | ASP | A | 64 | 83.749 | −39.014 | 24.819 | 1.00 | 3.02 | O |
| ATOM | 416 | C | ASP | A | 64 | 85.688 | −35.646 | 23.408 | 1.00 | 3.02 | C |
| ATOM | 417 | O | ASP | A | 64 | 85.345 | −35.443 | 22.251 | 1.00 | 3.02 | O |
| ATOM | 418 | N | VAL | A | 65 | 85.331 | −34.861 | 24.425 | 1.00 | 3.02 | N |
| ATOM | 419 | CA | VAL | A | 65 | 84.403 | −33.715 | 24.270 | 1.00 | 3.02 | C |
| ATOM | 420 | CB | VAL | A | 65 | 84.094 | −33.044 | 25.635 | 1.00 | 3.02 | C |
| ATOM | 421 | CG1 | VAL | A | 65 | 83.444 | −31.670 | 25.463 | 1.00 | 3.02 | C |
| ATOM | 422 | CG2 | VAL | A | 65 | 83.195 | −33.937 | 26.435 | 1.00 | 3.02 | C |
| ATOM | 423 | C | VAL | A | 65 | 84.887 | −32.696 | 23.226 | 1.00 | 3.02 | C |
| ATOM | 424 | O | VAL | A | 65 | 84.078 | −32.114 | 22.500 | 1.00 | 3.02 | O |
| ATOM | 425 | N | GLU | A | 66 | 86.205 | −32.501 | 23.171 | 1.00 | 3.02 | N |
| ATOM | 426 | CA | GLU | A | 66 | 86.860 | −31.827 | 22.065 | 1.00 | 3.02 | C |
| ATOM | 427 | CB | GLU | A | 66 | 88.352 | −31.652 | 22.359 | 1.00 | 3.02 | C |
| ATOM | 428 | CG | GLU | A | 66 | 89.085 | −30.733 | 21.401 | 1.00 | 3.02 | C |
| ATOM | 429 | CD | GLU | A | 66 | 90.595 | −30.697 | 21.643 | 1.00 | 3.02 | C |
| ATOM | 430 | OE1 | GLU | A | 66 | 91.056 | −31.306 | 22.642 | 1.00 | 3.02 | O |
| ATOM | 431 | OE2 | GLU | A | 66 | 91.321 | −30.070 | 20.829 | 1.00 | 3.02 | O |
| ATOM | 432 | C | GLU | A | 66 | 86.616 | −32.629 | 20.781 | 1.00 | 3.02 | C |
| ATOM | 433 | O | GLU | A | 66 | 85.945 | −32.137 | 19.891 | 1.00 | 3.02 | O |
| ATOM | 434 | N | ARG | A | 67 | 87.116 | −33.865 | 20.712 | 1.00 | 3.02 | N |
| ATOM | 435 | CA | ARG | A | 67 | 86.978 | −34.722 | 19.519 | 1.00 | 3.02 | C |
| ATOM | 436 | CB | ARG | A | 67 | 87.504 | −36.130 | 19.784 | 1.00 | 3.02 | C |
| ATOM | 437 | CG | ARG | A | 67 | 88.996 | −36.264 | 19.688 | 1.00 | 3.02 | C |
| ATOM | 438 | CD | ARG | A | 67 | 89.368 | −37.729 | 19.838 | 1.00 | 3.02 | C |
| ATOM | 439 | NE | ARG | A | 67 | 90.809 | −37.939 | 19.919 | 1.00 | 3.02 | N |
| ATOM | 440 | CZ | ARG | A | 67 | 91.364 | −39.059 | 20.356 | 1.00 | 3.02 | C |
| ATOM | 441 | NH1 | ARG | A | 67 | 90.593 | −40.062 | 20.757 | 1.00 | 3.02 | N |
| ATOM | 442 | NH2 | ARG | A | 67 | 92.684 | −39.170 | 20.402 | 1.00 | 3.02 | N |
| ATOM | 443 | C | ARG | A | 67 | 85.555 | −34.811 | 18.956 | 1.00 | 3.02 | C |
| ATOM | 444 | O | ARG | A | 67 | 85.339 | −34.598 | 17.767 | 1.00 | 3.02 | O |
| ATOM | 445 | N | CYS | A | 68 | 84.589 | −35.126 | 19.812 | 1.00 | 3.02 | N |
| ATOM | 446 | CA | CYS | A | 68 | 83.201 | −35.179 | 19.394 | 1.00 | 3.02 | C |
| ATOM | 447 | CB | CYS | A | 68 | 82.317 | −35.613 | 20.557 | 1.00 | 3.02 | C |
| ATOM | 448 | SG | CYS | A | 68 | 82.599 | −37.302 | 21.072 | 1.00 | 3.02 | S |
| ATOM | 449 | C | CYS | A | 68 | 82.769 | −33.821 | 18.840 | 1.00 | 3.02 | C |
| ATOM | 450 | O | CYS | A | 68 | 82.186 | −33.735 | 17.764 | 1.00 | 3.02 | O |
| ATOM | 451 | N | LEU | A | 69 | 83.084 | −32.760 | 19.573 | 1.00 | 3.02 | N |
| ATOM | 452 | CA | LEU | A | 69 | 82.833 | −31.406 | 19.088 | 1.00 | 3.02 | C |
| ATOM | 453 | CB | LEU | A | 69 | 83.026 | −30.366 | 20.210 | 1.00 | 3.02 | C |
| ATOM | 454 | CG | LEU | A | 69 | 82.070 | −30.526 | 21.412 | 1.00 | 3.02 | C |
| ATOM | 455 | CD1 | LEU | A | 69 | 82.278 | −29.440 | 22.451 | 1.00 | 3.02 | C |
| ATOM | 456 | CD2 | LEU | A | 69 | 80.595 | −30.568 | 20.998 | 1.00 | 3.02 | C |
| ATOM | 457 | C | LEU | A | 69 | 83.667 | −31.109 | 17.823 | 1.00 | 3.02 | C |
| ATOM | 458 | O | LEU | A | 69 | 83.099 | −30.967 | 16.738 | 1.00 | 3.02 | O |
| ATOM | 459 | N | ARG | A | 70 | 84.996 | −31.061 | 17.954 | 1.00 | 3.02 | N |
| ATOM | 460 | CA | ARG | A | 70 | 85.890 | −30.864 | 16.799 | 1.00 | 3.02 | C |
| ATOM | 461 | CB | ARG | A | 70 | 87.365 | −31.093 | 17.172 | 1.00 | 3.02 | C |
| ATOM | 462 | CG | ARG | A | 70 | 87.887 | −30.297 | 18.349 | 1.00 | 3.02 | C |
| ATOM | 463 | CD | ARG | A | 70 | 88.198 | −28.847 | 18.009 | 1.00 | 3.02 | C |
| ATOM | 464 | NE | ARG | A | 70 | 88.889 | −28.152 | 19.102 | 1.00 | 3.02 | N |
| ATOM | 465 | CZ | ARG | A | 70 | 88.916 | −26.828 | 19.276 | 1.00 | 3.02 | C |
| ATOM | 466 | NH1 | ARG | A | 70 | 88.296 | −26.007 | 18.422 | 1.00 | 3.02 | N |
| ATOM | 467 | NH2 | ARG | A | 70 | 89.588 | −26.312 | 20.299 | 1.00 | 3.02 | N |
| ATOM | 468 | C | ARG | A | 70 | 85.519 | −31.737 | 15.581 | 1.00 | 3.02 | C |
| ATOM | 469 | O | ARG | A | 70 | 85.215 | −31.208 | 14.503 | 1.00 | 3.02 | O |
| ATOM | 470 | N | ALA | A | 71 | 85.535 | −33.063 | 15.761 | 1.00 | 3.02 | N |
| ATOM | 471 | CA | ALA | A | 71 | 85.283 | −34.012 | 14.669 | 1.00 | 3.02 | C |
| ATOM | 472 | CB | ALA | A | 71 | 85.225 | −35.428 | 15.186 | 1.00 | 3.02 | C |
| ATOM | 473 | C | ALA | A | 71 | 83.992 | −33.685 | 13.957 | 1.00 | 3.02 | C |
| ATOM | 474 | O | ALA | A | 71 | 83.906 | −33.826 | 12.737 | 1.00 | 3.02 | O |
| ATOM | 475 | N | HIS | A | 72 | 82.990 | −33.275 | 14.742 | 1.00 | 3.02 | N |
| ATOM | 476 | CA | HIS | A | 72 | 81.731 | −32.718 | 14.231 | 1.00 | 3.02 | C |
| ATOM | 477 | CB | HIS | A | 72 | 80.700 | −32.618 | 15.343 | 1.00 | 3.02 | C |
| ATOM | 478 | CG | HIS | A | 72 | 79.939 | −33.875 | 15.558 | 1.00 | 3.02 | C |
| ATOM | 479 | ND1 | HIS | A | 72 | 80.230 | −34.757 | 16.573 | 1.00 | 3.02 | N |
| ATOM | 480 | CE1 | HIS | A | 72 | 79.399 | −35.779 | 16.511 | 1.00 | 3.02 | C |
| ATOM | 481 | NE2 | HIS | A | 72 | 78.585 | −35.595 | 15.487 | 1.00 | 3.02 | N |
| ATOM | 482 | CD2 | HIS | A | 72 | 78.909 | −34.413 | 14.870 | 1.00 | 3.02 | C |
| ATOM | 483 | C | HIS | A | 72 | 81.923 | −31.331 | 13.666 | 1.00 | 3.02 | C |
| ATOM | 484 | O | HIS | A | 72 | 81.310 | −30.996 | 12.652 | 1.00 | 3.02 | O |
| ATOM | 485 | N | ARG | A | 73 | 82.752 | −30.531 | 14.346 | 1.00 | 3.02 | N |
| ATOM | 486 | CA | ARG | A | 73 | 83.030 | −29.147 | 13.952 | 1.00 | 3.02 | C |
| ATOM | 487 | CB | ARG | A | 73 | 84.001 | −28.464 | 14.930 | 1.00 | 3.02 | C |
| ATOM | 488 | CG | ARG | A | 73 | 83.401 | −27.995 | 16.257 | 1.00 | 3.02 | C |
| ATOM | 489 | CD | ARG | A | 73 | 84.501 | −27.505 | 17.193 | 1.00 | 3.02 | C |
| ATOM | 490 | NE | ARG | A | 73 | 83.991 | −27.309 | 18.546 | 1.00 | 3.02 | N |
| ATOM | 491 | CZ | ARG | A | 73 | 84.634 | −26.672 | 19.526 | 1.00 | 3.02 | C |
| ATOM | 492 | NH1 | ARG | A | 73 | 85.840 | −26.142 | 19.334 | 1.00 | 3.02 | N |
| ATOM | 493 | NH2 | ARG | A | 73 | 84.050 | −26.556 | 20.717 | 1.00 | 3.02 | N |
| ATOM | 494 | C | ARG | A | 73 | 83.615 | −29.096 | 12.551 | 1.00 | 3.02 | C |
| ATOM | 495 | O | ARG | A | 73 | 83.096 | −28.389 | 11.677 | 1.00 | 3.02 | O |
| ATOM | 496 | N | ASN | A | 74 | 84.691 | −29.858 | 12.352 | 1.00 | 3.02 | N |
| ATOM | 497 | CA | ASN | A | 74 | 85.418 | −29.881 | 11.088 | 1.00 | 3.02 | C |
| ATOM | 498 | CB | ASN | A | 74 | 86.586 | −30.865 | 11.181 | 1.00 | 3.02 | C |
| ATOM | 499 | CG | ASN | A | 74 | 87.690 | −30.390 | 12.116 | 1.00 | 3.02 | C |
| ATOM | 500 | OD1 | ASN | A | 74 | 88.178 | −29.273 | 11.996 | 1.00 | 3.02 | O |
| ATOM | 501 | ND2 | ASN | A | 74 | 88.097 | −31.247 | 13.043 | 1.00 | 3.02 | N |
| ATOM | 502 | C | ASN | A | 74 | 84.519 | −30.225 | 9.897 | 1.00 | 3.02 | C |
| ATOM | 503 | O | ASN | A | 74 | 84.397 | −29.454 | 8.925 | 1.00 | 3.02 | O |
| ATOM | 504 | N | ASP | A | 75 | 83.859 | −31.371 | 10.014 | 1.00 | 3.02 | N |
| ATOM | 505 | CA | ASP | A | 75 | 82.974 | −31.876 | 8.983 | 1.00 | 3.02 | C |
| ATOM | 506 | CB | ASP | A | 75 | 82.594 | −33.330 | 9.284 | 1.00 | 3.02 | C |
| ATOM | 507 | CG | ASP | A | 75 | 83.714 | −34.119 | 9.954 | 1.00 | 3.02 | C |
| ATOM | 508 | OD1 | ASP | A | 75 | 84.837 | −33.596 | 10.082 | 1.00 | 3.02 | O |
| ATOM | 509 | OD2 | ASP | A | 75 | 83.466 | −35.275 | 10.365 | 1.00 | 3.02 | O |
| ATOM | 510 | C | ASP | A | 75 | 81.711 | −31.004 | 8.821 | 1.00 | 3.02 | C |
| ATOM | 511 | O | ASP | A | 75 | 81.084 | −30.999 | 7.748 | 1.00 | 3.02 | O |
| ATOM | 512 | N | MET | A | 76 | 81.326 | −30.291 | 9.882 | 1.00 | 3.02 | N |
| ATOM | 513 | CA | MET | A | 76 | 80.267 | −29.294 | 9.778 | 1.00 | 3.02 | C |
| ATOM | 514 | CB | MET | A | 76 | 79.855 | −28.779 | 11.157 | 1.00 | 3.02 | C |
| ATOM | 515 | CG | MET | A | 76 | 78.685 | −29.526 | 11.760 | 1.00 | 3.02 | C |
| ATOM | 516 | SD | MET | A | 76 | 78.081 | −28.728 | 13.253 | 1.00 | 3.02 | S |
| ATOM | 517 | CE | MET | A | 76 | 76.644 | −29.725 | 13.602 | 1.00 | 3.02 | C |
| ATOM | 518 | C | MET | A | 76 | 80.722 | −28.130 | 8.906 | 1.00 | 3.02 | C |
| ATOM | 519 | O | MET | A | 76 | 80.125 | −27.855 | 7.860 | 1.00 | 3.02 | O |
| ATOM | 520 | N | GLU | A | 77 | 81.812 | −27.484 | 9.329 | 1.00 | 3.02 | N |
| ATOM | 521 | CA | GLU | A | 77 | 82.260 | −26.194 | 8.757 | 1.00 | 3.02 | C |
| ATOM | 522 | CB | GLU | A | 77 | 83.462 | −25.614 | 9.523 | 1.00 | 3.02 | C |
| ATOM | 523 | CG | GLU | A | 77 | 83.115 | −25.214 | 10.941 | 1.00 | 3.02 | C |
| ATOM | 524 | CD | GLU | A | 77 | 84.224 | −24.505 | 11.665 | 1.00 | 3.02 | C |
| ATOM | 525 | OE1 | GLU | A | 77 | 85.197 | −25.168 | 12.085 | 1.00 | 3.02 | O |
| ATOM | 526 | OE2 | GLU | A | 77 | 84.090 | −23.281 | 11.849 | 1.00 | 3.02 | O |
| ATOM | 527 | C | GLU | A | 77 | 82.535 | −26.206 | 7.256 | 1.00 | 3.02 | C |
| ATOM | 528 | O | GLU | A | 77 | 81.954 | −25.390 | 6.524 | 1.00 | 3.02 | O |
| ATOM | 529 | N | THR | A | 78 | 83.405 | −27.118 | 6.803 | 1.00 | 3.02 | N |
| ATOM | 530 | CA | THR | A | 78 | 83.695 | −27.224 | 5.370 | 1.00 | 3.02 | C |
| ATOM | 531 | CB | THR | A | 78 | 84.798 | −28.247 | 5.041 | 1.00 | 3.02 | C |
| ATOM | 532 | OG1 | THR | A | 78 | 86.011 | −27.541 | 4.720 | 1.00 | 3.02 | O |
| ATOM | 533 | CG2 | THR | A | 78 | 84.401 | −29.121 | 3.836 | 1.00 | 3.02 | C |
| ATOM | 534 | C | THR | A | 78 | 82.468 | −27.534 | 4.544 | 1.00 | 3.02 | C |
| ATOM | 535 | O | THR | A | 78 | 82.200 | −26.876 | 3.533 | 1.00 | 3.02 | O |
| ATOM | 536 | N | ILE | A | 79 | 81.710 | −28.519 | 5.002 | 1.00 | 3.02 | N |
| ATOM | 537 | CA | ILE | A | 79 | 80.718 | −29.157 | 4.152 | 1.00 | 3.02 | C |
| ATOM | 538 | CB | ILE | A | 79 | 80.007 | −30.348 | 4.871 | 1.00 | 3.02 | C |
| ATOM | 539 | CG1 | ILE | A | 79 | 79.900 | −31.535 | 3.921 | 1.00 | 3.02 | C |
| ATOM | 540 | CD1 | ILE | A | 79 | 80.424 | −32.814 | 4.485 | 1.00 | 3.02 | C |
| ATOM | 541 | CG2 | ILE | A | 79 | 78.697 | −29.936 | 5.487 | 1.00 | 3.02 | C |
| ATOM | 542 | C | ILE | A | 79 | 79.821 | −28.149 | 3.416 | 1.00 | 3.02 | C |
| ATOM | 543 | O | ILE | A | 79 | 79.835 | −28.120 | 2.197 | 1.00 | 3.02 | O |
| ATOM | 544 | N | TYR | A | 80 | 79.126 | −27.275 | 4.130 | 1.00 | 3.02 | N |
| ATOM | 545 | CA | TYR | A | 80 | 78.161 | −26.359 | 3.491 | 1.00 | 3.02 | C |
| ATOM | 546 | CB | TYR | A | 80 | 77.422 | −25.533 | 4.546 | 1.00 | 3.02 | C |
| ATOM | 547 | CG | TYR | A | 80 | 77.337 | −26.225 | 5.896 | 1.00 | 3.02 | C |
| ATOM | 548 | CD1 | TYR | A | 80 | 78.078 | −25.755 | 6.988 | 1.00 | 3.02 | C |
| ATOM | 549 | CE1 | TYR | A | 80 | 77.998 | −26.387 | 8.231 | 1.00 | 3.02 | C |
| ATOM | 550 | CZ | TYR | A | 80 | 77.182 | −27.502 | 8.379 | 1.00 | 3.02 | C |
| ATOM | 551 | OH | TYR | A | 80 | 77.096 | −28.129 | 9.596 | 1.00 | 3.02 | O |
| ATOM | 552 | CE2 | TYR | A | 80 | 76.450 | −27.982 | 7.308 | 1.00 | 3.02 | C |
| ATOM | 553 | CD2 | TYR | A | 80 | 76.525 | −27.352 | 6.080 | 1.00 | 3.02 | C |
| ATOM | 554 | C | TYR | A | 80 | 78.722 | −25.480 | 2.344 | 1.00 | 3.02 | C |
| ATOM | 555 | O | TYR | A | 80 | 78.198 | −25.564 | 1.227 | 1.00 | 3.02 | O |
| ATOM | 556 | N | PRO | A | 81 | 79.753 | −24.617 | 2.620 | 1.00 | 3.02 | N |
| ATOM | 557 | CA | PRO | A | 81 | 80.571 | −23.941 | 1.582 | 1.00 | 3.02 | C |
| ATOM | 558 | CB | PRO | A | 81 | 81.869 | −23.554 | 2.333 | 1.00 | 3.02 | C |
| ATOM | 559 | CG | PRO | A | 81 | 81.644 | −23.857 | 3.780 | 1.00 | 3.02 | C |
| ATOM | 560 | CD | PRO | A | 81 | 80.168 | −24.151 | 3.961 | 1.00 | 3.02 | C |
| ATOM | 561 | C | PRO | A | 81 | 80.933 | −24.806 | 0.374 | 1.00 | 3.02 | C |
| ATOM | 562 | O | PRO | A | 81 | 80.897 | −24.339 | −0.758 | 1.00 | 3.02 | O |
| ATOM | 563 | N | PHE | A | 82 | 81.291 | −26.057 | 0.618 | 1.00 | 3.02 | N |
| ATOM | 564 | CA | PHE | A | 82 | 81.599 | −26.964 | −0.473 | 1.00 | 3.02 | C |
| ATOM | 565 | CB | PHE | A | 82 | 82.564 | −28.030 | −0.013 | 1.00 | 3.02 | C |
| ATOM | 566 | CG | PHE | A | 82 | 83.159 | −28.778 | −1.131 | 1.00 | 3.02 | C |
| ATOM | 567 | CD1 | PHE | A | 82 | 84.074 | −28.163 | −1.969 | 1.00 | 3.02 | C |
| ATOM | 568 | CE1 | PHE | A | 82 | 84.625 | −28.833 | −3.013 | 1.00 | 3.02 | C |
| ATOM | 569 | CZ | PHE | A | 82 | 84.245 | −30.145 | −3.269 | 1.00 | 3.02 | C |
| ATOM | 570 | CE2 | PHE | A | 82 | 83.305 | −30.776 | −2.460 | 1.00 | 3.02 | C |
| ATOM | 571 | CD2 | PHE | A | 82 | 82.760 | −30.081 | −1.397 | 1.00 | 3.02 | C |
| ATOM | 572 | C | PHE | A | 82 | 80.369 | −27.641 | −1.086 | 1.00 | 3.02 | C |
| ATOM | 573 | O | PHE | A | 82 | 80.408 | −28.140 | −2.221 | 1.00 | 3.02 | O |
| ATOM | 574 | N | LEU | A | 83 | 79.284 | −27.686 | −0.320 | 1.00 | 3.02 | N |
| ATOM | 575 | CA | LEU | A | 83 | 78.045 | −28.231 | −0.823 | 1.00 | 3.02 | C |
| ATOM | 576 | CB | LEU | A | 83 | 77.105 | −28.667 | 0.308 | 1.00 | 3.02 | C |
| ATOM | 577 | CG | LEU | A | 83 | 77.687 | −29.440 | 1.501 | 1.00 | 3.02 | C |
| ATOM | 578 | CD1 | LEU | A | 83 | 76.614 | −30.179 | 2.321 | 1.00 | 3.02 | C |
| ATOM | 579 | CD2 | LEU | A | 83 | 78.847 | −30.367 | 1.097 | 1.00 | 3.02 | C |
| ATOM | 580 | C | LEU | A | 83 | 77.414 | −27.165 | −1.690 | 1.00 | 3.02 | C |
| ATOM | 581 | O | LEU | A | 83 | 77.633 | −27.140 | −2.900 | 1.00 | 3.02 | O |
| ATOM | 582 | N | PHE | A | 84 | 76.703 | −26.241 | −1.057 | 1.00 | 3.02 | N |
| ATOM | 583 | CA | PHE | A | 84 | 75.857 | −25.274 | −1.761 | 1.00 | 3.02 | C |
| ATOM | 584 | CB | PHE | A | 84 | 75.000 | −24.495 | −0.739 | 1.00 | 3.02 | C |
| ATOM | 585 | CG | PHE | A | 84 | 74.888 | −23.006 | −0.993 | 1.00 | 3.02 | C |
| ATOM | 586 | CD1 | PHE | A | 84 | 73.817 | −22.300 | −0.478 | 1.00 | 3.02 | C |
| ATOM | 587 | CE1 | PHE | A | 84 | 73.701 | −20.945 | −0.685 | 1.00 | 3.02 | C |
| ATOM | 588 | CZ | PHE | A | 84 | 74.667 | −20.287 | −1.415 | 1.00 | 3.02 | C |
| ATOM | 589 | CE2 | PHE | A | 84 | 75.734 | −20.979 | −1.933 | 1.00 | 3.02 | C |
| ATOM | 590 | CD2 | PHE | A | 84 | 75.843 | −22.314 | −1.712 | 1.00 | 3.02 | C |
| ATOM | 591 | C | PHE | A | 84 | 76.557 | −24.372 | −2.788 | 1.00 | 3.02 | C |
| ATOM | 592 | O | PHE | A | 84 | 76.043 | −24.223 | −3.901 | 1.00 | 3.02 | O |
| ATOM | 593 | N | LEU | A | 85 | 77.709 | −23.784 | −2.434 | 1.00 | 3.02 | N |
| ATOM | 594 | CA | LEU | A | 85 | 78.415 | −22.858 | −3.348 | 1.00 | 3.02 | C |
| ATOM | 595 | CB | LEU | A | 85 | 79.734 | −22.345 | −2.762 | 1.00 | 3.02 | C |
| ATOM | 596 | CG | LEU | A | 85 | 79.689 | −21.503 | −1.486 | 1.00 | 3.02 | C |
| ATOM | 597 | CD1 | LEU | A | 85 | 81.085 | −21.208 | −1.015 | 1.00 | 3.02 | C |
| ATOM | 598 | CD2 | LEU | A | 85 | 78.956 | −20.205 | −1.718 | 1.00 | 3.02 | C |
| ATOM | 599 | C | LEU | A | 85 | 78.654 | −23.568 | −4.657 | 1.00 | 3.02 | C |
| ATOM | 600 | O | LEU | A | 85 | 78.272 | −23.066 | −5.721 | 1.00 | 3.02 | O |
| ATOM | 601 | N | GLY | A | 86 | 79.259 | −24.752 | −4.552 | 1.00 | 3.02 | N |
| ATOM | 602 | CA | GLY | A | 86 | 79.351 | −25.678 | −5.659 | 1.00 | 3.02 | C |
| ATOM | 603 | C | GLY | A | 86 | 77.979 | −25.965 | −6.226 | 1.00 | 3.02 | C |
| ATOM | 604 | O | GLY | A | 86 | 77.724 | −25.648 | −7.380 | 1.00 | 3.02 | O |
| ATOM | 605 | N | PHE | A | 87 | 77.086 | −26.522 | −5.402 | 1.00 | 3.02 | N |
| ATOM | 606 | CA | PHE | A | 87 | 75.812 | −27.099 | −5.863 | 1.00 | 3.02 | C |
| ATOM | 607 | CB | PHE | A | 87 | 75.005 | −27.642 | −4.680 | 1.00 | 3.02 | C |
| ATOM | 608 | CG | PHE | A | 87 | 75.402 | −29.037 | −4.262 | 1.00 | 3.02 | C |
| ATOM | 609 | CD1 | PHE | A | 87 | 76.337 | −29.235 | −3.270 | 1.00 | 3.02 | C |
| ATOM | 610 | CE1 | PHE | A | 87 | 76.715 | −30.511 | −2.872 | 1.00 | 3.02 | C |
| ATOM | 611 | CZ | PHE | A | 87 | 76.155 | −31.609 | −3.468 | 1.00 | 3.02 | C |
| ATOM | 612 | CE2 | PHE | A | 87 | 75.213 | −31.430 | −4.469 | 1.00 | 3.02 | C |
| ATOM | 613 | CD2 | PHE | A | 87 | 74.837 | −30.149 | −4.860 | 1.00 | 3.02 | C |
| ATOM | 614 | C | PHE | A | 87 | 74.952 | −26.184 | −6.729 | 1.00 | 3.02 | C |
| ATOM | 615 | O | PHE | A | 87 | 74.519 | −26.573 | −7.813 | 1.00 | 3.02 | O |
| ATOM | 616 | N | VAL | A | 88 | 74.720 | −24.966 | −6.251 | 1.00 | 3.02 | N |
| ATOM | 617 | CA | VAL | A | 88 | 73.943 | −23.965 | −7.002 | 1.00 | 3.02 | C |
| ATOM | 618 | CB | VAL | A | 88 | 73.456 | −22.813 | −6.107 | 1.00 | 3.02 | C |
| ATOM | 619 | CG1 | VAL | A | 88 | 72.151 | −22.222 | −6.661 | 1.00 | 3.02 | C |
| ATOM | 620 | CG2 | VAL | A | 88 | 73.303 | −23.279 | −4.672 | 1.00 | 3.02 | C |
| ATOM | 621 | C | VAL | A | 88 | 74.732 | −23.370 | −8.173 | 1.00 | 3.02 | C |
| ATOM | 622 | O | VAL | A | 88 | 74.215 | −22.560 | −8.950 | 1.00 | 3.02 | O |
| ATOM | 623 | N | TYR | A | 89 | 75.991 | −23.765 | −8.270 | 1.00 | 3.02 | N |
| ATOM | 624 | CA | TYR | A | 89 | 76.809 | −23.499 | −9.430 | 1.00 | 3.02 | C |
| ATOM | 625 | CB | TYR | A | 89 | 78.214 | −23.123 | −8.957 | 1.00 | 3.02 | C |
| ATOM | 626 | CG | TYR | A | 89 | 79.372 | −23.876 | −9.570 | 1.00 | 3.02 | C |
| ATOM | 627 | CD1 | TYR | A | 89 | 80.095 | −23.326 | −10.616 | 1.00 | 3.02 | C |
| ATOM | 628 | CE1 | TYR | A | 89 | 81.170 | −24.000 | −11.170 | 1.00 | 3.02 | C |
| ATOM | 629 | CZ | TYR | A | 89 | 81.528 | −25.232 | −10.675 | 1.00 | 3.02 | C |
| ATOM | 630 | OH | TYR | A | 89 | 82.592 | −25.892 | −11.238 | 1.00 | 3.02 | O |
| ATOM | 631 | CE2 | TYR | A | 89 | 80.826 | −25.795 | −9.628 | 1.00 | 3.02 | C |
| ATOM | 632 | CD2 | TYR | A | 89 | 79.766 | −25.114 | −9.077 | 1.00 | 3.02 | C |
| ATOM | 633 | C | TYR | A | 89 | 76.805 | −24.736 | −10.318 | 1.00 | 3.02 | C |
| ATOM | 634 | O | TYR | A | 89 | 77.010 | −24.646 | −11.527 | 1.00 | 3.02 | O |
| ATOM | 635 | N | SER | A | 90 | 76.520 | −25.877 | −9.698 | 1.00 | 3.02 | N |
| ATOM | 636 | CA | SER | A | 90 | 76.677 | −27.177 | −10.334 | 1.00 | 3.02 | C |
| ATOM | 637 | CB | SER | A | 90 | 76.903 | −28.252 | −9.274 | 1.00 | 3.02 | C |
| ATOM | 638 | OG | SER | A | 90 | 75.698 | −28.755 | −8.736 | 1.00 | 3.02 | O |
| ATOM | 639 | C | SER | A | 90 | 75.504 | −27.529 | −11.234 | 1.00 | 3.02 | C |
| ATOM | 640 | O | SER | A | 90 | 75.644 | −28.338 | −12.157 | 1.00 | 3.02 | O |
| ATOM | 641 | N | PHE | A | 91 | 74.358 | −26.912 | −10.958 | 1.00 | 3.02 | N |
| ATOM | 642 | CA | PHE | A | 91 | 73.178 | −27.057 | −11.800 | 1.00 | 3.02 | C |
| ATOM | 643 | CB | PHE | A | 91 | 71.941 | −27.374 | −10.941 | 1.00 | 3.02 | C |
| ATOM | 644 | CG | PHE | A | 91 | 72.139 | −28.532 | −9.967 | 1.00 | 3.02 | C |
| ATOM | 645 | CD1 | PHE | A | 91 | 72.250 | −28.297 | −8.596 | 1.00 | 3.02 | C |
| ATOM | 646 | CE1 | PHE | A | 91 | 72.424 | −29.355 | −7.694 | 1.00 | 3.02 | C |
| ATOM | 647 | CZ | PHE | A | 91 | 72.495 | −30.659 | −8.162 | 1.00 | 3.02 | C |
| ATOM | 648 | CE2 | PHE | A | 91 | 72.384 | −30.909 | −9.529 | 1.00 | 3.02 | C |
| ATOM | 649 | CD2 | PHE | A | 91 | 72.206 | −29.850 | −10.423 | 1.00 | 3.02 | C |
| ATOM | 650 | C | PHE | A | 91 | 73.019 | −25.778 | −12.646 | 1.00 | 3.02 | C |
| ATOM | 651 | O | PHE | A | 91 | 72.066 | −24.998 | −12.479 | 1.00 | 3.02 | O |
| ATOM | 652 | N | LEU | A | 92 | 73.991 | −25.579 | −13.544 | 1.00 | 3.02 | N |
| ATOM | 653 | CA | LEU | A | 92 | 74.137 | −24.361 | −14.358 | 1.00 | 3.02 | C |
| ATOM | 654 | CB | LEU | A | 92 | 74.707 | −23.215 | −13.506 | 1.00 | 3.02 | C |
| ATOM | 655 | CG | LEU | A | 92 | 74.812 | −21.785 | −14.072 | 1.00 | 3.02 | C |
| ATOM | 656 | CD1 | LEU | A | 92 | 73.495 | −21.024 | −13.981 | 1.00 | 3.02 | C |
| ATOM | 657 | CD2 | LEU | A | 92 | 75.910 | −21.009 | −13.355 | 1.00 | 3.02 | C |
| ATOM | 658 | C | LEU | A | 92 | 75.025 | −24.584 | −15.597 | 1.00 | 3.02 | C |
| ATOM | 659 | O | LEU | A | 92 | 75.063 | −23.750 | −16.494 | 1.00 | 3.02 | O |
| ATOM | 660 | N | GLY | A | 93 | 75.741 | −25.706 | −15.637 | 1.00 | 3.02 | N |
| ATOM | 661 | CA | GLY | A | 93 | 76.571 | −26.085 | −16.792 | 1.00 | 3.02 | C |
| ATOM | 662 | C | GLY | A | 93 | 78.052 | −25.764 | −16.647 | 1.00 | 3.02 | C |
| ATOM | 663 | O | GLY | A | 93 | 78.674 | −25.244 | −17.582 | 1.00 | 3.02 | O |
| ATOM | 664 | N | PRO | A | 94 | 78.646 | −26.106 | −15.486 | 1.00 | 3.02 | N |
| ATOM | 665 | CA | PRO | A | 94 | 79.987 | −25.641 | −15.143 | 1.00 | 3.02 | C |
| ATOM | 666 | CB | PRO | A | 94 | 80.009 | −25.723 | −13.612 | 1.00 | 3.02 | C |
| ATOM | 667 | CG | PRO | A | 94 | 78.773 | −26.476 | −13.210 | 1.00 | 3.02 | C |
| ATOM | 668 | CD | PRO | A | 94 | 78.110 | −26.992 | −14.440 | 1.00 | 3.02 | C |
| ATOM | 669 | C | PRO | A | 94 | 81.113 | −26.481 | −15.754 | 1.00 | 3.02 | C |
| ATOM | 670 | O | PRO | A | 94 | 80.869 | −27.324 | −16.621 | 1.00 | 3.02 | O |
| ATOM | 671 | N | ASN | A | 95 | 82.340 | −26.252 | −15.300 | 1.00 | 3.02 | N |
| ATOM | 672 | CA | ASN | A | 95 | 83.496 | −26.852 | −15.936 | 1.00 | 3.02 | C |
| ATOM | 673 | CB | ASN | A | 95 | 84.511 | −25.774 | −16.325 | 1.00 | 3.02 | C |
| ATOM | 674 | CG | ASN | A | 95 | 84.090 | −24.982 | −17.560 | 1.00 | 3.02 | C |
| ATOM | 675 | OD1 | ASN | A | 95 | 83.321 | −25.461 | −18.400 | 1.00 | 3.02 | O |
| ATOM | 676 | ND2 | ASN | A | 95 | 84.605 | −23.762 | −17.675 | 1.00 | 3.02 | N |
| ATOM | 677 | C | ASN | A | 95 | 84.151 | −27.958 | −15.115 | 1.00 | 3.02 | C |
| ATOM | 678 | O | ASN | A | 95 | 84.090 | −27.927 | −13.880 | 1.00 | 3.02 | O |
| ATOM | 679 | N | PRO | A | 96 | 84.741 | −28.963 | −15.816 | 1.00 | 3.02 | N |
| ATOM | 680 | CA | PRO | A | 96 | 85.509 | −30.097 | −15.294 | 1.00 | 3.02 | C |
| ATOM | 681 | CB | PRO | A | 96 | 86.040 | −30.771 | −16.565 | 1.00 | 3.02 | C |
| ATOM | 682 | CG | PRO | A | 96 | 85.872 | −29.767 | −17.648 | 1.00 | 3.02 | C |
| ATOM | 683 | CD | PRO | A | 96 | 84.634 | −29.056 | −17.283 | 1.00 | 3.02 | C |
| ATOM | 684 | C | PRO | A | 96 | 86.664 | −29.698 | −14.374 | 1.00 | 3.02 | C |
| ATOM | 685 | O | PRO | A | 96 | 86.407 | −29.223 | −13.271 | 1.00 | 3.02 | O |
| ATOM | 686 | N | PHE | A | 97 | 87.910 | −29.888 | −14.819 | 1.00 | 3.02 | N |
| ATOM | 687 | CA | PHE | A | 97 | 89.114 | −29.679 | −13.990 | 1.00 | 3.02 | C |
| ATOM | 688 | CB | PHE | A | 97 | 90.199 | −28.937 | −14.770 | 1.00 | 3.02 | C |
| ATOM | 689 | CG | PHE | A | 97 | 90.337 | −29.360 | −16.201 | 1.00 | 3.02 | C |
| ATOM | 690 | CD1 | PHE | A | 97 | 89.580 | −28.754 | −17.190 | 1.00 | 3.02 | C |
| ATOM | 691 | CE1 | PHE | A | 97 | 89.719 | −29.119 | −18.509 | 1.00 | 3.02 | C |
| ATOM | 692 | CZ | PHE | A | 97 | 90.630 | −30.096 | −18.862 | 1.00 | 3.02 | C |
| ATOM | 693 | CE2 | PHE | A | 97 | 91.405 | −30.705 | −17.887 | 1.00 | 3.02 | C |
| ATOM | 694 | CD2 | PHE | A | 97 | 91.257 | −30.334 | −16.563 | 1.00 | 3.02 | C |
| ATOM | 695 | C | PHE | A | 97 | 88.828 | −28.877 | −12.731 | 1.00 | 3.02 | C |
| ATOM | 696 | O | PHE | A | 97 | 89.097 | −29.327 | −11.609 | 1.00 | 3.02 | O |
| ATOM | 697 | N | VAL | A | 98 | 88.292 | −27.676 | −12.976 | 1.00 | 3.02 | N |
| ATOM | 698 | CA | VAL | A | 98 | 87.843 | −26.687 | −11.990 | 1.00 | 3.02 | C |
| ATOM | 699 | CB | VAL | A | 98 | 86.920 | −25.636 | −12.696 | 1.00 | 3.02 | C |
| ATOM | 700 | CG1 | VAL | A | 98 | 86.203 | −24.749 | −11.698 | 1.00 | 3.02 | C |
| ATOM | 701 | CG2 | VAL | A | 98 | 87.716 | −24.794 | −13.693 | 1.00 | 3.02 | C |
| ATOM | 702 | C | VAL | A | 98 | 87.147 | −27.320 | −10.779 | 1.00 | 3.02 | C |
| ATOM | 703 | O | VAL | A | 98 | 87.584 | −27.161 | −9.637 | 1.00 | 3.02 | O |
| ATOM | 704 | N | ALA | A | 99 | 86.069 | −28.042 | −11.041 | 1.00 | 3.02 | N |
| ATOM | 705 | CA | ALA | A | 99 | 85.323 | −28.705 | −9.994 | 1.00 | 3.02 | C |
| ATOM | 706 | CB | ALA | A | 99 | 84.118 | −29.423 | −10.593 | 1.00 | 3.02 | C |
| ATOM | 707 | C | ALA | A | 99 | 86.206 | −29.679 | −9.209 | 1.00 | 3.02 | C |
| ATOM | 708 | O | ALA | A | 99 | 86.306 | −29.580 | −7.985 | 1.00 | 3.02 | O |
| ATOM | 709 | N | TRP | A | 100 | 86.846 | −30.604 | −9.931 | 1.00 | 3.02 | N |
| ATOM | 710 | CA | TRP | A | 100 | 87.661 | −31.691 | −9.359 | 1.00 | 3.02 | C |
| ATOM | 711 | CB | TRP | A | 100 | 88.379 | −32.466 | −10.461 | 1.00 | 3.02 | C |
| ATOM | 712 | CG | TRP | A | 100 | 87.510 | −33.183 | −11.409 | 1.00 | 3.02 | C |
| ATOM | 713 | CD1 | TRP | A | 100 | 87.093 | −32.743 | −12.628 | 1.00 | 3.02 | C |
| ATOM | 714 | NE1 | TRP | A | 100 | 86.317 | −33.696 | −13.233 | 1.00 | 3.02 | N |
| ATOM | 715 | CE2 | TRP | A | 100 | 86.226 | −34.788 | −12.407 | 1.00 | 3.02 | C |
| ATOM | 716 | CD2 | TRP | A | 100 | 86.979 | −34.497 | −11.248 | 1.00 | 3.02 | C |
| ATOM | 717 | CE3 | TRP | A | 100 | 87.059 | −35.461 | −10.231 | 1.00 | 3.02 | C |
| ATOM | 718 | CZ3 | TRP | A | 100 | 86.381 | −36.679 | −10.400 | 1.00 | 3.02 | C |
| ATOM | 719 | CH2 | TRP | A | 100 | 85.639 | −36.939 | −11.569 | 1.00 | 3.02 | C |
| ATOM | 720 | CZ2 | TRP | A | 100 | 85.547 | −36.011 | −12.581 | 1.00 | 3.02 | C |
| ATOM | 721 | C | TRP | A | 100 | 88.691 | −31.236 | −8.327 | 1.00 | 3.02 | C |
| ATOM | 722 | O | TRP | A | 100 | 88.879 | −31.922 | −7.316 | 1.00 | 3.02 | O |
| ATOM | 723 | N | MET | A | 101 | 89.354 | −30.101 | −8.599 | 1.00 | 3.02 | N |
| ATOM | 724 | CA | MET | A | 101 | 90.364 | −29.516 | −7.693 | 1.00 | 3.02 | C |
| ATOM | 725 | CB | MET | A | 101 | 91.137 | −28.363 | −8.350 | 1.00 | 3.02 | C |
| ATOM | 726 | CG | MET | A | 101 | 91.433 | −28.462 | −9.861 | 1.00 | 3.02 | C |
| ATOM | 727 | SD | MET | A | 101 | 91.700 | −26.792 | −10.592 | 1.00 | 3.02 | S |
| ATOM | 728 | CE | MET | A | 101 | 93.349 | −26.422 | −9.973 | 1.00 | 3.02 | C |
| ATOM | 729 | C | MET | A | 101 | 89.798 | −28.997 | −6.360 | 1.00 | 3.02 | C |
| ATOM | 730 | O | MET | A | 101 | 90.566 | −28.837 | −5.415 | 1.00 | 3.02 | O |
| ATOM | 731 | N | HIS | A | 102 | 88.488 | −28.704 | −6.299 | 1.00 | 3.02 | N |
| ATOM | 732 | CA | HIS | A | 102 | 87.812 | −28.300 | −5.050 | 1.00 | 3.02 | C |
| ATOM | 733 | CB | HIS | A | 102 | 86.415 | −27.720 | −5.299 | 1.00 | 3.02 | C |
| ATOM | 734 | CG | HIS | A | 102 | 86.381 | −26.515 | −6.179 | 1.00 | 3.02 | C |
| ATOM | 735 | ND1 | HIS | A | 102 | 87.307 | −25.500 | −6.096 | 1.00 | 3.02 | N |
| ATOM | 736 | CE1 | HIS | A | 102 | 87.009 | −24.562 | −6.977 | 1.00 | 3.02 | C |
| ATOM | 737 | NE2 | HIS | A | 102 | 85.913 | −24.926 | −7.617 | 1.00 | 3.02 | N |
| ATOM | 738 | CD2 | HIS | A | 102 | 85.497 | −26.141 | −7.132 | 1.00 | 3.02 | C |
| ATOM | 739 | C | HIS | A | 102 | 87.645 | −29.489 | −4.116 | 1.00 | 3.02 | C |
| ATOM | 740 | O | HIS | A | 102 | 87.759 | −29.361 | −2.908 | 1.00 | 3.02 | O |
| ATOM | 741 | N | PHE | A | 103 | 87.327 | −30.642 | −4.687 | 1.00 | 3.02 | N |
| ATOM | 742 | CA | PHE | A | 103 | 87.214 | −31.885 | −3.927 | 1.00 | 3.02 | C |
| ATOM | 743 | CB | PHE | A | 103 | 86.638 | −32.994 | −4.817 | 1.00 | 3.02 | C |
| ATOM | 744 | CG | PHE | A | 103 | 85.532 | −32.519 | −5.715 | 1.00 | 3.02 | C |
| ATOM | 745 | CD1 | PHE | A | 103 | 85.732 | −32.368 | −7.079 | 1.00 | 3.02 | C |
| ATOM | 746 | CE1 | PHE | A | 103 | 84.696 | −31.911 | −7.909 | 1.00 | 3.02 | C |
| ATOM | 747 | CZ | PHE | A | 103 | 83.471 | −31.588 | −7.369 | 1.00 | 3.02 | C |
| ATOM | 748 | CE2 | PHE | A | 103 | 83.264 | −31.723 | −6.005 | 1.00 | 3.02 | C |
| ATOM | 749 | CD2 | PHE | A | 103 | 84.290 | −32.187 | −5.189 | 1.00 | 3.02 | C |
| ATOM | 750 | C | PHE | A | 103 | 88.572 | −32.297 | −3.385 | 1.00 | 3.02 | C |
| ATOM | 751 | O | PHE | A | 103 | 88.654 | −32.994 | −2.375 | 1.00 | 3.02 | O |
| ATOM | 752 | N | LEU | A | 104 | 89.626 | −31.864 | −4.076 | 1.00 | 3.02 | N |
| ATOM | 753 | CA | LEU | A | 104 | 91.004 | −32.084 | −3.657 | 1.00 | 3.02 | C |
| ATOM | 754 | CB | LEU | A | 104 | 91.913 | −32.202 | −4.890 | 1.00 | 3.02 | C |
| ATOM | 755 | CG | LEU | A | 104 | 91.779 | −33.488 | −5.719 | 1.00 | 3.02 | C |
| ATOM | 756 | CD1 | LEU | A | 104 | 92.253 | −33.340 | −7.165 | 1.00 | 3.02 | C |
| ATOM | 757 | CD2 | LEU | A | 104 | 92.522 | −34.636 | −5.039 | 1.00 | 3.02 | C |
| ATOM | 758 | C | LEU | A | 104 | 91.484 | −30.995 | −2.668 | 1.00 | 3.02 | C |
| ATOM | 759 | O | LEU | A | 104 | 92.245 | −31.284 | −1.744 | 1.00 | 3.02 | O |
| ATOM | 760 | N | VAL | A | 105 | 91.025 | −29.754 | −2.843 | 1.00 | 3.02 | N |
| ATOM | 761 | CA | VAL | A | 105 | 91.338 | −28.696 | −1.870 | 1.00 | 3.02 | C |
| ATOM | 762 | CB | VAL | A | 105 | 91.285 | −27.249 | −2.443 | 1.00 | 3.02 | C |
| ATOM | 763 | CG1 | VAL | A | 105 | 92.139 | −26.332 | −1.579 | 1.00 | 3.02 | C |
| ATOM | 764 | CG2 | VAL | A | 105 | 91.797 | −27.190 | −3.873 | 1.00 | 3.02 | C |
| ATOM | 765 | C | VAL | A | 105 | 90.466 | −28.768 | −0.609 | 1.00 | 3.02 | C |
| ATOM | 766 | O | VAL | A | 105 | 90.933 | −28.466 | 0.489 | 1.00 | 3.02 | O |
| ATOM | 767 | N | PHE | A | 106 | 89.203 | −29.151 | −0.763 | 1.00 | 3.02 | N |
| ATOM | 768 | CA | PHE | A | 106 | 88.294 | −29.225 | 0.379 | 1.00 | 3.02 | C |
| ATOM | 769 | CB | PHE | A | 106 | 86.833 | −29.120 | −0.060 | 1.00 | 3.02 | C |
| ATOM | 770 | CG | PHE | A | 106 | 86.331 | −27.716 | −0.105 | 1.00 | 3.02 | C |
| ATOM | 771 | CD1 | PHE | A | 106 | 86.670 | −26.874 | −1.158 | 1.00 | 3.02 | C |
| ATOM | 772 | CE1 | PHE | A | 106 | 86.205 | −25.561 | −1.193 | 1.00 | 3.02 | C |
| ATOM | 773 | CZ | PHE | A | 106 | 85.407 | −25.066 | −0.155 | 1.00 | 3.02 | C |
| ATOM | 774 | CE2 | PHE | A | 106 | 85.074 | −25.891 | 0.906 | 1.00 | 3.02 | C |
| ATOM | 775 | CD2 | PHE | A | 106 | 85.542 | −27.217 | 0.928 | 1.00 | 3.02 | C |
| ATOM | 776 | C | PHE | A | 106 | 88.519 | −30.459 | 1.232 | 1.00 | 3.02 | C |
| ATOM | 777 | O | PHE | A | 106 | 88.843 | −30.342 | 2.411 | 1.00 | 3.02 | O |
| ATOM | 778 | N | LEU | A | 107 | 88.344 | −31.635 | 0.633 | 1.00 | 3.02 | N |
| ATOM | 779 | CA | LEU | A | 107 | 88.523 | −32.904 | 1.339 | 1.00 | 3.02 | C |
| ATOM | 780 | CB | LEU | A | 107 | 88.129 | −34.082 | 0.450 | 1.00 | 3.02 | C |
| ATOM | 781 | CG | LEU | A | 107 | 86.622 | −34.245 | 0.299 | 1.00 | 3.02 | C |
| ATOM | 782 | CD1 | LEU | A | 107 | 86.290 | −34.794 | −1.067 | 1.00 | 3.02 | C |
| ATOM | 783 | CD2 | LEU | A | 107 | 86.083 | −35.145 | 1.399 | 1.00 | 3.02 | C |
| ATOM | 784 | C | LEU | A | 107 | 89.942 | −33.082 | 1.886 | 1.00 | 3.02 | C |
| ATOM | 785 | O | LEU | A | 107 | 90.112 | −33.574 | 3.002 | 1.00 | 3.02 | O |
| ATOM | 786 | N | VAL | A | 108 | 90.954 | −32.673 | 1.122 | 1.00 | 3.02 | N |
| ATOM | 787 | CA | VAL | A | 108 | 92.328 | −32.733 | 1.628 | 1.00 | 3.02 | C |
| ATOM | 788 | CB | VAL | A | 108 | 93.383 | −32.908 | 0.494 | 1.00 | 3.02 | C |
| ATOM | 789 | CG1 | VAL | A | 108 | 94.792 | −33.041 | 1.080 | 1.00 | 3.02 | C |
| ATOM | 790 | CG2 | VAL | A | 108 | 93.060 | −34.145 | −0.366 | 1.00 | 3.02 | C |
| ATOM | 791 | C | VAL | A | 108 | 92.637 | −31.521 | 2.525 | 1.00 | 3.02 | C |
| ATOM | 792 | O | VAL | A | 108 | 93.366 | −31.637 | 3.520 | 1.00 | 3.02 | O |
| ATOM | 793 | N | GLY | A | 109 | 92.066 | −30.370 | 2.174 | 1.00 | 3.02 | N |
| ATOM | 794 | CA | GLY | A | 109 | 92.165 | −29.169 | 3.006 | 1.00 | 3.02 | C |
| ATOM | 795 | C | GLY | A | 109 | 91.515 | −29.398 | 4.358 | 1.00 | 3.02 | C |
| ATOM | 796 | O | GLY | A | 109 | 92.030 | −28.959 | 5.394 | 1.00 | 3.02 | O |
| ATOM | 797 | N | ARG | A | 110 | 90.384 | −30.089 | 4.366 | 1.00 | 3.02 | N |
| ATOM | 798 | CA | ARG | A | 110 | 89.730 | −30.384 | 5.632 | 1.00 | 3.02 | C |
| ATOM | 799 | CB | ARG | A | 110 | 88.220 | −30.635 | 5.459 | 1.00 | 3.02 | C |
| ATOM | 800 | CG | ARG | A | 110 | 87.423 | −30.258 | 6.689 | 1.00 | 3.02 | C |
| ATOM | 801 | CD | ARG | A | 110 | 87.629 | −28.775 | 6.990 | 1.00 | 3.02 | C |
| ATOM | 802 | NE | ARG | A | 110 | 87.038 | −28.347 | 8.255 | 1.00 | 3.02 | N |
| ATOM | 803 | CZ | ARG | A | 110 | 86.574 | −27.126 | 8.489 | 1.00 | 3.02 | C |
| ATOM | 804 | NH1 | ARG | A | 110 | 86.613 | −26.198 | 7.534 | 1.00 | 3.02 | N |
| ATOM | 805 | NH2 | ARG | A | 110 | 86.059 | −26.848 | 9.677 | 1.00 | 3.02 | N |
| ATOM | 806 | C | ARG | A | 110 | 90.400 | −31.508 | 6.438 | 1.00 | 3.02 | C |
| ATOM | 807 | O | ARG | A | 110 | 90.612 | −31.342 | 7.629 | 1.00 | 3.02 | O |
| ATOM | 808 | N | VAL | A | 111 | 90.730 | −32.634 | 5.802 | 1.00 | 3.02 | N |
| ATOM | 809 | CA | VAL | A | 111 | 91.124 | −33.848 | 6.546 | 1.00 | 3.02 | C |
| ATOM | 810 | CB | VAL | A | 111 | 91.089 | −35.142 | 5.658 | 1.00 | 3.02 | C |
| ATOM | 811 | CG1 | VAL | A | 111 | 91.870 | −36.312 | 6.301 | 1.00 | 3.02 | C |
| ATOM | 812 | CG2 | VAL | A | 111 | 89.643 | −35.561 | 5.346 | 1.00 | 3.02 | C |
| ATOM | 813 | C | VAL | A | 111 | 92.459 | −33.692 | 7.276 | 1.00 | 3.02 | C |
| ATOM | 814 | O | VAL | A | 111 | 92.603 | −34.132 | 8.421 | 1.00 | 3.02 | O |
| ATOM | 815 | N | ALA | A | 112 | 93.420 | −33.059 | 6.605 | 1.00 | 3.02 | N |
| ATOM | 816 | CA | ALA | A | 112 | 94.648 | −32.600 | 7.250 | 1.00 | 3.02 | C |
| ATOM | 817 | CB | ALA | A | 112 | 95.635 | −32.142 | 6.213 | 1.00 | 3.02 | C |
| ATOM | 818 | C | ALA | A | 112 | 94.335 | −31.478 | 8.269 | 1.00 | 3.02 | C |
| ATOM | 819 | O | ALA | A | 112 | 95.086 | −31.279 | 9.252 | 1.00 | 3.02 | O |
| ATOM | 820 | N | HIS | A | 113 | 93.234 | −30.750 | 8.051 | 1.00 | 3.02 | N |
| ATOM | 821 | CA | HIS | A | 113 | 92.693 | −29.877 | 9.093 | 1.00 | 3.02 | C |
| ATOM | 822 | CB | HIS | A | 113 | 91.674 | −28.891 | 8.537 | 1.00 | 3.02 | C |
| ATOM | 823 | CG | HIS | A | 113 | 90.985 | −28.116 | 9.604 | 1.00 | 3.02 | C |
| ATOM | 824 | ND1 | HIS | A | 113 | 91.656 | −27.272 | 10.465 | 1.00 | 3.02 | N |
| ATOM | 825 | CE1 | HIS | A | 113 | 90.795 | −26.739 | 11.312 | 1.00 | 3.02 | C |
| ATOM | 826 | NE2 | HIS | A | 113 | 89.595 | −27.218 | 11.040 | 1.00 | 3.02 | N |
| ATOM | 827 | CD2 | HIS | A | 113 | 89.690 | −28.087 | 9.981 | 1.00 | 3.02 | C |
| ATOM | 828 | C | HIS | A | 113 | 92.063 | −30.641 | 10.265 | 1.00 | 3.02 | C |
| ATOM | 829 | O | HIS | A | 113 | 92.381 | −30.366 | 11.433 | 1.00 | 3.02 | O |
| ATOM | 830 | N | THR | A | 114 | 91.173 | −31.586 | 9.946 | 1.00 | 3.02 | N |
| ATOM | 831 | CA | THR | A | 114 | 90.450 | −32.366 | 10.953 | 1.00 | 3.02 | C |
| ATOM | 832 | CB | THR | A | 114 | 89.320 | −33.259 | 10.317 | 1.00 | 3.02 | C |
| ATOM | 833 | OG1 | THR | A | 114 | 88.409 | −32.450 | 9.555 | 1.00 | 3.02 | O |
| ATOM | 834 | CG2 | THR | A | 114 | 88.528 | −34.022 | 11.381 | 1.00 | 3.02 | C |
| ATOM | 835 | C | THR | A | 114 | 91.418 | −33.220 | 11.765 | 1.00 | 3.02 | C |
| ATOM | 836 | O | THR | A | 114 | 91.443 | −33.149 | 12.989 | 1.00 | 3.02 | O |
| ATOM | 837 | N | VAL | A | 115 | 92.221 | −34.017 | 11.081 | 1.00 | 3.02 | N |
| ATOM | 838 | CA | VAL | A | 115 | 93.081 | −34.980 | 11.766 | 1.00 | 3.02 | C |
| ATOM | 839 | CB | VAL | A | 115 | 93.667 | −36.015 | 10.763 | 1.00 | 3.02 | C |
| ATOM | 840 | CG1 | VAL | A | 115 | 94.878 | −36.731 | 11.332 | 1.00 | 3.02 | C |
| ATOM | 841 | CG2 | VAL | A | 115 | 92.580 | −37.009 | 10.362 | 1.00 | 3.02 | C |
| ATOM | 842 | C | VAL | A | 115 | 94.160 | −34.331 | 12.666 | 1.00 | 3.02 | C |
| ATOM | 843 | O | VAL | A | 115 | 94.461 | −34.834 | 13.762 | 1.00 | 3.02 | O |
| ATOM | 844 | N | ALA | A | 116 | 94.728 | −33.214 | 12.218 | 1.00 | 3.02 | N |
| ATOM | 845 | CA | ALA | A | 116 | 95.750 | −32.540 | 13.002 | 1.00 | 3.02 | C |
| ATOM | 846 | CB | ALA | A | 116 | 96.390 | −31.438 | 12.198 | 1.00 | 3.02 | C |
| ATOM | 847 | C | ALA | A | 116 | 95.147 | −32.005 | 14.299 | 1.00 | 3.02 | C |
| ATOM | 848 | O | ALA | A | 116 | 95.764 | −32.087 | 15.363 | 1.00 | 3.02 | O |
| ATOM | 849 | N | TYR | A | 117 | 93.919 | −31.494 | 14.197 | 1.00 | 3.02 | N |
| ATOM | 850 | CA | TYR | A | 117 | 93.184 | −30.893 | 15.322 | 1.00 | 3.02 | C |
| ATOM | 851 | CB | TYR | A | 117 | 92.157 | −29.888 | 14.780 | 1.00 | 3.02 | C |
| ATOM | 852 | CG | TYR | A | 117 | 91.810 | −28.745 | 15.719 | 1.00 | 3.02 | C |
| ATOM | 853 | CD1 | TYR | A | 117 | 92.365 | −28.653 | 16.993 | 1.00 | 3.02 | C |
| ATOM | 854 | CE1 | TYR | A | 117 | 92.027 | −27.620 | 17.840 | 1.00 | 3.02 | C |
| ATOM | 855 | CZ | TYR | A | 117 | 91.138 | −26.658 | 17.421 | 1.00 | 3.02 | C |
| ATOM | 856 | OH | TYR | A | 117 | 90.802 | −25.621 | 18.258 | 1.00 | 3.02 | O |
| ATOM | 857 | CE2 | TYR | A | 117 | 90.570 | −26.729 | 16.176 | 1.00 | 3.02 | C |
| ATOM | 858 | CD2 | TYR | A | 117 | 90.900 | −27.768 | 15.336 | 1.00 | 3.02 | C |
| ATOM | 859 | C | TYR | A | 117 | 92.481 | −31.908 | 16.267 | 1.00 | 3.02 | C |
| ATOM | 860 | O | TYR | A | 117 | 91.841 | −31.524 | 17.260 | 1.00 | 3.02 | O |
| ATOM | 861 | N | LEU | A | 118 | 92.581 | −33.195 | 15.960 | 1.00 | 3.02 | N |
| ATOM | 862 | CA | LEU | A | 118 | 92.011 | −34.195 | 16.840 | 1.00 | 3.02 | C |
| ATOM | 863 | CB | LEU | A | 118 | 91.313 | −35.294 | 16.032 | 1.00 | 3.02 | C |
| ATOM | 864 | CG | LEU | A | 118 | 90.000 | −34.990 | 15.284 | 1.00 | 3.02 | C |
| ATOM | 865 | CD1 | LEU | A | 118 | 89.726 | −36.093 | 14.283 | 1.00 | 3.02 | C |
| ATOM | 866 | CD2 | LEU | A | 118 | 88.799 | −34.820 | 16.216 | 1.00 | 3.02 | C |
| ATOM | 867 | C | LEU | A | 118 | 93.071 | −34.767 | 17.781 | 1.00 | 3.02 | C |
| ATOM | 868 | O | LEU | A | 118 | 92.862 | −34.807 | 18.992 | 1.00 | 3.02 | O |
| ATOM | 869 | N | GLY | A | 119 | 94.204 | −35.190 | 17.215 | 1.00 | 3.02 | N |
| ATOM | 870 | CA | GLY | A | 119 | 95.325 | −35.749 | 17.980 | 1.00 | 3.02 | C |
| ATOM | 871 | C | GLY | A | 119 | 95.945 | −34.733 | 18.920 | 1.00 | 3.02 | C |
| ATOM | 872 | O | GLY | A | 119 | 95.473 | −34.562 | 20.056 | 1.00 | 3.02 | O |
| ATOM | 873 | N | LYS | A | 120 | 96.992 | −34.046 | 18.448 | 1.00 | 3.02 | N |
| ATOM | 874 | CA | LYS | A | 120 | 97.614 | −32.979 | 19.235 | 1.00 | 3.02 | C |
| ATOM | 875 | CB | LYS | A | 120 | 98.639 | −32.171 | 18.429 | 1.00 | 3.02 | C |
| ATOM | 876 | CG | LYS | A | 120 | 100.098 | −32.524 | 18.700 | 1.00 | 3.02 | C |
| ATOM | 877 | CD | LYS | A | 120 | 101.018 | −31.456 | 18.070 | 1.00 | 3.02 | C |
| ATOM | 878 | CE | LYS | A | 120 | 102.526 | −31.758 | 18.182 | 1.00 | 3.02 | C |
| ATOM | 879 | NZ | LYS | A | 120 | 103.337 | −30.917 | 17.239 | 1.00 | 3.02 | N |
| ATOM | 880 | C | LYS | A | 120 | 96.468 | −32.104 | 19.701 | 1.00 | 3.02 | C |
| ATOM | 881 | O | LYS | A | 120 | 95.602 | −31.733 | 18.895 | 1.00 | 3.02 | O |
| ATOM | 882 | N | LEU | A | 121 | 96.444 | −31.826 | 21.008 | 1.00 | 3.02 | N |
| ATOM | 883 | CA | LEU | A | 121 | 95.315 | −31.141 | 21.662 | 1.00 | 3.02 | C |
| ATOM | 884 | CB | LEU | A | 121 | 95.428 | −31.249 | 23.193 | 1.00 | 3.02 | C |
| ATOM | 885 | CG | LEU | A | 121 | 95.799 | −32.558 | 23.893 | 1.00 | 3.02 | C |
| ATOM | 886 | CD1 | LEU | A | 121 | 95.558 | −32.386 | 25.373 | 1.00 | 3.02 | C |
| ATOM | 887 | CD2 | LEU | A | 121 | 94.995 | −33.742 | 23.349 | 1.00 | 3.02 | C |
| ATOM | 888 | C | LEU | A | 121 | 95.209 | −29.670 | 21.267 | 1.00 | 3.02 | C |
| ATOM | 889 | O | LEU | A | 121 | 94.113 | −29.097 | 21.246 | 1.00 | 3.02 | O |
| ATOM | 890 | N | ARG | A | 122 | 96.367 | −29.099 | 20.944 | 1.00 | 3.02 | N |
| ATOM | 891 | CA | ARG | A | 122 | 96.544 | −27.666 | 20.800 | 1.00 | 3.02 | C |
| ATOM | 892 | CB | ARG | A | 122 | 98.034 | −27.338 | 20.825 | 1.00 | 3.02 | C |
| ATOM | 893 | CG | ARG | A | 122 | 98.652 | −27.474 | 22.203 | 1.00 | 3.02 | C |
| ATOM | 894 | CD | ARG | A | 122 | 100.060 | −26.983 | 22.201 | 1.00 | 3.02 | C |
| ATOM | 895 | NE | ARG | A | 122 | 100.335 | −26.181 | 23.387 | 1.00 | 3.02 | N |
| ATOM | 896 | CZ | ARG | A | 122 | 100.799 | −26.652 | 24.546 | 1.00 | 3.02 | C |
| ATOM | 897 | NH1 | ARG | A | 122 | 101.042 | −27.949 | 24.714 | 1.00 | 3.02 | N |
| ATOM | 898 | NH2 | ARG | A | 122 | 101.020 | −25.816 | 25.548 | 1.00 | 3.02 | N |
| ATOM | 899 | C | ARG | A | 122 | 95.891 | −27.094 | 19.558 | 1.00 | 3.02 | C |
| ATOM | 900 | O | ARG | A | 122 | 96.136 | −27.579 | 18.455 | 1.00 | 3.02 | O |
| ATOM | 901 | N | ALA | A | 123 | 95.066 | −26.059 | 19.757 | 1.00 | 3.02 | N |
| ATOM | 902 | CA | ALA | A | 123 | 94.323 | −25.406 | 18.666 | 1.00 | 3.02 | C |
| ATOM | 903 | CB | ALA | A | 123 | 93.312 | −24.411 | 19.203 | 1.00 | 3.02 | C |
| ATOM | 904 | C | ALA | A | 123 | 95.206 | −24.743 | 17.616 | 1.00 | 3.02 | C |
| ATOM | 905 | O | ALA | A | 123 | 94.823 | −24.703 | 16.445 | 1.00 | 3.02 | O |
| ATOM | 906 | N | PRO | A | 124 | 96.387 | −24.212 | 18.026 | 1.00 | 3.02 | N |
| ATOM | 907 | CA | PRO | A | 124 | 97.310 | −23.666 | 17.039 | 1.00 | 3.02 | C |
| ATOM | 908 | CB | PRO | A | 124 | 98.569 | −23.360 | 17.860 | 1.00 | 3.02 | C |
| ATOM | 909 | CG | PRO | A | 124 | 98.403 | −24.100 | 19.127 | 1.00 | 3.02 | C |
| ATOM | 910 | CD | PRO | A | 124 | 96.939 | −24.058 | 19.383 | 1.00 | 3.02 | C |
| ATOM | 911 | C | PRO | A | 124 | 97.649 | −24.644 | 15.927 | 1.00 | 3.02 | C |
| ATOM | 912 | O | PRO | A | 124 | 97.854 | −24.205 | 14.802 | 1.00 | 3.02 | O |
| ATOM | 913 | N | ILE | A | 125 | 97.708 | −25.948 | 16.229 | 1.00 | 3.02 | N |
| ATOM | 914 | CA | ILE | A | 125 | 97.944 | −26.962 | 15.175 | 1.00 | 3.02 | C |
| ATOM | 915 | CB | ILE | A | 125 | 97.992 | −28.426 | 15.704 | 1.00 | 3.02 | C |
| ATOM | 916 | CG1 | ILE | A | 125 | 98.901 | −29.270 | 14.790 | 1.00 | 3.02 | C |
| ATOM | 917 | CD1 | ILE | A | 125 | 98.796 | −30.823 | 14.965 | 1.00 | 3.02 | C |
| ATOM | 918 | CG2 | ILE | A | 125 | 96.573 | −29.001 | 15.867 | 1.00 | 3.02 | C |
| ATOM | 919 | C | ILE | A | 125 | 96.929 | −26.833 | 14.016 | 1.00 | 3.02 | C |
| ATOM | 920 | O | ILE | A | 125 | 97.264 | −27.127 | 12.850 | 1.00 | 3.02 | O |
| ATOM | 921 | N | ARG | A | 126 | 95.709 | −26.385 | 14.337 | 1.00 | 3.02 | N |
| ATOM | 922 | CA | ARG | A | 126 | 94.687 | −26.155 | 13.324 | 1.00 | 3.02 | C |
| ATOM | 923 | CB | ARG | A | 126 | 93.308 | −25.930 | 13.959 | 1.00 | 3.02 | C |
| ATOM | 924 | CG | ARG | A | 126 | 92.940 | −24.503 | 14.368 | 1.00 | 3.02 | C |
| ATOM | 925 | CD | ARG | A | 126 | 91.960 | −23.869 | 13.376 | 1.00 | 3.02 | C |
| ATOM | 926 | NE | ARG | A | 126 | 91.182 | −22.755 | 13.924 | 1.00 | 3.02 | N |
| ATOM | 927 | CZ | ARG | A | 126 | 90.393 | −21.959 | 13.196 | 1.00 | 3.02 | C |
| ATOM | 928 | NH1 | ARG | A | 126 | 90.265 | −22.146 | 11.882 | 1.00 | 3.02 | N |
| ATOM | 929 | NH2 | ARG | A | 126 | 89.715 | −20.977 | 13.781 | 1.00 | 3.02 | N |
| ATOM | 930 | C | ARG | A | 126 | 95.114 | −25.017 | 12.406 | 1.00 | 3.02 | C |
| ATOM | 931 | O | ARG | A | 126 | 94.391 | −24.625 | 11.501 | 1.00 | 3.02 | O |
| ATOM | 932 | N | SER | A | 127 | 96.329 | −24.537 | 12.638 | 1.00 | 3.02 | N |
| ATOM | 933 | CA | SER | A | 127 | 96.960 | −23.485 | 11.857 | 1.00 | 3.02 | C |
| ATOM | 934 | CB | SER | A | 127 | 98.412 | −23.355 | 12.288 | 1.00 | 3.02 | C |
| ATOM | 935 | OG | SER | A | 127 | 99.029 | −24.631 | 12.312 | 1.00 | 3.02 | O |
| ATOM | 936 | C | SER | A | 127 | 96.930 | −23.642 | 10.343 | 1.00 | 3.02 | C |
| ATOM | 937 | O | SER | A | 127 | 96.325 | −22.828 | 9.647 | 1.00 | 3.02 | O |
| ATOM | 938 | N | VAL | A | 128 | 97.601 | −24.672 | 9.833 | 1.00 | 3.02 | N |
| ATOM | 939 | CA | VAL | A | 128 | 98.033 | −24.633 | 8.438 | 1.00 | 3.02 | C |
| ATOM | 940 | CB | VAL | A | 128 | 99.304 | −25.453 | 8.171 | 1.00 | 3.02 | C |
| ATOM | 941 | CG1 | VAL | A | 128 | 100.266 | −24.645 | 7.293 | 1.00 | 3.02 | C |
| ATOM | 942 | CG2 | VAL | A | 128 | 99.985 | −25.830 | 9.475 | 1.00 | 3.02 | C |
| ATOM | 943 | C | VAL | A | 128 | 96.953 | −25.009 | 7.460 | 1.00 | 3.02 | C |
| ATOM | 944 | O | VAL | A | 128 | 96.863 | −24.417 | 6.380 | 1.00 | 3.02 | O |
| ATOM | 945 | N | THR | A | 129 | 96.143 | −25.991 | 7.848 | 1.00 | 3.02 | N |
| ATOM | 946 | CA | THR | A | 129 | 95.068 | −26.518 | 6.995 | 1.00 | 3.02 | C |
| ATOM | 947 | CB | THR | A | 129 | 94.715 | −27.991 | 7.325 | 1.00 | 3.02 | C |
| ATOM | 948 | OG1 | THR | A | 129 | 94.548 | −28.145 | 8.751 | 1.00 | 3.02 | O |
| ATOM | 949 | CG2 | THR | A | 129 | 95.801 | −28.941 | 6.836 | 1.00 | 3.02 | C |
| ATOM | 950 | C | THR | A | 129 | 93.796 | −25.686 | 7.062 | 1.00 | 3.02 | C |
| ATOM | 951 | O | THR | A | 129 | 93.037 | −25.630 | 6.093 | 1.00 | 3.02 | O |
| ATOM | 952 | N | TYR | A | 130 | 93.576 | −25.038 | 8.204 | 1.00 | 3.02 | N |
| ATOM | 953 | CA | TYR | A | 130 | 92.411 | −24.182 | 8.386 | 1.00 | 3.02 | C |
| ATOM | 954 | CB | TYR | A | 130 | 92.329 | −23.590 | 9.810 | 1.00 | 3.02 | C |
| ATOM | 955 | CG | TYR | A | 130 | 92.907 | −22.187 | 10.014 | 1.00 | 3.02 | C |
| ATOM | 956 | CD1 | TYR | A | 130 | 92.293 | −21.067 | 9.467 | 1.00 | 3.02 | C |
| ATOM | 957 | CE1 | TYR | A | 130 | 92.821 | −19.793 | 9.657 | 1.00 | 3.02 | C |
| ATOM | 958 | CZ | TYR | A | 130 | 93.954 | −19.627 | 10.421 | 1.00 | 3.02 | C |
| ATOM | 959 | OH | TYR | A | 130 | 94.470 | −18.364 | 10.613 | 1.00 | 3.02 | O |
| ATOM | 960 | CE2 | TYR | A | 130 | 94.563 | −20.722 | 10.998 | 1.00 | 3.02 | C |
| ATOM | 961 | CD2 | TYR | A | 130 | 94.033 | −21.985 | 10.803 | 1.00 | 3.02 | C |
| ATOM | 962 | C | TYR | A | 130 | 92.358 | −23.086 | 7.336 | 1.00 | 3.02 | C |
| ATOM | 963 | O | TYR | A | 130 | 91.277 | −22.613 | 6.982 | 1.00 | 3.02 | O |
| ATOM | 964 | N | THR | A | 131 | 93.522 | −22.678 | 6.848 | 1.00 | 3.02 | N |
| ATOM | 965 | CA | THR | A | 131 | 93.558 | −21.704 | 5.782 | 1.00 | 3.02 | C |
| ATOM | 966 | CB | THR | A | 131 | 94.798 | −20.805 | 5.867 | 1.00 | 3.02 | C |
| ATOM | 967 | OG1 | THR | A | 131 | 94.984 | −20.403 | 7.232 | 1.00 | 3.02 | O |
| ATOM | 968 | CG2 | THR | A | 131 | 94.616 | −19.550 | 5.010 | 1.00 | 3.02 | C |
| ATOM | 969 | C | THR | A | 131 | 93.392 | −22.397 | 4.429 | 1.00 | 3.02 | C |
| ATOM | 970 | O | THR | A | 131 | 92.759 | −21.838 | 3.533 | 1.00 | 3.02 | O |
| ATOM | 971 | N | LEU | A | 132 | 93.911 | −23.617 | 4.290 | 1.00 | 3.02 | N |
| ATOM | 972 | CA | LEU | A | 132 | 93.662 | −24.396 | 3.081 | 1.00 | 3.02 | C |
| ATOM | 973 | CB | LEU | A | 132 | 94.548 | −25.656 | 3.022 | 1.00 | 3.02 | C |
| ATOM | 974 | CG | LEU | A | 132 | 96.082 | −25.444 | 2.865 | 1.00 | 3.02 | C |
| ATOM | 975 | CD1 | LEU | A | 132 | 96.894 | −26.772 | 2.867 | 1.00 | 3.02 | C |
| ATOM | 976 | CD2 | LEU | A | 132 | 96.477 | −24.574 | 1.638 | 1.00 | 3.02 | C |
| ATOM | 977 | C | LEU | A | 132 | 92.150 | −24.681 | 2.873 | 1.00 | 3.02 | C |
| ATOM | 978 | O | LEU | A | 132 | 91.705 | −24.837 | 1.733 | 1.00 | 3.02 | O |
| ATOM | 979 | N | ALA | A | 133 | 91.365 | −24.726 | 3.954 | 1.00 | 3.02 | N |
| ATOM | 980 | CA | ALA | A | 133 | 89.911 | −24.827 | 3.856 | 1.00 | 3.02 | C |
| ATOM | 981 | CB | ALA | A | 133 | 89.348 | −25.308 | 5.163 | 1.00 | 3.02 | C |
| ATOM | 982 | C | ALA | A | 133 | 89.275 | −23.487 | 3.467 | 1.00 | 3.02 | C |
| ATOM | 983 | O | ALA | A | 133 | 88.365 | −23.449 | 2.643 | 1.00 | 3.02 | O |
| ATOM | 984 | N | GLN | A | 134 | 89.755 | −22.392 | 4.066 | 1.00 | 3.02 | N |
| ATOM | 985 | CA | GLN | A | 134 | 89.269 | −21.017 | 3.789 | 1.00 | 3.02 | C |
| ATOM | 986 | CB | GLN | A | 134 | 89.967 | −20.013 | 4.715 | 1.00 | 3.02 | C |
| ATOM | 987 | CG | GLN | A | 134 | 89.631 | −20.138 | 6.185 | 1.00 | 3.02 | C |
| ATOM | 988 | CD | GLN | A | 134 | 90.198 | −19.003 | 7.007 | 1.00 | 3.02 | C |
| ATOM | 989 | OE1 | GLN | A | 134 | 90.695 | −18.009 | 6.472 | 1.00 | 3.02 | O |
| ATOM | 990 | NE2 | GLN | A | 134 | 90.122 | −19.143 | 8.320 | 1.00 | 3.02 | N |
| ATOM | 991 | C | GLN | A | 134 | 89.471 | −20.525 | 2.349 | 1.00 | 3.02 | C |
| ATOM | 992 | O | GLN | A | 134 | 88.702 | −19.711 | 1.845 | 1.00 | 3.02 | O |
| ATOM | 993 | N | LEU | A | 135 | 90.525 | −21.020 | 1.707 | 1.00 | 3.02 | N |
| ATOM | 994 | CA | LEU | A | 135 | 90.999 | −20.503 | 0.416 | 1.00 | 3.02 | C |
| ATOM | 995 | CB | LEU | A | 135 | 92.534 | −20.457 | 0.408 | 1.00 | 3.02 | C |
| ATOM | 996 | CG | LEU | A | 135 | 93.171 | −19.561 | 1.472 | 1.00 | 3.02 | C |
| ATOM | 997 | CD1 | LEU | A | 135 | 94.502 | −20.086 | 1.854 | 1.00 | 3.02 | C |
| ATOM | 998 | CD2 | LEU | A | 135 | 93.288 | −18.130 | 1.019 | 1.00 | 3.02 | C |
| ATOM | 999 | C | LEU | A | 135 | 90.473 | −21.154 | −0.895 | 1.00 | 3.02 | C |
| ATOM | 1000 | O | LEU | A | 135 | 90.718 | −20.597 | −1.984 | 1.00 | 3.02 | O |
| ATOM | 1001 | N | PRO | A | 136 | 89.818 | −22.346 | −0.819 | 1.00 | 3.02 | N |
| ATOM | 1002 | CA | PRO | A | 136 | 89.038 | −22.768 | −1.985 | 1.00 | 3.02 | C |
| ATOM | 1003 | CB | PRO | A | 136 | 89.018 | −24.293 | −1.872 | 1.00 | 3.02 | C |
| ATOM | 1004 | CG | PRO | A | 136 | 89.317 | −24.598 | −0.439 | 1.00 | 3.02 | C |
| ATOM | 1005 | CD | PRO | A | 136 | 89.786 | −23.360 | 0.251 | 1.00 | 3.02 | C |
| ATOM | 1006 | C | PRO | A | 136 | 87.625 | −22.179 | −1.967 | 1.00 | 3.02 | C |
| ATOM | 1007 | O | PRO | A | 136 | 86.851 | −22.402 | −2.897 | 1.00 | 3.02 | O |
| ATOM | 1008 | N | CYS | A | 137 | 87.317 | −21.431 | −0.909 | 1.00 | 3.02 | N |
| ATOM | 1009 | CA | CYS | A | 137 | 86.096 | −20.625 | −0.843 | 1.00 | 3.02 | C |
| ATOM | 1010 | CB | CYS | A | 137 | 85.644 | −20.410 | 0.607 | 1.00 | 3.02 | C |
| ATOM | 1011 | SG | CYS | A | 137 | 85.160 | −21.922 | 1.479 | 1.00 | 3.02 | S |
| ATOM | 1012 | C | CYS | A | 137 | 86.275 | −19.280 | −1.557 | 1.00 | 3.02 | C |
| ATOM | 1013 | O | CYS | A | 137 | 85.289 | −18.637 | −1.934 | 1.00 | 3.02 | O |
| ATOM | 1014 | N | ALA | A | 138 | 87.533 | −18.864 | −1.706 | 1.00 | 3.02 | N |
| ATOM | 1015 | CA | ALA | A | 138 | 87.911 | −17.777 | −2.595 | 1.00 | 3.02 | C |
| ATOM | 1016 | CB | ALA | A | 138 | 89.255 | −17.199 | −2.185 | 1.00 | 3.02 | C |
| ATOM | 1017 | C | ALA | A | 138 | 87.962 | −18.293 | −4.038 | 1.00 | 3.02 | C |
| ATOM | 1018 | O | ALA | A | 138 | 87.720 | −17.523 | −4.972 | 1.00 | 3.02 | O |
| ATOM | 1019 | N | SER | A | 139 | 88.273 | −19.587 | −4.210 | 1.00 | 3.02 | N |
| ATOM | 1020 | CA | SER | A | 139 | 88.323 | −20.229 | −5.536 | 1.00 | 3.02 | C |
| ATOM | 1021 | CB | SER | A | 139 | 89.310 | −21.410 | −5.570 | 1.00 | 3.02 | C |
| ATOM | 1022 | OG | SER | A | 139 | 88.694 | −22.636 | −5.211 | 1.00 | 3.02 | O |
| ATOM | 1023 | C | SER | A | 139 | 86.936 | −20.640 | −6.062 | 1.00 | 3.02 | C |
| ATOM | 1024 | O | SER | A | 139 | 86.589 | −20.323 | −7.205 | 1.00 | 3.02 | O |
| ATOM | 1025 | N | MET | A | 140 | 86.148 | −21.325 | −5.229 | 1.00 | 3.02 | N |
| ATOM | 1026 | CA | MET | A | 140 | 84.759 | −21.690 | −5.580 | 1.00 | 3.02 | C |
| ATOM | 1027 | CB | MET | A | 140 | 84.115 | −22.515 | −4.465 | 1.00 | 3.02 | C |
| ATOM | 1028 | CG | MET | A | 140 | 84.605 | −23.924 | −4.351 | 1.00 | 3.02 | C |
| ATOM | 1029 | SD | MET | A | 140 | 83.446 | −24.886 | −3.370 | 1.00 | 3.02 | S |
| ATOM | 1030 | CE | MET | A | 140 | 82.255 | −25.384 | −4.634 | 1.00 | 3.02 | C |
| ATOM | 1031 | C | MET | A | 140 | 83.827 | −20.504 | −5.886 | 1.00 | 3.02 | C |
| ATOM | 1032 | O | MET | A | 140 | 83.313 | −20.374 | −6.997 | 1.00 | 3.02 | O |
| ATOM | 1033 | N | ALA | A | 141 | 83.609 | −19.655 | −4.888 | 1.00 | 3.02 | N |
| ATOM | 1034 | CA | ALA | A | 141 | 82.583 | −18.625 | −4.952 | 1.00 | 3.02 | C |
| ATOM | 1035 | CB | ALA | A | 141 | 82.629 | −17.761 | −3.707 | 1.00 | 3.02 | C |
| ATOM | 1036 | C | ALA | A | 141 | 82.606 | −17.757 | −6.221 | 1.00 | 3.02 | C |
| ATOM | 1037 | O | ALA | A | 141 | 81.550 | −17.507 | −6.815 | 1.00 | 3.02 | O |
| ATOM | 1038 | N | LEU | A | 142 | 83.797 | −17.310 | −6.627 | 1.00 | 3.02 | N |
| ATOM | 1039 | CA | LEU | A | 142 | 83.972 | −16.475 | −7.823 | 1.00 | 3.02 | C |
| ATOM | 1040 | CB | LEU | A | 142 | 85.403 | −15.929 | −7.867 | 1.00 | 3.02 | C |
| ATOM | 1041 | CG | LEU | A | 142 | 85.804 | −14.897 | −6.803 | 1.00 | 3.02 | C |
| ATOM | 1042 | CD1 | LEU | A | 142 | 86.999 | −15.349 | −5.999 | 1.00 | 3.02 | C |
| ATOM | 1043 | CD2 | LEU | A | 142 | 86.068 | −13.515 | −7.418 | 1.00 | 3.02 | C |
| ATOM | 1044 | C | LEU | A | 142 | 83.655 | −17.239 | −9.120 | 1.00 | 3.02 | C |
| ATOM | 1045 | O | LEU | A | 142 | 83.037 | −16.692 | −10.030 | 1.00 | 3.02 | O |
| ATOM | 1046 | N | GLN | A | 143 | 84.074 | −18.508 | −9.184 | 1.00 | 3.02 | N |
| ATOM | 1047 | CA | GLN | A | 143 | 83.911 | −19.357 | −10.383 | 1.00 | 3.02 | C |
| ATOM | 1048 | CB | GLN | A | 143 | 84.614 | −20.697 | −10.215 | 1.00 | 3.02 | C |
| ATOM | 1049 | CG | GLN | A | 143 | 86.109 | −20.666 | −10.384 | 1.00 | 3.02 | C |
| ATOM | 1050 | CD | GLN | A | 143 | 86.739 | −21.928 | −9.840 | 1.00 | 3.02 | C |
| ATOM | 1051 | OE1 | GLN | A | 143 | 86.105 | −22.683 | −9.101 | 1.00 | 3.02 | O |
| ATOM | 1052 | NE2 | GLN | A | 143 | 87.992 | −22.170 | −10.199 | 1.00 | 3.02 | N |
| ATOM | 1053 | C | GLN | A | 143 | 82.459 | −19.601 | −10.714 | 1.00 | 3.02 | C |
| ATOM | 1054 | O | GLN | A | 143 | 82.106 | −19.835 | −11.878 | 1.00 | 3.02 | O |
| ATOM | 1055 | N | ILE | A | 144 | 81.635 | −19.571 | −9.672 | 1.00 | 3.02 | N |
| ATOM | 1056 | CA | ILE | A | 144 | 80.199 | −19.444 | −9.831 | 1.00 | 3.02 | C |
| ATOM | 1057 | CB | ILE | A | 144 | 79.466 | −19.514 | −8.467 | 1.00 | 3.02 | C |
| ATOM | 1058 | CG1 | ILE | A | 144 | 79.858 | −20.781 | −7.708 | 1.00 | 3.02 | C |
| ATOM | 1059 | CD1 | ILE | A | 144 | 79.703 | −20.658 | −6.225 | 1.00 | 3.02 | C |
| ATOM | 1060 | CG2 | ILE | A | 144 | 77.949 | −19.458 | −8.676 | 1.00 | 3.02 | C |
| ATOM | 1061 | C | ILE | A | 144 | 79.937 | −18.088 | −10.504 | 1.00 | 3.02 | C |
| ATOM | 1062 | O | ILE | A | 144 | 79.505 | −18.045 | −11.658 | 1.00 | 3.02 | O |
| ATOM | 1063 | N | LEU | A | 145 | 80.268 | −17.001 | −9.799 | 1.00 | 3.02 | N |
| ATOM | 1064 | CA | LEU | A | 145 | 79.825 | −15.639 | −10.152 | 1.00 | 3.02 | C |
| ATOM | 1065 | CB | LEU | A | 145 | 80.461 | −14.577 | −9.243 | 1.00 | 3.02 | C |
| ATOM | 1066 | CG | LEU | A | 145 | 80.445 | −14.634 | −7.711 | 1.00 | 3.02 | C |
| ATOM | 1067 | CD1 | LEU | A | 145 | 80.740 | −13.242 | −7.175 | 1.00 | 3.02 | C |
| ATOM | 1068 | CD2 | LEU | A | 145 | 79.118 | −15.098 | −7.165 | 1.00 | 3.02 | C |
| ATOM | 1069 | C | LEU | A | 145 | 80.022 | −15.202 | −11.602 | 1.00 | 3.02 | C |
| ATOM | 1070 | O | LEU | A | 145 | 79.171 | −14.523 | −12.162 | 1.00 | 3.02 | O |
| ATOM | 1071 | N | TRP | A | 146 | 81.154 | −15.560 | −12.190 | 1.00 | 3.02 | N |
| ATOM | 1072 | CA | TRP | A | 146 | 81.479 | −15.134 | −13.543 | 1.00 | 3.02 | C |
| ATOM | 1073 | CB | TRP | A | 146 | 82.988 | −15.209 | −13.779 | 1.00 | 3.02 | C |
| ATOM | 1074 | CG | TRP | A | 146 | 83.825 | −14.500 | −12.746 | 1.00 | 3.02 | C |
| ATOM | 1075 | CD1 | TRP | A | 146 | 84.663 | −15.072 | −11.827 | 1.00 | 3.02 | C |
| ATOM | 1076 | NE1 | TRP | A | 146 | 85.251 | −14.100 | −11.060 | 1.00 | 3.02 | N |
| ATOM | 1077 | CE2 | TRP | A | 146 | 84.805 | −12.871 | −11.474 | 1.00 | 3.02 | C |
| ATOM | 1078 | CD2 | TRP | A | 146 | 83.905 | −13.085 | −12.536 | 1.00 | 3.02 | C |
| ATOM | 1079 | CE3 | TRP | A | 146 | 83.298 | −11.974 | −13.144 | 1.00 | 3.02 | C |
| ATOM | 1080 | CZ3 | TRP | A | 146 | 83.601 | −10.694 | −12.675 | 1.00 | 3.02 | C |
| ATOM | 1081 | CH2 | TRP | A | 146 | 84.505 | −10.510 | −11.615 | 1.00 | 3.02 | C |
| ATOM | 1082 | CZ2 | TRP | A | 146 | 85.117 | −11.585 | −11.003 | 1.00 | 3.02 | C |
| ATOM | 1083 | C | TRP | A | 146 | 80.765 | −16.015 | −14.557 | 1.00 | 3.02 | C |
| ATOM | 1084 | O | TRP | A | 146 | 80.474 | −15.584 | −15.672 | 1.00 | 3.02 | O |
| ATOM | 1085 | N | GLU | A | 147 | 80.496 | −17.259 | −14.170 | 1.00 | 3.02 | N |
| ATOM | 1086 | CA | GLU | A | 147 | 79.848 | −18.203 | −15.064 | 1.00 | 3.02 | C |
| ATOM | 1087 | CB | GLU | A | 147 | 80.091 | −19.651 | −14.621 | 1.00 | 3.02 | C |
| ATOM | 1088 | CG | GLU | A | 147 | 79.986 | −20.680 | −15.750 | 1.00 | 3.02 | C |
| ATOM | 1089 | CD | GLU | A | 147 | 80.206 | −22.107 | −15.269 | 1.00 | 3.02 | C |
| ATOM | 1090 | OE1 | GLU | A | 147 | 81.118 | −22.353 | −14.446 | 1.00 | 3.02 | O |
| ATOM | 1091 | OE2 | GLU | A | 147 | 79.464 | −22.992 | −15.726 | 1.00 | 3.02 | O |
| ATOM | 1092 | C | GLU | A | 147 | 78.361 | −17.878 | −15.163 | 1.00 | 3.02 | C |
| ATOM | 1093 | O | GLU | A | 147 | 77.896 | −17.470 | −16.222 | 1.00 | 3.02 | O |
| ATOM | 1094 | N | ALA | A | 148 | 77.632 | −18.035 | −14.056 | 1.00 | 3.02 | N |
| ATOM | 1095 | CA | ALA | A | 148 | 76.210 | −17.657 | −13.979 | 1.00 | 3.02 | C |
| ATOM | 1096 | CB | ALA | A | 148 | 75.729 | −17.668 | −12.535 | 1.00 | 3.02 | C |
| ATOM | 1097 | C | ALA | A | 148 | 75.913 | −16.309 | −14.643 | 1.00 | 3.02 | C |
| ATOM | 1098 | O | ALA | A | 148 | 74.946 | −16.195 | −15.388 | 1.00 | 3.02 | O |
| ATOM | 1099 | N | ALA | A | 149 | 76.759 | −15.310 | −14.390 | 1.00 | 3.02 | N |
| ATOM | 1100 | CA | ALA | A | 149 | 76.688 | −14.018 | −15.084 | 1.00 | 3.02 | C |
| ATOM | 1101 | CB | ALA | A | 149 | 77.641 | −13.008 | −14.435 | 1.00 | 3.02 | C |
| ATOM | 1102 | C | ALA | A | 149 | 76.975 | −14.125 | −16.589 | 1.00 | 3.02 | C |
| ATOM | 1103 | O | ALA | A | 149 | 76.548 | −13.260 | −17.372 | 1.00 | 3.02 | O |
| ATOM | 1104 | N | ARG | A | 150 | 77.718 | −15.170 | −16.971 | 1.00 | 3.02 | N |
| ATOM | 1105 | CA | ARG | A | 150 | 78.026 | −15.481 | −18.373 | 1.00 | 3.02 | C |
| ATOM | 1106 | CB | ARG | A | 150 | 79.277 | −16.363 | −18.469 | 1.00 | 3.02 | C |
| ATOM | 1107 | CG | ARG | A | 150 | 79.580 | −16.867 | −19.879 | 1.00 | 3.02 | C |
| ATOM | 1108 | CD | ARG | A | 150 | 80.705 | −17.887 | −19.907 | 1.00 | 3.02 | C |
| ATOM | 1109 | NE | ARG | A | 150 | 81.437 | −17.829 | −21.170 | 1.00 | 3.02 | N |
| ATOM | 1110 | CZ | ARG | A | 150 | 82.180 | −18.815 | −21.655 | 1.00 | 3.02 | C |
| ATOM | 1111 | NH1 | ARG | A | 150 | 82.287 | −19.964 | −20.994 | 1.00 | 3.02 | N |
| ATOM | 1112 | NH2 | ARG | A | 150 | 82.794 | −18.655 | −22.817 | 1.00 | 3.02 | N |
| ATOM | 1113 | C | ARG | A | 150 | 76.850 | −16.178 | −19.060 | 1.00 | 3.02 | C |
| ATOM | 1114 | O | ARG | A | 150 | 76.632 | −16.003 | −20.263 | 1.00 | 3.02 | O |
| ATOM | 1115 | N | HIS | A | 151 | 76.105 | −16.960 | −18.283 | 1.00 | 3.02 | N |
| ATOM | 1116 | CA | HIS | A | 151 | 74.955 | −17.678 | −18.784 | 1.00 | 3.02 | C |
| ATOM | 1117 | CB | HIS | A | 151 | 74.533 | −18.744 | −17.764 | 1.00 | 3.02 | C |
| ATOM | 1118 | CG | HIS | A | 151 | 75.437 | −19.941 | −17.737 | 1.00 | 3.02 | C |
| ATOM | 1119 | ND1 | HIS | A | 151 | 75.195 | −21.077 | −18.483 | 1.00 | 3.02 | N |
| ATOM | 1120 | CE1 | HIS | A | 151 | 76.163 | −21.952 | −18.272 | 1.00 | 3.02 | C |
| ATOM | 1121 | NE2 | HIS | A | 151 | 77.021 | −21.428 | −17.411 | 1.00 | 3.02 | N |
| ATOM | 1122 | CD2 | HIS | A | 151 | 76.591 | −20.172 | −17.062 | 1.00 | 3.02 | C |
| ATOM | 1123 | C | HIS | A | 151 | 73.794 | −16.739 | −19.149 | 1.00 | 3.02 | C |
| ATOM | 1124 | O | HIS | A | 151 | 72.986 | −17.039 | −20.030 | 1.00 | 3.02 | O |
| ATOM | 1125 | N | LEU | A | 152 | 73.731 | −15.595 | −18.484 | 1.00 | 3.02 | N |
| ATOM | 1126 | CA | LEU | A | 152 | 72.659 | −14.627 | −18.703 | 1.00 | 3.02 | C |
| ATOM | 1127 | CB | LEU | A | 152 | 71.812 | −14.501 | −17.433 | 1.00 | 3.02 | C |
| ATOM | 1128 | CG | LEU | A | 152 | 71.943 | −15.684 | −16.470 | 1.00 | 3.02 | C |
| ATOM | 1129 | CD1 | LEU | A | 152 | 71.809 | −15.243 | −15.039 | 1.00 | 3.02 | C |
| ATOM | 1130 | CD2 | LEU | A | 152 | 70.974 | −16.797 | −16.805 | 1.00 | 3.02 | C |
| ATOM | 1131 | C | LEU | A | 152 | 73.261 | −13.266 | −19.099 | 1.00 | 3.02 | C |
| ATOM | 1132 | O | LEU | A | 152 | 72.563 | −12.293 | −19.432 | 1.00 | 3.02 | O |
| ATOM | 1133 | N | PRO | B | 11 | 66.179 | −14.807 | −14.893 | 1.00 | 3.02 | N |
| ATOM | 1134 | CA | PRO | B | 11 | 65.041 | −14.732 | −13.964 | 1.00 | 3.02 | C |
| ATOM | 1135 | CB | PRO | B | 11 | 63.831 | −15.101 | −14.839 | 1.00 | 3.02 | C |
| ATOM | 1136 | CG | PRO | B | 11 | 64.403 | −15.709 | −16.096 | 1.00 | 3.02 | C |
| ATOM | 1137 | CD | PRO | B | 11 | 65.771 | −15.119 | −16.276 | 1.00 | 3.02 | C |
| ATOM | 1138 | C | PRO | B | 11 | 65.162 | −15.694 | −12.773 | 1.00 | 3.02 | C |
| ATOM | 1139 | O | PRO | B | 11 | 64.915 | −15.294 | −11.632 | 1.00 | 3.02 | O |
| ATOM | 1140 | N | ALA | B | 12 | 65.540 | −16.945 | −13.047 | 1.00 | 3.02 | N |
| ATOM | 1141 | CA | ALA | B | 12 | 65.717 | −17.966 | −12.009 | 1.00 | 3.02 | C |
| ATOM | 1142 | CB | ALA | B | 12 | 65.293 | −19.322 | −12.539 | 1.00 | 3.02 | C |
| ATOM | 1143 | C | ALA | B | 12 | 67.165 | −18.026 | −11.511 | 1.00 | 3.02 | C |
| ATOM | 1144 | O | ALA | B | 12 | 67.442 | −17.859 | −10.319 | 1.00 | 3.02 | O |
| ATOM | 1145 | N | LEU | B | 13 | 68.080 | −18.248 | −12.453 | 1.00 | 3.02 | N |
| ATOM | 1146 | CA | LEU | B | 13 | 69.502 | −18.457 | −12.172 | 1.00 | 3.02 | C |
| ATOM | 1147 | CB | LEU | B | 13 | 70.145 | −19.249 | −13.315 | 1.00 | 3.02 | C |
| ATOM | 1148 | CG | LEU | B | 13 | 69.388 | −20.542 | −13.629 | 1.00 | 3.02 | C |
| ATOM | 1149 | CD1 | LEU | B | 13 | 69.185 | −20.750 | −15.134 | 1.00 | 3.02 | C |
| ATOM | 1150 | CD2 | LEU | B | 13 | 70.084 | −21.730 | −12.973 | 1.00 | 3.02 | C |
| ATOM | 1151 | C | LEU | B | 13 | 70.297 | −17.167 | −11.873 | 1.00 | 3.02 | C |
| ATOM | 1152 | O | LEU | B | 13 | 71.376 | −17.240 | −11.266 | 1.00 | 3.02 | O |
| ATOM | 1153 | N | PRO | B | 14 | 69.804 | −15.989 | −12.327 | 1.00 | 3.02 | N |
| ATOM | 1154 | CA | PRO | B | 14 | 70.399 | −14.788 | −11.734 | 1.00 | 3.02 | C |
| ATOM | 1155 | CB | PRO | B | 14 | 69.848 | −13.655 | −12.600 | 1.00 | 3.02 | C |
| ATOM | 1156 | CG | PRO | B | 14 | 68.578 | −14.195 | −13.152 | 1.00 | 3.02 | C |
| ATOM | 1157 | CD | PRO | B | 14 | 68.802 | −15.655 | −13.356 | 1.00 | 3.02 | C |
| ATOM | 1158 | C | PRO | B | 14 | 69.960 | −14.603 | −10.277 | 1.00 | 3.02 | C |
| ATOM | 1159 | O | PRO | B | 14 | 70.698 | −14.021 | −9.477 | 1.00 | 3.02 | O |
| ATOM | 1160 | N | ALA | B | 15 | 68.775 | −15.121 | −9.948 | 1.00 | 3.02 | N |
| ATOM | 1161 | CA | ALA | B | 15 | 68.138 | −14.892 | −8.648 | 1.00 | 3.02 | C |
| ATOM | 1162 | CB | ALA | B | 15 | 66.626 | −15.231 | −8.699 | 1.00 | 3.02 | C |
| ATOM | 1163 | C | ALA | B | 15 | 68.823 | −15.672 | −7.550 | 1.00 | 3.02 | C |
| ATOM | 1164 | O | ALA | B | 15 | 69.192 | −15.104 | −6.525 | 1.00 | 3.02 | O |
| ATOM | 1165 | N | PHE | B | 16 | 68.969 | −16.978 | −7.775 | 1.00 | 3.02 | N |
| ATOM | 1166 | CA | PHE | B | 16 | 69.769 | −17.831 | −6.909 | 1.00 | 3.02 | C |
| ATOM | 1167 | CB | PHE | B | 16 | 69.440 | −19.307 | −7.141 | 1.00 | 3.02 | C |
| ATOM | 1168 | CG | PHE | B | 16 | 68.000 | −19.662 | −6.847 | 1.00 | 3.02 | C |
| ATOM | 1169 | CD1 | PHE | B | 16 | 67.036 | −19.613 | −7.855 | 1.00 | 3.02 | C |
| ATOM | 1170 | CE1 | PHE | B | 16 | 65.701 | −19.938 | −7.593 | 1.00 | 3.02 | C |
| ATOM | 1171 | CZ | PHE | B | 16 | 65.315 | −20.318 | −6.310 | 1.00 | 3.02 | C |
| ATOM | 1172 | CE2 | PHE | B | 16 | 66.264 | −20.373 | −5.289 | 1.00 | 3.02 | C |
| ATOM | 1173 | CD2 | PHE | B | 16 | 67.605 | −20.048 | −5.561 | 1.00 | 3.02 | C |
| ATOM | 1174 | C | PHE | B | 16 | 71.268 | −17.538 | −7.105 | 1.00 | 3.02 | C |
| ATOM | 1175 | O | PHE | B | 16 | 72.121 | −18.071 | −6.361 | 1.00 | 3.02 | O |
| ATOM | 1176 | N | LEU | B | 17 | 71.573 | −16.667 | −8.079 | 1.00 | 3.02 | N |
| ATOM | 1177 | CA | LEU | B | 17 | 72.943 | −16.223 | −8.341 | 1.00 | 3.02 | C |
| ATOM | 1178 | CB | LEU | B | 17 | 73.005 | −15.250 | −9.519 | 1.00 | 3.02 | C |
| ATOM | 1179 | CG | LEU | B | 17 | 74.144 | −15.378 | −10.536 | 1.00 | 3.02 | C |
| ATOM | 1180 | CD1 | LEU | B | 17 | 73.766 | −14.578 | −11.778 | 1.00 | 3.02 | C |
| ATOM | 1181 | CD2 | LEU | B | 17 | 75.543 | −14.949 | −9.999 | 1.00 | 3.02 | C |
| ATOM | 1182 | C | LEU | B | 17 | 73.571 | −15.580 | −7.108 | 1.00 | 3.02 | C |
| ATOM | 1183 | O | LEU | B | 17 | 74.468 | −16.167 | −6.493 | 1.00 | 3.02 | O |
| ATOM | 1184 | N | LEU | B | 18 | 73.099 | −14.392 | −6.740 | 1.00 | 3.02 | N |
| ATOM | 1185 | CA | LEU | B | 18 | 73.789 | −13.604 | −5.712 | 1.00 | 3.02 | C |
| ATOM | 1186 | CB | LEU | B | 18 | 73.406 | −12.115 | −5.792 | 1.00 | 3.02 | C |
| ATOM | 1187 | CG | LEU | B | 18 | 74.441 | −11.288 | −6.573 | 1.00 | 3.02 | C |
| ATOM | 1188 | CD1 | LEU | B | 18 | 73.815 | −10.076 | −7.242 | 1.00 | 3.02 | C |
| ATOM | 1189 | CD2 | LEU | B | 18 | 75.605 | −10.887 | −5.671 | 1.00 | 3.02 | C |
| ATOM | 1190 | C | LEU | B | 18 | 73.654 | −14.169 | −4.297 | 1.00 | 3.02 | C |
| ATOM | 1191 | O | LEU | B | 18 | 74.133 | −13.583 | −3.320 | 1.00 | 3.02 | O |
| ATOM | 1192 | N | CYS | B | 19 | 73.056 | −15.352 | −4.233 | 1.00 | 3.02 | N |
| ATOM | 1193 | CA | CYS | B | 19 | 72.762 | −16.057 | −3.000 | 1.00 | 3.02 | C |
| ATOM | 1194 | CB | CYS | B | 19 | 71.600 | −16.974 | −3.271 | 1.00 | 3.02 | C |
| ATOM | 1195 | SG | CYS | B | 19 | 70.470 | −16.153 | −4.415 | 1.00 | 3.02 | S |
| ATOM | 1196 | C | CYS | B | 19 | 73.936 | −16.886 | −2.532 | 1.00 | 3.02 | C |
| ATOM | 1197 | O | CYS | B | 19 | 73.951 | −17.350 | −1.390 | 1.00 | 3.02 | O |
| ATOM | 1198 | N | SER | B | 20 | 74.899 | −17.091 | −3.434 | 1.00 | 3.02 | N |
| ATOM | 1199 | CA | SER | B | 20 | 76.193 | −17.672 | −3.088 | 1.00 | 3.02 | C |
| ATOM | 1200 | CB | SER | B | 20 | 76.939 | −18.176 | −4.329 | 1.00 | 3.02 | C |
| ATOM | 1201 | OG | SER | B | 20 | 76.856 | −17.263 | −5.406 | 1.00 | 3.02 | O |
| ATOM | 1202 | C | SER | B | 20 | 76.991 | −16.617 | −2.368 | 1.00 | 3.02 | C |
| ATOM | 1203 | O | SER | B | 20 | 77.447 | −16.855 | −1.256 | 1.00 | 3.02 | O |
| ATOM | 1204 | N | THR | B | 21 | 77.085 | −15.435 | −2.987 | 1.00 | 3.02 | N |
| ATOM | 1205 | CA | THR | B | 21 | 77.971 | −14.334 | −2.556 | 1.00 | 3.02 | C |
| ATOM | 1206 | CB | THR | B | 21 | 78.075 | −13.210 | −3.633 | 1.00 | 3.02 | C |
| ATOM | 1207 | OG1 | THR | B | 21 | 77.655 | −13.704 | −4.917 | 1.00 | 3.02 | O |
| ATOM | 1208 | CG2 | THR | B | 21 | 79.512 | −12.660 | −3.711 | 1.00 | 3.02 | C |
| ATOM | 1209 | C | THR | B | 21 | 77.559 | −13.692 | −1.228 | 1.00 | 3.02 | C |
| ATOM | 1210 | O | THR | B | 21 | 78.423 | −13.267 | −0.448 | 1.00 | 3.02 | O |
| ATOM | 1211 | N | LEU | B | 22 | 76.247 | −13.624 | −0.982 | 1.00 | 3.02 | N |
| ATOM | 1212 | CA | LEU | B | 22 | 75.737 | −13.145 | 0.299 | 1.00 | 3.02 | C |
| ATOM | 1213 | CB | LEU | B | 22 | 74.207 | −13.034 | 0.292 | 1.00 | 3.02 | C |
| ATOM | 1214 | CG | LEU | B | 22 | 73.475 | −12.205 | −0.767 | 1.00 | 3.02 | C |
| ATOM | 1215 | CD1 | LEU | B | 22 | 72.119 | −11.783 | −0.259 | 1.00 | 3.02 | C |
| ATOM | 1216 | CD2 | LEU | B | 22 | 74.258 | −10.985 | −1.232 | 1.00 | 3.02 | C |
| ATOM | 1217 | C | LEU | B | 22 | 76.199 | −14.061 | 1.430 | 1.00 | 3.02 | C |
| ATOM | 1218 | O | LEU | B | 22 | 76.496 | −13.597 | 2.525 | 1.00 | 3.02 | O |
| ATOM | 1219 | N | LEU | B | 23 | 76.286 | −15.356 | 1.138 | 1.00 | 3.02 | N |
| ATOM | 1220 | CA | LEU | B | 23 | 76.589 | −16.355 | 2.157 | 1.00 | 3.02 | C |
| ATOM | 1221 | CB | LEU | B | 23 | 75.506 | −17.454 | 2.153 | 1.00 | 3.02 | C |
| ATOM | 1222 | CG | LEU | B | 23 | 74.066 | −16.973 | 2.476 | 1.00 | 3.02 | C |
| ATOM | 1223 | CD1 | LEU | B | 23 | 72.963 | −17.659 | 1.653 | 1.00 | 3.02 | C |
| ATOM | 1224 | CD2 | LEU | B | 23 | 73.746 | −17.050 | 3.974 | 1.00 | 3.02 | C |
| ATOM | 1225 | C | LEU | B | 23 | 78.037 | −16.912 | 2.085 | 1.00 | 3.02 | C |
| ATOM | 1226 | O | LEU | B | 23 | 78.388 | −17.838 | 2.822 | 1.00 | 3.02 | O |
| ATOM | 1227 | N | VAL | B | 24 | 78.873 | −16.341 | 1.203 | 1.00 | 3.02 | N |
| ATOM | 1228 | CA | VAL | B | 24 | 80.350 | −16.527 | 1.260 | 1.00 | 3.02 | C |
| ATOM | 1229 | CB | VAL | B | 24 | 81.057 | −16.435 | −0.130 | 1.00 | 3.02 | C |
| ATOM | 1230 | CG1 | VAL | B | 24 | 82.529 | −16.870 | −0.018 | 1.00 | 3.02 | C |
| ATOM | 1231 | CG2 | VAL | B | 24 | 80.348 | −17.259 | −1.168 | 1.00 | 3.02 | C |
| ATOM | 1232 | C | VAL | B | 24 | 80.970 | −15.430 | 2.135 | 1.00 | 3.02 | C |
| ATOM | 1233 | O | VAL | B | 24 | 82.032 | −15.624 | 2.745 | 1.00 | 3.02 | O |
| ATOM | 1234 | N | ILE | B | 25 | 80.318 | −14.261 | 2.132 | 1.00 | 3.02 | N |
| ATOM | 1235 | CA | ILE | B | 25 | 80.652 | −13.174 | 3.042 | 1.00 | 3.02 | C |
| ATOM | 1236 | CB | ILE | B | 25 | 80.144 | −11.787 | 2.519 | 1.00 | 3.02 | C |
| ATOM | 1237 | CG1 | ILE | B | 25 | 80.579 | −11.552 | 1.067 | 1.00 | 3.02 | C |
| ATOM | 1238 | CD1 | ILE | B | 25 | 80.023 | −10.281 | 0.458 | 1.00 | 3.02 | C |
| ATOM | 1239 | CG2 | ILE | B | 25 | 80.639 | −10.638 | 3.412 | 1.00 | 3.02 | C |
| ATOM | 1240 | C | ILE | B | 25 | 80.052 | −13.497 | 4.409 | 1.00 | 3.02 | C |
| ATOM | 1241 | O | ILE | B | 25 | 80.649 | −13.192 | 5.444 | 1.00 | 3.02 | O |
| ATOM | 1242 | N | LYS | B | 26 | 78.891 | −14.154 | 4.399 | 1.00 | 3.02 | N |
| ATOM | 1243 | CA | LYS | B | 26 | 78.092 | −14.339 | 5.617 | 1.00 | 3.02 | C |
| ATOM | 1244 | CB | LYS | B | 26 | 76.601 | −14.170 | 5.321 | 1.00 | 3.02 | C |
| ATOM | 1245 | CG | LYS | B | 26 | 75.710 | −14.219 | 6.534 | 1.00 | 3.02 | C |
| ATOM | 1246 | CD | LYS | B | 26 | 76.000 | −13.119 | 7.535 | 1.00 | 3.02 | C |
| ATOM | 1247 | CE | LYS | B | 26 | 74.932 | −13.168 | 8.619 | 1.00 | 3.02 | C |
| ATOM | 1248 | NZ | LYS | B | 26 | 75.523 | −13.293 | 9.967 | 1.00 | 3.02 | N |
| ATOM | 1249 | C | LYS | B | 26 | 78.375 | −15.648 | 6.357 | 1.00 | 3.02 | C |
| ATOM | 1250 | O | LYS | B | 26 | 78.546 | −15.659 | 7.583 | 1.00 | 3.02 | O |
| ATOM | 1251 | N | MET | B | 27 | 78.421 | −16.755 | 5.631 | 1.00 | 3.02 | N |
| ATOM | 1252 | CA | MET | B | 27 | 78.981 | −17.943 | 6.237 | 1.00 | 3.02 | C |
| ATOM | 1253 | CB | MET | B | 27 | 78.461 | −19.215 | 5.594 | 1.00 | 3.02 | C |
| ATOM | 1254 | CG | MET | B | 27 | 77.111 | −19.579 | 6.128 | 1.00 | 3.02 | C |
| ATOM | 1255 | SD | MET | B | 27 | 77.104 | −21.242 | 6.760 | 1.00 | 3.02 | S |
| ATOM | 1256 | CE | MET | B | 27 | 78.370 | −21.139 | 8.046 | 1.00 | 3.02 | C |
| ATOM | 1257 | C | MET | B | 27 | 80.509 | −17.880 | 6.240 | 1.00 | 3.02 | C |
| ATOM | 1258 | O | MET | B | 27 | 81.129 | −18.036 | 7.298 | 1.00 | 3.02 | O |
| ATOM | 1259 | N | TYR | B | 28 | 81.123 | −17.602 | 5.091 | 1.00 | 3.02 | N |
| ATOM | 1260 | CA | TYR | B | 28 | 82.583 | −17.644 | 5.055 | 1.00 | 3.02 | C |
| ATOM | 1261 | CB | TYR | B | 28 | 83.092 | −18.710 | 4.069 | 1.00 | 3.02 | C |
| ATOM | 1262 | CG | TYR | B | 28 | 83.757 | −19.862 | 4.793 | 1.00 | 3.02 | C |
| ATOM | 1263 | CD1 | TYR | B | 28 | 83.002 | −20.832 | 5.444 | 1.00 | 3.02 | C |
| ATOM | 1264 | CE1 | TYR | B | 28 | 83.612 | −21.877 | 6.122 | 1.00 | 3.02 | C |
| ATOM | 1265 | CZ | TYR | B | 28 | 85.001 | −21.956 | 6.163 | 1.00 | 3.02 | C |
| ATOM | 1266 | OH | TYR | B | 28 | 85.626 | −22.996 | 6.832 | 1.00 | 3.02 | O |
| ATOM | 1267 | CE2 | TYR | B | 28 | 85.769 | −20.995 | 5.530 | 1.00 | 3.02 | C |
| ATOM | 1268 | CD2 | TYR | B | 28 | 85.145 | −19.954 | 4.859 | 1.00 | 3.02 | C |
| ATOM | 1269 | C | TYR | B | 28 | 83.472 | −16.359 | 5.069 | 1.00 | 3.02 | C |
| ATOM | 1270 | O | TYR | B | 28 | 84.680 | −16.476 | 5.279 | 1.00 | 3.02 | O |
| ATOM | 1271 | N | VAL | B | 29 | 82.952 | −15.143 | 4.859 | 1.00 | 3.02 | N |
| ATOM | 1272 | CA | VAL | B | 29 | 83.829 | −13.989 | 5.183 | 1.00 | 3.02 | C |
| ATOM | 1273 | CB | VAL | B | 29 | 83.487 | −12.684 | 4.402 | 1.00 | 3.02 | C |
| ATOM | 1274 | CG1 | VAL | B | 29 | 83.769 | −11.417 | 5.231 | 1.00 | 3.02 | C |
| ATOM | 1275 | CG2 | VAL | B | 29 | 84.242 | −12.646 | 3.075 | 1.00 | 3.02 | C |
| ATOM | 1276 | C | VAL | B | 29 | 83.914 | −13.753 | 6.698 | 1.00 | 3.02 | C |
| ATOM | 1277 | O | VAL | B | 29 | 85.017 | −13.543 | 7.233 | 1.00 | 3.02 | O |
| ATOM | 1278 | N | VAL | B | 30 | 82.751 | −13.816 | 7.368 | 1.00 | 3.02 | N |
| ATOM | 1279 | CA | VAL | B | 30 | 82.626 | −13.651 | 8.831 | 1.00 | 3.02 | C |
| ATOM | 1280 | CB | VAL | B | 30 | 81.160 | −13.785 | 9.294 | 1.00 | 3.02 | C |
| ATOM | 1281 | CG1 | VAL | B | 30 | 81.016 | −13.350 | 10.741 | 1.00 | 3.02 | C |
| ATOM | 1282 | CG2 | VAL | B | 30 | 80.253 | −12.976 | 8.422 | 1.00 | 3.02 | C |
| ATOM | 1283 | C | VAL | B | 30 | 83.466 | −14.672 | 9.608 | 1.00 | 3.02 | C |
| ATOM | 1284 | O | VAL | B | 30 | 84.209 | −14.307 | 10.520 | 1.00 | 3.02 | O |
| ATOM | 1285 | N | ALA | B | 31 | 83.323 | −15.944 | 9.235 | 1.00 | 3.02 | N |
| ATOM | 1286 | CA | ALA | B | 31 | 84.103 | −17.032 | 9.801 | 1.00 | 3.02 | C |
| ATOM | 1287 | CB | ALA | B | 31 | 83.649 | −18.357 | 9.207 | 1.00 | 3.02 | C |
| ATOM | 1288 | C | ALA | B | 31 | 85.603 | −16.814 | 9.579 | 1.00 | 3.02 | C |
| ATOM | 1289 | O | ALA | B | 31 | 86.418 | −16.985 | 10.494 | 1.00 | 3.02 | O |
| ATOM | 1290 | N | ILE | B | 32 | 85.960 | −16.418 | 8.363 | 1.00 | 3.02 | N |
| ATOM | 1291 | CA | ILE | B | 32 | 87.354 | −16.118 | 8.051 | 1.00 | 3.02 | C |
| ATOM | 1292 | CB | ILE | B | 32 | 87.585 | −15.863 | 6.528 | 1.00 | 3.02 | C |
| ATOM | 1293 | CG1 | ILE | B | 32 | 87.538 | −17.228 | 5.791 | 1.00 | 3.02 | C |
| ATOM | 1294 | CD1 | ILE | B | 32 | 87.797 | −17.222 | 4.278 | 1.00 | 3.02 | C |
| ATOM | 1295 | CG2 | ILE | B | 32 | 88.895 | −15.097 | 6.294 | 1.00 | 3.02 | C |
| ATOM | 1296 | C | ILE | B | 32 | 87.940 | −15.044 | 8.981 | 1.00 | 3.02 | C |
| ATOM | 1297 | O | ILE | B | 32 | 88.982 | −15.292 | 9.594 | 1.00 | 3.02 | O |
| ATOM | 1298 | N | ILE | B | 33 | 87.261 | −13.895 | 9.117 | 1.00 | 3.02 | N |
| ATOM | 1299 | CA | ILE | B | 33 | 87.674 | −12.840 | 10.077 | 1.00 | 3.02 | C |
| ATOM | 1300 | CB | ILE | B | 33 | 86.820 | −11.541 | 9.980 | 1.00 | 3.02 | C |
| ATOM | 1301 | CG1 | ILE | B | 33 | 87.171 | −10.733 | 8.724 | 1.00 | 3.02 | C |
| ATOM | 1302 | CD1 | ILE | B | 33 | 86.130 | −9.621 | 8.375 | 1.00 | 3.02 | C |
| ATOM | 1303 | CG2 | ILE | B | 33 | 87.025 | −10.661 | 11.219 | 1.00 | 3.02 | C |
| ATOM | 1304 | C | ILE | B | 33 | 87.666 | −13.320 | 11.533 | 1.00 | 3.02 | C |
| ATOM | 1305 | O | ILE | B | 33 | 88.688 | −13.214 | 12.225 | 1.00 | 3.02 | O |
| ATOM | 1306 | N | THR | B | 34 | 86.518 | −13.837 | 11.989 | 1.00 | 3.02 | N |
| ATOM | 1307 | CA | THR | B | 34 | 86.391 | −14.353 | 13.355 | 1.00 | 3.02 | C |
| ATOM | 1308 | CB | THR | B | 34 | 84.945 | −14.741 | 13.730 | 1.00 | 3.02 | C |
| ATOM | 1309 | OG1 | THR | B | 34 | 84.335 | −15.450 | 12.647 | 1.00 | 3.02 | O |
| ATOM | 1310 | CG2 | THR | B | 34 | 84.112 | −13.488 | 14.011 | 1.00 | 3.02 | C |
| ATOM | 1311 | C | THR | B | 34 | 87.391 | −15.477 | 13.597 | 1.00 | 3.02 | C |
| ATOM | 1312 | O | THR | B | 34 | 87.830 | −15.688 | 14.732 | 1.00 | 3.02 | O |
| ATOM | 1313 | N | GLY | B | 35 | 87.775 | −16.174 | 12.529 | 1.00 | 3.02 | N |
| ATOM | 1314 | CA | GLY | B | 35 | 89.017 | −16.942 | 12.542 | 1.00 | 3.02 | C |
| ATOM | 1315 | C | GLY | B | 35 | 90.215 | −16.014 | 12.676 | 1.00 | 3.02 | C |
| ATOM | 1316 | O | GLY | B | 35 | 90.817 | −15.940 | 13.739 | 1.00 | 3.02 | O |
| ATOM | 1317 | N | GLN | B | 36 | 90.505 | −15.272 | 11.605 | 1.00 | 3.02 | N |
| ATOM | 1318 | CA | GLN | B | 36 | 91.765 | −14.512 | 11.419 | 1.00 | 3.02 | C |
| ATOM | 1319 | CB | GLN | B | 36 | 91.725 | −13.708 | 10.104 | 1.00 | 3.02 | C |
| ATOM | 1320 | CG | GLN | B | 36 | 91.623 | −14.544 | 8.827 | 1.00 | 3.02 | C |
| ATOM | 1321 | CD | GLN | B | 36 | 92.654 | −15.640 | 8.760 | 1.00 | 3.02 | C |
| ATOM | 1322 | OE1 | GLN | B | 36 | 93.777 | −15.480 | 9.258 | 1.00 | 3.02 | O |
| ATOM | 1323 | NE2 | GLN | B | 36 | 92.285 | −16.769 | 8.149 | 1.00 | 3.02 | N |
| ATOM | 1324 | C | GLN | B | 36 | 92.253 | −13.593 | 12.565 | 1.00 | 3.02 | C |
| ATOM | 1325 | O | GLN | B | 36 | 93.446 | −13.595 | 12.907 | 1.00 | 3.02 | O |
| ATOM | 1326 | N | VAL | B | 37 | 91.331 | −12.811 | 13.128 | 1.00 | 3.02 | N |
| ATOM | 1327 | CA | VAL | B | 37 | 91.618 | −11.881 | 14.228 | 1.00 | 3.02 | C |
| ATOM | 1328 | CB | VAL | B | 37 | 90.565 | −10.730 | 14.297 | 1.00 | 3.02 | C |
| ATOM | 1329 | CG1 | VAL | B | 37 | 90.749 | −9.890 | 15.572 | 1.00 | 3.02 | C |
| ATOM | 1330 | CG2 | VAL | B | 37 | 90.622 | −9.840 | 13.045 | 1.00 | 3.02 | C |
| ATOM | 1331 | C | VAL | B | 37 | 91.652 | −12.590 | 15.590 | 1.00 | 3.02 | C |
| ATOM | 1332 | O | VAL | B | 37 | 92.357 | −12.142 | 16.495 | 1.00 | 3.02 | O |
| ATOM | 1333 | N | ARG | B | 38 | 90.871 | −13.667 | 15.738 | 1.00 | 3.02 | N |
| ATOM | 1334 | CA | ARG | B | 38 | 90.818 | −14.436 | 16.986 | 1.00 | 3.02 | C |
| ATOM | 1335 | CB | ARG | B | 38 | 89.765 | −15.560 | 16.900 | 1.00 | 3.02 | C |
| ATOM | 1336 | CG | ARG | B | 38 | 89.651 | −16.452 | 18.155 | 1.00 | 3.02 | C |
| ATOM | 1337 | CD | ARG | B | 38 | 89.200 | −17.896 | 17.847 | 1.00 | 3.02 | C |
| ATOM | 1338 | NE | ARG | B | 38 | 89.020 | −18.701 | 19.066 | 1.00 | 3.02 | N |
| ATOM | 1339 | CZ | ARG | B | 38 | 88.710 | −20.000 | 19.095 | 1.00 | 3.02 | C |
| ATOM | 1340 | NH1 | ARG | B | 38 | 88.550 | −20.693 | 17.975 | 1.00 | 3.02 | N |
| ATOM | 1341 | NH2 | ARG | B | 38 | 88.563 | −20.612 | 20.259 | 1.00 | 3.02 | N |
| ATOM | 1342 | C | ARG | B | 38 | 92.183 | −15.028 | 17.251 | 1.00 | 3.02 | C |
| ATOM | 1343 | O | ARG | B | 38 | 92.685 | −14.937 | 18.370 | 1.00 | 3.02 | O |
| ATOM | 1344 | N | LEU | B | 39 | 92.762 | −15.588 | 16.180 | 1.00 | 3.02 | N |
| ATOM | 1345 | CA | LEU | B | 39 | 93.959 | −16.434 | 16.177 | 1.00 | 3.02 | C |
| ATOM | 1346 | CB | LEU | B | 39 | 93.904 | −17.415 | 15.004 | 1.00 | 3.02 | C |
| ATOM | 1347 | CG | LEU | B | 39 | 93.509 | −18.880 | 15.174 | 1.00 | 3.02 | C |
| ATOM | 1348 | CD1 | LEU | B | 39 | 92.922 | −19.354 | 13.873 | 1.00 | 3.02 | C |
| ATOM | 1349 | CD2 | LEU | B | 39 | 94.687 | −19.771 | 15.550 | 1.00 | 3.02 | C |
| ATOM | 1350 | C | LEU | B | 39 | 95.249 | −15.645 | 16.065 | 1.00 | 3.02 | C |
| ATOM | 1351 | O | LEU | B | 39 | 96.307 | −16.149 | 16.467 | 1.00 | 3.02 | O |
| ATOM | 1352 | N | ARG | B | 40 | 95.181 | −14.436 | 15.498 | 1.00 | 3.02 | N |
| ATOM | 1353 | CA | ARG | B | 40 | 96.372 | −13.575 | 15.433 | 1.00 | 3.02 | C |
| ATOM | 1354 | CB | ARG | B | 40 | 96.188 | −12.374 | 14.498 | 1.00 | 3.02 | C |
| ATOM | 1355 | CG | ARG | B | 40 | 97.508 | −11.881 | 13.903 | 1.00 | 3.02 | C |
| ATOM | 1356 | CD | ARG | B | 40 | 97.670 | −10.350 | 13.881 | 1.00 | 3.02 | C |
| ATOM | 1357 | NE | ARG | B | 40 | 99.073 | −10.036 | 13.595 | 1.00 | 3.02 | N |
| ATOM | 1358 | CZ | ARG | B | 40 | 99.557 | −8.903 | 13.082 | 1.00 | 3.02 | C |
| ATOM | 1359 | NH1 | ARG | B | 40 | 98.766 | −7.882 | 12.756 | 1.00 | 3.02 | N |
| ATOM | 1360 | NH2 | ARG | B | 40 | 100.866 | −8.813 | 12.879 | 1.00 | 3.02 | N |
| ATOM | 1361 | C | ARG | B | 40 | 96.761 | −13.095 | 16.834 | 1.00 | 3.02 | C |
| ATOM | 1362 | O | ARG | B | 40 | 97.921 | −13.231 | 17.257 | 1.00 | 3.02 | O |
| ATOM | 1363 | N | LYS | B | 41 | 95.784 | −12.539 | 17.548 | 1.00 | 3.02 | N |
| ATOM | 1364 | CA | LYS | B | 41 | 95.964 | −12.174 | 18.949 | 1.00 | 3.02 | C |
| ATOM | 1365 | CB | LYS | B | 41 | 94.998 | −11.055 | 19.341 | 1.00 | 3.02 | C |
| ATOM | 1366 | CG | LYS | B | 41 | 94.546 | −10.156 | 18.206 | 1.00 | 3.02 | C |
| ATOM | 1367 | CD | LYS | B | 41 | 93.218 | −9.481 | 18.571 | 1.00 | 3.02 | C |
| ATOM | 1368 | CE | LYS | B | 41 | 93.077 | −8.109 | 17.908 | 1.00 | 3.02 | C |
| ATOM | 1369 | NZ | LYS | B | 41 | 92.346 | −7.119 | 18.766 | 1.00 | 3.02 | N |
| ATOM | 1370 | C | LYS | B | 41 | 95.729 | −13.407 | 19.846 | 1.00 | 3.02 | C |
| ATOM | 1371 | O | LYS | B | 41 | 95.241 | −13.279 | 20.980 | 1.00 | 3.02 | O |
| ATOM | 1372 | N | LYS | B | 42 | 96.084 | −14.591 | 19.342 | 1.00 | 3.02 | N |
| ATOM | 1373 | CA | LYS | B | 42 | 95.740 | −15.847 | 20.000 | 1.00 | 3.02 | C |
| ATOM | 1374 | CB | LYS | B | 42 | 96.544 | −17.022 | 19.421 | 1.00 | 3.02 | C |
| ATOM | 1375 | CG | LYS | B | 42 | 96.152 | −18.408 | 19.937 | 1.00 | 3.02 | C |
| ATOM | 1376 | CD | LYS | B | 42 | 94.927 | −18.985 | 19.237 | 1.00 | 3.02 | C |
| ATOM | 1377 | CE | LYS | B | 42 | 94.885 | −20.526 | 19.375 | 1.00 | 3.02 | C |
| ATOM | 1378 | NZ | LYS | B | 42 | 93.531 | −21.158 | 19.143 | 1.00 | 3.02 | N |
| ATOM | 1379 | C | LYS | B | 42 | 95.882 | −15.732 | 21.519 | 1.00 | 3.02 | C |
| ATOM | 1380 | O | LYS | B | 42 | 96.993 | −15.635 | 22.054 | 1.00 | 3.02 | O |
| ATOM | 1381 | N | ALA | B | 43 | 94.732 | −15.687 | 22.187 | 1.00 | 3.02 | N |
| ATOM | 1382 | CA | ALA | B | 43 | 94.666 | −15.710 | 23.639 | 1.00 | 3.02 | C |
| ATOM | 1383 | CB | ALA | B | 43 | 93.511 | −14.842 | 24.132 | 1.00 | 3.02 | C |
| ATOM | 1384 | C | ALA | B | 43 | 94.534 | −17.153 | 24.139 | 1.00 | 3.02 | C |
| ATOM | 1385 | O | ALA | B | 43 | 93.689 | −17.456 | 24.980 | 1.00 | 3.02 | O |
| ATOM | 1386 | N | PHE | B | 44 | 95.368 | −18.037 | 23.593 | 1.00 | 3.02 | N |
| ATOM | 1387 | CA | PHE | B | 44 | 95.465 | −19.420 | 24.050 | 1.00 | 3.02 | C |
| ATOM | 1388 | CB | PHE | B | 44 | 94.880 | −20.386 | 23.019 | 1.00 | 3.02 | C |
| ATOM | 1389 | CG | PHE | B | 44 | 93.364 | −20.339 | 22.932 | 1.00 | 3.02 | C |
| ATOM | 1390 | CD1 | PHE | B | 44 | 92.705 | −19.235 | 22.382 | 1.00 | 3.02 | C |
| ATOM | 1391 | CE1 | PHE | B | 44 | 91.310 | −19.194 | 22.313 | 1.00 | 3.02 | C |
| ATOM | 1392 | CZ | PHE | B | 44 | 90.560 | −20.268 | 22.789 | 1.00 | 3.02 | C |
| ATOM | 1393 | CE2 | PHE | B | 44 | 91.204 | −21.371 | 23.331 | 1.00 | 3.02 | C |
| ATOM | 1394 | CD2 | PHE | B | 44 | 92.598 | −21.399 | 23.398 | 1.00 | 3.02 | C |
| ATOM | 1395 | C | PHE | B | 44 | 96.929 | −19.690 | 24.381 | 1.00 | 3.02 | C |
| ATOM | 1396 | O | PHE | B | 44 | 97.732 | −18.762 | 24.331 | 1.00 | 3.02 | O |
| ATOM | 1397 | N | ALA | B | 45 | 97.280 | −20.935 | 24.707 | 1.00 | 3.02 | N |
| ATOM | 1398 | CA | ALA | B | 45 | 98.491 | −21.235 | 25.494 | 1.00 | 3.02 | C |
| ATOM | 1399 | CB | ALA | B | 45 | 99.747 | −20.667 | 24.848 | 1.00 | 3.02 | C |
| ATOM | 1400 | C | ALA | B | 45 | 98.281 | −20.678 | 26.904 | 1.00 | 3.02 | C |
| ATOM | 1401 | O | ALA | B | 45 | 99.131 | −20.020 | 27.475 | 1.00 | 3.02 | O |
| ATOM | 1402 | N | ASN | B | 46 | 97.100 | −20.978 | 27.433 | 1.00 | 3.02 | N |
| ATOM | 1403 | CA | ASN | B | 46 | 96.576 | −20.481 | 28.696 | 1.00 | 3.02 | C |
| ATOM | 1404 | CB | ASN | B | 46 | 95.052 | −20.605 | 28.664 | 1.00 | 3.02 | C |
| ATOM | 1405 | CG | ASN | B | 46 | 94.469 | −20.185 | 27.325 | 1.00 | 3.02 | C |
| ATOM | 1406 | OD1 | ASN | B | 46 | 93.846 | −20.967 | 26.622 | 1.00 | 3.02 | O |
| ATOM | 1407 | ND2 | ASN | B | 46 | 94.706 | −18.938 | 26.957 | 1.00 | 3.02 | N |
| ATOM | 1408 | C | ASN | B | 46 | 97.156 | −21.216 | 29.906 | 1.00 | 3.02 | C |
| ATOM | 1409 | O | ASN | B | 46 | 97.458 | −22.398 | 29.822 | 1.00 | 3.02 | O |
| ATOM | 1410 | N | PRO | B | 47 | 97.247 | −20.525 | 31.059 | 1.00 | 3.02 | N |
| ATOM | 1411 | CA | PRO | B | 47 | 98.040 | −20.863 | 32.237 | 1.00 | 3.02 | C |
| ATOM | 1412 | CB | PRO | B | 47 | 96.944 | −20.935 | 33.326 | 1.00 | 3.02 | C |
| ATOM | 1413 | CG | PRO | B | 47 | 95.835 | −19.861 | 32.781 | 1.00 | 3.02 | C |
| ATOM | 1414 | CD | PRO | B | 47 | 96.354 | −19.400 | 31.401 | 1.00 | 3.02 | C |
| ATOM | 1415 | C | PRO | B | 47 | 98.826 | −22.160 | 32.161 | 1.00 | 3.02 | C |
| ATOM | 1416 | O | PRO | B | 47 | 98.779 | −22.943 | 33.111 | 1.00 | 3.02 | O |
| ATOM | 1417 | N | GLU | B | 48 | 99.550 | −22.371 | 31.058 | 1.00 | 3.02 | N |
| ATOM | 1418 | CA | GLU | B | 48 | 100.254 | −23.642 | 30.802 | 1.00 | 3.02 | C |
| ATOM | 1419 | CB | GLU | B | 48 | 101.361 | −23.875 | 31.830 | 1.00 | 3.02 | C |
| ATOM | 1420 | CG | GLU | B | 48 | 102.482 | −24.774 | 31.372 | 1.00 | 3.02 | C |
| ATOM | 1421 | CD | GLU | B | 48 | 103.502 | −25.000 | 32.480 | 1.00 | 3.02 | C |
| ATOM | 1422 | OE1 | GLU | B | 48 | 103.821 | −23.998 | 33.186 | 1.00 | 3.02 | O |
| ATOM | 1423 | OE2 | GLU | B | 48 | 103.982 | −26.169 | 32.658 | 1.00 | 3.02 | O |
| ATOM | 1424 | C | GLU | B | 48 | 99.243 | −24.797 | 30.804 | 1.00 | 3.02 | C |
| ATOM | 1425 | O | GLU | B | 48 | 99.136 | −25.571 | 31.769 | 1.00 | 3.02 | O |
| ATOM | 1426 | N | ASP | B | 49 | 98.505 | −24.881 | 29.700 | 1.00 | 3.02 | N |
| ATOM | 1427 | CA | ASP | B | 49 | 97.347 | −25.740 | 29.565 | 1.00 | 3.02 | C |
| ATOM | 1428 | CB | ASP | B | 49 | 96.089 | −24.867 | 29.487 | 1.00 | 3.02 | C |
| ATOM | 1429 | CG | ASP | B | 49 | 94.810 | −25.660 | 29.586 | 1.00 | 3.02 | C |
| ATOM | 1430 | OD1 | ASP | B | 49 | 94.700 | −26.512 | 30.486 | 1.00 | 3.02 | O |
| ATOM | 1431 | OD2 | ASP | B | 49 | 93.903 | −25.407 | 28.770 | 1.00 | 3.02 | O |
| ATOM | 1432 | C | ASP | B | 49 | 97.479 | −26.543 | 28.288 | 1.00 | 3.02 | C |
| ATOM | 1433 | O | ASP | B | 49 | 97.869 | −26.006 | 27.246 | 1.00 | 3.02 | O |
| ATOM | 1434 | N | ALA | B | 50 | 97.188 | −27.837 | 28.392 | 1.00 | 3.02 | N |
| ATOM | 1435 | CA | ALA | B | 50 | 96.829 | −28.652 | 27.246 | 1.00 | 3.02 | C |
| ATOM | 1436 | CB | ALA | B | 50 | 96.692 | −30.102 | 27.672 | 1.00 | 3.02 | C |
| ATOM | 1437 | C | ALA | B | 50 | 95.494 | −28.096 | 26.757 | 1.00 | 3.02 | C |
| ATOM | 1438 | O | ALA | B | 50 | 94.567 | −27.898 | 27.554 | 1.00 | 3.02 | O |
| ATOM | 1439 | N | LEU | B | 51 | 95.401 | −27.825 | 25.461 | 1.00 | 3.02 | N |
| ATOM | 1440 | CA | LEU | B | 51 | 94.308 | −27.000 | 24.951 | 1.00 | 3.02 | C |
| ATOM | 1441 | CB | LEU | B | 51 | 94.658 | −26.416 | 23.577 | 1.00 | 3.02 | C |
| ATOM | 1442 | CG | LEU | B | 51 | 93.755 | −25.273 | 23.091 | 1.00 | 3.02 | C |
| ATOM | 1443 | CD1 | LEU | B | 51 | 92.603 | −25.795 | 22.220 | 1.00 | 3.02 | C |
| ATOM | 1444 | CD2 | LEU | B | 51 | 93.213 | −24.448 | 24.254 | 1.00 | 3.02 | C |
| ATOM | 1445 | C | LEU | B | 51 | 92.874 | −27.574 | 24.957 | 1.00 | 3.02 | C |
| ATOM | 1446 | O | LEU | B | 51 | 92.547 | −28.515 | 24.203 | 1.00 | 3.02 | O |
| ATOM | 1447 | N | ARG | B | 52 | 92.059 | −26.977 | 25.841 | 1.00 | 3.02 | N |
| ATOM | 1448 | CA | ARG | B | 52 | 90.579 | −26.894 | 25.739 | 1.00 | 3.02 | C |
| ATOM | 1449 | CB | ARG | B | 52 | 89.855 | −28.174 | 26.191 | 1.00 | 3.02 | C |
| ATOM | 1450 | CG | ARG | B | 52 | 90.715 | −29.412 | 26.213 | 1.00 | 3.02 | C |
| ATOM | 1451 | CD | ARG | B | 52 | 89.917 | −30.663 | 25.914 | 1.00 | 3.02 | C |
| ATOM | 1452 | NE | ARG | B | 52 | 90.720 | −31.654 | 25.191 | 1.00 | 3.02 | N |
| ATOM | 1453 | CZ | ARG | B | 52 | 91.617 | −32.469 | 25.754 | 1.00 | 3.02 | C |
| ATOM | 1454 | NH1 | ARG | B | 52 | 91.845 | −32.434 | 27.071 | 1.00 | 3.02 | N |
| ATOM | 1455 | NH2 | ARG | B | 52 | 92.280 | −33.339 | 24.998 | 1.00 | 3.02 | N |
| ATOM | 1456 | C | ARG | B | 52 | 90.099 | −25.677 | 26.562 | 1.00 | 3.02 | C |
| ATOM | 1457 | O | ARG | B | 52 | 90.882 | −24.719 | 26.783 | 1.00 | 3.02 | O |
| ATOM | 1458 | N | HIS | B | 53 | 88.824 | −25.723 | 26.988 | 1.00 | 3.02 | N |
| ATOM | 1459 | CA | HIS | B | 53 | 88.204 | −24.769 | 27.934 | 1.00 | 3.02 | C |
| ATOM | 1460 | CB | HIS | B | 53 | 89.006 | −24.706 | 29.247 | 1.00 | 3.02 | C |
| ATOM | 1461 | CG | HIS | B | 53 | 89.992 | −25.825 | 29.406 | 1.00 | 3.02 | C |
| ATOM | 1462 | ND1 | HIS | B | 53 | 89.631 | −27.157 | 29.357 | 1.00 | 3.02 | N |
| ATOM | 1463 | CE1 | HIS | B | 53 | 90.704 | −27.913 | 29.514 | 1.00 | 3.02 | C |
| ATOM | 1464 | NE2 | HIS | B | 53 | 91.753 | −27.122 | 29.657 | 1.00 | 3.02 | N |
| ATOM | 1465 | CD2 | HIS | B | 53 | 91.335 | −25.811 | 29.593 | 1.00 | 3.02 | C |
| ATOM | 1466 | C | HIS | B | 53 | 87.946 | −23.373 | 27.327 | 1.00 | 3.02 | C |
| ATOM | 1467 | O | HIS | B | 53 | 88.045 | −23.201 | 26.112 | 1.00 | 3.02 | O |
| ATOM | 1468 | N | GLY | B | 54 | 87.600 | −22.390 | 28.161 | 1.00 | 3.02 | N |
| ATOM | 1469 | CA | GLY | B | 54 | 87.279 | −21.054 | 27.660 | 1.00 | 3.02 | C |
| ATOM | 1470 | C | GLY | B | 54 | 86.374 | −20.185 | 28.518 | 1.00 | 3.02 | C |
| ATOM | 1471 | O | GLY | B | 54 | 85.459 | −19.509 | 28.003 | 1.00 | 3.02 | O |
| ATOM | 1472 | N | GLY | B | 55 | 86.632 | −20.190 | 29.824 | 1.00 | 3.02 | N |
| ATOM | 1473 | CA | GLY | B | 55 | 85.902 | −19.322 | 30.740 | 1.00 | 3.02 | C |
| ATOM | 1474 | C | GLY | B | 55 | 86.670 | −18.904 | 31.985 | 1.00 | 3.02 | C |
| ATOM | 1475 | O | GLY | B | 55 | 86.133 | −19.039 | 33.088 | 1.00 | 3.02 | O |
| ATOM | 1476 | N | PRO | B | 56 | 87.928 | −18.410 | 31.827 | 1.00 | 3.02 | N |
| ATOM | 1477 | CA | PRO | B | 56 | 88.701 | −17.818 | 32.904 | 1.00 | 3.02 | C |
| ATOM | 1478 | CB | PRO | B | 56 | 90.066 | −18.481 | 32.720 | 1.00 | 3.02 | C |
| ATOM | 1479 | CG | PRO | B | 56 | 90.137 | −18.799 | 31.193 | 1.00 | 3.02 | C |
| ATOM | 1480 | CD | PRO | B | 56 | 88.755 | −18.465 | 30.611 | 1.00 | 3.02 | C |
| ATOM | 1481 | C | PRO | B | 56 | 88.837 | −16.285 | 32.806 | 1.00 | 3.02 | C |
| ATOM | 1482 | O | PRO | B | 56 | 88.201 | −15.575 | 33.586 | 1.00 | 3.02 | O |
| ATOM | 1483 | N | GLN | B | 57 | 89.671 | −15.794 | 31.879 | 1.00 | 3.02 | N |
| ATOM | 1484 | CA | GLN | B | 57 | 89.951 | −14.353 | 31.714 | 1.00 | 3.02 | C |
| ATOM | 1485 | CB | GLN | B | 57 | 91.396 | −14.119 | 31.253 | 1.00 | 3.02 | C |
| ATOM | 1486 | CG | GLN | B | 57 | 92.367 | −13.631 | 32.338 | 1.00 | 3.02 | C |
| ATOM | 1487 | CD | GLN | B | 57 | 93.603 | −12.951 | 31.760 | 1.00 | 3.02 | C |
| ATOM | 1488 | OE1 | GLN | B | 57 | 93.488 | −11.976 | 31.016 | 1.00 | 3.02 | O |
| ATOM | 1489 | NE2 | GLN | B | 57 | 94.787 | −13.460 | 32.109 | 1.00 | 3.02 | N |
| ATOM | 1490 | C | GLN | B | 57 | 89.017 | −13.662 | 30.724 | 1.00 | 3.02 | C |
| ATOM | 1491 | O | GLN | B | 57 | 88.947 | −14.051 | 29.545 | 1.00 | 3.02 | O |
| ATOM | 1492 | N | TYR | B | 58 | 88.332 | −12.617 | 31.202 | 1.00 | 3.02 | N |
| ATOM | 1493 | CA | TYR | B | 58 | 87.421 | −11.818 | 30.383 | 1.00 | 3.02 | C |
| ATOM | 1494 | CB | TYR | B | 58 | 86.750 | −10.735 | 31.235 | 1.00 | 3.02 | C |
| ATOM | 1495 | CG | TYR | B | 58 | 87.661 | −9.574 | 31.628 | 1.00 | 3.02 | C |
| ATOM | 1496 | CD1 | TYR | B | 58 | 87.667 | −8.377 | 30.892 | 1.00 | 3.02 | C |
| ATOM | 1497 | CE1 | TYR | B | 58 | 88.495 | −7.324 | 31.242 | 1.00 | 3.02 | C |
| ATOM | 1498 | CZ | TYR | B | 58 | 89.329 | −7.451 | 32.345 | 1.00 | 3.02 | C |
| ATOM | 1499 | OH | TYR | B | 58 | 90.153 | −6.408 | 32.706 | 1.00 | 3.02 | O |
| ATOM | 1500 | CE2 | TYR | B | 58 | 89.344 | −8.611 | 33.094 | 1.00 | 3.02 | C |
| ATOM | 1501 | CD2 | TYR | B | 58 | 88.516 | −9.665 | 32.733 | 1.00 | 3.02 | C |
| ATOM | 1502 | C | TYR | B | 58 | 88.154 | −11.194 | 29.189 | 1.00 | 3.02 | C |
| ATOM | 1503 | O | TYR | B | 58 | 87.574 | −11.046 | 28.109 | 1.00 | 3.02 | O |
| ATOM | 1504 | N | CYS | B | 59 | 89.422 | −10.817 | 29.402 | 1.00 | 3.02 | N |
| ATOM | 1505 | CA | CYS | B | 59 | 90.328 | −10.349 | 28.338 | 1.00 | 3.02 | C |
| ATOM | 1506 | CB | CYS | B | 59 | 91.673 | −9.878 | 28.931 | 1.00 | 3.02 | C |
| ATOM | 1507 | SG | CYS | B | 59 | 91.853 | −8.075 | 29.285 | 1.00 | 3.02 | S |
| ATOM | 1508 | C | CYS | B | 59 | 90.592 | −11.435 | 27.280 | 1.00 | 3.02 | C |
| ATOM | 1509 | O | CYS | B | 59 | 90.938 | −11.120 | 26.134 | 1.00 | 3.02 | O |
| ATOM | 1510 | N | ARG | B | 60 | 90.426 | −12.702 | 27.671 | 1.00 | 3.02 | N |
| ATOM | 1511 | CA | ARG | B | 60 | 90.752 | −13.834 | 26.817 | 1.00 | 3.02 | C |
| ATOM | 1512 | CB | ARG | B | 60 | 91.393 | −14.946 | 27.647 | 1.00 | 3.02 | C |
| ATOM | 1513 | CG | ARG | B | 60 | 91.460 | −16.286 | 26.943 | 1.00 | 3.02 | C |
| ATOM | 1514 | CD | ARG | B | 60 | 90.903 | −17.371 | 27.824 | 1.00 | 3.02 | C |
| ATOM | 1515 | NE | ARG | B | 60 | 90.425 | −18.495 | 27.038 | 1.00 | 3.02 | N |
| ATOM | 1516 | CZ | ARG | B | 60 | 90.637 | −19.766 | 27.364 | 1.00 | 3.02 | C |
| ATOM | 1517 | NH1 | ARG | B | 60 | 91.326 | −20.066 | 28.459 | 1.00 | 3.02 | N |
| ATOM | 1518 | NH2 | ARG | B | 60 | 90.181 | −20.744 | 26.588 | 1.00 | 3.02 | N |
| ATOM | 1519 | C | ARG | B | 60 | 89.544 | −14.384 | 26.069 | 1.00 | 3.02 | C |
| ATOM | 1520 | O | ARG | B | 60 | 89.528 | −14.390 | 24.830 | 1.00 | 3.02 | O |
| ATOM | 1521 | N | SER | B | 61 | 88.546 | −14.856 | 26.819 | 1.00 | 3.02 | N |
| ATOM | 1522 | CA | SER | B | 61 | 87.466 | −15.655 | 26.229 | 1.00 | 3.02 | C |
| ATOM | 1523 | CB | SER | B | 61 | 87.369 | −17.059 | 26.872 | 1.00 | 3.02 | C |
| ATOM | 1524 | OG | SER | B | 61 | 86.540 | −17.085 | 28.030 | 1.00 | 3.02 | O |
| ATOM | 1525 | C | SER | B | 61 | 86.117 | −14.941 | 26.196 | 1.00 | 3.02 | C |
| ATOM | 1526 | O | SER | B | 61 | 85.607 | −14.692 | 25.117 | 1.00 | 3.02 | O |
| ATOM | 1527 | N | ASP | B | 62 | 85.560 | −14.605 | 27.358 | 1.00 | 3.02 | N |
| ATOM | 1528 | CA | ASP | B | 62 | 84.178 | −14.105 | 27.444 | 1.00 | 3.02 | C |
| ATOM | 1529 | CB | ASP | B | 62 | 83.993 | −13.149 | 28.627 | 1.00 | 3.02 | C |
| ATOM | 1530 | CG | ASP | B | 62 | 83.794 | −13.871 | 29.937 | 1.00 | 3.02 | C |
| ATOM | 1531 | OD1 | ASP | B | 62 | 84.782 | −14.436 | 30.479 | 1.00 | 3.02 | O |
| ATOM | 1532 | OD2 | ASP | B | 62 | 82.650 | −13.836 | 30.435 | 1.00 | 3.02 | O |
| ATOM | 1533 | C | ASP | B | 62 | 83.633 | −13.483 | 26.142 | 1.00 | 3.02 | C |
| ATOM | 1534 | O | ASP | B | 62 | 82.674 | −14.024 | 25.576 | 1.00 | 3.02 | O |
| ATOM | 1535 | N | PRO | B | 63 | 84.239 | −12.367 | 25.651 | 1.00 | 3.02 | N |
| ATOM | 1536 | CA | PRO | B | 63 | 83.760 | −11.874 | 24.356 | 1.00 | 3.02 | C |
| ATOM | 1537 | CB | PRO | B | 63 | 84.577 | −10.592 | 24.131 | 1.00 | 3.02 | C |
| ATOM | 1538 | CG | PRO | B | 63 | 85.816 | −10.788 | 24.956 | 1.00 | 3.02 | C |
| ATOM | 1539 | CD | PRO | B | 63 | 85.336 | −11.526 | 26.172 | 1.00 | 3.02 | C |
| ATOM | 1540 | C | PRO | B | 63 | 84.016 | −12.900 | 23.245 | 1.00 | 3.02 | C |
| ATOM | 1541 | O | PRO | B | 63 | 83.057 | −13.394 | 22.663 | 1.00 | 3.02 | O |
| ATOM | 1542 | N | ASP | B | 64 | 85.288 | −13.245 | 23.004 | 1.00 | 3.02 | N |
| ATOM | 1543 | CA | ASP | B | 64 | 85.692 | −14.193 | 21.951 | 1.00 | 3.02 | C |
| ATOM | 1544 | CB | ASP | B | 64 | 87.183 | −14.557 | 22.090 | 1.00 | 3.02 | C |
| ATOM | 1545 | CG | ASP | B | 64 | 87.775 | −15.195 | 20.821 | 1.00 | 3.02 | C |
| ATOM | 1546 | OD1 | ASP | B | 64 | 87.395 | −16.325 | 20.440 | 1.00 | 3.02 | O |
| ATOM | 1547 | OD2 | ASP | B | 64 | 88.666 | −14.566 | 20.225 | 1.00 | 3.02 | O |
| ATOM | 1548 | C | ASP | B | 64 | 84.823 | −15.450 | 21.952 | 1.00 | 3.02 | C |
| ATOM | 1549 | O | ASP | B | 64 | 84.075 | −15.669 | 21.005 | 1.00 | 3.02 | O |
| ATOM | 1550 | N | VAL | B | 65 | 84.908 | −16.258 | 23.013 | 1.00 | 3.02 | N |
| ATOM | 1551 | CA | VAL | B | 65 | 84.059 | −17.455 | 23.152 | 1.00 | 3.02 | C |
| ATOM | 1552 | CB | VAL | B | 65 | 84.189 | −18.140 | 24.545 | 1.00 | 3.02 | C |
| ATOM | 1553 | CG1 | VAL | B | 65 | 82.884 | −18.860 | 24.937 | 1.00 | 3.02 | C |
| ATOM | 1554 | CG2 | VAL | B | 65 | 85.379 | −19.116 | 24.570 | 1.00 | 3.02 | C |
| ATOM | 1555 | C | VAL | B | 65 | 82.580 | −17.167 | 22.834 | 1.00 | 3.02 | C |
| ATOM | 1556 | O | VAL | B | 65 | 81.919 | −17.969 | 22.153 | 1.00 | 3.02 | O |
| ATOM | 1557 | N | GLU | B | 66 | 82.049 | −16.047 | 23.336 | 1.00 | 3.02 | N |
| ATOM | 1558 | CA | GLU | B | 66 | 80.753 | −15.575 | 22.844 | 1.00 | 3.02 | C |
| ATOM | 1559 | CB | GLU | B | 66 | 80.210 | −14.359 | 23.629 | 1.00 | 3.02 | C |
| ATOM | 1560 | CG | GLU | B | 66 | 78.713 | −14.073 | 23.319 | 1.00 | 3.02 | C |
| ATOM | 1561 | CD | GLU | B | 66 | 78.215 | −12.635 | 23.663 | 1.00 | 3.02 | C |
| ATOM | 1562 | OE1 | GLU | B | 66 | 78.932 | −11.878 | 24.372 | 1.00 | 3.02 | O |
| ATOM | 1563 | OE2 | GLU | B | 66 | 77.085 | −12.273 | 23.220 | 1.00 | 3.02 | O |
| ATOM | 1564 | C | GLU | B | 66 | 80.968 | −15.246 | 21.364 | 1.00 | 3.02 | C |
| ATOM | 1565 | O | GLU | B | 66 | 80.625 | −16.063 | 20.504 | 1.00 | 3.02 | O |
| ATOM | 1566 | N | ARG | B | 67 | 81.607 | −14.093 | 21.102 | 1.00 | 3.02 | N |
| ATOM | 1567 | CA | ARG | B | 67 | 81.850 | −13.516 | 19.750 | 1.00 | 3.02 | C |
| ATOM | 1568 | CB | ARG | B | 67 | 83.121 | −12.644 | 19.731 | 1.00 | 3.02 | C |
| ATOM | 1569 | CG | ARG | B | 67 | 82.932 | −11.190 | 20.138 | 1.00 | 3.02 | C |
| ATOM | 1570 | CD | ARG | B | 67 | 84.281 | −10.468 | 20.251 | 1.00 | 3.02 | C |
| ATOM | 1571 | NE | ARG | B | 67 | 84.095 | −9.143 | 20.822 | 1.00 | 3.02 | N |
| ATOM | 1572 | CZ | ARG | B | 67 | 84.128 | −8.007 | 20.127 | 1.00 | 3.02 | C |
| ATOM | 1573 | NH1 | ARG | B | 67 | 84.381 | −8.023 | 18.825 | 1.00 | 3.02 | N |
| ATOM | 1574 | NH2 | ARG | B | 67 | 83.909 | −6.846 | 20.736 | 1.00 | 3.02 | N |
| ATOM | 1575 | C | ARG | B | 67 | 81.959 | −14.514 | 18.605 | 1.00 | 3.02 | C |
| ATOM | 1576 | O | ARG | B | 67 | 81.247 | −14.377 | 17.606 | 1.00 | 3.02 | O |
| ATOM | 1577 | N | CYS | B | 68 | 82.863 | −15.489 | 18.752 | 1.00 | 3.02 | N |
| ATOM | 1578 | CA | CYS | B | 68 | 83.137 | −16.491 | 17.716 | 1.00 | 3.02 | C |
| ATOM | 1579 | CB | CYS | B | 68 | 84.499 | −17.175 | 17.940 | 1.00 | 3.02 | C |
| ATOM | 1580 | SG | CYS | B | 68 | 85.917 | −16.388 | 17.082 | 1.00 | 3.02 | S |
| ATOM | 1581 | C | CYS | B | 68 | 82.018 | −17.523 | 17.548 | 1.00 | 3.02 | C |
| ATOM | 1582 | O | CYS | B | 68 | 81.609 | −17.824 | 16.424 | 1.00 | 3.02 | O |
| ATOM | 1583 | N | LEU | B | 69 | 81.523 | −18.063 | 18.658 | 1.00 | 3.02 | N |
| ATOM | 1584 | CA | LEU | B | 69 | 80.411 | −19.007 | 18.595 | 1.00 | 3.02 | C |
| ATOM | 1585 | CB | LEU | B | 69 | 80.318 | −19.859 | 19.864 | 1.00 | 3.02 | C |
| ATOM | 1586 | CG | LEU | B | 69 | 81.122 | −21.153 | 19.925 | 1.00 | 3.02 | C |
| ATOM | 1587 | CD1 | LEU | B | 69 | 80.993 | −21.784 | 21.304 | 1.00 | 3.02 | C |
| ATOM | 1588 | CD2 | LEU | B | 69 | 80.643 | −22.112 | 18.859 | 1.00 | 3.02 | C |
| ATOM | 1589 | C | LEU | B | 69 | 79.092 | −18.286 | 18.321 | 1.00 | 3.02 | C |
| ATOM | 1590 | O | LEU | B | 69 | 78.171 | −18.882 | 17.776 | 1.00 | 3.02 | O |
| ATOM | 1591 | N | ARG | B | 70 | 79.013 | −17.009 | 18.702 | 1.00 | 3.02 | N |
| ATOM | 1592 | CA | ARG | B | 70 | 77.906 | −16.132 | 18.317 | 1.00 | 3.02 | C |
| ATOM | 1593 | CB | ARG | B | 70 | 78.162 | −14.686 | 18.764 | 1.00 | 3.02 | C |
| ATOM | 1594 | CG | ARG | B | 70 | 77.942 | −14.416 | 20.247 | 1.00 | 3.02 | C |
| ATOM | 1595 | CD | ARG | B | 70 | 76.541 | −14.855 | 20.710 | 1.00 | 3.02 | C |
| ATOM | 1596 | NE | ARG | B | 70 | 76.407 | −14.841 | 22.167 | 1.00 | 3.02 | N |
| ATOM | 1597 | CZ | ARG | B | 70 | 75.491 | −15.528 | 22.846 | 1.00 | 3.02 | C |
| ATOM | 1598 | NH1 | ARG | B | 70 | 74.608 | −16.308 | 22.203 | 1.00 | 3.02 | N |
| ATOM | 1599 | NH2 | ARG | B | 70 | 75.458 | −15.452 | 24.168 | 1.00 | 3.02 | N |
| ATOM | 1600 | C | ARG | B | 70 | 77.722 | −16.147 | 16.811 | 1.00 | 3.02 | C |
| ATOM | 1601 | O | ARG | B | 70 | 76.640 | −16.470 | 16.314 | 1.00 | 3.02 | O |
| ATOM | 1602 | N | ALA | B | 71 | 78.789 | −15.809 | 16.092 | 1.00 | 3.02 | N |
| ATOM | 1603 | CA | ALA | B | 71 | 78.758 | −15.757 | 14.636 | 1.00 | 3.02 | C |
| ATOM | 1604 | CB | ALA | B | 71 | 80.088 | −15.248 | 14.094 | 1.00 | 3.02 | C |
| ATOM | 1605 | C | ALA | B | 71 | 78.363 | −17.099 | 13.993 | 1.00 | 3.02 | C |
| ATOM | 1606 | O | ALA | B | 71 | 77.337 | −17.168 | 13.313 | 1.00 | 3.02 | O |
| ATOM | 1607 | N | HIS | B | 72 | 79.165 | −18.149 | 14.230 | 1.00 | 3.02 | N |
| ATOM | 1608 | CA | HIS | B | 72 | 78.922 | −19.519 | 13.711 | 1.00 | 3.02 | C |
| ATOM | 1609 | CB | HIS | B | 72 | 79.925 | −20.539 | 14.312 | 1.00 | 3.02 | C |
| ATOM | 1610 | CG | HIS | B | 72 | 81.361 | −20.337 | 13.894 | 1.00 | 3.02 | C |
| ATOM | 1611 | ND1 | HIS | B | 72 | 82.241 | −19.525 | 14.586 | 1.00 | 3.02 | N |
| ATOM | 1612 | CE1 | HIS | B | 72 | 83.421 | −19.546 | 13.995 | 1.00 | 3.02 | C |
| ATOM | 1613 | NE2 | HIS | B | 72 | 83.346 | −20.351 | 12.950 | 1.00 | 3.02 | N |
| ATOM | 1614 | CD2 | HIS | B | 72 | 82.076 | −20.871 | 12.873 | 1.00 | 3.02 | C |
| ATOM | 1615 | C | HIS | B | 72 | 77.462 | −19.978 | 13.969 | 1.00 | 3.02 | C |
| ATOM | 1616 | O | HIS | B | 72 | 76.729 | −20.350 | 13.034 | 1.00 | 3.02 | O |
| ATOM | 1617 | N | ARG | B | 73 | 77.049 | −19.917 | 15.239 | 1.00 | 3.02 | N |
| ATOM | 1618 | CA | ARG | B | 73 | 75.688 | −20.258 | 15.670 | 1.00 | 3.02 | C |
| ATOM | 1619 | CB | ARG | B | 73 | 75.488 | −19.939 | 17.155 | 1.00 | 3.02 | C |
| ATOM | 1620 | CG | ARG | B | 73 | 76.300 | −20.809 | 18.117 | 1.00 | 3.02 | C |
| ATOM | 1621 | CD | ARG | B | 73 | 75.968 | −20.475 | 19.572 | 1.00 | 3.02 | C |
| ATOM | 1622 | NE | ARG | B | 73 | 76.855 | −21.150 | 20.519 | 1.00 | 3.02 | N |
| ATOM | 1623 | CZ | ARG | B | 73 | 76.665 | −21.189 | 21.835 | 1.00 | 3.02 | C |
| ATOM | 1624 | NH1 | ARG | B | 73 | 75.612 | −20.589 | 22.381 | 1.00 | 3.02 | N |
| ATOM | 1625 | NH2 | ARG | B | 73 | 77.531 | −21.831 | 22.608 | 1.00 | 3.02 | N |
| ATOM | 1626 | C | ARG | B | 73 | 74.602 | −19.552 | 14.879 | 1.00 | 3.02 | C |
| ATOM | 1627 | O | ARG | B | 73 | 73.510 | −20.090 | 14.712 | 1.00 | 3.02 | O |
| ATOM | 1628 | N | ASN | B | 74 | 74.900 | −18.343 | 14.415 | 1.00 | 3.02 | N |
| ATOM | 1629 | CA | ASN | B | 74 | 73.958 | −17.556 | 13.611 | 1.00 | 3.02 | C |
| ATOM | 1630 | CB | ASN | B | 74 | 74.164 | −16.049 | 13.849 | 1.00 | 3.02 | C |
| ATOM | 1631 | CG | ASN | B | 74 | 73.580 | −15.558 | 15.174 | 1.00 | 3.02 | C |
| ATOM | 1632 | OD1 | ASN | B | 74 | 72.559 | −16.063 | 15.652 | 1.00 | 3.02 | O |
| ATOM | 1633 | ND2 | ASN | B | 74 | 74.227 | −14.548 | 15.762 | 1.00 | 3.02 | N |
| ATOM | 1634 | C | ASN | B | 74 | 74.080 | −17.844 | 12.114 | 1.00 | 3.02 | C |
| ATOM | 1635 | O | ASN | B | 74 | 73.070 | −17.876 | 11.392 | 1.00 | 3.02 | O |
| ATOM | 1636 | N | ASP | B | 75 | 75.333 | −18.029 | 11.677 | 1.00 | 3.02 | N |
| ATOM | 1637 | CA | ASP | B | 75 | 75.683 | −18.305 | 10.284 | 1.00 | 3.02 | C |
| ATOM | 1638 | CB | ASP | B | 75 | 77.191 | −18.141 | 10.041 | 1.00 | 3.02 | C |
| ATOM | 1639 | CG | ASP | B | 75 | 77.692 | −16.709 | 10.283 | 1.00 | 3.02 | C |
| ATOM | 1640 | OD1 | ASP | B | 75 | 76.874 | −15.774 | 10.434 | 1.00 | 3.02 | O |
| ATOM | 1641 | OD2 | ASP | B | 75 | 78.927 | −16.523 | 10.315 | 1.00 | 3.02 | O |
| ATOM | 1642 | C | ASP | B | 75 | 75.214 | −19.692 | 9.860 | 1.00 | 3.02 | C |
| ATOM | 1643 | O | ASP | B | 75 | 74.804 | −19.878 | 8.705 | 1.00 | 3.02 | O |
| ATOM | 1644 | N | MET | B | 76 | 75.257 | −20.657 | 10.778 | 1.00 | 3.02 | N |
| ATOM | 1645 | CA | MET | B | 76 | 74.665 | −21.991 | 10.520 | 1.00 | 3.02 | C |
| ATOM | 1646 | CB | MET | B | 76 | 75.236 | −23.027 | 11.495 | 1.00 | 3.02 | C |
| ATOM | 1647 | CG | MET | B | 76 | 76.753 | −23.067 | 11.554 | 1.00 | 3.02 | C |
| ATOM | 1648 | SD | MET | B | 76 | 77.332 | −24.201 | 12.829 | 1.00 | 3.02 | S |
| ATOM | 1649 | CE | MET | B | 76 | 76.922 | −25.753 | 12.054 | 1.00 | 3.02 | C |
| ATOM | 1650 | C | MET | B | 76 | 73.100 | −22.068 | 10.469 | 1.00 | 3.02 | C |
| ATOM | 1651 | O | MET | B | 76 | 72.540 | −22.465 | 9.452 | 1.00 | 3.02 | O |
| ATOM | 1652 | N | GLU | B | 77 | 72.425 | −21.656 | 11.545 | 1.00 | 3.02 | N |
| ATOM | 1653 | CA | GLU | B | 77 | 70.979 | −21.910 | 11.793 | 1.00 | 3.02 | C |
| ATOM | 1654 | CB | GLU | B | 77 | 70.578 | −21.267 | 13.117 | 1.00 | 3.02 | C |
| ATOM | 1655 | CG | GLU | B | 77 | 69.927 | −22.191 | 14.101 | 1.00 | 3.02 | C |
| ATOM | 1656 | CD | GLU | B | 77 | 70.399 | −21.900 | 15.506 | 1.00 | 3.02 | C |
| ATOM | 1657 | OE1 | GLU | B | 77 | 70.239 | −20.764 | 15.969 | 1.00 | 3.02 | O |
| ATOM | 1658 | OE2 | GLU | B | 77 | 70.957 | −22.794 | 16.159 | 1.00 | 3.02 | O |
| ATOM | 1659 | C | GLU | B | 77 | 69.949 | −21.499 | 10.719 | 1.00 | 3.02 | C |
| ATOM | 1660 | O | GLU | B | 77 | 69.137 | −22.321 | 10.279 | 1.00 | 3.02 | O |
| ATOM | 1661 | N | THR | B | 78 | 69.964 | −20.221 | 10.351 | 1.00 | 3.02 | N |
| ATOM | 1662 | CA | THR | B | 78 | 69.131 | −19.673 | 9.277 | 1.00 | 3.02 | C |
| ATOM | 1663 | CB | THR | B | 78 | 69.125 | −18.126 | 9.336 | 1.00 | 3.02 | C |
| ATOM | 1664 | OG1 | THR | B | 78 | 67.904 | −17.659 | 9.930 | 1.00 | 3.02 | O |
| ATOM | 1665 | CG2 | THR | B | 78 | 69.297 | −17.504 | 7.961 | 1.00 | 3.02 | C |
| ATOM | 1666 | C | THR | B | 78 | 69.578 | −20.165 | 7.893 | 1.00 | 3.02 | C |
| ATOM | 1667 | O | THR | B | 78 | 68.761 | −20.240 | 6.971 | 1.00 | 3.02 | O |
| ATOM | 1668 | N | ILE | B | 79 | 70.869 | −20.503 | 7.762 | 1.00 | 3.02 | N |
| ATOM | 1669 | CA | ILE | B | 79 | 71.420 | −21.033 | 6.515 | 1.00 | 3.02 | C |
| ATOM | 1670 | CB | ILE | B | 79 | 72.951 | −21.256 | 6.609 | 1.00 | 3.02 | C |
| ATOM | 1671 | CG1 | ILE | B | 79 | 73.690 | −20.081 | 5.956 | 1.00 | 3.02 | C |
| ATOM | 1672 | CD1 | ILE | B | 79 | 73.941 | −20.211 | 4.464 | 1.00 | 3.02 | C |
| ATOM | 1673 | CG2 | ILE | B | 79 | 73.391 | −22.602 | 6.006 | 1.00 | 3.02 | C |
| ATOM | 1674 | C | ILE | B | 79 | 70.665 | −22.278 | 6.102 | 1.00 | 3.02 | C |
| ATOM | 1675 | O | ILE | B | 79 | 70.099 | −22.318 | 5.012 | 1.00 | 3.02 | O |
| ATOM | 1676 | N | TYR | B | 80 | 70.599 | −23.261 | 6.997 | 1.00 | 3.02 | N |
| ATOM | 1677 | CA | TYR | B | 80 | 70.044 | −24.561 | 6.644 | 1.00 | 3.02 | C |
| ATOM | 1678 | CB | TYR | B | 80 | 69.886 | −25.476 | 7.871 | 1.00 | 3.02 | C |
| ATOM | 1679 | CG | TYR | B | 80 | 71.202 | −25.819 | 8.547 | 1.00 | 3.02 | C |
| ATOM | 1680 | CD1 | TYR | B | 80 | 71.393 | −25.557 | 9.900 | 1.00 | 3.02 | C |
| ATOM | 1681 | CE1 | TYR | B | 80 | 72.580 | −25.854 | 10.522 | 1.00 | 3.02 | C |
| ATOM | 1682 | CZ | TYR | B | 80 | 73.603 | −26.419 | 9.792 | 1.00 | 3.02 | C |
| ATOM | 1683 | OH | TYR | B | 80 | 74.794 | −26.716 | 10.419 | 1.00 | 3.02 | O |
| ATOM | 1684 | CE2 | TYR | B | 80 | 73.446 | −26.682 | 8.441 | 1.00 | 3.02 | C |
| ATOM | 1685 | CD2 | TYR | B | 80 | 72.259 | −26.383 | 7.828 | 1.00 | 3.02 | C |
| ATOM | 1686 | C | TYR | B | 80 | 68.748 | −24.423 | 5.847 | 1.00 | 3.02 | C |
| ATOM | 1687 | O | TYR | B | 80 | 68.698 | −24.886 | 4.702 | 1.00 | 3.02 | O |
| ATOM | 1688 | N | PRO | B | 81 | 67.725 | −23.734 | 6.416 | 1.00 | 3.02 | N |
| ATOM | 1689 | CA | PRO | B | 81 | 66.404 | −23.744 | 5.785 | 1.00 | 3.02 | C |
| ATOM | 1690 | CB | PRO | B | 81 | 65.464 | −23.399 | 6.941 | 1.00 | 3.02 | C |
| ATOM | 1691 | CG | PRO | B | 81 | 66.273 | −22.521 | 7.814 | 1.00 | 3.02 | C |
| ATOM | 1692 | CD | PRO | B | 81 | 67.727 | −22.914 | 7.644 | 1.00 | 3.02 | C |
| ATOM | 1693 | C | PRO | B | 81 | 66.325 | −22.710 | 4.679 | 1.00 | 3.02 | C |
| ATOM | 1694 | O | PRO | B | 81 | 65.280 | −22.517 | 4.073 | 1.00 | 3.02 | O |
| ATOM | 1695 | N | PHE | B | 82 | 67.439 | −22.046 | 4.414 | 1.00 | 3.02 | N |
| ATOM | 1696 | CA | PHE | B | 82 | 67.501 | −21.119 | 3.298 | 1.00 | 3.02 | C |
| ATOM | 1697 | CB | PHE | B | 82 | 68.405 | −19.951 | 3.628 | 1.00 | 3.02 | C |
| ATOM | 1698 | CG | PHE | B | 82 | 68.493 | −18.955 | 2.542 | 1.00 | 3.02 | C |
| ATOM | 1699 | CD1 | PHE | B | 82 | 67.558 | −17.939 | 2.444 | 1.00 | 3.02 | C |
| ATOM | 1700 | CE1 | PHE | B | 82 | 67.636 | −17.004 | 1.436 | 1.00 | 3.02 | C |
| ATOM | 1701 | CZ | PHE | B | 82 | 68.658 | −17.084 | 0.506 | 1.00 | 3.02 | C |
| ATOM | 1702 | CE2 | PHE | B | 82 | 69.588 | −18.105 | 0.581 | 1.00 | 3.02 | C |
| ATOM | 1703 | CD2 | PHE | B | 82 | 69.498 | −19.037 | 1.597 | 1.00 | 3.02 | C |
| ATOM | 1704 | C | PHE | B | 82 | 68.037 | −21.812 | 2.059 | 1.00 | 3.02 | C |
| ATOM | 1705 | O | PHE | B | 82 | 67.570 | −21.577 | 0.937 | 1.00 | 3.02 | O |
| ATOM | 1706 | N | LEU | B | 83 | 69.049 | −22.647 | 2.279 | 1.00 | 3.02 | N |
| ATOM | 1707 | CA | LEU | B | 83 | 69.705 | −23.361 | 1.201 | 1.00 | 3.02 | C |
| ATOM | 1708 | CB | LEU | B | 83 | 71.109 | −23.814 | 1.607 | 1.00 | 3.02 | C |
| ATOM | 1709 | CG | LEU | B | 83 | 71.957 | −22.870 | 2.473 | 1.00 | 3.02 | C |
| ATOM | 1710 | CD1 | LEU | B | 83 | 73.363 | −23.417 | 2.651 | 1.00 | 3.02 | C |
| ATOM | 1711 | CD2 | LEU | B | 83 | 72.006 | −21.437 | 1.956 | 1.00 | 3.02 | C |
| ATOM | 1712 | C | LEU | B | 83 | 68.841 | −24.536 | 0.798 | 1.00 | 3.02 | C |
| ATOM | 1713 | O | LEU | B | 83 | 68.273 | −24.547 | −0.296 | 1.00 | 3.02 | O |
| ATOM | 1714 | N | PHE | B | 84 | 68.736 | −25.513 | 1.695 | 1.00 | 3.02 | N |
| ATOM | 1715 | CA | PHE | B | 84 | 67.823 | −26.642 | 1.538 | 1.00 | 3.02 | C |
| ATOM | 1716 | CB | PHE | B | 84 | 67.709 | −27.379 | 2.883 | 1.00 | 3.02 | C |
| ATOM | 1717 | CG | PHE | B | 84 | 66.422 | −28.122 | 3.078 | 1.00 | 3.02 | C |
| ATOM | 1718 | CD1 | PHE | B | 84 | 66.401 | −29.322 | 3.748 | 1.00 | 3.02 | C |
| ATOM | 1719 | CE1 | PHE | B | 84 | 65.216 | −29.995 | 3.929 | 1.00 | 3.02 | C |
| ATOM | 1720 | CZ | PHE | B | 84 | 64.043 | −29.465 | 3.449 | 1.00 | 3.02 | C |
| ATOM | 1721 | CE2 | PHE | B | 84 | 64.056 | −28.279 | 2.785 | 1.00 | 3.02 | C |
| ATOM | 1722 | CD2 | PHE | B | 84 | 65.230 | −27.615 | 2.609 | 1.00 | 3.02 | C |
| ATOM | 1723 | C | PHE | B | 84 | 66.452 | −26.200 | 0.991 | 1.00 | 3.02 | C |
| ATOM | 1724 | O | PHE | B | 84 | 66.081 | −26.575 | −0.122 | 1.00 | 3.02 | O |
| ATOM | 1725 | N | LEU | B | 85 | 65.740 | −25.379 | 1.766 | 1.00 | 3.02 | N |
| ATOM | 1726 | CA | LEU | B | 85 | 64.385 | −24.941 | 1.429 | 1.00 | 3.02 | C |
| ATOM | 1727 | CB | LEU | B | 85 | 63.816 | −24.024 | 2.520 | 1.00 | 3.02 | C |
| ATOM | 1728 | CG | LEU | B | 85 | 63.284 | −24.667 | 3.810 | 1.00 | 3.02 | C |
| ATOM | 1729 | CD1 | LEU | B | 85 | 62.481 | −23.682 | 4.641 | 1.00 | 3.02 | C |
| ATOM | 1730 | CD2 | LEU | B | 85 | 62.420 | −25.850 | 3.499 | 1.00 | 3.02 | C |
| ATOM | 1731 | C | LEU | B | 85 | 64.271 | −24.294 | 0.056 | 1.00 | 3.02 | C |
| ATOM | 1732 | O | LEU | B | 85 | 63.405 | −24.669 | −0.746 | 1.00 | 3.02 | O |
| ATOM | 1733 | N | GLY | B | 86 | 65.146 | −23.328 | −0.213 | 1.00 | 3.02 | N |
| ATOM | 1734 | CA | GLY | B | 86 | 65.207 | −22.702 | −1.528 | 1.00 | 3.02 | C |
| ATOM | 1735 | C | GLY | B | 86 | 65.432 | −23.695 | −2.660 | 1.00 | 3.02 | C |
| ATOM | 1736 | O | GLY | B | 86 | 64.707 | −23.684 | −3.655 | 1.00 | 3.02 | O |
| ATOM | 1737 | N | PHE | B | 87 | 66.422 | −24.573 | −2.492 | 1.00 | 3.02 | N |
| ATOM | 1738 | CA | PHE | B | 87 | 66.968 | −25.329 | −3.618 | 1.00 | 3.02 | C |
| ATOM | 1739 | CB | PHE | B | 87 | 68.431 | −25.691 | −3.386 | 1.00 | 3.02 | C |
| ATOM | 1740 | CG | PHE | B | 87 | 69.319 | −24.503 | −3.304 | 1.00 | 3.02 | C |
| ATOM | 1741 | CD1 | PHE | B | 87 | 69.698 | −23.989 | −2.079 | 1.00 | 3.02 | C |
| ATOM | 1742 | CE1 | PHE | B | 87 | 70.513 | −22.864 | −1.988 | 1.00 | 3.02 | C |
| ATOM | 1743 | CZ | PHE | B | 87 | 70.939 | −22.229 | −3.137 | 1.00 | 3.02 | C |
| ATOM | 1744 | CE2 | PHE | B | 87 | 70.548 | −22.720 | −4.374 | 1.00 | 3.02 | C |
| ATOM | 1745 | CD2 | PHE | B | 87 | 69.746 | −23.860 | −4.455 | 1.00 | 3.02 | C |
| ATOM | 1746 | C | PHE | B | 87 | 66.156 | −26.511 | −4.086 | 1.00 | 3.02 | C |
| ATOM | 1747 | O | PHE | B | 87 | 66.186 | −26.822 | −5.278 | 1.00 | 3.02 | O |
| ATOM | 1748 | N | VAL | B | 88 | 65.445 | −27.176 | −3.170 | 1.00 | 3.02 | N |
| ATOM | 1749 | CA | VAL | B | 88 | 64.456 | −28.189 | −3.568 | 1.00 | 3.02 | C |
| ATOM | 1750 | CB | VAL | B | 88 | 63.664 | −28.773 | −2.368 | 1.00 | 3.02 | C |
| ATOM | 1751 | CG1 | VAL | B | 88 | 63.246 | −30.188 | −2.671 | 1.00 | 3.02 | C |
| ATOM | 1752 | CG2 | VAL | B | 88 | 64.479 | −28.757 | −1.100 | 1.00 | 3.02 | C |
| ATOM | 1753 | C | VAL | B | 88 | 63.445 | −27.541 | −4.526 | 1.00 | 3.02 | C |
| ATOM | 1754 | O | VAL | B | 88 | 62.810 | −28.220 | −5.347 | 1.00 | 3.02 | O |
| ATOM | 1755 | N | TYR | B | 89 | 63.332 | −26.214 | −4.414 | 1.00 | 3.02 | N |
| ATOM | 1756 | CA | TYR | B | 89 | 62.347 | −25.406 | −5.130 | 1.00 | 3.02 | C |
| ATOM | 1757 | CB | TYR | B | 89 | 61.512 | −24.603 | −4.105 | 1.00 | 3.02 | C |
| ATOM | 1758 | CG | TYR | B | 89 | 61.431 | −23.081 | −4.250 | 1.00 | 3.02 | C |
| ATOM | 1759 | CD1 | TYR | B | 89 | 60.207 | −22.442 | −4.490 | 1.00 | 3.02 | C |
| ATOM | 1760 | CE1 | TYR | B | 89 | 60.127 | −21.043 | −4.591 | 1.00 | 3.02 | C |
| ATOM | 1761 | CZ | TYR | B | 89 | 61.280 | −20.290 | −4.444 | 1.00 | 3.02 | C |
| ATOM | 1762 | OH | TYR | B | 89 | 61.247 | −18.924 | −4.545 | 1.00 | 3.02 | O |
| ATOM | 1763 | CE2 | TYR | B | 89 | 62.490 | −20.900 | −4.200 | 1.00 | 3.02 | C |
| ATOM | 1764 | CD2 | TYR | B | 89 | 62.563 | −22.277 | −4.093 | 1.00 | 3.02 | C |
| ATOM | 1765 | C | TYR | B | 89 | 62.940 | −24.522 | −6.232 | 1.00 | 3.02 | C |
| ATOM | 1766 | O | TYR | B | 89 | 62.197 | −23.835 | −6.944 | 1.00 | 3.02 | O |
| ATOM | 1767 | N | SER | B | 90 | 64.263 | −24.529 | −6.381 | 1.00 | 3.02 | N |
| ATOM | 1768 | CA | SER | B | 90 | 64.901 | −23.697 | −7.401 | 1.00 | 3.02 | C |
| ATOM | 1769 | CB | SER | B | 90 | 66.361 | −23.413 | −7.043 | 1.00 | 3.02 | C |
| ATOM | 1770 | OG | SER | B | 90 | 67.073 | −24.609 | −6.782 | 1.00 | 3.02 | O |
| ATOM | 1771 | C | SER | B | 90 | 64.814 | −24.361 | −8.763 | 1.00 | 3.02 | C |
| ATOM | 1772 | O | SER | B | 90 | 64.170 | −23.848 | −9.681 | 1.00 | 3.02 | O |
| ATOM | 1773 | N | PHE | B | 91 | 65.482 | −25.502 | −8.884 | 1.00 | 3.02 | N |
| ATOM | 1774 | CA | PHE | B | 91 | 65.512 | −26.276 | −10.117 | 1.00 | 3.02 | C |
| ATOM | 1775 | CB | PHE | B | 91 | 66.702 | −27.242 | −10.076 | 1.00 | 3.02 | C |
| ATOM | 1776 | CG | PHE | B | 91 | 67.913 | −26.668 | −9.374 | 1.00 | 3.02 | C |
| ATOM | 1777 | CD1 | PHE | B | 91 | 68.219 | −27.026 | −8.065 | 1.00 | 3.02 | C |
| ATOM | 1778 | CE1 | PHE | B | 91 | 69.325 | −26.485 | −7.407 | 1.00 | 3.02 | C |
| ATOM | 1779 | CZ | PHE | B | 91 | 70.127 | −25.562 | −8.059 | 1.00 | 3.02 | C |
| ATOM | 1780 | CE2 | PHE | B | 91 | 69.826 | −25.184 | −9.362 | 1.00 | 3.02 | C |
| ATOM | 1781 | CD2 | PHE | B | 91 | 68.723 | −25.736 | −10.011 | 1.00 | 3.02 | C |
| ATOM | 1782 | C | PHE | B | 91 | 64.143 | −26.969 | −10.281 | 1.00 | 3.02 | C |
| ATOM | 1783 | O | PHE | B | 91 | 64.002 | −28.206 | −10.184 | 1.00 | 3.02 | O |
| ATOM | 1784 | N | LEU | B | 92 | 63.139 | −26.126 | −10.537 | 1.00 | 3.02 | N |
| ATOM | 1785 | CA | LEU | B | 92 | 61.735 | −26.480 | −10.414 | 1.00 | 3.02 | C |
| ATOM | 1786 | CB | LEU | B | 92 | 61.351 | −26.467 | −8.937 | 1.00 | 3.02 | C |
| ATOM | 1787 | CG | LEU | B | 92 | 60.167 | −27.309 | −8.490 | 1.00 | 3.02 | C |
| ATOM | 1788 | CD1 | LEU | B | 92 | 60.673 | −28.687 | −8.128 | 1.00 | 3.02 | C |
| ATOM | 1789 | CD2 | LEU | B | 92 | 59.471 | −26.663 | −7.302 | 1.00 | 3.02 | C |
| ATOM | 1790 | C | LEU | B | 92 | 60.852 | −25.491 | −11.178 | 1.00 | 3.02 | C |
| ATOM | 1791 | O | LEU | B | 92 | 59.835 | −25.896 | −11.744 | 1.00 | 3.02 | O |
| ATOM | 1792 | N | GLY | B | 93 | 61.239 | −24.206 | −11.173 | 1.00 | 3.02 | N |
| ATOM | 1793 | CA | GLY | B | 93 | 60.530 | −23.122 | −11.893 | 1.00 | 3.02 | C |
| ATOM | 1794 | C | GLY | B | 93 | 59.971 | −21.994 | −11.022 | 1.00 | 3.02 | C |
| ATOM | 1795 | O | GLY | B | 93 | 58.778 | −21.981 | −10.747 | 1.00 | 3.02 | O |
| ATOM | 1796 | N | PRO | B | 94 | 60.817 | −21.012 | −10.626 | 1.00 | 3.02 | N |
| ATOM | 1797 | CA | PRO | B | 94 | 60.510 | −20.044 | −9.563 | 1.00 | 3.02 | C |
| ATOM | 1798 | CB | PRO | B | 94 | 61.883 | −19.827 | −8.914 | 1.00 | 3.02 | C |
| ATOM | 1799 | CG | PRO | B | 94 | 62.856 | −19.975 | −10.056 | 1.00 | 3.02 | C |
| ATOM | 1800 | CD | PRO | B | 94 | 62.151 | −20.736 | −11.189 | 1.00 | 3.02 | C |
| ATOM | 1801 | C | PRO | B | 94 | 59.978 | −18.678 | −10.032 | 1.00 | 3.02 | C |
| ATOM | 1802 | O | PRO | B | 94 | 59.798 | −18.461 | −11.232 | 1.00 | 3.02 | O |
| ATOM | 1803 | N | ASN | B | 95 | 59.756 | −17.768 | −9.080 | 1.00 | 3.02 | N |
| ATOM | 1804 | CA | ASN | B | 95 | 59.331 | −16.397 | −9.384 | 1.00 | 3.02 | C |
| ATOM | 1805 | CB | ASN | B | 95 | 57.838 | −16.236 | −9.110 | 1.00 | 3.02 | C |
| ATOM | 1806 | CG | ASN | B | 95 | 56.982 | −17.035 | −10.083 | 1.00 | 3.02 | C |
| ATOM | 1807 | OD1 | ASN | B | 95 | 57.377 | −17.297 | −11.221 | 1.00 | 3.02 | O |
| ATOM | 1808 | ND2 | ASN | B | 95 | 55.800 | −17.420 | −9.640 | 1.00 | 3.02 | N |
| ATOM | 1809 | C | ASN | B | 95 | 60.193 | −15.299 | −8.705 | 1.00 | 3.02 | C |
| ATOM | 1810 | O | ASN | B | 95 | 60.732 | −15.523 | −7.606 | 1.00 | 3.02 | O |
| ATOM | 1811 | N | PRO | B | 96 | 60.303 | −14.107 | −9.350 | 1.00 | 3.02 | N |
| ATOM | 1812 | CA | PRO | B | 96 | 61.450 | −13.219 | −9.104 | 1.00 | 3.02 | C |
| ATOM | 1813 | CB | PRO | B | 96 | 61.470 | −12.284 | −10.336 | 1.00 | 3.02 | C |
| ATOM | 1814 | CG | PRO | B | 96 | 60.234 | −12.625 | −11.143 | 1.00 | 3.02 | C |
| ATOM | 1815 | CD | PRO | B | 96 | 59.349 | −13.497 | −10.294 | 1.00 | 3.02 | C |
| ATOM | 1816 | C | PRO | B | 96 | 61.381 | −12.432 | −7.790 | 1.00 | 3.02 | C |
| ATOM | 1817 | O | PRO | B | 96 | 62.191 | −12.691 | −6.898 | 1.00 | 3.02 | O |
| ATOM | 1818 | N | PHE | B | 97 | 60.434 | −11.501 | −7.663 | 1.00 | 3.02 | N |
| ATOM | 1819 | CA | PHE | B | 97 | 60.285 | −10.703 | −6.442 | 1.00 | 3.02 | C |
| ATOM | 1820 | CB | PHE | B | 97 | 59.032 | −9.829 | −6.514 | 1.00 | 3.02 | C |
| ATOM | 1821 | CG | PHE | B | 97 | 58.973 | −8.932 | −7.722 | 1.00 | 3.02 | C |
| ATOM | 1822 | CD1 | PHE | B | 97 | 58.301 | −9.339 | −8.867 | 1.00 | 3.02 | C |
| ATOM | 1823 | CE1 | PHE | B | 97 | 58.233 | −8.511 | −9.986 | 1.00 | 3.02 | C |
| ATOM | 1824 | CZ | PHE | B | 97 | 58.832 | −7.265 | −9.957 | 1.00 | 3.02 | C |
| ATOM | 1825 | CE2 | PHE | B | 97 | 59.501 | −6.852 | −8.819 | 1.00 | 3.02 | C |
| ATOM | 1826 | CD2 | PHE | B | 97 | 59.564 | −7.678 | −7.708 | 1.00 | 3.02 | C |
| ATOM | 1827 | C | PHE | B | 97 | 60.224 | −11.575 | −5.182 | 1.00 | 3.02 | C |
| ATOM | 1828 | O | PHE | B | 97 | 60.509 | −11.115 | −4.080 | 1.00 | 3.02 | O |
| ATOM | 1829 | N | VAL | B | 98 | 59.849 | −12.842 | −5.359 | 1.00 | 3.02 | N |
| ATOM | 1830 | CA | VAL | B | 98 | 59.782 | −13.811 | −4.255 | 1.00 | 3.02 | C |
| ATOM | 1831 | CB | VAL | B | 98 | 58.659 | −14.863 | −4.477 | 1.00 | 3.02 | C |
| ATOM | 1832 | CG1 | VAL | B | 98 | 58.965 | −16.184 | −3.781 | 1.00 | 3.02 | C |
| ATOM | 1833 | CG2 | VAL | B | 98 | 57.318 | −14.293 | −4.003 | 1.00 | 3.02 | C |
| ATOM | 1834 | C | VAL | B | 98 | 61.148 | −14.448 | −3.966 | 1.00 | 3.02 | C |
| ATOM | 1835 | O | VAL | B | 98 | 61.611 | −14.457 | −2.817 | 1.00 | 3.02 | O |
| ATOM | 1836 | N | ALA | B | 99 | 61.803 | −14.960 | −5.001 | 1.00 | 3.02 | N |
| ATOM | 1837 | CA | ALA | B | 99 | 63.194 | −15.361 | −4.855 | 1.00 | 3.02 | C |
| ATOM | 1838 | CB | ALA | B | 99 | 63.829 | −15.600 | −6.217 | 1.00 | 3.02 | C |
| ATOM | 1839 | C | ALA | B | 99 | 63.924 | −14.245 | −4.135 | 1.00 | 3.02 | C |
| ATOM | 1840 | O | ALA | B | 99 | 64.420 | −14.425 | −3.022 | 1.00 | 3.02 | O |
| ATOM | 1841 | N | TRP | B | 100 | 63.906 | −13.077 | −4.778 | 1.00 | 3.02 | N |
| ATOM | 1842 | CA | TRP | B | 100 | 64.700 | −11.886 | −4.432 | 1.00 | 3.02 | C |
| ATOM | 1843 | CB | TRP | B | 100 | 64.587 | −10.861 | −5.566 | 1.00 | 3.02 | C |
| ATOM | 1844 | CG | TRP | B | 100 | 65.546 | −11.100 | −6.710 | 1.00 | 3.02 | C |
| ATOM | 1845 | CD1 | TRP | B | 100 | 65.249 | −11.597 | −7.950 | 1.00 | 3.02 | C |
| ATOM | 1846 | NE1 | TRP | B | 100 | 66.385 | −11.660 | −8.720 | 1.00 | 3.02 | N |
| ATOM | 1847 | CE2 | TRP | B | 100 | 67.447 | −11.203 | −7.982 | 1.00 | 3.02 | C |
| ATOM | 1848 | CD2 | TRP | B | 100 | 66.955 | −10.837 | −6.711 | 1.00 | 3.02 | C |
| ATOM | 1849 | CE3 | TRP | B | 100 | 67.852 | −10.322 | −5.759 | 1.00 | 3.02 | C |
| ATOM | 1850 | CZ3 | TRP | B | 100 | 69.195 | −10.186 | −6.102 | 1.00 | 3.02 | C |
| ATOM | 1851 | CH2 | TRP | B | 100 | 69.655 | −10.559 | −7.375 | 1.00 | 3.02 | C |
| ATOM | 1852 | CZ2 | TRP | B | 100 | 68.802 | −11.072 | −8.326 | 1.00 | 3.02 | C |
| ATOM | 1853 | C | TRP | B | 100 | 64.429 | −11.204 | −3.072 | 1.00 | 3.02 | C |
| ATOM | 1854 | O | TRP | B | 100 | 65.280 | −10.467 | −2.565 | 1.00 | 3.02 | O |
| ATOM | 1855 | N | MET | B | 101 | 63.247 | −11.430 | −2.501 | 1.00 | 3.02 | N |
| ATOM | 1856 | CA | MET | B | 101 | 62.975 | −11.054 | −1.108 | 1.00 | 3.02 | C |
| ATOM | 1857 | CB | MET | B | 101 | 61.461 | −10.979 | −0.834 | 1.00 | 3.02 | C |
| ATOM | 1858 | CG | MET | B | 101 | 60.809 | −9.625 | −1.114 | 1.00 | 3.02 | C |
| ATOM | 1859 | SD | MET | B | 101 | 59.029 | −9.754 | −1.458 | 1.00 | 3.02 | S |
| ATOM | 1860 | CE | MET | B | 101 | 58.334 | −9.330 | 0.147 | 1.00 | 3.02 | C |
| ATOM | 1861 | C | MET | B | 101 | 63.639 | −12.056 | −0.147 | 1.00 | 3.02 | C |
| ATOM | 1862 | O | MET | B | 101 | 64.354 | −11.652 | 0.773 | 1.00 | 3.02 | O |
| ATOM | 1863 | N | HIS | B | 102 | 63.415 | −13.353 | −0.382 | 1.00 | 3.02 | N |
| ATOM | 1864 | CA | HIS | B | 102 | 63.923 | −14.407 | 0.497 | 1.00 | 3.02 | C |
| ATOM | 1865 | CB | HIS | B | 102 | 63.619 | −15.789 | −0.090 | 1.00 | 3.02 | C |
| ATOM | 1866 | CG | HIS | B | 102 | 62.160 | −16.124 | −0.150 | 1.00 | 3.02 | C |
| ATOM | 1867 | ND1 | HIS | B | 102 | 61.301 | −15.897 | 0.901 | 1.00 | 3.02 | N |
| ATOM | 1868 | CE1 | HIS | B | 102 | 60.090 | −16.312 | 0.575 | 1.00 | 3.02 | C |
| ATOM | 1869 | NE2 | HIS | B | 102 | 60.135 | −16.812 | −0.645 | 1.00 | 3.02 | N |
| ATOM | 1870 | CD2 | HIS | B | 102 | 61.421 | −16.716 | −1.118 | 1.00 | 3.02 | C |
| ATOM | 1871 | C | HIS | B | 102 | 65.423 | −14.283 | 0.834 | 1.00 | 3.02 | C |
| ATOM | 1872 | O | HIS | B | 102 | 65.839 | −14.579 | 1.956 | 1.00 | 3.02 | O |
| ATOM | 1873 | N | PHE | B | 103 | 66.222 | −13.839 | −0.131 | 1.00 | 3.02 | N |
| ATOM | 1874 | CA | PHE | B | 103 | 67.646 | −13.591 | 0.098 | 1.00 | 3.02 | C |
| ATOM | 1875 | CB | PHE | B | 103 | 68.393 | −13.460 | −1.232 | 1.00 | 3.02 | C |
| ATOM | 1876 | CG | PHE | B | 103 | 68.194 | −14.629 | −2.141 | 1.00 | 3.02 | C |
| ATOM | 1877 | CD1 | PHE | B | 103 | 67.264 | −14.572 | −3.174 | 1.00 | 3.02 | C |
| ATOM | 1878 | CE1 | PHE | B | 103 | 67.052 | −15.667 | −4.018 | 1.00 | 3.02 | C |
| ATOM | 1879 | CZ | PHE | B | 103 | 67.771 | −16.838 | −3.824 | 1.00 | 3.02 | C |
| ATOM | 1880 | CE2 | PHE | B | 103 | 68.696 | −16.910 | −2.779 | 1.00 | 3.02 | C |
| ATOM | 1881 | CD2 | PHE | B | 103 | 68.908 | −15.799 | −1.948 | 1.00 | 3.02 | C |
| ATOM | 1882 | C | PHE | B | 103 | 67.890 | −12.361 | 0.962 | 1.00 | 3.02 | C |
| ATOM | 1883 | O | PHE | B | 103 | 68.789 | −12.368 | 1.800 | 1.00 | 3.02 | O |
| ATOM | 1884 | N | LEU | B | 104 | 67.082 | −11.318 | 0.744 | 1.00 | 3.02 | N |
| ATOM | 1885 | CA | LEU | B | 104 | 67.146 | −10.057 | 1.507 | 1.00 | 3.02 | C |
| ATOM | 1886 | CB | LEU | B | 104 | 66.675 | −8.888 | 0.651 | 1.00 | 3.02 | C |
| ATOM | 1887 | CG | LEU | B | 104 | 67.404 | −8.696 | −0.672 | 1.00 | 3.02 | C |
| ATOM | 1888 | CD1 | LEU | B | 104 | 66.658 | −7.752 | −1.595 | 1.00 | 3.02 | C |
| ATOM | 1889 | CD2 | LEU | B | 104 | 68.835 | −8.215 | −0.429 | 1.00 | 3.02 | C |
| ATOM | 1890 | C | LEU | B | 104 | 66.345 | −10.072 | 2.806 | 1.00 | 3.02 | C |
| ATOM | 1891 | O | LEU | B | 104 | 66.743 | −9.432 | 3.779 | 1.00 | 3.02 | O |
| ATOM | 1892 | N | VAL | B | 105 | 65.211 | −10.776 | 2.813 | 1.00 | 3.02 | N |
| ATOM | 1893 | CA | VAL | B | 105 | 64.402 | −10.915 | 4.023 | 1.00 | 3.02 | C |
| ATOM | 1894 | CB | VAL | B | 105 | 62.955 | −11.431 | 3.725 | 1.00 | 3.02 | C |
| ATOM | 1895 | CG1 | VAL | B | 105 | 62.161 | −11.623 | 5.004 | 1.00 | 3.02 | C |
| ATOM | 1896 | CG2 | VAL | B | 105 | 62.206 | −10.464 | 2.809 | 1.00 | 3.02 | C |
| ATOM | 1897 | C | VAL | B | 105 | 65.146 | −11.811 | 5.015 | 1.00 | 3.02 | C |
| ATOM | 1898 | O | VAL | B | 105 | 65.505 | −11.365 | 6.099 | 1.00 | 3.02 | O |
| ATOM | 1899 | N | PHE | B | 106 | 65.394 | −13.062 | 4.635 | 1.00 | 3.02 | N |
| ATOM | 1900 | CA | PHE | B | 106 | 66.240 | −13.953 | 5.429 | 1.00 | 3.02 | C |
| ATOM | 1901 | CB | PHE | B | 106 | 66.758 | −15.115 | 4.575 | 1.00 | 3.02 | C |
| ATOM | 1902 | CG | PHE | B | 106 | 66.258 | −16.464 | 4.999 | 1.00 | 3.02 | C |
| ATOM | 1903 | CD1 | PHE | B | 106 | 64.929 | −16.821 | 4.833 | 1.00 | 3.02 | C |
| ATOM | 1904 | CE1 | PHE | B | 106 | 64.471 | −18.081 | 5.225 | 1.00 | 3.02 | C |
| ATOM | 1905 | CZ | PHE | B | 106 | 65.345 | −18.994 | 5.763 | 1.00 | 3.02 | C |
| ATOM | 1906 | CE2 | PHE | B | 106 | 66.670 | −18.655 | 5.928 | 1.00 | 3.02 | C |
| ATOM | 1907 | CD2 | PHE | B | 106 | 67.129 | −17.397 | 5.539 | 1.00 | 3.02 | C |
| ATOM | 1908 | C | PHE | B | 106 | 67.432 | −13.212 | 6.025 | 1.00 | 3.02 | C |
| ATOM | 1909 | O | PHE | B | 106 | 67.565 | −13.140 | 7.254 | 1.00 | 3.02 | O |
| ATOM | 1910 | N | LEU | B | 107 | 68.264 | −12.644 | 5.135 | 1.00 | 3.02 | N |
| ATOM | 1911 | CA | LEU | B | 107 | 69.613 | −12.173 | 5.477 | 1.00 | 3.02 | C |
| ATOM | 1912 | CB | LEU | B | 107 | 70.455 | −11.896 | 4.227 | 1.00 | 3.02 | C |
| ATOM | 1913 | CG | LEU | B | 107 | 71.815 | −12.600 | 4.087 | 1.00 | 3.02 | C |
| ATOM | 1914 | CD1 | LEU | B | 107 | 72.670 | −11.838 | 3.070 | 1.00 | 3.02 | C |
| ATOM | 1915 | CD2 | LEU | B | 107 | 72.564 | −12.775 | 5.428 | 1.00 | 3.02 | C |
| ATOM | 1916 | C | LEU | B | 107 | 69.607 | −10.952 | 6.355 | 1.00 | 3.02 | C |
| ATOM | 1917 | O | LEU | B | 107 | 70.223 | −10.954 | 7.408 | 1.00 | 3.02 | O |
| ATOM | 1918 | N | VAL | B | 108 | 68.931 | −9.895 | 5.928 | 1.00 | 3.02 | N |
| ATOM | 1919 | CA | VAL | B | 108 | 68.817 | −8.727 | 6.805 | 1.00 | 3.02 | C |
| ATOM | 1920 | CB | VAL | B | 108 | 68.344 | −7.414 | 6.089 | 1.00 | 3.02 | C |
| ATOM | 1921 | CG1 | VAL | B | 108 | 68.934 | −6.207 | 6.802 | 1.00 | 3.02 | C |
| ATOM | 1922 | CG2 | VAL | B | 108 | 68.794 | −7.372 | 4.628 | 1.00 | 3.02 | C |
| ATOM | 1923 | C | VAL | B | 108 | 67.953 | −9.110 | 8.025 | 1.00 | 3.02 | C |
| ATOM | 1924 | O | VAL | B | 108 | 67.985 | −8.404 | 9.080 | 1.00 | 3.02 | O |
| ATOM | 1925 | N | GLY | B | 109 | 67.212 | −10.217 | 7.887 | 1.00 | 3.02 | N |
| ATOM | 1926 | CA | GLY | B | 109 | 66.622 | −10.914 | 9.033 | 1.00 | 3.02 | C |
| ATOM | 1927 | C | GLY | B | 109 | 67.678 | −11.601 | 9.894 | 1.00 | 3.02 | C |
| ATOM | 1928 | O | GLY | B | 109 | 67.394 | −11.986 | 11.104 | 1.00 | 3.02 | O |
| ATOM | 1929 | N | ARG | B | 110 | 68.868 | −11.831 | 9.333 | 1.00 | 3.02 | N |
| ATOM | 1930 | CA | ARG | B | 110 | 69.953 | −12.503 | 10.074 | 1.00 | 3.02 | C |
| ATOM | 1931 | CB | ARG | B | 110 | 70.590 | −13.617 | 9.230 | 1.00 | 3.02 | C |
| ATOM | 1932 | CG | ARG | B | 110 | 71.389 | −14.610 | 10.044 | 1.00 | 3.02 | C |
| ATOM | 1933 | CD | ARG | B | 110 | 70.492 | −15.218 | 11.090 | 1.00 | 3.02 | C |
| ATOM | 1934 | NE | ARG | B | 110 | 71.168 | −16.170 | 11.963 | 1.00 | 3.02 | N |
| ATOM | 1935 | CZ | ARG | B | 110 | 70.528 | −17.006 | 12.772 | 1.00 | 3.02 | C |
| ATOM | 1936 | NH1 | ARG | B | 110 | 69.194 | −17.030 | 12.798 | 1.00 | 3.02 | N |
| ATOM | 1937 | NH2 | ARG | B | 110 | 71.227 | −17.828 | 13.534 | 1.00 | 3.02 | N |
| ATOM | 1938 | C | ARG | B | 110 | 71.064 | −11.579 | 10.610 | 1.00 | 3.02 | C |
| ATOM | 1939 | O | ARG | B | 110 | 71.482 | −11.716 | 11.760 | 1.00 | 3.02 | O |
| ATOM | 1940 | N | VAL | B | 111 | 71.528 | −10.647 | 9.771 | 1.00 | 3.02 | N |
| ATOM | 1941 | CA | VAL | B | 111 | 72.775 | −9.894 | 10.000 | 1.00 | 3.02 | C |
| ATOM | 1942 | CB | VAL | B | 111 | 73.277 | −9.206 | 8.699 | 1.00 | 3.02 | C |
| ATOM | 1943 | CG1 | VAL | B | 111 | 74.640 | −8.560 | 8.896 | 1.00 | 3.02 | C |
| ATOM | 1944 | CG2 | VAL | B | 111 | 73.371 | −10.198 | 7.570 | 1.00 | 3.02 | C |
| ATOM | 1945 | C | VAL | B | 111 | 72.631 | −8.849 | 11.094 | 1.00 | 3.02 | C |
| ATOM | 1946 | O | VAL | B | 111 | 73.587 | −8.540 | 11.822 | 1.00 | 3.02 | O |
| ATOM | 1947 | N | ALA | B | 112 | 71.431 | −8.288 | 11.190 | 1.00 | 3.02 | N |
| ATOM | 1948 | CA | ALA | B | 112 | 71.073 | −7.403 | 12.302 | 1.00 | 3.02 | C |
| ATOM | 1949 | CB | ALA | B | 112 | 70.044 | −6.364 | 11.877 | 1.00 | 3.02 | C |
| ATOM | 1950 | C | ALA | B | 112 | 70.567 | −8.245 | 13.486 | 1.00 | 3.02 | C |
| ATOM | 1951 | O | ALA | B | 112 | 70.309 | −7.697 | 14.596 | 1.00 | 3.02 | O |
| ATOM | 1952 | N | HIS | B | 113 | 70.380 | −9.550 | 13.257 | 1.00 | 3.02 | N |
| ATOM | 1953 | CA | HIS | B | 113 | 70.277 | −10.509 | 14.354 | 1.00 | 3.02 | C |
| ATOM | 1954 | CB | HIS | B | 113 | 69.438 | −11.728 | 13.942 | 1.00 | 3.02 | C |
| ATOM | 1955 | CG | HIS | B | 113 | 69.692 | −12.949 | 14.774 | 1.00 | 3.02 | C |
| ATOM | 1956 | ND1 | HIS | B | 113 | 68.806 | −13.404 | 15.726 | 1.00 | 3.02 | N |
| ATOM | 1957 | CE1 | HIS | B | 113 | 69.296 | −14.487 | 16.300 | 1.00 | 3.02 | C |
| ATOM | 1958 | NE2 | HIS | B | 113 | 70.468 | −14.752 | 15.756 | 1.00 | 3.02 | N |
| ATOM | 1959 | CD2 | HIS | B | 113 | 70.740 | −13.806 | 14.799 | 1.00 | 3.02 | C |
| ATOM | 1960 | C | HIS | B | 113 | 71.678 | −10.924 | 14.826 | 1.00 | 3.02 | C |
| ATOM | 1961 | O | HIS | B | 113 | 71.923 | −11.060 | 16.029 | 1.00 | 3.02 | O |
| ATOM | 1962 | N | THR | B | 114 | 72.597 | −11.101 | 13.876 | 1.00 | 3.02 | N |
| ATOM | 1963 | CA | THR | B | 114 | 73.954 | −11.620 | 14.164 | 1.00 | 3.02 | C |
| ATOM | 1964 | CB | THR | B | 114 | 74.582 | −12.340 | 12.936 | 1.00 | 3.02 | C |
| ATOM | 1965 | OG1 | THR | B | 114 | 73.755 | −13.443 | 12.548 | 1.00 | 3.02 | O |
| ATOM | 1966 | CG2 | THR | B | 114 | 75.979 | −12.847 | 13.259 | 1.00 | 3.02 | C |
| ATOM | 1967 | C | THR | B | 114 | 74.986 | −10.628 | 14.751 | 1.00 | 3.02 | C |
| ATOM | 1968 | O | THR | B | 114 | 75.823 | −11.033 | 15.566 | 1.00 | 3.02 | O |
| ATOM | 1969 | N | VAL | B | 115 | 74.963 | −9.364 | 14.325 | 1.00 | 3.02 | N |
| ATOM | 1970 | CA | VAL | B | 115 | 75.862 | −8.368 | 14.944 | 1.00 | 3.02 | C |
| ATOM | 1971 | CB | VAL | B | 115 | 76.393 | −7.258 | 13.959 | 1.00 | 3.02 | C |
| ATOM | 1972 | CG1 | VAL | B | 115 | 76.744 | −5.987 | 14.703 | 1.00 | 3.02 | C |
| ATOM | 1973 | CG2 | VAL | B | 115 | 77.626 | −7.729 | 13.221 | 1.00 | 3.02 | C |
| ATOM | 1974 | C | VAL | B | 115 | 75.216 | −7.780 | 16.197 | 1.00 | 3.02 | C |
| ATOM | 1975 | O | VAL | B | 115 | 75.906 | −7.182 | 17.039 | 1.00 | 3.02 | O |
| ATOM | 1976 | N | ALA | B | 116 | 73.901 | −7.966 | 16.316 | 1.00 | 3.02 | N |
| ATOM | 1977 | CA | ALA | B | 116 | 73.183 | −7.554 | 17.509 | 1.00 | 3.02 | C |
| ATOM | 1978 | CB | ALA | B | 116 | 71.678 | −7.702 | 17.315 | 1.00 | 3.02 | C |
| ATOM | 1979 | C | ALA | B | 116 | 73.666 | −8.424 | 18.648 | 1.00 | 3.02 | C |
| ATOM | 1980 | O | ALA | B | 116 | 74.371 | −7.958 | 19.538 | 1.00 | 3.02 | O |
| ATOM | 1981 | N | TYR | B | 117 | 73.319 | −9.703 | 18.593 | 1.00 | 3.02 | N |
| ATOM | 1982 | CA | TYR | B | 117 | 73.760 | −10.642 | 19.608 | 1.00 | 3.02 | C |
| ATOM | 1983 | CB | TYR | B | 117 | 73.328 | −12.068 | 19.265 | 1.00 | 3.02 | C |
| ATOM | 1984 | CG | TYR | B | 117 | 73.142 | −12.951 | 20.474 | 1.00 | 3.02 | C |
| ATOM | 1985 | CD1 | TYR | B | 117 | 72.694 | −14.252 | 20.333 | 1.00 | 3.02 | C |
| ATOM | 1986 | CE1 | TYR | B | 117 | 72.506 | −15.070 | 21.438 | 1.00 | 3.02 | C |
| ATOM | 1987 | CZ | TYR | B | 117 | 72.756 | −14.585 | 22.707 | 1.00 | 3.02 | C |
| ATOM | 1988 | OH | TYR | B | 117 | 72.571 | −15.415 | 23.795 | 1.00 | 3.02 | O |
| ATOM | 1989 | CE2 | TYR | B | 117 | 73.210 | −13.289 | 22.876 | 1.00 | 3.02 | C |
| ATOM | 1990 | CD2 | TYR | B | 117 | 73.388 | −12.479 | 21.765 | 1.00 | 3.02 | C |
| ATOM | 1991 | C | TYR | B | 117 | 75.286 | −10.576 | 19.775 | 1.00 | 3.02 | C |
| ATOM | 1992 | O | TYR | B | 117 | 75.819 | −10.903 | 20.841 | 1.00 | 3.02 | O |
| ATOM | 1993 | N | LEU | B | 118 | 75.990 | −10.158 | 18.724 | 1.00 | 3.02 | N |
| ATOM | 1994 | CA | LEU | B | 118 | 77.431 | −10.024 | 18.811 | 1.00 | 3.02 | C |
| ATOM | 1995 | CB | LEU | B | 118 | 78.052 | −9.665 | 17.455 | 1.00 | 3.02 | C |
| ATOM | 1996 | CG | LEU | B | 118 | 78.653 | −10.777 | 16.578 | 1.00 | 3.02 | C |
| ATOM | 1997 | CD1 | LEU | B | 118 | 78.837 | −10.316 | 15.138 | 1.00 | 3.02 | C |
| ATOM | 1998 | CD2 | LEU | B | 118 | 80.001 | −11.294 | 17.127 | 1.00 | 3.02 | C |
| ATOM | 1999 | C | LEU | B | 118 | 77.793 | −8.987 | 19.868 | 1.00 | 3.02 | C |
| ATOM | 2000 | O | LEU | B | 118 | 77.995 | −9.329 | 21.030 | 1.00 | 3.02 | O |
| ATOM | 2001 | N | GLY | B | 119 | 77.841 | −7.721 | 19.460 | 1.00 | 3.02 | N |
| ATOM | 2002 | CA | GLY | B | 119 | 78.287 | −6.609 | 20.310 | 1.00 | 3.02 | C |
| ATOM | 2003 | C | GLY | B | 119 | 77.828 | −6.624 | 21.759 | 1.00 | 3.02 | C |
| ATOM | 2004 | O | GLY | B | 119 | 78.638 | −6.865 | 22.660 | 1.00 | 3.02 | O |
| ATOM | 2005 | N | LYS | B | 120 | 76.539 | −6.359 | 21.983 | 1.00 | 3.02 | N |
| ATOM | 2006 | CA | LYS | B | 120 | 75.980 | −6.315 | 23.329 | 1.00 | 3.02 | C |
| ATOM | 2007 | CB | LYS | B | 120 | 74.791 | −5.358 | 23.391 | 1.00 | 3.02 | C |
| ATOM | 2008 | CG | LYS | B | 120 | 75.155 | −3.876 | 23.242 | 1.00 | 3.02 | C |
| ATOM | 2009 | CD | LYS | B | 120 | 73.949 | −2.990 | 23.558 | 1.00 | 3.02 | C |
| ATOM | 2010 | CE | LYS | B | 120 | 74.315 | −1.522 | 23.689 | 1.00 | 3.02 | C |
| ATOM | 2011 | NZ | LYS | B | 120 | 73.214 | −0.754 | 24.341 | 1.00 | 3.02 | N |
| ATOM | 2012 | C | LYS | B | 120 | 75.584 | −7.715 | 23.761 | 1.00 | 3.02 | C |
| ATOM | 2013 | O | LYS | B | 120 | 74.795 | −8.376 | 23.092 | 1.00 | 3.02 | O |
| ATOM | 2014 | N | LEU | B | 121 | 76.150 | −8.146 | 24.886 | 1.00 | 3.02 | N |
| ATOM | 2015 | CA | LEU | B | 121 | 76.127 | −9.546 | 25.332 | 1.00 | 3.02 | C |
| ATOM | 2016 | CB | LEU | B | 121 | 76.937 | −9.705 | 26.616 | 1.00 | 3.02 | C |
| ATOM | 2017 | CG | LEU | B | 121 | 78.200 | −8.860 | 26.744 | 1.00 | 3.02 | C |
| ATOM | 2018 | CD1 | LEU | B | 121 | 78.761 | −8.962 | 28.162 | 1.00 | 3.02 | C |
| ATOM | 2019 | CD2 | LEU | B | 121 | 79.244 | −9.252 | 25.681 | 1.00 | 3.02 | C |
| ATOM | 2020 | C | LEU | B | 121 | 74.738 | −10.080 | 25.575 | 1.00 | 3.02 | C |
| ATOM | 2021 | O | LEU | B | 121 | 74.443 | −11.212 | 25.197 | 1.00 | 3.02 | O |
| ATOM | 2022 | N | ARG | B | 122 | 73.919 | −9.244 | 26.216 | 1.00 | 3.02 | N |
| ATOM | 2023 | CA | ARG | B | 122 | 72.537 | −9.538 | 26.616 | 1.00 | 3.02 | C |
| ATOM | 2024 | CB | ARG | B | 122 | 71.899 | −8.299 | 27.279 | 1.00 | 3.02 | C |
| ATOM | 2025 | CG | ARG | B | 122 | 72.844 | −7.527 | 28.218 | 1.00 | 3.02 | C |
| ATOM | 2026 | CD | ARG | B | 122 | 72.152 | −6.432 | 29.019 | 1.00 | 3.02 | C |
| ATOM | 2027 | NE | ARG | B | 122 | 71.486 | −6.953 | 30.215 | 1.00 | 3.02 | N |
| ATOM | 2028 | CZ | ARG | B | 122 | 70.928 | −6.196 | 31.163 | 1.00 | 3.02 | C |
| ATOM | 2029 | NH1 | ARG | B | 122 | 70.957 | −4.860 | 31.074 | 1.00 | 3.02 | N |
| ATOM | 2030 | NH2 | ARG | B | 122 | 70.343 | −6.780 | 32.208 | 1.00 | 3.02 | N |
| ATOM | 2031 | C | ARG | B | 122 | 71.667 | −10.019 | 25.452 | 1.00 | 3.02 | C |
| ATOM | 2032 | O | ARG | B | 122 | 71.594 | −9.368 | 24.409 | 1.00 | 3.02 | O |
| ATOM | 2033 | N | ALA | B | 123 | 71.005 | −11.158 | 25.652 | 1.00 | 3.02 | N |
| ATOM | 2034 | CA | ALA | B | 123 | 70.214 | −11.798 | 24.600 | 1.00 | 3.02 | C |
| ATOM | 2035 | CB | ALA | B | 123 | 69.738 | −13.168 | 25.035 | 1.00 | 3.02 | C |
| ATOM | 2036 | C | ALA | B | 123 | 69.039 | −10.967 | 24.072 | 1.00 | 3.02 | C |
| ATOM | 2037 | O | ALA | B | 123 | 68.818 | −10.956 | 22.856 | 1.00 | 3.02 | O |
| ATOM | 2038 | N | PRO | B | 124 | 68.288 | −10.267 | 24.965 | 1.00 | 3.02 | N |
| ATOM | 2039 | CA | PRO | B | 124 | 67.135 | −9.505 | 24.497 | 1.00 | 3.02 | C |
| ATOM | 2040 | CB | PRO | B | 124 | 67.108 | −8.304 | 25.455 | 1.00 | 3.02 | C |
| ATOM | 2041 | CG | PRO | B | 124 | 68.090 | −8.640 | 26.582 | 1.00 | 3.02 | C |
| ATOM | 2042 | CD | PRO | B | 124 | 68.446 | −10.082 | 26.416 | 1.00 | 3.02 | C |
| ATOM | 2043 | C | PRO | B | 124 | 67.322 | −9.027 | 23.062 | 1.00 | 3.02 | C |
| ATOM | 2044 | O | PRO | B | 124 | 66.469 | −9.264 | 22.211 | 1.00 | 3.02 | O |
| ATOM | 2045 | N | ILE | B | 125 | 68.464 | −8.399 | 22.812 | 1.00 | 3.02 | N |
| ATOM | 2046 | CA | ILE | B | 125 | 68.838 | −7.866 | 21.504 | 1.00 | 3.02 | C |
| ATOM | 2047 | CB | ILE | B | 125 | 70.374 | −7.498 | 21.465 | 1.00 | 3.02 | C |
| ATOM | 2048 | CG1 | ILE | B | 125 | 70.693 | −6.521 | 20.330 | 1.00 | 3.02 | C |
| ATOM | 2049 | CD1 | ILE | B | 125 | 71.829 | −5.610 | 20.638 | 1.00 | 3.02 | C |
| ATOM | 2050 | CG2 | ILE | B | 125 | 71.261 | −8.746 | 21.417 | 1.00 | 3.02 | C |
| ATOM | 2051 | C | ILE | B | 125 | 68.406 | −8.713 | 20.293 | 1.00 | 3.02 | C |
| ATOM | 2052 | O | ILE | B | 125 | 67.719 | −8.209 | 19.396 | 1.00 | 3.02 | O |
| ATOM | 2053 | N | ARG | B | 126 | 68.778 | −9.994 | 20.286 | 1.00 | 3.02 | N |
| ATOM | 2054 | CA | ARG | B | 126 | 68.491 | −10.872 | 19.144 | 1.00 | 3.02 | C |
| ATOM | 2055 | CB | ARG | B | 126 | 69.316 | −12.177 | 19.207 | 1.00 | 3.02 | C |
| ATOM | 2056 | CG | ARG | B | 126 | 68.749 | −13.310 | 20.077 | 1.00 | 3.02 | C |
| ATOM | 2057 | CD | ARG | B | 126 | 67.809 | −14.227 | 19.260 | 1.00 | 3.02 | C |
| ATOM | 2058 | NE | ARG | B | 126 | 67.484 | −15.508 | 19.896 | 1.00 | 3.02 | N |
| ATOM | 2059 | CZ | ARG | B | 126 | 67.147 | −16.608 | 19.228 | 1.00 | 3.02 | C |
| ATOM | 2060 | NH1 | ARG | B | 126 | 67.098 | −16.603 | 17.905 | 1.00 | 3.02 | N |
| ATOM | 2061 | NH2 | ARG | B | 126 | 66.874 | −17.719 | 19.882 | 1.00 | 3.02 | N |
| ATOM | 2062 | C | ARG | B | 126 | 66.981 | −11.126 | 18.944 | 1.00 | 3.02 | C |
| ATOM | 2063 | O | ARG | B | 126 | 66.565 | −11.589 | 17.878 | 1.00 | 3.02 | O |
| ATOM | 2064 | N | SER | B | 127 | 66.178 | −10.772 | 19.952 | 1.00 | 3.02 | N |
| ATOM | 2065 | CA | SER | B | 127 | 64.754 | −11.123 | 20.008 | 1.00 | 3.02 | C |
| ATOM | 2066 | CB | SER | B | 127 | 64.146 | −10.760 | 21.378 | 1.00 | 3.02 | C |
| ATOM | 2067 | OG | SER | B | 127 | 64.119 | −9.364 | 21.621 | 1.00 | 3.02 | O |
| ATOM | 2068 | C | SER | B | 127 | 63.866 | −10.628 | 18.844 | 1.00 | 3.02 | C |
| ATOM | 2069 | O | SER | B | 127 | 62.955 | −11.353 | 18.411 | 1.00 | 3.02 | O |
| ATOM | 2070 | N | VAL | B | 128 | 64.111 | −9.414 | 18.338 | 1.00 | 3.02 | N |
| ATOM | 2071 | CA | VAL | B | 128 | 63.297 | −8.879 | 17.222 | 1.00 | 3.02 | C |
| ATOM | 2072 | CB | VAL | B | 128 | 63.166 | −7.312 | 17.234 | 1.00 | 3.02 | C |
| ATOM | 2073 | CG1 | VAL | B | 128 | 62.410 | −6.808 | 16.002 | 1.00 | 3.02 | C |
| ATOM | 2074 | CG2 | VAL | B | 128 | 62.463 | −6.822 | 18.511 | 1.00 | 3.02 | C |
| ATOM | 2075 | C | VAL | B | 128 | 63.748 | −9.435 | 15.856 | 1.00 | 3.02 | C |
| ATOM | 2076 | O | VAL | B | 128 | 63.045 | −10.262 | 15.267 | 1.00 | 3.02 | O |
| ATOM | 2077 | N | THR | B | 129 | 64.916 | −9.012 | 15.372 | 1.00 | 3.02 | N |
| ATOM | 2078 | CA | THR | B | 129 | 65.424 | −9.478 | 14.071 | 1.00 | 3.02 | C |
| ATOM | 2079 | CB | THR | B | 129 | 66.824 | −8.903 | 13.741 | 1.00 | 3.02 | C |
| ATOM | 2080 | OG1 | THR | B | 129 | 67.721 | −9.112 | 14.846 | 1.00 | 3.02 | O |
| ATOM | 2081 | CG2 | THR | B | 129 | 66.712 | −7.429 | 13.406 | 1.00 | 3.02 | C |
| ATOM | 2082 | C | THR | B | 129 | 65.399 | −11.009 | 13.842 | 1.00 | 3.02 | C |
| ATOM | 2083 | O | THR | B | 129 | 65.176 | −11.463 | 12.713 | 1.00 | 3.02 | O |
| ATOM | 2084 | N | TYR | B | 130 | 65.612 | −11.800 | 14.893 | 1.00 | 3.02 | N |
| ATOM | 2085 | CA | TYR | B | 130 | 65.450 | −13.261 | 14.777 | 1.00 | 3.02 | C |
| ATOM | 2086 | CB | TYR | B | 130 | 65.891 | −13.988 | 16.052 | 1.00 | 3.02 | C |
| ATOM | 2087 | CG | TYR | B | 130 | 64.778 | −14.563 | 16.900 | 1.00 | 3.02 | C |
| ATOM | 2088 | CD1 | TYR | B | 130 | 64.460 | −15.912 | 16.821 | 1.00 | 3.02 | C |
| ATOM | 2089 | CE1 | TYR | B | 130 | 63.447 | −16.460 | 17.605 | 1.00 | 3.02 | C |
| ATOM | 2090 | CZ | TYR | B | 130 | 62.736 | −15.654 | 18.463 | 1.00 | 3.02 | C |
| ATOM | 2091 | OH | TYR | B | 130 | 61.752 | −16.241 | 19.216 | 1.00 | 3.02 | O |
| ATOM | 2092 | CE2 | TYR | B | 130 | 63.027 | −14.298 | 18.559 | 1.00 | 3.02 | C |
| ATOM | 2093 | CD2 | TYR | B | 130 | 64.050 | −13.765 | 17.784 | 1.00 | 3.02 | C |
| ATOM | 2094 | C | TYR | B | 130 | 64.011 | −13.658 | 14.411 | 1.00 | 3.02 | C |
| ATOM | 2095 | O | TYR | B | 130 | 63.801 | −14.673 | 13.725 | 1.00 | 3.02 | O |
| ATOM | 2096 | N | THR | B | 131 | 63.030 | −12.878 | 14.887 | 1.00 | 3.02 | N |
| ATOM | 2097 | CA | THR | B | 131 | 61.636 | −13.068 | 14.470 | 1.00 | 3.02 | C |
| ATOM | 2098 | CB | THR | B | 131 | 60.590 | −12.357 | 15.409 | 1.00 | 3.02 | C |
| ATOM | 2099 | OG1 | THR | B | 131 | 60.355 | −13.162 | 16.577 | 1.00 | 3.02 | O |
| ATOM | 2100 | CG2 | THR | B | 131 | 59.236 | −12.147 | 14.702 | 1.00 | 3.02 | C |
| ATOM | 2101 | C | THR | B | 131 | 61.494 | −12.602 | 13.020 | 1.00 | 3.02 | C |
| ATOM | 2102 | O | THR | B | 131 | 60.748 | −13.209 | 12.229 | 1.00 | 3.02 | O |
| ATOM | 2103 | N | LEU | B | 132 | 62.232 | −11.549 | 12.668 | 1.00 | 3.02 | N |
| ATOM | 2104 | CA | LEU | B | 132 | 62.193 | −11.028 | 11.306 | 1.00 | 3.02 | C |
| ATOM | 2105 | CB | LEU | B | 132 | 62.896 | −9.672 | 11.201 | 1.00 | 3.02 | C |
| ATOM | 2106 | CG | LEU | B | 132 | 62.333 | −8.498 | 12.024 | 1.00 | 3.02 | C |
| ATOM | 2107 | CD1 | LEU | B | 132 | 62.937 | −7.186 | 11.556 | 1.00 | 3.02 | C |
| ATOM | 2108 | CD2 | LEU | B | 132 | 60.805 | −8.399 | 12.013 | 1.00 | 3.02 | C |
| ATOM | 2109 | C | LEU | B | 132 | 62.753 | −12.017 | 10.282 | 1.00 | 3.02 | C |
| ATOM | 2110 | O | LEU | B | 132 | 62.519 | −11.854 | 9.084 | 1.00 | 3.02 | O |
| ATOM | 2111 | N | ALA | B | 133 | 63.467 | −13.043 | 10.754 | 1.00 | 3.02 | N |
| ATOM | 2112 | CA | ALA | B | 133 | 64.022 | −14.077 | 9.875 | 1.00 | 3.02 | C |
| ATOM | 2113 | CB | ALA | B | 133 | 65.490 | −14.346 | 10.200 | 1.00 | 3.02 | C |
| ATOM | 2114 | C | ALA | B | 133 | 63.229 | −15.372 | 9.896 | 1.00 | 3.02 | C |
| ATOM | 2115 | O | ALA | B | 133 | 63.135 | −16.062 | 8.876 | 1.00 | 3.02 | O |
| ATOM | 2116 | N | GLN | B | 134 | 62.653 | −15.700 | 11.048 | 1.00 | 3.02 | N |
| ATOM | 2117 | CA | GLN | B | 134 | 61.939 | −16.976 | 11.206 | 1.00 | 3.02 | C |
| ATOM | 2118 | CB | GLN | B | 134 | 61.475 | −17.157 | 12.649 | 1.00 | 3.02 | C |
| ATOM | 2119 | CG | GLN | B | 134 | 62.562 | −17.651 | 13.587 | 1.00 | 3.02 | C |
| ATOM | 2120 | CD | GLN | B | 134 | 62.079 | −17.758 | 15.013 | 1.00 | 3.02 | C |
| ATOM | 2121 | OE1 | GLN | B | 134 | 61.215 | −16.990 | 15.443 | 1.00 | 3.02 | O |
| ATOM | 2122 | NE2 | GLN | B | 134 | 62.647 | −18.697 | 15.766 | 1.00 | 3.02 | N |
| ATOM | 2123 | C | GLN | B | 134 | 60.766 | −17.130 | 10.242 | 1.00 | 3.02 | C |
| ATOM | 2124 | O | GLN | B | 134 | 60.626 | −18.180 | 9.611 | 1.00 | 3.02 | O |
| ATOM | 2125 | N | LEU | B | 135 | 59.955 | −16.066 | 10.144 | 1.00 | 3.02 | N |
| ATOM | 2126 | CA | LEU | B | 135 | 58.771 | −15.986 | 9.260 | 1.00 | 3.02 | C |
| ATOM | 2127 | CB | LEU | B | 135 | 58.043 | −14.625 | 9.434 | 1.00 | 3.02 | C |
| ATOM | 2128 | CG | LEU | B | 135 | 57.083 | −14.373 | 10.625 | 1.00 | 3.02 | C |
| ATOM | 2129 | CD1 | LEU | B | 135 | 56.756 | −12.893 | 10.833 | 1.00 | 3.02 | C |
| ATOM | 2130 | CD2 | LEU | B | 135 | 55.773 | −15.165 | 10.530 | 1.00 | 3.02 | C |
| ATOM | 2131 | C | LEU | B | 135 | 59.060 | −16.303 | 7.764 | 1.00 | 3.02 | C |
| ATOM | 2132 | O | LEU | B | 135 | 58.266 | −17.003 | 7.122 | 1.00 | 3.02 | O |
| ATOM | 2133 | N | PRO | B | 136 | 60.165 | −15.744 | 7.202 | 1.00 | 3.02 | N |
| ATOM | 2134 | CA | PRO | B | 136 | 60.776 | −16.125 | 5.919 | 1.00 | 3.02 | C |
| ATOM | 2135 | CB | PRO | B | 136 | 62.147 | −15.461 | 5.983 | 1.00 | 3.02 | C |
| ATOM | 2136 | CG | PRO | B | 136 | 61.942 | −14.278 | 6.769 | 1.00 | 3.02 | C |
| ATOM | 2137 | CD | PRO | B | 136 | 60.845 | −14.559 | 7.760 | 1.00 | 3.02 | C |
| ATOM | 2138 | C | PRO | B | 136 | 60.954 | −17.617 | 5.648 | 1.00 | 3.02 | C |
| ATOM | 2139 | O | PRO | B | 136 | 60.882 | −18.023 | 4.489 | 1.00 | 3.02 | O |
| ATOM | 2140 | N | CYS | B | 137 | 61.210 | −18.416 | 6.687 | 1.00 | 3.02 | N |
| ATOM | 2141 | CA | CYS | B | 137 | 61.234 | −19.885 | 6.542 | 1.00 | 3.02 | C |
| ATOM | 2142 | CB | CYS | B | 137 | 61.900 | −20.558 | 7.748 | 1.00 | 3.02 | C |
| ATOM | 2143 | SG | CYS | B | 137 | 63.670 | −20.139 | 7.957 | 1.00 | 3.02 | S |
| ATOM | 2144 | C | CYS | B | 137 | 59.818 | −20.446 | 6.295 | 1.00 | 3.02 | C |
| ATOM | 2145 | O | CYS | B | 137 | 59.651 | −21.503 | 5.691 | 1.00 | 3.02 | O |
| ATOM | 2146 | N | ALA | B | 138 | 58.802 | −19.713 | 6.751 | 1.00 | 3.02 | N |
| ATOM | 2147 | CA | ALA | B | 138 | 57.403 | −20.008 | 6.433 | 1.00 | 3.02 | C |
| ATOM | 2148 | CB | ALA | B | 138 | 56.481 | −19.489 | 7.544 | 1.00 | 3.02 | C |
| ATOM | 2149 | C | ALA | B | 138 | 56.972 | −19.433 | 5.071 | 1.00 | 3.02 | C |
| ATOM | 2150 | O | ALA | B | 138 | 56.263 | −20.098 | 4.309 | 1.00 | 3.02 | O |
| ATOM | 2151 | N | SER | B | 139 | 57.377 | −18.192 | 4.782 | 1.00 | 3.02 | N |
| ATOM | 2152 | CA | SER | B | 139 | 57.125 | −17.589 | 3.465 | 1.00 | 3.02 | C |
| ATOM | 2153 | CB | SER | B | 139 | 57.706 | −16.186 | 3.382 | 1.00 | 3.02 | C |
| ATOM | 2154 | OG | SER | B | 139 | 59.087 | −16.257 | 3.126 | 1.00 | 3.02 | O |
| ATOM | 2155 | C | SER | B | 139 | 57.716 | −18.470 | 2.359 | 1.00 | 3.02 | C |
| ATOM | 2156 | O | SER | B | 139 | 56.995 | −18.886 | 1.448 | 1.00 | 3.02 | O |
| ATOM | 2157 | N | MET | B | 140 | 59.020 | −18.762 | 2.460 | 1.00 | 3.02 | N |
| ATOM | 2158 | CA | MET | B | 140 | 59.657 | −19.823 | 1.656 | 1.00 | 3.02 | C |
| ATOM | 2159 | CB | MET | B | 140 | 61.113 | −20.084 | 2.065 | 1.00 | 3.02 | C |
| ATOM | 2160 | CG | MET | B | 140 | 62.047 | −18.893 | 1.922 | 1.00 | 3.02 | C |
| ATOM | 2161 | SD | MET | B | 140 | 63.814 | −19.275 | 2.082 | 1.00 | 3.02 | S |
| ATOM | 2162 | CE | MET | B | 140 | 64.239 | −19.739 | 0.397 | 1.00 | 3.02 | C |
| ATOM | 2163 | C | MET | B | 140 | 58.857 | −21.122 | 1.748 | 1.00 | 3.02 | C |
| ATOM | 2164 | O | MET | B | 140 | 58.463 | −21.670 | 0.726 | 1.00 | 3.02 | O |
| ATOM | 2165 | N | ALA | B | 141 | 58.575 | −21.595 | 2.959 | 1.00 | 3.02 | N |
| ATOM | 2166 | CA | ALA | B | 141 | 57.853 | −22.855 | 3.106 | 1.00 | 3.02 | C |
| ATOM | 2167 | CB | ALA | B | 141 | 57.489 | −23.106 | 4.561 | 1.00 | 3.02 | C |
| ATOM | 2168 | C | ALA | B | 141 | 56.609 | −22.931 | 2.208 | 1.00 | 3.02 | C |
| ATOM | 2169 | O | ALA | B | 141 | 56.439 | −23.907 | 1.468 | 1.00 | 3.02 | O |
| ATOM | 2170 | N | LEU | B | 142 | 55.772 | −21.885 | 2.266 | 1.00 | 3.02 | N |
| ATOM | 2171 | CA | LEU | B | 142 | 54.486 | −21.827 | 1.538 | 1.00 | 3.02 | C |
| ATOM | 2172 | CB | LEU | B | 142 | 53.506 | −20.855 | 2.206 | 1.00 | 3.02 | C |
| ATOM | 2173 | CG | LEU | B | 142 | 52.938 | −21.246 | 3.574 | 1.00 | 3.02 | C |
| ATOM | 2174 | CD1 | LEU | B | 142 | 52.579 | −20.007 | 4.372 | 1.00 | 3.02 | C |
| ATOM | 2175 | CD2 | LEU | B | 142 | 51.732 | −22.154 | 3.450 | 1.00 | 3.02 | C |
| ATOM | 2176 | C | LEU | B | 142 | 54.674 | −21.457 | 0.072 | 1.00 | 3.02 | C |
| ATOM | 2177 | O | LEU | B | 142 | 54.015 | −22.020 | −0.805 | 1.00 | 3.02 | O |
| ATOM | 2178 | N | GLN | B | 143 | 55.583 | −20.522 | −0.191 | 1.00 | 3.02 | N |
| ATOM | 2179 | CA | GLN | B | 143 | 55.919 | −20.174 | −1.565 | 1.00 | 3.02 | C |
| ATOM | 2180 | CB | GLN | B | 143 | 56.604 | −18.796 | −1.653 | 1.00 | 3.02 | C |
| ATOM | 2181 | CG | GLN | B | 143 | 55.575 | −17.622 | −1.538 | 1.00 | 3.02 | C |
| ATOM | 2182 | CD | GLN | B | 143 | 56.183 | −16.252 | −1.207 | 1.00 | 3.02 | C |
| ATOM | 2183 | OE1 | GLN | B | 143 | 57.405 | −16.096 | −1.121 | 1.00 | 3.02 | O |
| ATOM | 2184 | NE2 | GLN | B | 143 | 55.319 | −15.256 | −1.018 | 1.00 | 3.02 | N |
| ATOM | 2185 | C | GLN | B | 143 | 56.656 | −21.324 | −2.298 | 1.00 | 3.02 | C |
| ATOM | 2186 | O | GLN | B | 143 | 56.481 | −21.496 | −3.512 | 1.00 | 3.02 | O |
| ATOM | 2187 | N | ILE | B | 144 | 57.404 | −22.148 | −1.548 | 1.00 | 3.02 | N |
| ATOM | 2188 | CA | ILE | B | 144 | 57.941 | −23.432 | −2.069 | 1.00 | 3.02 | C |
| ATOM | 2189 | CB | ILE | B | 144 | 58.989 | −24.075 | −1.117 | 1.00 | 3.02 | C |
| ATOM | 2190 | CG1 | ILE | B | 144 | 60.228 | −23.197 | −0.984 | 1.00 | 3.02 | C |
| ATOM | 2191 | CD1 | ILE | B | 144 | 61.019 | −23.475 | 0.262 | 1.00 | 3.02 | C |
| ATOM | 2192 | CG2 | ILE | B | 144 | 59.396 | −25.474 | −1.601 | 1.00 | 3.02 | C |
| ATOM | 2193 | C | ILE | B | 144 | 56.855 | −24.496 | −2.328 | 1.00 | 3.02 | C |
| ATOM | 2194 | O | ILE | B | 144 | 56.830 | −25.140 | −3.391 | 1.00 | 3.02 | O |
| ATOM | 2195 | N | LEU | B | 145 | 55.980 | −24.697 | −1.346 | 1.00 | 3.02 | N |
| ATOM | 2196 | CA | LEU | B | 145 | 55.000 | −25.760 | −1.425 | 1.00 | 3.02 | C |
| ATOM | 2197 | CB | LEU | B | 145 | 54.318 | −25.960 | −0.073 | 1.00 | 3.02 | C |
| ATOM | 2198 | CG | LEU | B | 145 | 54.893 | −27.025 | 0.862 | 1.00 | 3.02 | C |
| ATOM | 2199 | CD1 | LEU | B | 145 | 54.182 | −26.979 | 2.193 | 1.00 | 3.02 | C |
| ATOM | 2200 | CD2 | LEU | B | 145 | 54.776 | −28.403 | 0.250 | 1.00 | 3.02 | C |
| ATOM | 2201 | C | LEU | B | 145 | 53.953 | −25.561 | −2.518 | 1.00 | 3.02 | C |
| ATOM | 2202 | O | LEU | B | 145 | 53.404 | −26.534 | −3.037 | 1.00 | 3.02 | O |
| ATOM | 2203 | N | TRP | B | 146 | 53.654 | −24.312 | −2.862 | 1.00 | 3.02 | N |
| ATOM | 2204 | CA | TRP | B | 146 | 52.617 | −24.066 | −3.874 | 1.00 | 3.02 | C |
| ATOM | 2205 | CB | TRP | B | 146 | 52.082 | −22.633 | −3.817 | 1.00 | 3.02 | C |
| ATOM | 2206 | CG | TRP | B | 146 | 51.586 | −22.195 | −2.467 | 1.00 | 3.02 | C |
| ATOM | 2207 | CD1 | TRP | B | 146 | 51.720 | −20.956 | −1.921 | 1.00 | 3.02 | C |
| ATOM | 2208 | NE1 | TRP | B | 146 | 51.155 | −20.924 | −0.671 | 1.00 | 3.02 | N |
| ATOM | 2209 | CE2 | TRP | B | 146 | 50.641 | −22.159 | −0.382 | 1.00 | 3.02 | C |
| ATOM | 2210 | CD2 | TRP | B | 146 | 50.897 | −22.991 | −1.491 | 1.00 | 3.02 | C |
| ATOM | 2211 | CE3 | TRP | B | 146 | 50.470 | −24.327 | −1.445 | 1.00 | 3.02 | C |
| ATOM | 2212 | CZ3 | TRP | B | 146 | 49.805 | −24.783 | −0.301 | 1.00 | 3.02 | C |
| ATOM | 2213 | CH2 | TRP | B | 146 | 49.558 | −23.919 | 0.783 | 1.00 | 3.02 | C |
| ATOM | 2214 | CZ2 | TRP | B | 146 | 49.964 | −22.611 | 0.759 | 1.00 | 3.02 | C |
| ATOM | 2215 | C | TRP | B | 146 | 53.102 | −24.416 | −5.281 | 1.00 | 3.02 | C |
| ATOM | 2216 | O | TRP | B | 146 | 52.357 | −25.031 | −6.045 | 1.00 | 3.02 | O |
| ATOM | 2217 | N | GLU | B | 147 | 54.333 | −23.987 | −5.608 | 1.00 | 3.02 | N |
| ATOM | 2218 | CA | GLU | B | 147 | 55.079 | −24.431 | −6.803 | 1.00 | 3.02 | C |
| ATOM | 2219 | CB | GLU | B | 147 | 56.498 | −23.824 | −6.789 | 1.00 | 3.02 | C |
| ATOM | 2220 | CG | GLU | B | 147 | 57.269 | −23.861 | −8.123 | 1.00 | 3.02 | C |
| ATOM | 2221 | CD | GLU | B | 147 | 58.655 | −23.163 | −8.067 | 1.00 | 3.02 | C |
| ATOM | 2222 | OE1 | GLU | B | 147 | 58.853 | −22.225 | −7.262 | 1.00 | 3.02 | O |
| ATOM | 2223 | OE2 | GLU | B | 147 | 59.562 | −23.542 | −8.849 | 1.00 | 3.02 | O |
| ATOM | 2224 | C | GLU | B | 147 | 55.119 | −25.967 | −6.791 | 1.00 | 3.02 | C |
| ATOM | 2225 | O | GLU | B | 147 | 54.812 | −26.626 | −7.781 | 1.00 | 3.02 | O |
| ATOM | 2226 | N | ALA | B | 148 | 55.440 | −26.516 | −5.630 | 1.00 | 3.02 | N |
| ATOM | 2227 | CA | ALA | B | 148 | 55.415 | −27.948 | −5.406 | 1.00 | 3.02 | C |
| ATOM | 2228 | CB | ALA | B | 148 | 56.000 | −28.240 | −4.038 | 1.00 | 3.02 | C |
| ATOM | 2229 | C | ALA | B | 148 | 54.009 | −28.582 | −5.544 | 1.00 | 3.02 | C |
| ATOM | 2230 | O | ALA | B | 148 | 53.854 | −29.812 | −5.557 | 1.00 | 3.02 | O |
| ATOM | 2231 | N | ALA | B | 149 | 52.982 | −27.743 | −5.634 | 1.00 | 3.02 | N |
| ATOM | 2232 | CA | ALA | B | 149 | 51.610 | −28.235 | −5.752 | 1.00 | 3.02 | C |
| ATOM | 2233 | CB | ALA | B | 149 | 50.726 | −27.579 | −4.709 | 1.00 | 3.02 | C |
| ATOM | 2234 | C | ALA | B | 149 | 51.063 | −27.984 | −7.152 | 1.00 | 3.02 | C |
| ATOM | 2235 | O | ALA | B | 149 | 50.171 | −28.674 | −7.624 | 1.00 | 3.02 | O |
| ATOM | 2236 | N | ARG | B | 150 | 51.617 | −26.978 | −7.809 | 1.00 | 3.02 | N |
| ATOM | 2237 | CA | ARG | B | 150 | 51.275 | −26.648 | −9.179 | 1.00 | 3.02 | C |
| ATOM | 2238 | CB | ARG | B | 150 | 51.878 | −25.289 | −9.546 | 1.00 | 3.02 | C |
| ATOM | 2239 | CG | ARG | B | 150 | 51.424 | −24.774 | −10.881 | 1.00 | 3.02 | C |
| ATOM | 2240 | CD | ARG | B | 150 | 52.086 | −23.478 | −11.216 | 1.00 | 3.02 | C |
| ATOM | 2241 | NE | ARG | B | 150 | 51.503 | −22.913 | −12.429 | 1.00 | 3.02 | N |
| ATOM | 2242 | CZ | ARG | B | 150 | 52.021 | −21.895 | −13.105 | 1.00 | 3.02 | C |
| ATOM | 2243 | NH1 | ARG | B | 150 | 53.149 | −21.320 | −12.690 | 1.00 | 3.02 | N |
| ATOM | 2244 | NH2 | ARG | B | 150 | 51.409 | −21.460 | −14.202 | 1.00 | 3.02 | N |
| ATOM | 2245 | C | ARG | B | 150 | 51.838 | −27.722 | −10.088 | 1.00 | 3.02 | C |
| ATOM | 2246 | O | ARG | B | 150 | 51.220 | −28.141 | −11.078 | 1.00 | 3.02 | O |
| ATOM | 2247 | N | HIS | B | 151 | 53.032 | −28.171 | −9.731 | 1.00 | 3.02 | N |
| ATOM | 2248 | CA | HIS | B | 151 | 53.713 | −29.190 | −10.497 | 1.00 | 3.02 | C |
| ATOM | 2249 | CB | HIS | B | 151 | 55.149 | −29.327 | −10.023 | 1.00 | 3.02 | C |
| ATOM | 2250 | CG | HIS | B | 151 | 55.980 | −28.128 | −10.338 | 1.00 | 3.02 | C |
| ATOM | 2251 | ND1 | HIS | B | 151 | 55.904 | −27.473 | −11.548 | 1.00 | 3.02 | N |
| ATOM | 2252 | CE1 | HIS | B | 151 | 56.744 | −26.455 | −11.548 | 1.00 | 3.02 | C |
| ATOM | 2253 | NE2 | HIS | B | 151 | 57.365 | −26.428 | −10.383 | 1.00 | 3.02 | N |
| ATOM | 2254 | CD2 | HIS | B | 151 | 56.903 | −27.462 | −9.607 | 1.00 | 3.02 | C |
| ATOM | 2255 | C | HIS | B | 151 | 52.958 | −30.515 | −10.492 | 1.00 | 3.02 | C |
| ATOM | 2256 | O | HIS | B | 151 | 52.829 | −31.143 | −11.526 | 1.00 | 3.02 | O |
| ATOM | 2257 | N | LEU | B | 152 | 52.444 | −30.928 | −9.341 | 1.00 | 3.02 | N |
| ATOM | 2258 | CA | LEU | B | 152 | 51.535 | −32.064 | −9.289 | 1.00 | 3.02 | C |
| ATOM | 2259 | CB | LEU | B | 152 | 51.579 | −32.731 | −7.910 | 1.00 | 3.02 | C |
| ATOM | 2260 | CG | LEU | B | 152 | 52.891 | −33.412 | −7.511 | 1.00 | 3.02 | C |
| ATOM | 2261 | CD1 | LEU | B | 152 | 52.838 | −33.945 | −6.085 | 1.00 | 3.02 | C |
| ATOM | 2262 | CD2 | LEU | B | 152 | 53.205 | −34.532 | −8.480 | 1.00 | 3.02 | C |
| ATOM | 2263 | C | LEU | B | 152 | 50.110 | −31.616 | −9.627 | 1.00 | 3.02 | C |
| ATOM | 2264 | O | LEU | B | 152 | 49.204 | −32.438 | −9.809 | 1.00 | 3.02 | O |
| ATOM | 2265 | N | PRO | C | 11 | 57.480 | −37.090 | −11.546 | 1.00 | 3.02 | N |
| ATOM | 2266 | CA | PRO | C | 11 | 57.476 | −37.944 | −10.365 | 1.00 | 3.02 | C |
| ATOM | 2267 | CB | PRO | C | 11 | 57.241 | −39.331 | −10.954 | 1.00 | 3.02 | C |
| ATOM | 2268 | CG | PRO | C | 11 | 56.304 | −39.047 | −12.095 | 1.00 | 3.02 | C |
| ATOM | 2269 | CD | PRO | C | 11 | 56.635 | −37.652 | −12.610 | 1.00 | 3.02 | C |
| ATOM | 2270 | C | PRO | C | 11 | 58.757 | −37.867 | −9.539 | 1.00 | 3.02 | C |
| ATOM | 2271 | O | PRO | C | 11 | 58.786 | −38.385 | −8.427 | 1.00 | 3.02 | O |
| ATOM | 2272 | N | ALA | C | 12 | 59.778 | −37.187 | −10.061 | 1.00 | 3.02 | N |
| ATOM | 2273 | CA | ALA | C | 12 | 60.980 | −36.866 | −9.292 | 1.00 | 3.02 | C |
| ATOM | 2274 | CB | ALA | C | 12 | 62.027 | −36.218 | −10.188 | 1.00 | 3.02 | C |
| ATOM | 2275 | C | ALA | C | 12 | 60.580 | −35.927 | −8.163 | 1.00 | 3.02 | C |
| ATOM | 2276 | O | ALA | C | 12 | 60.474 | −36.354 | −7.010 | 1.00 | 3.02 | O |
| ATOM | 2277 | N | LEU | C | 13 | 60.341 | −34.660 | −8.531 | 1.00 | 3.02 | N |
| ATOM | 2278 | CA | LEU | C | 13 | 59.763 | −33.594 | −7.671 | 1.00 | 3.02 | C |
| ATOM | 2279 | CB | LEU | C | 13 | 59.286 | −32.391 | −8.518 | 1.00 | 3.02 | C |
| ATOM | 2280 | CG | LEU | C | 13 | 59.710 | −32.215 | −9.983 | 1.00 | 3.02 | C |
| ATOM | 2281 | CD1 | LEU | C | 13 | 58.991 | −31.052 | −10.661 | 1.00 | 3.02 | C |
| ATOM | 2282 | CD2 | LEU | C | 13 | 61.218 | −32.075 | −10.132 | 1.00 | 3.02 | C |
| ATOM | 2283 | C | LEU | C | 13 | 58.612 | −34.058 | −6.743 | 1.00 | 3.02 | C |
| ATOM | 2284 | O | LEU | C | 13 | 58.555 | −33.646 | −5.585 | 1.00 | 3.02 | O |
| ATOM | 2285 | N | PRO | C | 14 | 57.672 | −34.880 | −7.254 | 1.00 | 3.02 | N |
| ATOM | 2286 | CA | PRO | C | 14 | 56.732 | −35.560 | −6.364 | 1.00 | 3.02 | C |
| ATOM | 2287 | CB | PRO | C | 14 | 56.214 | −36.699 | −7.235 | 1.00 | 3.02 | C |
| ATOM | 2288 | CG | PRO | C | 14 | 56.157 | −36.081 | −8.568 | 1.00 | 3.02 | C |
| ATOM | 2289 | CD | PRO | C | 14 | 57.356 | −35.166 | −8.665 | 1.00 | 3.02 | C |
| ATOM | 2290 | C | PRO | C | 14 | 57.354 | −36.097 | −5.071 | 1.00 | 3.02 | C |
| ATOM | 2291 | O | PRO | C | 14 | 56.857 | −35.780 | −3.974 | 1.00 | 3.02 | O |
| ATOM | 2292 | N | ALA | C | 15 | 58.438 | −36.867 | −5.190 | 1.00 | 3.02 | N |
| ATOM | 2293 | CA | ALA | C | 15 | 59.155 | −37.340 | −4.009 | 1.00 | 3.02 | C |
| ATOM | 2294 | CB | ALA | C | 15 | 60.271 | −38.288 | −4.406 | 1.00 | 3.02 | C |
| ATOM | 2295 | C | ALA | C | 15 | 59.708 | −36.163 | −3.201 | 1.00 | 3.02 | C |
| ATOM | 2296 | O | ALA | C | 15 | 59.615 | −36.141 | −1.969 | 1.00 | 3.02 | O |
| ATOM | 2297 | N | PHE | C | 16 | 60.235 | −35.174 | −3.924 | 1.00 | 3.02 | N |
| ATOM | 2298 | CA | PHE | C | 16 | 60.989 | −34.046 | −3.363 | 1.00 | 3.02 | C |
| ATOM | 2299 | CB | PHE | C | 16 | 61.909 | −33.462 | −4.438 | 1.00 | 3.02 | C |
| ATOM | 2300 | CG | PHE | C | 16 | 62.943 | −34.430 | −4.936 | 1.00 | 3.02 | C |
| ATOM | 2301 | CD1 | PHE | C | 16 | 62.695 | −35.212 | −6.050 | 1.00 | 3.02 | C |
| ATOM | 2302 | CE1 | PHE | C | 16 | 63.644 | −36.111 | −6.521 | 1.00 | 3.02 | C |
| ATOM | 2303 | CZ | PHE | C | 16 | 64.856 | −36.237 | −5.869 | 1.00 | 3.02 | C |
| ATOM | 2304 | CE2 | PHE | C | 16 | 65.117 | −35.462 | −4.750 | 1.00 | 3.02 | C |
| ATOM | 2305 | CD2 | PHE | C | 16 | 64.166 | −34.562 | −4.292 | 1.00 | 3.02 | C |
| ATOM | 2306 | C | PHE | C | 16 | 60.205 | −32.906 | −2.698 | 1.00 | 3.02 | C |
| ATOM | 2307 | O | PHE | C | 16 | 60.774 | −32.162 | −1.913 | 1.00 | 3.02 | O |
| ATOM | 2308 | N | LEU | C | 17 | 58.919 | −32.762 | −2.997 | 1.00 | 3.02 | N |
| ATOM | 2309 | CA | LEU | C | 17 | 58.163 | −31.572 | −2.554 | 1.00 | 3.02 | C |
| ATOM | 2310 | CB | LEU | C | 17 | 56.879 | −31.392 | −3.386 | 1.00 | 3.02 | C |
| ATOM | 2311 | CG | LEU | C | 17 | 57.095 | −31.590 | −4.908 | 1.00 | 3.02 | C |
| ATOM | 2312 | CD1 | LEU | C | 17 | 55.811 | −31.705 | −5.691 | 1.00 | 3.02 | C |
| ATOM | 2313 | CD2 | LEU | C | 17 | 57.989 | −30.540 | −5.553 | 1.00 | 3.02 | C |
| ATOM | 2314 | C | LEU | C | 17 | 57.897 | −31.493 | −1.037 | 1.00 | 3.02 | C |
| ATOM | 2315 | O | LEU | C | 17 | 57.834 | −30.403 | −0.471 | 1.00 | 3.02 | O |
| ATOM | 2316 | N | LEU | C | 18 | 57.777 | −32.649 | −0.388 | 1.00 | 3.02 | N |
| ATOM | 2317 | CA | LEU | C | 18 | 57.646 | −32.713 | 1.066 | 1.00 | 3.02 | C |
| ATOM | 2318 | CB | LEU | C | 18 | 56.603 | −33.762 | 1.474 | 1.00 | 3.02 | C |
| ATOM | 2319 | CG | LEU | C | 18 | 55.128 | −33.435 | 1.177 | 1.00 | 3.02 | C |
| ATOM | 2320 | CD1 | LEU | C | 18 | 54.287 | −34.708 | 1.119 | 1.00 | 3.02 | C |
| ATOM | 2321 | CD2 | LEU | C | 18 | 54.523 | −32.452 | 2.186 | 1.00 | 3.02 | C |
| ATOM | 2322 | C | LEU | C | 18 | 58.988 | −32.966 | 1.764 | 1.00 | 3.02 | C |
| ATOM | 2323 | O | LEU | C | 18 | 59.040 | −33.034 | 2.999 | 1.00 | 3.02 | O |
| ATOM | 2324 | N | CYS | C | 19 | 60.055 | −33.098 | 0.966 | 1.00 | 3.02 | N |
| ATOM | 2325 | CA | CYS | C | 19 | 61.434 | −33.137 | 1.469 | 1.00 | 3.02 | C |
| ATOM | 2326 | CB | CYS | C | 19 | 62.416 | −33.400 | 0.336 | 1.00 | 3.02 | C |
| ATOM | 2327 | SG | CYS | C | 19 | 61.897 | −34.728 | −0.731 | 1.00 | 3.02 | S |
| ATOM | 2328 | C | CYS | C | 19 | 61.738 | −31.793 | 2.091 | 1.00 | 3.02 | C |
| ATOM | 2329 | O | CYS | C | 19 | 62.100 | −31.717 | 3.258 | 1.00 | 3.02 | O |
| ATOM | 2330 | N | SER | C | 20 | 61.558 | −30.738 | 1.295 | 1.00 | 3.02 | N |
| ATOM | 2331 | CA | SER | C | 20 | 61.681 | −29.350 | 1.742 | 1.00 | 3.02 | C |
| ATOM | 2332 | CB | SER | C | 20 | 61.379 | −28.375 | 0.591 | 1.00 | 3.02 | C |
| ATOM | 2333 | OG | SER | C | 20 | 60.037 | −28.481 | 0.126 | 1.00 | 3.02 | O |
| ATOM | 2334 | C | SER | C | 20 | 60.822 | −29.004 | 2.976 | 1.00 | 3.02 | C |
| ATOM | 2335 | O | SER | C | 20 | 61.324 | −28.405 | 3.937 | 1.00 | 3.02 | O |
| ATOM | 2336 | N | THR | C | 21 | 59.550 | −29.405 | 2.955 | 1.00 | 3.02 | N |
| ATOM | 2337 | CA | THR | C | 21 | 58.595 | −28.970 | 3.965 | 1.00 | 3.02 | C |
| ATOM | 2338 | CB | THR | C | 21 | 57.159 | −29.080 | 3.444 | 1.00 | 3.02 | C |
| ATOM | 2339 | OG1 | THR | C | 21 | 57.092 | −28.476 | 2.149 | 1.00 | 3.02 | O |
| ATOM | 2340 | CG2 | THR | C | 21 | 56.179 | −28.384 | 4.391 | 1.00 | 3.02 | C |
| ATOM | 2341 | C | THR | C | 21 | 58.744 | −29.707 | 5.288 | 1.00 | 3.02 | C |
| ATOM | 2342 | O | THR | C | 21 | 58.954 | −29.069 | 6.311 | 1.00 | 3.02 | O |
| ATOM | 2343 | N | LEU | C | 22 | 58.640 | −31.038 | 5.275 | 1.00 | 3.02 | N |
| ATOM | 2344 | CA | LEU | C | 22 | 58.700 | −31.816 | 6.524 | 1.00 | 3.02 | C |
| ATOM | 2345 | CB | LEU | C | 22 | 58.644 | −33.313 | 6.246 | 1.00 | 3.02 | C |
| ATOM | 2346 | CG | LEU | C | 22 | 57.246 | −33.841 | 5.950 | 1.00 | 3.02 | C |
| ATOM | 2347 | CD1 | LEU | C | 22 | 57.327 | −35.266 | 5.392 | 1.00 | 3.02 | C |
| ATOM | 2348 | CD2 | LEU | C | 22 | 56.347 | −33.764 | 7.204 | 1.00 | 3.02 | C |
| ATOM | 2349 | C | LEU | C | 22 | 59.916 | −31.477 | 7.390 | 1.00 | 3.02 | C |
| ATOM | 2350 | O | LEU | C | 22 | 59.838 | −31.477 | 8.627 | 1.00 | 3.02 | O |
| ATOM | 2351 | N | LEU | C | 23 | 61.030 | −31.168 | 6.730 | 1.00 | 3.02 | N |
| ATOM | 2352 | CA | LEU | C | 23 | 62.266 | −30.800 | 7.412 | 1.00 | 3.02 | C |
| ATOM | 2353 | CB | LEU | C | 23 | 63.479 | −31.135 | 6.538 | 1.00 | 3.02 | C |
| ATOM | 2354 | CG | LEU | C | 23 | 63.609 | −32.620 | 6.169 | 1.00 | 3.02 | C |
| ATOM | 2355 | CD1 | LEU | C | 23 | 64.219 | −32.815 | 4.802 | 1.00 | 3.02 | C |
| ATOM | 2356 | CD2 | LEU | C | 23 | 64.433 | −33.347 | 7.213 | 1.00 | 3.02 | C |
| ATOM | 2357 | C | LEU | C | 23 | 62.291 | −29.337 | 7.876 | 1.00 | 3.02 | C |
| ATOM | 2358 | O | LEU | C | 23 | 63.238 | −28.929 | 8.557 | 1.00 | 3.02 | O |
| ATOM | 2359 | N | VAL | C | 24 | 61.271 | −28.554 | 7.504 | 1.00 | 3.02 | N |
| ATOM | 2360 | CA | VAL | C | 24 | 61.041 | −27.243 | 8.137 | 1.00 | 3.02 | C |
| ATOM | 2361 | CB | VAL | C | 24 | 60.007 | −26.367 | 7.389 | 1.00 | 3.02 | C |
| ATOM | 2362 | CG1 | VAL | C | 24 | 59.891 | −25.018 | 8.035 | 1.00 | 3.02 | C |
| ATOM | 2363 | CG2 | VAL | C | 24 | 60.402 | −26.180 | 5.962 | 1.00 | 3.02 | C |
| ATOM | 2364 | C | VAL | C | 24 | 60.553 | −27.509 | 9.559 | 1.00 | 3.02 | C |
| ATOM | 2365 | O | VAL | C | 24 | 61.080 | −26.912 | 10.506 | 1.00 | 3.02 | O |
| ATOM | 2366 | N | ILE | C | 25 | 59.574 | −28.424 | 9.689 | 1.00 | 3.02 | N |
| ATOM | 2367 | CA | ILE | C | 25 | 59.111 | −28.974 | 10.983 | 1.00 | 3.02 | C |
| ATOM | 2368 | CB | ILE | C | 25 | 58.066 | −30.111 | 10.790 | 1.00 | 3.02 | C |
| ATOM | 2369 | CG1 | ILE | C | 25 | 56.870 | −29.623 | 9.955 | 1.00 | 3.02 | C |
| ATOM | 2370 | CD1 | ILE | C | 25 | 55.773 | −30.680 | 9.732 | 1.00 | 3.02 | C |
| ATOM | 2371 | CG2 | ILE | C | 25 | 57.607 | −30.685 | 12.140 | 1.00 | 3.02 | C |
| ATOM | 2372 | C | ILE | C | 25 | 60.284 | −29.494 | 11.825 | 1.00 | 3.02 | C |
| ATOM | 2373 | O | ILE | C | 25 | 60.204 | −29.568 | 13.057 | 1.00 | 3.02 | O |
| ATOM | 2374 | N | LYS | C | 26 | 61.374 | −29.829 | 11.143 | 1.00 | 3.02 | N |
| ATOM | 2375 | CA | LYS | C | 26 | 62.533 | −30.438 | 11.778 | 1.00 | 3.02 | C |
| ATOM | 2376 | CB | LYS | C | 26 | 63.058 | −31.621 | 10.934 | 1.00 | 3.02 | C |
| ATOM | 2377 | CG | LYS | C | 26 | 63.844 | −32.679 | 11.709 | 1.00 | 3.02 | C |
| ATOM | 2378 | CD | LYS | C | 26 | 62.991 | −33.356 | 12.778 | 1.00 | 3.02 | C |
| ATOM | 2379 | CE | LYS | C | 26 | 63.860 | −34.037 | 13.852 | 1.00 | 3.02 | C |
| ATOM | 2380 | NZ | LYS | C | 26 | 63.237 | −33.884 | 15.217 | 1.00 | 3.02 | N |
| ATOM | 2381 | C | LYS | C | 26 | 63.636 | −29.426 | 12.108 | 1.00 | 3.02 | C |
| ATOM | 2382 | O | LYS | C | 26 | 63.937 | −29.222 | 13.281 | 1.00 | 3.02 | O |
| ATOM | 2383 | N | MET | C | 27 | 64.226 | −28.790 | 11.095 | 1.00 | 3.02 | N |
| ATOM | 2384 | CA | MET | C | 27 | 65.299 | −27.828 | 11.355 | 1.00 | 3.02 | C |
| ATOM | 2385 | CB | MET | C | 27 | 66.381 | −27.875 | 10.278 | 1.00 | 3.02 | C |
| ATOM | 2386 | CG | MET | C | 27 | 67.463 | −28.904 | 10.581 | 1.00 | 3.02 | C |
| ATOM | 2387 | SD | MET | C | 27 | 69.172 | −28.335 | 10.350 | 1.00 | 3.02 | S |
| ATOM | 2388 | CE | MET | C | 27 | 69.395 | −27.279 | 11.789 | 1.00 | 3.02 | C |
| ATOM | 2389 | C | MET | C | 27 | 64.875 | −26.383 | 11.661 | 1.00 | 3.02 | C |
| ATOM | 2390 | O | MET | C | 27 | 65.621 | −25.668 | 12.347 | 1.00 | 3.02 | O |
| ATOM | 2391 | N | TYR | C | 28 | 63.712 | −25.932 | 11.179 | 1.00 | 3.02 | N |
| ATOM | 2392 | CA | TYR | C | 28 | 63.292 | −24.557 | 11.528 | 1.00 | 3.02 | C |
| ATOM | 2393 | CB | TYR | C | 28 | 63.554 | −23.553 | 10.397 | 1.00 | 3.02 | C |
| ATOM | 2394 | CG | TYR | C | 28 | 64.097 | −22.251 | 10.930 | 1.00 | 3.02 | C |
| ATOM | 2395 | CD1 | TYR | C | 28 | 65.437 | −22.100 | 11.220 | 1.00 | 3.02 | C |
| ATOM | 2396 | CE1 | TYR | C | 28 | 65.926 | −20.901 | 11.722 | 1.00 | 3.02 | C |
| ATOM | 2397 | CZ | TYR | C | 28 | 65.070 | −19.838 | 11.944 | 1.00 | 3.02 | C |
| ATOM | 2398 | OH | TYR | C | 28 | 65.533 | −18.649 | 12.440 | 1.00 | 3.02 | O |
| ATOM | 2399 | CE2 | TYR | C | 28 | 63.743 | −19.959 | 11.672 | 1.00 | 3.02 | C |
| ATOM | 2400 | CD2 | TYR | C | 28 | 63.254 | −21.171 | 11.175 | 1.00 | 3.02 | C |
| ATOM | 2401 | C | TYR | C | 28 | 61.952 | −24.291 | 12.255 | 1.00 | 3.02 | C |
| ATOM | 2402 | O | TYR | C | 28 | 61.803 | −23.221 | 12.835 | 1.00 | 3.02 | O |
| ATOM | 2403 | N | VAL | C | 29 | 61.005 | −25.232 | 12.260 | 1.00 | 3.02 | N |
| ATOM | 2404 | CA | VAL | C | 29 | 59.855 | −25.073 | 13.166 | 1.00 | 3.02 | C |
| ATOM | 2405 | CB | VAL | C | 29 | 58.548 | −25.795 | 12.705 | 1.00 | 3.02 | C |
| ATOM | 2406 | CG1 | VAL | C | 29 | 57.371 | −25.295 | 13.517 | 1.00 | 3.02 | C |
| ATOM | 2407 | CG2 | VAL | C | 29 | 58.253 | −25.552 | 11.238 | 1.00 | 3.02 | C |
| ATOM | 2408 | C | VAL | C | 29 | 60.271 | −25.504 | 14.581 | 1.00 | 3.02 | C |
| ATOM | 2409 | O | VAL | C | 29 | 59.624 | −25.151 | 15.567 | 1.00 | 3.02 | O |
| ATOM | 2410 | N | VAL | C | 30 | 61.371 | −26.249 | 14.668 | 1.00 | 3.02 | N |
| ATOM | 2411 | CA | VAL | C | 30 | 61.954 | −26.645 | 15.960 | 1.00 | 3.02 | C |
| ATOM | 2412 | CB | VAL | C | 30 | 62.549 | −28.095 | 15.923 | 1.00 | 3.02 | C |
| ATOM | 2413 | CG1 | VAL | C | 30 | 63.656 | −28.285 | 16.945 | 1.00 | 3.02 | C |
| ATOM | 2414 | CG2 | VAL | C | 30 | 61.456 | −29.120 | 16.162 | 1.00 | 3.02 | C |
| ATOM | 2415 | C | VAL | C | 30 | 62.984 | −25.615 | 16.442 | 1.00 | 3.02 | C |
| ATOM | 2416 | O | VAL | C | 30 | 63.046 | −25.291 | 17.629 | 1.00 | 3.02 | O |
| ATOM | 2417 | N | ALA | C | 31 | 63.787 | −25.109 | 15.514 | 1.00 | 3.02 | N |
| ATOM | 2418 | CA | ALA | C | 31 | 64.562 | −23.904 | 15.756 | 1.00 | 3.02 | C |
| ATOM | 2419 | CB | ALA | C | 31 | 65.304 | −23.490 | 14.506 | 1.00 | 3.02 | C |
| ATOM | 2420 | C | ALA | C | 31 | 63.657 | −22.766 | 16.225 | 1.00 | 3.02 | C |
| ATOM | 2421 | O | ALA | C | 31 | 64.066 | −21.999 | 17.081 | 1.00 | 3.02 | O |
| ATOM | 2422 | N | ILE | C | 32 | 62.436 | −22.676 | 15.676 | 1.00 | 3.02 | N |
| ATOM | 2423 | CA | ILE | C | 32 | 61.480 | −21.606 | 16.021 | 1.00 | 3.02 | C |
| ATOM | 2424 | CB | ILE | C | 32 | 60.329 | −21.469 | 14.973 | 1.00 | 3.02 | C |
| ATOM | 2425 | CG1 | ILE | C | 32 | 60.831 | −20.753 | 13.717 | 1.00 | 3.02 | C |
| ATOM | 2426 | CD1 | ILE | C | 32 | 59.826 | −20.731 | 12.540 | 1.00 | 3.02 | C |
| ATOM | 2427 | CG2 | ILE | C | 32 | 59.127 | −20.696 | 15.536 | 1.00 | 3.02 | C |
| ATOM | 2428 | C | ILE | C | 32 | 60.966 | −21.659 | 17.476 | 1.00 | 3.02 | C |
| ATOM | 2429 | O | ILE | C | 32 | 61.409 | −20.866 | 18.300 | 1.00 | 3.02 | O |
| ATOM | 2430 | N | ILE | C | 33 | 60.055 | −22.582 | 17.796 | 1.00 | 3.02 | N |
| ATOM | 2431 | CA | ILE | C | 33 | 59.476 | −22.672 | 19.158 | 1.00 | 3.02 | C |
| ATOM | 2432 | CB | ILE | C | 33 | 58.626 | −23.968 | 19.359 | 1.00 | 3.02 | C |
| ATOM | 2433 | CG1 | ILE | C | 33 | 57.213 | −23.782 | 18.780 | 1.00 | 3.02 | C |
| ATOM | 2434 | CD1 | ILE | C | 33 | 56.403 | −25.132 | 18.563 | 1.00 | 3.02 | C |
| ATOM | 2435 | CG2 | ILE | C | 33 | 58.553 | −24.358 | 20.838 | 1.00 | 3.02 | C |
| ATOM | 2436 | C | ILE | C | 33 | 60.558 | −22.555 | 20.245 | 1.00 | 3.02 | C |
| ATOM | 2437 | O | ILE | C | 33 | 60.397 | −21.826 | 21.228 | 1.00 | 3.02 | O |
| ATOM | 2438 | N | THR | C | 34 | 61.673 | −23.252 | 20.023 | 1.00 | 3.02 | N |
| ATOM | 2439 | CA | THR | C | 34 | 62.764 | −23.347 | 20.984 | 1.00 | 3.02 | C |
| ATOM | 2440 | CB | THR | C | 34 | 63.815 | −24.405 | 20.560 | 1.00 | 3.02 | C |
| ATOM | 2441 | OG1 | THR | C | 34 | 64.051 | −24.314 | 19.148 | 1.00 | 3.02 | O |
| ATOM | 2442 | CG2 | THR | C | 34 | 63.328 | −25.813 | 20.889 | 1.00 | 3.02 | C |
| ATOM | 2443 | C | THR | C | 34 | 63.421 | −22.000 | 21.293 | 1.00 | 3.02 | C |
| ATOM | 2444 | O | THR | C | 34 | 64.027 | −21.856 | 22.354 | 1.00 | 3.02 | O |
| ATOM | 2445 | N | GLY | C | 35 | 63.303 | −21.021 | 20.390 | 1.00 | 3.02 | N |
| ATOM | 2446 | CA | GLY | C | 35 | 63.768 | −19.632 | 20.640 | 1.00 | 3.02 | C |
| ATOM | 2447 | C | GLY | C | 35 | 62.656 | −18.695 | 21.079 | 1.00 | 3.02 | C |
| ATOM | 2448 | O | GLY | C | 35 | 62.785 | −18.000 | 22.097 | 1.00 | 3.02 | O |
| ATOM | 2449 | N | GLN | C | 36 | 61.576 | −18.682 | 20.285 | 1.00 | 3.02 | N |
| ATOM | 2450 | CA | GLN | C | 36 | 60.278 | −18.117 | 20.677 | 1.00 | 3.02 | C |
| ATOM | 2451 | CB | GLN | C | 36 | 59.140 | −18.675 | 19.805 | 1.00 | 3.02 | C |
| ATOM | 2452 | CG | GLN | C | 36 | 59.297 | −18.478 | 18.314 | 1.00 | 3.02 | C |
| ATOM | 2453 | CD | GLN | C | 36 | 59.093 | −17.049 | 17.872 | 1.00 | 3.02 | C |
| ATOM | 2454 | OE1 | GLN | C | 36 | 58.676 | −16.180 | 18.649 | 1.00 | 3.02 | O |
| ATOM | 2455 | NE2 | GLN | C | 36 | 59.379 | −16.791 | 16.604 | 1.00 | 3.02 | N |
| ATOM | 2456 | C | GLN | C | 36 | 59.978 | −18.421 | 22.141 | 1.00 | 3.02 | C |
| ATOM | 2457 | O | GLN | C | 36 | 59.745 | −17.495 | 22.927 | 1.00 | 3.02 | O |
| ATOM | 2458 | N | VAL | C | 37 | 60.002 | −19.715 | 22.487 | 1.00 | 3.02 | N |
| ATOM | 2459 | CA | VAL | C | 37 | 59.752 | −20.203 | 23.847 | 1.00 | 3.02 | C |
| ATOM | 2460 | CB | VAL | C | 37 | 59.506 | −21.746 | 23.855 | 1.00 | 3.02 | C |
| ATOM | 2461 | CG1 | VAL | C | 37 | 60.200 | −22.435 | 25.025 | 1.00 | 3.02 | C |
| ATOM | 2462 | CG2 | VAL | C | 37 | 58.014 | −22.057 | 23.851 | 1.00 | 3.02 | C |
| ATOM | 2463 | C | VAL | C | 37 | 60.849 | −19.803 | 24.845 | 1.00 | 3.02 | C |
| ATOM | 2464 | O | VAL | C | 37 | 60.545 | −19.409 | 25.979 | 1.00 | 3.02 | O |
| ATOM | 2465 | N | ARG | C | 38 | 62.113 | −19.870 | 24.426 | 1.00 | 3.02 | N |
| ATOM | 2466 | CA | ARG | C | 38 | 63.206 | −19.605 | 25.354 | 1.00 | 3.02 | C |
| ATOM | 2467 | CB | ARG | C | 38 | 64.533 | −20.197 | 24.881 | 1.00 | 3.02 | C |
| ATOM | 2468 | CG | ARG | C | 38 | 65.654 | −20.086 | 25.906 | 1.00 | 3.02 | C |
| ATOM | 2469 | CD | ARG | C | 38 | 67.003 | −20.104 | 25.216 | 1.00 | 3.02 | C |
| ATOM | 2470 | NE | ARG | C | 38 | 68.106 | −20.296 | 26.157 | 1.00 | 3.02 | N |
| ATOM | 2471 | CZ | ARG | C | 38 | 69.382 | −20.003 | 25.901 | 1.00 | 3.02 | C |
| ATOM | 2472 | NH1 | ARG | C | 38 | 69.737 | −19.488 | 24.727 | 1.00 | 3.02 | N |
| ATOM | 2473 | NH2 | ARG | C | 38 | 70.306 | −20.205 | 26.829 | 1.00 | 3.02 | N |
| ATOM | 2474 | C | ARG | C | 38 | 63.353 | −18.127 | 25.645 | 1.00 | 3.02 | C |
| ATOM | 2475 | O | ARG | C | 38 | 63.157 | −17.713 | 26.783 | 1.00 | 3.02 | O |
| ATOM | 2476 | N | LEU | C | 39 | 63.682 | −17.331 | 24.628 | 1.00 | 3.02 | N |
| ATOM | 2477 | CA | LEU | C | 39 | 63.864 | −15.888 | 24.825 | 1.00 | 3.02 | C |
| ATOM | 2478 | CB | LEU | C | 39 | 63.971 | −15.138 | 23.494 | 1.00 | 3.02 | C |
| ATOM | 2479 | CG | LEU | C | 39 | 65.195 | −15.315 | 22.583 | 1.00 | 3.02 | C |
| ATOM | 2480 | CD1 | LEU | C | 39 | 64.903 | −14.681 | 21.256 | 1.00 | 3.02 | C |
| ATOM | 2481 | CD2 | LEU | C | 39 | 66.466 | −14.722 | 23.161 | 1.00 | 3.02 | C |
| ATOM | 2482 | C | LEU | C | 39 | 62.723 | −15.323 | 25.660 | 1.00 | 3.02 | C |
| ATOM | 2483 | O | LEU | C | 39 | 62.928 | −14.380 | 26.423 | 1.00 | 3.02 | O |
| ATOM | 2484 | N | ARG | C | 40 | 61.537 | −15.935 | 25.517 | 1.00 | 3.02 | N |
| ATOM | 2485 | CA | ARG | C | 40 | 60.311 | −15.602 | 26.270 | 1.00 | 3.02 | C |
| ATOM | 2486 | CB | ARG | C | 40 | 59.094 | −16.182 | 25.562 | 1.00 | 3.02 | C |
| ATOM | 2487 | CG | ARG | C | 40 | 58.486 | −15.258 | 24.545 | 1.00 | 3.02 | C |
| ATOM | 2488 | CD | ARG | C | 40 | 56.994 | −15.480 | 24.489 | 1.00 | 3.02 | C |
| ATOM | 2489 | NE | ARG | C | 40 | 56.362 | −14.671 | 23.460 | 1.00 | 3.02 | N |
| ATOM | 2490 | CZ | ARG | C | 40 | 55.244 | −15.011 | 22.830 | 1.00 | 3.02 | C |
| ATOM | 2491 | NH1 | ARG | C | 40 | 54.625 | −16.155 | 23.118 | 1.00 | 3.02 | N |
| ATOM | 2492 | NH2 | ARG | C | 40 | 54.743 | −14.205 | 21.906 | 1.00 | 3.02 | N |
| ATOM | 2493 | C | ARG | C | 40 | 60.283 | −16.043 | 27.742 | 1.00 | 3.02 | C |
| ATOM | 2494 | O | ARG | C | 40 | 60.245 | −15.191 | 28.646 | 1.00 | 3.02 | O |
| ATOM | 2495 | N | LYS | C | 41 | 60.278 | −17.364 | 27.971 | 1.00 | 3.02 | N |
| ATOM | 2496 | CA | LYS | C | 41 | 60.349 | −17.941 | 29.328 | 1.00 | 3.02 | C |
| ATOM | 2497 | CB | LYS | C | 41 | 60.321 | −19.480 | 29.289 | 1.00 | 3.02 | C |
| ATOM | 2498 | CG | LYS | C | 41 | 58.979 | −20.048 | 28.811 | 1.00 | 3.02 | C |
| ATOM | 2499 | CD | LYS | C | 41 | 58.757 | −21.530 | 29.132 | 1.00 | 3.02 | C |
| ATOM | 2500 | CE | LYS | C | 41 | 57.401 | −22.008 | 28.559 | 1.00 | 3.02 | C |
| ATOM | 2501 | NZ | LYS | C | 41 | 56.742 | −23.125 | 29.310 | 1.00 | 3.02 | N |
| ATOM | 2502 | C | LYS | C | 41 | 61.605 | −17.452 | 30.040 | 1.00 | 3.02 | C |
| ATOM | 2503 | O | LYS | C | 41 | 61.638 | −17.307 | 31.270 | 1.00 | 3.02 | O |
| ATOM | 2504 | N | LYS | C | 42 | 62.619 | −17.183 | 29.222 | 1.00 | 3.02 | N |
| ATOM | 2505 | CA | LYS | C | 42 | 63.940 | −16.739 | 29.642 | 1.00 | 3.02 | C |
| ATOM | 2506 | CB | LYS | C | 42 | 64.539 | −15.793 | 28.594 | 1.00 | 3.02 | C |
| ATOM | 2507 | CG | LYS | C | 42 | 66.063 | −15.780 | 28.565 | 1.00 | 3.02 | C |
| ATOM | 2508 | CD | LYS | C | 42 | 66.641 | −16.765 | 27.546 | 1.00 | 3.02 | C |
| ATOM | 2509 | CE | LYS | C | 42 | 67.309 | −16.043 | 26.382 | 1.00 | 3.02 | C |
| ATOM | 2510 | NZ | LYS | C | 42 | 68.588 | −15.380 | 26.847 | 1.00 | 3.02 | N |
| ATOM | 2511 | C | LYS | C | 42 | 63.976 | −16.072 | 31.007 | 1.00 | 3.02 | C |
| ATOM | 2512 | O | LYS | C | 42 | 63.337 | −15.035 | 31.226 | 1.00 | 3.02 | O |
| ATOM | 2513 | N | ALA | C | 43 | 64.718 | −16.697 | 31.917 | 1.00 | 3.02 | N |
| ATOM | 2514 | CA | ALA | C | 43 | 65.089 | −16.080 | 33.177 | 1.00 | 3.02 | C |
| ATOM | 2515 | CB | ALA | C | 43 | 64.989 | −17.071 | 34.315 | 1.00 | 3.02 | C |
| ATOM | 2516 | C | ALA | C | 43 | 66.504 | −15.545 | 33.041 | 1.00 | 3.02 | C |
| ATOM | 2517 | O | ALA | C | 43 | 67.286 | −15.569 | 33.991 | 1.00 | 3.02 | O |
| ATOM | 2518 | N | PHE | C | 44 | 66.818 | −15.074 | 31.835 | 1.00 | 3.02 | N |
| ATOM | 2519 | CA | PHE | C | 44 | 68.053 | −14.343 | 31.576 | 1.00 | 3.02 | C |
| ATOM | 2520 | CB | PHE | C | 44 | 68.491 | −14.479 | 30.117 | 1.00 | 3.02 | C |
| ATOM | 2521 | CG | PHE | C | 44 | 69.582 | −15.499 | 29.897 | 1.00 | 3.02 | C |
| ATOM | 2522 | CD1 | PHE | C | 44 | 69.278 | −16.849 | 29.804 | 1.00 | 3.02 | C |
| ATOM | 2523 | CE1 | PHE | C | 44 | 70.281 | −17.798 | 29.582 | 1.00 | 3.02 | C |
| ATOM | 2524 | CZ | PHE | C | 44 | 71.612 | −17.388 | 29.459 | 1.00 | 3.02 | C |
| ATOM | 2525 | CE2 | PHE | C | 44 | 71.916 | −16.037 | 29.556 | 1.00 | 3.02 | C |
| ATOM | 2526 | CD2 | PHE | C | 44 | 70.907 | −15.104 | 29.770 | 1.00 | 3.02 | C |
| ATOM | 2527 | C | PHE | C | 44 | 67.936 | −12.876 | 31.999 | 1.00 | 3.02 | C |
| ATOM | 2528 | O | PHE | C | 44 | 66.841 | −12.310 | 32.017 | 1.00 | 3.02 | O |
| ATOM | 2529 | N | ALA | C | 45 | 69.093 | −12.275 | 32.284 | 1.00 | 3.02 | N |
| ATOM | 2530 | CA | ALA | C | 45 | 69.253 | −11.241 | 33.315 | 1.00 | 3.02 | C |
| ATOM | 2531 | CB | ALA | C | 45 | 67.939 | −10.455 | 33.580 | 1.00 | 3.02 | C |
| ATOM | 2532 | C | ALA | C | 45 | 69.752 | −11.983 | 34.578 | 1.00 | 3.02 | C |
| ATOM | 2533 | O | ALA | C | 45 | 69.796 | −11.429 | 35.691 | 1.00 | 3.02 | O |
| ATOM | 2534 | N | ASN | C | 46 | 70.145 | −13.240 | 34.347 | 1.00 | 3.02 | N |
| ATOM | 2535 | CA | ASN | C | 46 | 70.550 | −14.215 | 35.359 | 1.00 | 3.02 | C |
| ATOM | 2536 | CB | ASN | C | 46 | 71.381 | −15.319 | 34.703 | 1.00 | 3.02 | C |
| ATOM | 2537 | CG | ASN | C | 46 | 70.709 | −15.901 | 33.467 | 1.00 | 3.02 | C |
| ATOM | 2538 | OD1 | ASN | C | 46 | 71.275 | −15.890 | 32.382 | 1.00 | 3.02 | O |
| ATOM | 2539 | ND2 | ASN | C | 46 | 69.499 | −16.406 | 33.629 | 1.00 | 3.02 | N |
| ATOM | 2540 | C | ASN | C | 46 | 71.322 | −13.633 | 36.528 | 1.00 | 3.02 | C |
| ATOM | 2541 | O | ASN | C | 46 | 72.368 | −13.029 | 36.324 | 1.00 | 3.02 | O |
| ATOM | 2542 | N | PRO | C | 47 | 70.871 | −13.910 | 37.764 | 1.00 | 3.02 | N |
| ATOM | 2543 | CA | PRO | C | 47 | 71.267 | −13.257 | 38.993 | 1.00 | 3.02 | C |
| ATOM | 2544 | CB | PRO | C | 47 | 71.841 | −14.457 | 39.793 | 1.00 | 3.02 | C |
| ATOM | 2545 | CG | PRO | C | 47 | 71.054 | −15.742 | 39.149 | 1.00 | 3.02 | C |
| ATOM | 2546 | CD | PRO | C | 47 | 70.120 | −15.124 | 38.097 | 1.00 | 3.02 | C |
| ATOM | 2547 | C | PRO | C | 47 | 72.311 | −12.175 | 38.764 | 1.00 | 3.02 | C |
| ATOM | 2548 | O | PRO | C | 47 | 73.492 | −12.400 | 39.051 | 1.00 | 3.02 | O |
| ATOM | 2549 | N | GLU | C | 48 | 71.870 | −11.027 | 38.232 | 1.00 | 3.02 | N |
| ATOM | 2550 | CA | GLU | C | 48 | 72.765 | −9.951 | 37.705 | 1.00 | 3.02 | C |
| ATOM | 2551 | CB | GLU | C | 48 | 73.482 | −9.173 | 38.834 | 1.00 | 3.02 | C |
| ATOM | 2552 | CG | GLU | C | 48 | 74.315 | −7.973 | 38.370 | 1.00 | 3.02 | C |
| ATOM | 2553 | CD | GLU | C | 48 | 74.874 | −7.139 | 39.533 | 1.00 | 3.02 | C |
| ATOM | 2554 | OE1 | GLU | C | 48 | 74.842 | −7.630 | 40.685 | 1.00 | 3.02 | O |
| ATOM | 2555 | OE2 | GLU | C | 48 | 75.347 | −5.992 | 39.302 | 1.00 | 3.02 | O |
| ATOM | 2556 | C | GLU | C | 48 | 73.765 | −10.483 | 36.651 | 1.00 | 3.02 | C |
| ATOM | 2557 | O | GLU | C | 48 | 74.793 | −11.101 | 36.989 | 1.00 | 3.02 | O |
| ATOM | 2558 | N | ASP | C | 49 | 73.451 | −10.257 | 35.374 | 1.00 | 3.02 | N |
| ATOM | 2559 | CA | ASP | C | 49 | 74.182 | −10.942 | 34.307 | 1.00 | 3.02 | C |
| ATOM | 2560 | CB | ASP | C | 49 | 73.654 | −12.375 | 34.140 | 1.00 | 3.02 | C |
| ATOM | 2561 | CG | ASP | C | 49 | 74.649 | −13.300 | 33.481 | 1.00 | 3.02 | C |
| ATOM | 2562 | OD1 | ASP | C | 49 | 75.841 | −12.930 | 33.408 | 1.00 | 3.02 | O |
| ATOM | 2563 | OD2 | ASP | C | 49 | 74.232 | −14.399 | 33.043 | 1.00 | 3.02 | O |
| ATOM | 2564 | C | ASP | C | 49 | 74.201 | −10.245 | 32.951 | 1.00 | 3.02 | C |
| ATOM | 2565 | O | ASP | C | 49 | 73.158 | −9.945 | 32.349 | 1.00 | 3.02 | O |
| ATOM | 2566 | N | ALA | C | 50 | 75.428 | −9.975 | 32.520 | 1.00 | 3.02 | N |
| ATOM | 2567 | CA | ALA | C | 50 | 75.784 | −9.822 | 31.129 | 1.00 | 3.02 | C |
| ATOM | 2568 | CB | ALA | C | 50 | 77.011 | −8.941 | 31.009 | 1.00 | 3.02 | C |
| ATOM | 2569 | C | ALA | C | 50 | 76.098 | −11.249 | 30.699 | 1.00 | 3.02 | C |
| ATOM | 2570 | O | ALA | C | 50 | 77.049 | −11.867 | 31.202 | 1.00 | 3.02 | O |
| ATOM | 2571 | N | LEU | C | 51 | 75.299 | −11.778 | 29.778 | 1.00 | 3.02 | N |
| ATOM | 2572 | CA | LEU | C | 51 | 75.230 | −13.236 | 29.589 | 1.00 | 3.02 | C |
| ATOM | 2573 | CB | LEU | C | 51 | 73.934 | −13.671 | 28.877 | 1.00 | 3.02 | C |
| ATOM | 2574 | CG | LEU | C | 51 | 73.531 | −13.353 | 27.437 | 1.00 | 3.02 | C |
| ATOM | 2575 | CD1 | LEU | C | 51 | 74.291 | −14.187 | 26.421 | 1.00 | 3.02 | C |
| ATOM | 2576 | CD2 | LEU | C | 51 | 72.042 | −13.591 | 27.300 | 1.00 | 3.02 | C |
| ATOM | 2577 | C | LEU | C | 51 | 76.464 | −13.959 | 29.033 | 1.00 | 3.02 | C |
| ATOM | 2578 | O | LEU | C | 51 | 77.583 | −13.437 | 29.092 | 1.00 | 3.02 | O |
| ATOM | 2579 | N | ARG | C | 52 | 76.202 | −15.138 | 28.459 | 1.00 | 3.02 | N |
| ATOM | 2580 | CA | ARG | C | 52 | 77.091 | −16.323 | 28.383 | 1.00 | 3.02 | C |
| ATOM | 2581 | CB | ARG | C | 52 | 78.498 | −16.064 | 27.809 | 1.00 | 3.02 | C |
| ATOM | 2582 | CG | ARG | C | 52 | 79.162 | −14.769 | 28.212 | 1.00 | 3.02 | C |
| ATOM | 2583 | CD | ARG | C | 52 | 80.223 | −14.381 | 27.218 | 1.00 | 3.02 | C |
| ATOM | 2584 | NE | ARG | C | 52 | 80.311 | −12.941 | 26.961 | 1.00 | 3.02 | N |
| ATOM | 2585 | CZ | ARG | C | 52 | 80.662 | −12.018 | 27.861 | 1.00 | 3.02 | C |
| ATOM | 2586 | NH1 | ARG | C | 52 | 80.931 | −12.355 | 29.121 | 1.00 | 3.02 | N |
| ATOM | 2587 | NH2 | ARG | C | 52 | 80.751 | −10.746 | 27.504 | 1.00 | 3.02 | N |
| ATOM | 2588 | C | ARG | C | 52 | 77.099 | −17.025 | 29.739 | 1.00 | 3.02 | C |
| ATOM | 2589 | O | ARG | C | 52 | 76.772 | −16.401 | 30.740 | 1.00 | 3.02 | O |
| ATOM | 2590 | N | HIS | C | 53 | 77.423 | −18.323 | 29.749 | 1.00 | 3.02 | N |
| ATOM | 2591 | CA | HIS | C | 53 | 77.292 | −19.241 | 30.923 | 1.00 | 3.02 | C |
| ATOM | 2592 | CB | HIS | C | 53 | 78.208 | −18.835 | 32.112 | 1.00 | 3.02 | C |
| ATOM | 2593 | CG | HIS | C | 53 | 78.195 | −17.368 | 32.431 | 1.00 | 3.02 | C |
| ATOM | 2594 | ND1 | HIS | C | 53 | 79.099 | −16.475 | 31.888 | 1.00 | 3.02 | N |
| ATOM | 2595 | CE1 | HIS | C | 53 | 78.836 | −15.256 | 32.330 | 1.00 | 3.02 | C |
| ATOM | 2596 | NE2 | HIS | C | 53 | 77.793 | −15.322 | 33.139 | 1.00 | 3.02 | N |
| ATOM | 2597 | CD2 | HIS | C | 53 | 77.374 | −16.631 | 33.221 | 1.00 | 3.02 | C |
| ATOM | 2598 | C | HIS | C | 53 | 75.839 | −19.545 | 31.380 | 1.00 | 3.02 | C |
| ATOM | 2599 | O | HIS | C | 53 | 75.172 | −18.709 | 32.009 | 1.00 | 3.02 | O |
| ATOM | 2600 | N | GLY | C | 54 | 75.353 | −20.744 | 31.066 | 1.00 | 3.02 | N |
| ATOM | 2601 | CA | GLY | C | 54 | 73.950 | −21.097 | 31.348 | 1.00 | 3.02 | C |
| ATOM | 2602 | C | GLY | C | 54 | 73.762 | −22.354 | 32.181 | 1.00 | 3.02 | C |
| ATOM | 2603 | O | GLY | C | 54 | 73.382 | −23.411 | 31.650 | 1.00 | 3.02 | O |
| ATOM | 2604 | N | GLY | C | 55 | 74.028 | −22.236 | 33.488 | 1.00 | 3.02 | N |
| ATOM | 2605 | CA | GLY | C | 55 | 73.971 | −23.382 | 34.416 | 1.00 | 3.02 | C |
| ATOM | 2606 | C | GLY | C | 55 | 73.959 | −23.162 | 35.934 | 1.00 | 3.02 | C |
| ATOM | 2607 | O | GLY | C | 55 | 74.485 | −24.004 | 36.668 | 1.00 | 3.02 | O |
| ATOM | 2608 | N | PRO | C | 56 | 73.374 | −22.042 | 36.426 | 1.00 | 3.02 | N |
| ATOM | 2609 | CA | PRO | C | 56 | 73.159 | −21.943 | 37.880 | 1.00 | 3.02 | C |
| ATOM | 2610 | CB | PRO | C | 56 | 72.857 | −20.458 | 38.082 | 1.00 | 3.02 | C |
| ATOM | 2611 | CG | PRO | C | 56 | 72.200 | −20.053 | 36.814 | 1.00 | 3.02 | C |
| ATOM | 2612 | CD | PRO | C | 56 | 72.925 | −20.818 | 35.736 | 1.00 | 3.02 | C |
| ATOM | 2613 | C | PRO | C | 56 | 71.971 | −22.810 | 38.344 | 1.00 | 3.02 | C |
| ATOM | 2614 | O | PRO | C | 56 | 72.146 | −24.027 | 38.598 | 1.00 | 3.02 | O |
| ATOM | 2615 | N | GLN | C | 57 | 70.784 | −22.189 | 38.440 | 1.00 | 3.02 | N |
| ATOM | 2616 | CA | GLN | C | 57 | 69.542 | −22.894 | 38.783 | 1.00 | 3.02 | C |
| ATOM | 2617 | CB | GLN | C | 57 | 68.515 | −21.961 | 39.424 | 1.00 | 3.02 | C |
| ATOM | 2618 | CG | GLN | C | 57 | 68.837 | −21.622 | 40.870 | 1.00 | 3.02 | C |
| ATOM | 2619 | CD | GLN | C | 57 | 67.823 | −20.710 | 41.522 | 1.00 | 3.02 | C |
| ATOM | 2620 | OE1 | GLN | C | 57 | 67.181 | −19.893 | 40.861 | 1.00 | 3.02 | O |
| ATOM | 2621 | NE2 | GLN | C | 57 | 67.685 | −20.835 | 42.836 | 1.00 | 3.02 | N |
| ATOM | 2622 | C | GLN | C | 57 | 68.930 | −23.610 | 37.592 | 1.00 | 3.02 | C |
| ATOM | 2623 | O | GLN | C | 57 | 68.759 | −23.034 | 36.518 | 1.00 | 3.02 | O |
| ATOM | 2624 | N | TYR | C | 58 | 68.619 | −24.884 | 37.830 | 1.00 | 3.02 | N |
| ATOM | 2625 | CA | TYR | C | 58 | 68.014 | −25.848 | 36.890 | 1.00 | 3.02 | C |
| ATOM | 2626 | CB | TYR | C | 58 | 67.599 | −27.089 | 37.689 | 1.00 | 3.02 | C |
| ATOM | 2627 | CG | TYR | C | 58 | 66.430 | −26.780 | 38.597 | 1.00 | 3.02 | C |
| ATOM | 2628 | CD1 | TYR | C | 58 | 65.123 | −27.021 | 38.177 | 1.00 | 3.02 | C |
| ATOM | 2629 | CE1 | TYR | C | 58 | 64.040 | −26.714 | 38.984 | 1.00 | 3.02 | C |
| ATOM | 2630 | CZ | TYR | C | 58 | 64.250 | −26.138 | 40.234 | 1.00 | 3.02 | C |
| ATOM | 2631 | OH | TYR | C | 58 | 63.169 | −25.836 | 41.029 | 1.00 | 3.02 | O |
| ATOM | 2632 | CE2 | TYR | C | 58 | 65.535 | −25.875 | 40.676 | 1.00 | 3.02 | C |
| ATOM | 2633 | CD2 | TYR | C | 58 | 66.622 | −26.194 | 39.851 | 1.00 | 3.02 | C |
| ATOM | 2634 | C | TYR | C | 58 | 66.798 | −25.345 | 36.074 | 1.00 | 3.02 | C |
| ATOM | 2635 | O | TYR | C | 58 | 66.776 | −25.473 | 34.857 | 1.00 | 3.02 | O |
| ATOM | 2636 | N | CYS | C | 59 | 65.788 | −24.793 | 36.751 | 1.00 | 3.02 | N |
| ATOM | 2637 | CA | CYS | C | 59 | 64.565 | −24.272 | 36.103 | 1.00 | 3.02 | C |
| ATOM | 2638 | CB | CYS | C | 59 | 63.619 | −23.612 | 37.128 | 1.00 | 3.02 | C |
| ATOM | 2639 | SG | CYS | C | 59 | 62.858 | −24.662 | 38.408 | 1.00 | 3.02 | S |
| ATOM | 2640 | C | CYS | C | 59 | 64.877 | −23.222 | 35.044 | 1.00 | 3.02 | C |
| ATOM | 2641 | O | CYS | C | 59 | 64.043 | −22.931 | 34.175 | 1.00 | 3.02 | O |
| ATOM | 2642 | N | ARG | C | 60 | 66.076 | −22.650 | 35.161 | 1.00 | 3.02 | N |
| ATOM | 2643 | CA | ARG | C | 60 | 66.545 | −21.540 | 34.352 | 1.00 | 3.02 | C |
| ATOM | 2644 | CB | ARG | C | 60 | 67.267 | −20.546 | 35.270 | 1.00 | 3.02 | C |
| ATOM | 2645 | CG | ARG | C | 60 | 67.625 | −19.188 | 34.690 | 1.00 | 3.02 | C |
| ATOM | 2646 | CD | ARG | C | 60 | 69.127 | −18.959 | 34.633 | 1.00 | 3.02 | C |
| ATOM | 2647 | NE | ARG | C | 60 | 69.661 | −19.416 | 33.362 | 1.00 | 3.02 | N |
| ATOM | 2648 | CZ | ARG | C | 60 | 70.899 | −19.195 | 32.949 | 1.00 | 3.02 | C |
| ATOM | 2649 | NH1 | ARG | C | 60 | 71.740 | −18.510 | 33.706 | 1.00 | 3.02 | N |
| ATOM | 2650 | NH2 | ARG | C | 60 | 71.302 | −19.669 | 31.778 | 1.00 | 3.02 | N |
| ATOM | 2651 | C | ARG | C | 60 | 67.459 | −22.097 | 33.263 | 1.00 | 3.02 | C |
| ATOM | 2652 | O | ARG | C | 60 | 67.186 | −21.932 | 32.067 | 1.00 | 3.02 | O |
| ATOM | 2653 | N | SER | C | 61 | 68.502 | −22.818 | 33.669 | 1.00 | 3.02 | N |
| ATOM | 2654 | CA | SER | C | 61 | 69.557 | −23.222 | 32.747 | 1.00 | 3.02 | C |
| ATOM | 2655 | CB | SER | C | 61 | 70.908 | −22.878 | 33.354 | 1.00 | 3.02 | C |
| ATOM | 2656 | OG | SER | C | 61 | 70.907 | −23.154 | 34.741 | 1.00 | 3.02 | O |
| ATOM | 2657 | C | SER | C | 61 | 69.530 | −24.681 | 32.261 | 1.00 | 3.02 | C |
| ATOM | 2658 | O | SER | C | 61 | 69.328 | −24.932 | 31.069 | 1.00 | 3.02 | O |
| ATOM | 2659 | N | ASP | C | 62 | 69.721 | −25.632 | 33.172 | 1.00 | 3.02 | N |
| ATOM | 2660 | CA | ASP | C | 62 | 69.999 | −27.032 | 32.797 | 1.00 | 3.02 | C |
| ATOM | 2661 | CB | ASP | C | 62 | 70.179 | −27.918 | 34.042 | 1.00 | 3.02 | C |
| ATOM | 2662 | CG | ASP | C | 62 | 71.638 | −28.026 | 34.483 | 1.00 | 3.02 | C |
| ATOM | 2663 | OD1 | ASP | C | 62 | 72.402 | −27.066 | 34.274 | 1.00 | 3.02 | O |
| ATOM | 2664 | OD2 | ASP | C | 62 | 72.021 | −29.069 | 35.044 | 1.00 | 3.02 | O |
| ATOM | 2665 | C | ASP | C | 62 | 69.132 | −27.739 | 31.717 | 1.00 | 3.02 | C |
| ATOM | 2666 | O | ASP | C | 62 | 69.700 | −28.385 | 30.833 | 1.00 | 3.02 | O |
| ATOM | 2667 | N | PRO | C | 63 | 67.778 | −27.636 | 31.775 | 1.00 | 3.02 | N |
| ATOM | 2668 | CA | PRO | C | 63 | 67.034 | −28.322 | 30.709 | 1.00 | 3.02 | C |
| ATOM | 2669 | CB | PRO | C | 63 | 65.634 | −28.522 | 31.306 | 1.00 | 3.02 | C |
| ATOM | 2670 | CG | PRO | C | 63 | 65.534 | −27.584 | 32.468 | 1.00 | 3.02 | C |
| ATOM | 2671 | CD | PRO | C | 63 | 66.872 | −26.926 | 32.698 | 1.00 | 3.02 | C |
| ATOM | 2672 | C | PRO | C | 63 | 66.968 | −27.457 | 29.462 | 1.00 | 3.02 | C |
| ATOM | 2673 | O | PRO | C | 63 | 67.121 | −27.954 | 28.354 | 1.00 | 3.02 | O |
| ATOM | 2674 | N | ASP | C | 64 | 66.750 | −26.164 | 29.664 | 1.00 | 3.02 | N |
| ATOM | 2675 | CA | ASP | C | 64 | 66.803 | −25.188 | 28.600 | 1.00 | 3.02 | C |
| ATOM | 2676 | CB | ASP | C | 64 | 66.765 | −23.775 | 29.197 | 1.00 | 3.02 | C |
| ATOM | 2677 | CG | ASP | C | 64 | 66.860 | −22.690 | 28.141 | 1.00 | 3.02 | C |
| ATOM | 2678 | OD1 | ASP | C | 64 | 66.136 | −22.786 | 27.118 | 1.00 | 3.02 | O |
| ATOM | 2679 | OD2 | ASP | C | 64 | 67.660 | −21.743 | 28.338 | 1.00 | 3.02 | O |
| ATOM | 2680 | C | ASP | C | 64 | 68.053 | −25.397 | 27.729 | 1.00 | 3.02 | C |
| ATOM | 2681 | O | ASP | C | 64 | 67.956 | −26.044 | 26.675 | 1.00 | 3.02 | O |
| ATOM | 2682 | N | VAL | C | 65 | 69.209 | −24.889 | 28.197 | 1.00 | 3.02 | N |
| ATOM | 2683 | CA | VAL | C | 65 | 70.483 | −24.903 | 27.450 | 1.00 | 3.02 | C |
| ATOM | 2684 | CB | VAL | C | 65 | 71.720 | −24.557 | 28.348 | 1.00 | 3.02 | C |
| ATOM | 2685 | CG1 | VAL | C | 65 | 73.029 | −25.028 | 27.707 | 1.00 | 3.02 | C |
| ATOM | 2686 | CG2 | VAL | C | 65 | 71.803 | −23.056 | 28.631 | 1.00 | 3.02 | C |
| ATOM | 2687 | C | VAL | C | 65 | 70.688 | −26.237 | 26.753 | 1.00 | 3.02 | C |
| ATOM | 2688 | O | VAL | C | 65 | 71.148 | −26.271 | 25.599 | 1.00 | 3.02 | O |
| ATOM | 2689 | N | GLU | C | 66 | 70.333 | −27.315 | 27.461 | 1.00 | 3.02 | N |
| ATOM | 2690 | CA | GLU | C | 66 | 70.302 | −28.664 | 26.898 | 1.00 | 3.02 | C |
| ATOM | 2691 | CB | GLU | C | 66 | 70.009 | −29.701 | 27.983 | 1.00 | 3.02 | C |
| ATOM | 2692 | CG | GLU | C | 66 | 69.812 | −31.115 | 27.483 | 1.00 | 3.02 | C |
| ATOM | 2693 | CD | GLU | C | 66 | 70.300 | −32.139 | 28.475 | 1.00 | 3.02 | C |
| ATOM | 2694 | OE1 | GLU | C | 66 | 70.303 | −31.804 | 29.681 | 1.00 | 3.02 | O |
| ATOM | 2695 | OE2 | GLU | C | 66 | 70.695 | −33.257 | 28.054 | 1.00 | 3.02 | O |
| ATOM | 2696 | C | GLU | C | 66 | 69.290 | −28.739 | 25.753 | 1.00 | 3.02 | C |
| ATOM | 2697 | O | GLU | C | 66 | 69.698 | −28.817 | 24.600 | 1.00 | 3.02 | O |
| ATOM | 2698 | N | ARG | C | 67 | 67.993 | −28.675 | 26.061 | 1.00 | 3.02 | N |
| ATOM | 2699 | CA | ARG | C | 67 | 66.951 | −28.919 | 25.057 | 1.00 | 3.02 | C |
| ATOM | 2700 | CB | ARG | C | 67 | 65.550 | −28.697 | 25.620 | 1.00 | 3.02 | C |
| ATOM | 2701 | CG | ARG | C | 67 | 64.880 | −29.903 | 26.255 | 1.00 | 3.02 | C |
| ATOM | 2702 | CD | ARG | C | 67 | 63.407 | −29.564 | 26.416 | 1.00 | 3.02 | C |
| ATOM | 2703 | NE | ARG | C | 67 | 62.822 | −30.081 | 27.649 | 1.00 | 3.02 | N |
| ATOM | 2704 | CZ | ARG | C | 67 | 61.788 | −29.526 | 28.269 | 1.00 | 3.02 | C |
| ATOM | 2705 | NH1 | ARG | C | 67 | 61.237 | −28.423 | 27.784 | 1.00 | 3.02 | N |
| ATOM | 2706 | NH2 | ARG | C | 67 | 61.318 | −30.058 | 29.386 | 1.00 | 3.02 | N |
| ATOM | 2707 | C | ARG | C | 67 | 67.137 | −28.058 | 23.824 | 1.00 | 3.02 | C |
| ATOM | 2708 | O | ARG | C | 67 | 67.078 | −28.581 | 22.704 | 1.00 | 3.02 | O |
| ATOM | 2709 | N | CYS | C | 68 | 67.333 | −26.749 | 24.047 | 1.00 | 3.02 | N |
| ATOM | 2710 | CA | CYS | C | 68 | 67.706 | −25.786 | 23.002 | 1.00 | 3.02 | C |
| ATOM | 2711 | CB | CYS | C | 68 | 68.416 | −24.562 | 23.614 | 1.00 | 3.02 | C |
| ATOM | 2712 | SG | CYS | C | 68 | 67.417 | −23.251 | 24.372 | 1.00 | 3.02 | S |
| ATOM | 2713 | C | CYS | C | 68 | 68.675 | −26.452 | 22.037 | 1.00 | 3.02 | C |
| ATOM | 2714 | O | CYS | C | 68 | 68.385 | −26.572 | 20.845 | 1.00 | 3.02 | O |
| ATOM | 2715 | N | LEU | C | 69 | 69.811 | −26.895 | 22.595 | 1.00 | 3.02 | N |
| ATOM | 2716 | CA | LEU | C | 69 | 70.911 | −27.530 | 21.868 | 1.00 | 3.02 | C |
| ATOM | 2717 | CB | LEU | C | 69 | 72.232 | −27.390 | 22.636 | 1.00 | 3.02 | C |
| ATOM | 2718 | CG | LEU | C | 69 | 72.929 | −26.037 | 22.875 | 1.00 | 3.02 | C |
| ATOM | 2719 | CD1 | LEU | C | 69 | 74.210 | −26.283 | 23.673 | 1.00 | 3.02 | C |
| ATOM | 2720 | CD2 | LEU | C | 69 | 73.242 | −25.231 | 21.590 | 1.00 | 3.02 | C |
| ATOM | 2721 | C | LEU | C | 69 | 70.672 | −29.004 | 21.570 | 1.00 | 3.02 | C |
| ATOM | 2722 | O | LEU | C | 69 | 71.244 | −29.531 | 20.621 | 1.00 | 3.02 | O |
| ATOM | 2723 | N | ARG | C | 70 | 69.829 | −29.662 | 22.377 | 1.00 | 3.02 | N |
| ATOM | 2724 | CA | ARG | C | 70 | 69.450 | −31.081 | 22.175 | 1.00 | 3.02 | C |
| ATOM | 2725 | CB | ARG | C | 70 | 68.679 | −31.653 | 23.375 | 1.00 | 3.02 | C |
| ATOM | 2726 | CG | ARG | C | 70 | 69.561 | −32.174 | 24.507 | 1.00 | 3.02 | C |
| ATOM | 2727 | CD | ARG | C | 70 | 70.504 | −33.264 | 24.008 | 1.00 | 3.02 | C |
| ATOM | 2728 | NE | ARG | C | 70 | 71.611 | −33.593 | 24.926 | 1.00 | 3.02 | N |
| ATOM | 2729 | CZ | ARG | C | 70 | 72.725 | −34.263 | 24.578 | 1.00 | 3.02 | C |
| ATOM | 2730 | NH1 | ARG | C | 70 | 72.902 | −34.692 | 23.323 | 1.00 | 3.02 | N |
| ATOM | 2731 | NH2 | ARG | C | 70 | 73.676 | −34.487 | 25.485 | 1.00 | 3.02 | N |
| ATOM | 2732 | C | ARG | C | 70 | 68.668 | −31.315 | 20.890 | 1.00 | 3.02 | C |
| ATOM | 2733 | O | ARG | C | 70 | 69.017 | −32.204 | 20.112 | 1.00 | 3.02 | O |
| ATOM | 2734 | N | ALA | C | 71 | 67.615 | −30.524 | 20.681 | 1.00 | 3.02 | N |
| ATOM | 2735 | CA | ALA | C | 71 | 66.916 | −30.499 | 19.402 | 1.00 | 3.02 | C |
| ATOM | 2736 | CB | ALA | C | 71 | 65.704 | −29.601 | 19.487 | 1.00 | 3.02 | C |
| ATOM | 2737 | C | ALA | C | 71 | 67.868 | −30.052 | 18.268 | 1.00 | 3.02 | C |
| ATOM | 2738 | O | ALA | C | 71 | 67.997 | −30.755 | 17.257 | 1.00 | 3.02 | O |
| ATOM | 2739 | N | HIS | C | 72 | 68.551 | −28.915 | 18.476 | 1.00 | 3.02 | N |
| ATOM | 2740 | CA | HIS | C | 72 | 69.425 | −28.258 | 17.484 | 1.00 | 3.02 | C |
| ATOM | 2741 | CB | HIS | C | 72 | 70.175 | −27.086 | 18.127 | 1.00 | 3.02 | C |
| ATOM | 2742 | CG | HIS | C | 72 | 69.467 | −25.769 | 18.026 | 1.00 | 3.02 | C |
| ATOM | 2743 | ND1 | HIS | C | 72 | 68.396 | −25.434 | 18.827 | 1.00 | 3.02 | N |
| ATOM | 2744 | CE1 | HIS | C | 72 | 67.996 | −24.211 | 18.529 | 1.00 | 3.02 | C |
| ATOM | 2745 | NE2 | HIS | C | 72 | 68.769 | −23.740 | 17.567 | 1.00 | 3.02 | N |
| ATOM | 2746 | CD2 | HIS | C | 72 | 69.699 | −24.694 | 17.238 | 1.00 | 3.02 | C |
| ATOM | 2747 | C | HIS | C | 72 | 70.445 | −29.175 | 16.829 | 1.00 | 3.02 | C |
| ATOM | 2748 | O | HIS | C | 72 | 70.417 | −29.370 | 15.612 | 1.00 | 3.02 | O |
| ATOM | 2749 | N | ARG | C | 73 | 71.346 | −29.712 | 17.652 | 1.00 | 3.02 | N |
| ATOM | 2750 | CA | ARG | C | 73 | 72.432 | −30.595 | 17.207 | 1.00 | 3.02 | C |
| ATOM | 2751 | CB | ARG | C | 73 | 73.238 | −31.106 | 18.402 | 1.00 | 3.02 | C |
| ATOM | 2752 | CG | ARG | C | 73 | 74.057 | −30.059 | 19.109 | 1.00 | 3.02 | C |
| ATOM | 2753 | CD | ARG | C | 73 | 74.729 | −30.659 | 20.315 | 1.00 | 3.02 | C |
| ATOM | 2754 | NE | ARG | C | 73 | 75.185 | −29.611 | 21.217 | 1.00 | 3.02 | N |
| ATOM | 2755 | CZ | ARG | C | 73 | 76.101 | −29.758 | 22.175 | 1.00 | 3.02 | C |
| ATOM | 2756 | NH1 | ARG | C | 73 | 76.696 | −30.935 | 22.388 | 1.00 | 3.02 | N |
| ATOM | 2757 | NH2 | ARG | C | 73 | 76.415 | −28.709 | 22.932 | 1.00 | 3.02 | N |
| ATOM | 2758 | C | ARG | C | 73 | 71.893 | −31.788 | 16.440 | 1.00 | 3.02 | C |
| ATOM | 2759 | O | ARG | C | 73 | 72.553 | −32.297 | 15.526 | 1.00 | 3.02 | O |
| ATOM | 2760 | N | ASN | C | 74 | 70.696 | −32.221 | 16.840 | 1.00 | 3.02 | N |
| ATOM | 2761 | CA | ASN | C | 74 | 69.985 | −33.322 | 16.208 | 1.00 | 3.02 | C |
| ATOM | 2762 | CB | ASN | C | 74 | 68.947 | −33.895 | 17.166 | 1.00 | 3.02 | C |
| ATOM | 2763 | CG | ASN | C | 74 | 69.568 | −34.743 | 18.264 | 1.00 | 3.02 | C |
| ATOM | 2764 | OD1 | ASN | C | 74 | 70.471 | −35.532 | 18.009 | 1.00 | 3.02 | O |
| ATOM | 2765 | ND2 | ASN | C | 74 | 69.074 | −34.590 | 19.491 | 1.00 | 3.02 | N |
| ATOM | 2766 | C | ASN | C | 74 | 69.345 | −32.912 | 14.891 | 1.00 | 3.02 | C |
| ATOM | 2767 | O | ASN | C | 74 | 69.433 | −33.645 | 13.904 | 1.00 | 3.02 | O |
| ATOM | 2768 | N | ASP | C | 75 | 68.721 | −31.734 | 14.885 | 1.00 | 3.02 | N |
| ATOM | 2769 | CA | ASP | C | 75 | 68.229 | −31.101 | 13.654 | 1.00 | 3.02 | C |
| ATOM | 2770 | CB | ASP | C | 75 | 67.615 | −29.731 | 13.955 | 1.00 | 3.02 | C |
| ATOM | 2771 | CG | ASP | C | 75 | 66.387 | −29.823 | 14.816 | 1.00 | 3.02 | C |
| ATOM | 2772 | OD1 | ASP | C | 75 | 66.010 | −30.958 | 15.186 | 1.00 | 3.02 | O |
| ATOM | 2773 | OD2 | ASP | C | 75 | 65.813 | −28.757 | 15.124 | 1.00 | 3.02 | O |
| ATOM | 2774 | C | ASP | C | 75 | 69.352 | −30.945 | 12.630 | 1.00 | 3.02 | C |
| ATOM | 2775 | O | ASP | C | 75 | 69.204 | −31.334 | 11.459 | 1.00 | 3.02 | O |
| ATOM | 2776 | N | MET | C | 76 | 70.477 | −30.400 | 13.090 | 1.00 | 3.02 | N |
| ATOM | 2777 | CA | MET | C | 76 | 71.676 | −30.236 | 12.263 | 1.00 | 3.02 | C |
| ATOM | 2778 | CB | MET | C | 76 | 72.823 | −29.648 | 13.099 | 1.00 | 3.02 | C |
| ATOM | 2779 | CG | MET | C | 76 | 72.869 | −28.119 | 13.048 | 1.00 | 3.02 | C |
| ATOM | 2780 | SD | MET | C | 76 | 73.239 | −27.242 | 14.587 | 1.00 | 3.02 | S |
| ATOM | 2781 | CE | MET | C | 76 | 75.008 | −27.065 | 14.473 | 1.00 | 3.02 | C |
| ATOM | 2782 | C | MET | C | 76 | 72.122 | −31.498 | 11.502 | 1.00 | 3.02 | C |
| ATOM | 2783 | O | MET | C | 76 | 72.253 | −31.476 | 10.277 | 1.00 | 3.02 | O |
| ATOM | 2784 | N | GLU | C | 77 | 72.310 | −32.601 | 12.219 | 1.00 | 3.02 | N |
| ATOM | 2785 | CA | GLU | C | 77 | 72.976 | −33.781 | 11.642 | 1.00 | 3.02 | C |
| ATOM | 2786 | CB | GLU | C | 77 | 73.685 | −34.604 | 12.725 | 1.00 | 3.02 | C |
| ATOM | 2787 | CG | GLU | C | 77 | 74.675 | −33.777 | 13.530 | 1.00 | 3.02 | C |
| ATOM | 2788 | CD | GLU | C | 77 | 75.796 | −34.595 | 14.146 | 1.00 | 3.02 | C |
| ATOM | 2789 | OE1 | GLU | C | 77 | 75.517 | −35.602 | 14.847 | 1.00 | 3.02 | O |
| ATOM | 2790 | OE2 | GLU | C | 77 | 76.967 | −34.204 | 13.935 | 1.00 | 3.02 | O |
| ATOM | 2791 | C | GLU | C | 77 | 72.167 | −34.674 | 10.687 | 1.00 | 3.02 | C |
| ATOM | 2792 | O | GLU | C | 77 | 72.776 | −35.404 | 9.900 | 1.00 | 3.02 | O |
| ATOM | 2793 | N | THR | C | 78 | 70.832 | −34.632 | 10.747 | 1.00 | 3.02 | N |
| ATOM | 2794 | CA | THR | C | 78 | 70.004 | −35.356 | 9.763 | 1.00 | 3.02 | C |
| ATOM | 2795 | CB | THR | C | 78 | 68.548 | −35.611 | 10.268 | 1.00 | 3.02 | C |
| ATOM | 2796 | OG1 | THR | C | 78 | 68.472 | −36.926 | 10.818 | 1.00 | 3.02 | O |
| ATOM | 2797 | CG2 | THR | C | 78 | 67.498 | −35.508 | 9.142 | 1.00 | 3.02 | C |
| ATOM | 2798 | C | THR | C | 78 | 70.006 | −34.622 | 8.431 | 1.00 | 3.02 | C |
| ATOM | 2799 | O | THR | C | 78 | 70.072 | −35.247 | 7.370 | 1.00 | 3.02 | O |
| ATOM | 2800 | N | ILE | C | 79 | 69.935 | −33.291 | 8.505 | 1.00 | 3.02 | N |
| ATOM | 2801 | CA | ILE | C | 79 | 69.910 | −32.457 | 7.308 | 1.00 | 3.02 | C |
| ATOM | 2802 | CB | ILE | C | 79 | 69.973 | −30.907 | 7.653 | 1.00 | 3.02 | C |
| ATOM | 2803 | CG1 | ILE | C | 79 | 68.621 | −30.243 | 7.368 | 1.00 | 3.02 | C |
| ATOM | 2804 | CD1 | ILE | C | 79 | 68.676 | −28.714 | 7.049 | 1.00 | 3.02 | C |
| ATOM | 2805 | CG2 | ILE | C | 79 | 71.082 | −30.179 | 6.911 | 1.00 | 3.02 | C |
| ATOM | 2806 | C | ILE | C | 79 | 70.921 | −32.974 | 6.247 | 1.00 | 3.02 | C |
| ATOM | 2807 | O | ILE | C | 79 | 70.499 | −33.645 | 5.293 | 1.00 | 3.02 | O |
| ATOM | 2808 | N | TYR | C | 80 | 72.225 | −32.733 | 6.467 | 1.00 | 3.02 | N |
| ATOM | 2809 | CA | TYR | C | 80 | 73.291 | −32.830 | 5.438 | 1.00 | 3.02 | C |
| ATOM | 2810 | CB | TYR | C | 80 | 74.689 | −32.596 | 6.049 | 1.00 | 3.02 | C |
| ATOM | 2811 | CG | TYR | C | 80 | 74.686 | −31.821 | 7.340 | 1.00 | 3.02 | C |
| ATOM | 2812 | CD1 | TYR | C | 80 | 75.026 | −32.432 | 8.544 | 1.00 | 3.02 | C |
| ATOM | 2813 | CE1 | TYR | C | 80 | 75.021 | −31.721 | 9.733 | 1.00 | 3.02 | C |
| ATOM | 2814 | CZ | TYR | C | 80 | 74.662 | −30.388 | 9.723 | 1.00 | 3.02 | C |
| ATOM | 2815 | OH | TYR | C | 80 | 74.637 | −29.664 | 10.889 | 1.00 | 3.02 | O |
| ATOM | 2816 | CE2 | TYR | C | 80 | 74.316 | −29.767 | 8.542 | 1.00 | 3.02 | C |
| ATOM | 2817 | CD2 | TYR | C | 80 | 74.336 | −30.484 | 7.359 | 1.00 | 3.02 | C |
| ATOM | 2818 | C | TYR | C | 80 | 73.300 | −34.095 | 4.564 | 1.00 | 3.02 | C |
| ATOM | 2819 | O | TYR | C | 80 | 73.219 | −33.988 | 3.333 | 1.00 | 3.02 | O |
| ATOM | 2820 | N | PRO | C | 81 | 73.369 | −35.294 | 5.186 | 1.00 | 3.02 | N |
| ATOM | 2821 | CA | PRO | C | 81 | 73.439 | −36.492 | 4.348 | 1.00 | 3.02 | C |
| ATOM | 2822 | CB | PRO | C | 81 | 73.430 | −37.644 | 5.353 | 1.00 | 3.02 | C |
| ATOM | 2823 | CG | PRO | C | 81 | 73.647 | −37.066 | 6.655 | 1.00 | 3.02 | C |
| ATOM | 2824 | CD | PRO | C | 81 | 73.322 | −35.618 | 6.624 | 1.00 | 3.02 | C |
| ATOM | 2825 | C | PRO | C | 81 | 72.184 | −36.573 | 3.497 | 1.00 | 3.02 | C |
| ATOM | 2826 | O | PRO | C | 81 | 72.240 | −36.710 | 2.280 | 1.00 | 3.02 | O |
| ATOM | 2827 | N | PHE | C | 82 | 71.051 | −36.419 | 4.170 | 1.00 | 3.02 | N |
| ATOM | 2828 | CA | PHE | C | 82 | 69.715 | −36.387 | 3.591 | 1.00 | 3.02 | C |
| ATOM | 2829 | CB | PHE | C | 82 | 68.763 | −36.018 | 4.708 | 1.00 | 3.02 | C |
| ATOM | 2830 | CG | PHE | C | 82 | 67.361 | −36.347 | 4.422 | 1.00 | 3.02 | C |
| ATOM | 2831 | CD1 | PHE | C | 82 | 66.939 | −37.665 | 4.438 | 1.00 | 3.02 | C |
| ATOM | 2832 | CE1 | PHE | C | 82 | 65.653 | −37.976 | 4.188 | 1.00 | 3.02 | C |
| ATOM | 2833 | CZ | PHE | C | 82 | 64.749 | −36.965 | 3.917 | 1.00 | 3.02 | C |
| ATOM | 2834 | CE2 | PHE | C | 82 | 65.160 | −35.637 | 3.885 | 1.00 | 3.02 | C |
| ATOM | 2835 | CD2 | PHE | C | 82 | 66.455 | −35.341 | 4.138 | 1.00 | 3.02 | C |
| ATOM | 2836 | C | PHE | C | 82 | 69.458 | −35.451 | 2.392 | 1.00 | 3.02 | C |
| ATOM | 2837 | O | PHE | C | 82 | 69.074 | −35.916 | 1.316 | 1.00 | 3.02 | O |
| ATOM | 2838 | N | LEU | C | 83 | 69.632 | −34.143 | 2.593 | 1.00 | 3.02 | N |
| ATOM | 2839 | CA | LEU | C | 83 | 69.348 | −33.144 | 1.555 | 1.00 | 3.02 | C |
| ATOM | 2840 | CB | LEU | C | 83 | 69.212 | −31.758 | 2.151 | 1.00 | 3.02 | C |
| ATOM | 2841 | CG | LEU | C | 83 | 69.418 | −31.587 | 3.641 | 1.00 | 3.02 | C |
| ATOM | 2842 | CD1 | LEU | C | 83 | 69.893 | −30.180 | 3.866 | 1.00 | 3.02 | C |
| ATOM | 2843 | CD2 | LEU | C | 83 | 68.138 | −31.873 | 4.399 | 1.00 | 3.02 | C |
| ATOM | 2844 | C | LEU | C | 83 | 70.436 | −33.083 | 0.503 | 1.00 | 3.02 | C |
| ATOM | 2845 | O | LEU | C | 83 | 70.142 | −33.201 | −0.680 | 1.00 | 3.02 | O |
| ATOM | 2846 | N | PHE | C | 84 | 71.682 | −32.873 | 0.951 | 1.00 | 3.02 | N |
| ATOM | 2847 | CA | PHE | C | 84 | 72.886 | −32.814 | 0.094 | 1.00 | 3.02 | C |
| ATOM | 2848 | CB | PHE | C | 84 | 74.126 | −32.469 | 0.928 | 1.00 | 3.02 | C |
| ATOM | 2849 | CG | PHE | C | 84 | 75.432 | −33.022 | 0.380 | 1.00 | 3.02 | C |
| ATOM | 2850 | CD1 | PHE | C | 84 | 76.541 | −32.216 | 0.269 | 1.00 | 3.02 | C |
| ATOM | 2851 | CE1 | PHE | C | 84 | 77.746 | −32.703 | −0.213 | 1.00 | 3.02 | C |
| ATOM | 2852 | CZ | PHE | C | 84 | 77.855 | −33.995 | −0.583 | 1.00 | 3.02 | C |
| ATOM | 2853 | CE2 | PHE | C | 84 | 76.751 | −34.831 | −0.485 | 1.00 | 3.02 | C |
| ATOM | 2854 | CD2 | PHE | C | 84 | 75.558 | −34.347 | 0.007 | 1.00 | 3.02 | C |
| ATOM | 2855 | C | PHE | C | 84 | 73.137 | −34.087 | −0.720 | 1.00 | 3.02 | C |
| ATOM | 2856 | O | PHE | C | 84 | 73.445 | −34.002 | −1.910 | 1.00 | 3.02 | O |
| ATOM | 2857 | N | LEU | C | 85 | 73.030 | −35.257 | −0.091 | 1.00 | 3.02 | N |
| ATOM | 2858 | CA | LEU | C | 85 | 73.107 | −36.492 | −0.863 | 1.00 | 3.02 | C |
| ATOM | 2859 | CB | LEU | C | 85 | 73.214 | −37.730 | 0.040 | 1.00 | 3.02 | C |
| ATOM | 2860 | CG | LEU | C | 85 | 74.460 | −37.949 | 0.932 | 1.00 | 3.02 | C |
| ATOM | 2861 | CD1 | LEU | C | 85 | 74.337 | −39.250 | 1.718 | 1.00 | 3.02 | C |
| ATOM | 2862 | CD2 | LEU | C | 85 | 75.755 | −37.938 | 0.149 | 1.00 | 3.02 | C |
| ATOM | 2863 | C | LEU | C | 85 | 71.880 | −36.555 | −1.770 | 1.00 | 3.02 | C |
| ATOM | 2864 | O | LEU | C | 85 | 71.990 | −36.925 | −2.949 | 1.00 | 3.02 | O |
| ATOM | 2865 | N | GLY | C | 86 | 70.732 | −36.150 | −1.213 | 1.00 | 3.02 | N |
| ATOM | 2866 | CA | GLY | C | 86 | 69.451 | −36.110 | −1.927 | 1.00 | 3.02 | C |
| ATOM | 2867 | C | GLY | C | 86 | 69.327 | −35.053 | −3.015 | 1.00 | 3.02 | C |
| ATOM | 2868 | O | GLY | C | 86 | 68.556 | −35.249 | −3.955 | 1.00 | 3.02 | O |
| ATOM | 2869 | N | PHE | C | 87 | 70.069 | −33.945 | −2.889 | 1.00 | 3.02 | N |
| ATOM | 2870 | CA | PHE | C | 87 | 70.127 | −32.920 | −3.944 | 1.00 | 3.02 | C |
| ATOM | 2871 | CB | PHE | C | 87 | 70.643 | −31.571 | −3.415 | 1.00 | 3.02 | C |
| ATOM | 2872 | CG | PHE | C | 87 | 69.599 | −30.745 | −2.704 | 1.00 | 3.02 | C |
| ATOM | 2873 | CD1 | PHE | C | 87 | 69.338 | −30.938 | −1.355 | 1.00 | 3.02 | C |
| ATOM | 2874 | CE1 | PHE | C | 87 | 68.381 | −30.175 | −0.688 | 1.00 | 3.02 | C |
| ATOM | 2875 | CZ | PHE | C | 87 | 67.684 | −29.196 | −1.368 | 1.00 | 3.02 | C |
| ATOM | 2876 | CE2 | PHE | C | 87 | 67.933 | −28.992 | −2.711 | 1.00 | 3.02 | C |
| ATOM | 2877 | CD2 | PHE | C | 87 | 68.892 | −29.758 | −3.375 | 1.00 | 3.02 | C |
| ATOM | 2878 | C | PHE | C | 87 | 70.959 | −33.368 | −5.159 | 1.00 | 3.02 | C |
| ATOM | 2879 | O | PHE | C | 87 | 70.467 | −33.285 | −6.292 | 1.00 | 3.02 | O |
| ATOM | 2880 | N | VAL | C | 88 | 72.195 | −33.841 | −4.934 | 1.00 | 3.02 | N |
| ATOM | 2881 | CA | VAL | C | 88 | 73.107 | −34.182 | −6.044 | 1.00 | 3.02 | C |
| ATOM | 2882 | CB | VAL | C | 88 | 74.400 | −34.907 | −5.615 | 1.00 | 3.02 | C |
| ATOM | 2883 | CG1 | VAL | C | 88 | 75.525 | −34.617 | −6.607 | 1.00 | 3.02 | C |
| ATOM | 2884 | CG2 | VAL | C | 88 | 74.813 | −34.562 | −4.209 | 1.00 | 3.02 | C |
| ATOM | 2885 | C | VAL | C | 88 | 72.451 | −35.163 | −6.980 | 1.00 | 3.02 | C |
| ATOM | 2886 | O | VAL | C | 88 | 72.364 | −34.923 | −8.189 | 1.00 | 3.02 | O |
| ATOM | 2887 | N | TYR | C | 89 | 71.997 | −36.270 | −6.393 | 1.00 | 3.02 | N |
| ATOM | 2888 | CA | TYR | C | 89 | 71.444 | −37.399 | −7.127 | 1.00 | 3.02 | C |
| ATOM | 2889 | CB | TYR | C | 89 | 71.588 | −38.682 | −6.284 | 1.00 | 3.02 | C |
| ATOM | 2890 | CG | TYR | C | 89 | 70.311 | −39.229 | −5.663 | 1.00 | 3.02 | C |
| ATOM | 2891 | CD1 | TYR | C | 89 | 69.866 | −40.511 | −5.982 | 1.00 | 3.02 | C |
| ATOM | 2892 | CE1 | TYR | C | 89 | 68.701 | −41.023 | −5.439 | 1.00 | 3.02 | C |
| ATOM | 2893 | CZ | TYR | C | 89 | 67.971 | −40.262 | −4.560 | 1.00 | 3.02 | C |
| ATOM | 2894 | OH | TYR | C | 89 | 66.822 | −40.777 | −4.021 | 1.00 | 3.02 | O |
| ATOM | 2895 | CE2 | TYR | C | 89 | 68.382 | −38.986 | −4.216 | 1.00 | 3.02 | C |
| ATOM | 2896 | CD2 | TYR | C | 89 | 69.545 | −38.472 | −4.767 | 1.00 | 3.02 | C |
| ATOM | 2897 | C | TYR | C | 89 | 69.995 | −37.132 | −7.582 | 1.00 | 3.02 | C |
| ATOM | 2898 | O | TYR | C | 89 | 69.382 | −37.959 | −8.262 | 1.00 | 3.02 | O |
| ATOM | 2899 | N | SER | C | 90 | 69.471 | −35.962 | −7.216 | 1.00 | 3.02 | N |
| ATOM | 2900 | CA | SER | C | 90 | 68.095 | −35.592 | −7.520 | 1.00 | 3.02 | C |
| ATOM | 2901 | CB | SER | C | 90 | 67.612 | −34.504 | −6.557 | 1.00 | 3.02 | C |
| ATOM | 2902 | OG | SER | C | 90 | 68.395 | −33.326 | −6.657 | 1.00 | 3.02 | O |
| ATOM | 2903 | C | SER | C | 90 | 67.929 | −35.102 | −8.950 | 1.00 | 3.02 | C |
| ATOM | 2904 | O | SER | C | 90 | 67.155 | −35.667 | −9.729 | 1.00 | 3.02 | O |
| ATOM | 2905 | N | PHE | C | 91 | 68.636 | −34.024 | −9.277 | 1.00 | 3.02 | N |
| ATOM | 2906 | CA | PHE | C | 91 | 68.545 | −33.436 | −10.604 | 1.00 | 3.02 | C |
| ATOM | 2907 | CB | PHE | C | 91 | 68.920 | −31.941 | −10.590 | 1.00 | 3.02 | C |
| ATOM | 2908 | CG | PHE | C | 91 | 68.285 | −31.169 | −9.463 | 1.00 | 3.02 | C |
| ATOM | 2909 | CD1 | PHE | C | 91 | 69.052 | −30.714 | −8.399 | 1.00 | 3.02 | C |
| ATOM | 2910 | CE1 | PHE | C | 91 | 68.473 | −30.021 | −7.346 | 1.00 | 3.02 | C |
| ATOM | 2911 | CZ | PHE | C | 91 | 67.098 | −29.771 | −7.345 | 1.00 | 3.02 | C |
| ATOM | 2912 | CE2 | PHE | C | 91 | 66.313 | −30.228 | −8.400 | 1.00 | 3.02 | C |
| ATOM | 2913 | CD2 | PHE | C | 91 | 66.911 | −30.923 | −9.454 | 1.00 | 3.02 | C |
| ATOM | 2914 | C | PHE | C | 91 | 69.470 | −34.277 | −11.457 | 1.00 | 3.02 | C |
| ATOM | 2915 | O | PHE | C | 91 | 70.680 | −33.989 | −11.568 | 1.00 | 3.02 | O |
| ATOM | 2916 | N | LEU | C | 92 | 68.887 | −35.336 | −12.034 | 1.00 | 3.02 | N |
| ATOM | 2917 | CA | LEU | C | 92 | 69.660 | −36.487 | −12.489 | 1.00 | 3.02 | C |
| ATOM | 2918 | CB | LEU | C | 92 | 70.532 | −36.972 | −11.322 | 1.00 | 3.02 | C |
| ATOM | 2919 | CG | LEU | C | 92 | 71.638 | −37.983 | −11.558 | 1.00 | 3.02 | C |
| ATOM | 2920 | CD1 | LEU | C | 92 | 72.568 | −37.515 | −12.649 | 1.00 | 3.02 | C |
| ATOM | 2921 | CD2 | LEU | C | 92 | 72.375 | −38.226 | −10.274 | 1.00 | 3.02 | C |
| ATOM | 2922 | C | LEU | C | 92 | 68.795 | −37.651 | −13.001 | 1.00 | 3.02 | C |
| ATOM | 2923 | O | LEU | C | 92 | 69.233 | −38.470 | −13.807 | 1.00 | 3.02 | O |
| ATOM | 2924 | N | GLY | C | 93 | 67.563 | −37.729 | −12.524 | 1.00 | 3.02 | N |
| ATOM | 2925 | CA | GLY | C | 93 | 66.679 | −38.836 | −12.876 | 1.00 | 3.02 | C |
| ATOM | 2926 | C | GLY | C | 93 | 66.810 | −39.929 | −11.838 | 1.00 | 3.02 | C |
| ATOM | 2927 | O | GLY | C | 93 | 67.703 | −40.770 | −11.958 | 1.00 | 3.02 | O |
| ATOM | 2928 | N | PRO | C | 94 | 65.972 | −39.870 | −10.774 | 1.00 | 3.02 | N |
| ATOM | 2929 | CA | PRO | C | 94 | 65.825 | −40.884 | −9.739 | 1.00 | 3.02 | C |
| ATOM | 2930 | CB | PRO | C | 94 | 65.884 | −40.042 | −8.458 | 1.00 | 3.02 | C |
| ATOM | 2931 | CG | PRO | C | 94 | 65.325 | −38.636 | −8.902 | 1.00 | 3.02 | C |
| ATOM | 2932 | CD | PRO | C | 94 | 65.159 | −38.693 | −10.425 | 1.00 | 3.02 | C |
| ATOM | 2933 | C | PRO | C | 94 | 64.480 | −41.603 | −9.828 | 1.00 | 3.02 | C |
| ATOM | 2934 | O | PRO | C | 94 | 63.868 | −41.624 | −10.898 | 1.00 | 3.02 | O |
| ATOM | 2935 | N | ASN | C | 95 | 64.042 | −42.188 | −8.712 | 1.00 | 3.02 | N |
| ATOM | 2936 | CA | ASN | C | 95 | 62.772 | −42.922 | −8.640 | 1.00 | 3.02 | C |
| ATOM | 2937 | CB | ASN | C | 95 | 63.029 | −44.415 | −8.829 | 1.00 | 3.02 | C |
| ATOM | 2938 | CG | ASN | C | 95 | 63.404 | −44.767 | −10.253 | 1.00 | 3.02 | C |
| ATOM | 2939 | OD1 | ASN | C | 95 | 62.794 | −44.291 | −11.206 | 1.00 | 3.02 | O |
| ATOM | 2940 | ND2 | ASN | C | 95 | 64.397 | −45.633 | −10.405 | 1.00 | 3.02 | N |
| ATOM | 2941 | C | ASN | C | 95 | 62.011 | −42.671 | −7.323 | 1.00 | 3.02 | C |
| ATOM | 2942 | O | ASN | C | 95 | 62.657 | −42.571 | −6.275 | 1.00 | 3.02 | O |
| ATOM | 2943 | N | PRO | C | 96 | 60.654 | −42.596 | −7.369 | 1.00 | 3.02 | N |
| ATOM | 2944 | CA | PRO | C | 96 | 59.819 | −42.204 | −6.212 | 1.00 | 3.02 | C |
| ATOM | 2945 | CB | PRO | C | 96 | 58.373 | −42.241 | −6.759 | 1.00 | 3.02 | C |
| ATOM | 2946 | CG | PRO | C | 96 | 58.502 | −42.276 | −8.233 | 1.00 | 3.02 | C |
| ATOM | 2947 | CD | PRO | C | 96 | 59.833 | −42.917 | −8.550 | 1.00 | 3.02 | C |
| ATOM | 2948 | C | PRO | C | 96 | 59.968 | −43.121 | −4.983 | 1.00 | 3.02 | C |
| ATOM | 2949 | O | PRO | C | 96 | 60.551 | −42.693 | −3.995 | 1.00 | 3.02 | O |
| ATOM | 2950 | N | PHE | C | 97 | 59.433 | −44.347 | −5.045 | 1.00 | 3.02 | N |
| ATOM | 2951 | CA | PHE | C | 97 | 59.560 | −45.354 | −3.980 | 1.00 | 3.02 | C |
| ATOM | 2952 | CB | PHE | C | 97 | 59.645 | −46.770 | −4.574 | 1.00 | 3.02 | C |
| ATOM | 2953 | CG | PHE | C | 97 | 58.318 | −47.428 | −4.891 | 1.00 | 3.02 | C |
| ATOM | 2954 | CD1 | PHE | C | 97 | 57.978 | −47.703 | −6.213 | 1.00 | 3.02 | C |
| ATOM | 2955 | CE1 | PHE | C | 97 | 56.785 | −48.339 | −6.531 | 1.00 | 3.02 | C |
| ATOM | 2956 | CZ | PHE | C | 97 | 55.917 | −48.725 | −5.517 | 1.00 | 3.02 | C |
| ATOM | 2957 | CE2 | PHE | C | 97 | 56.257 | −48.481 | −4.185 | 1.00 | 3.02 | C |
| ATOM | 2958 | CD2 | PHE | C | 97 | 57.457 | −47.847 | −3.879 | 1.00 | 3.02 | C |
| ATOM | 2959 | C | PHE | C | 97 | 60.855 | −45.125 | −3.224 | 1.00 | 3.02 | C |
| ATOM | 2960 | O | PHE | C | 97 | 60.850 | −44.871 | −2.021 | 1.00 | 3.02 | O |
| ATOM | 2961 | N | VAL | C | 98 | 61.955 | −45.240 | −3.973 | 1.00 | 3.02 | N |
| ATOM | 2962 | CA | VAL | C | 98 | 63.328 | −45.120 | −3.502 | 1.00 | 3.02 | C |
| ATOM | 2963 | CB | VAL | C | 98 | 64.275 | −45.127 | −4.723 | 1.00 | 3.02 | C |
| ATOM | 2964 | CG1 | VAL | C | 98 | 65.487 | −44.230 | −4.514 | 1.00 | 3.02 | C |
| ATOM | 2965 | CG2 | VAL | C | 98 | 64.674 | −46.573 | −5.084 | 1.00 | 3.02 | C |
| ATOM | 2966 | C | VAL | C | 98 | 63.533 | −43.882 | −2.645 | 1.00 | 3.02 | C |
| ATOM | 2967 | O | VAL | C | 98 | 63.808 | −44.000 | −1.450 | 1.00 | 3.02 | O |
| ATOM | 2968 | N | ALA | C | 99 | 63.361 | −42.712 | −3.273 | 1.00 | 3.02 | N |
| ATOM | 2969 | CA | ALA | C | 99 | 63.432 | −41.382 | −2.637 | 1.00 | 3.02 | C |
| ATOM | 2970 | CB | ALA | C | 99 | 63.504 | −40.285 | −3.700 | 1.00 | 3.02 | C |
| ATOM | 2971 | C | ALA | C | 99 | 62.267 | −41.123 | −1.676 | 1.00 | 3.02 | C |
| ATOM | 2972 | O | ALA | C | 99 | 62.264 | −40.132 | −0.955 | 1.00 | 3.02 | O |
| ATOM | 2973 | N | TRP | C | 100 | 61.278 | −42.016 | −1.672 | 1.00 | 3.02 | N |
| ATOM | 2974 | CA | TRP | C | 100 | 60.233 | −42.024 | −0.650 | 1.00 | 3.02 | C |
| ATOM | 2975 | CB | TRP | C | 100 | 58.955 | −42.651 | −1.188 | 1.00 | 3.02 | C |
| ATOM | 2976 | CG | TRP | C | 100 | 58.071 | −41.732 | −1.948 | 1.00 | 3.02 | C |
| ATOM | 2977 | CD1 | TRP | C | 100 | 58.029 | −41.574 | −3.296 | 1.00 | 3.02 | C |
| ATOM | 2978 | NE1 | TRP | C | 100 | 57.079 | −40.659 | −3.642 | 1.00 | 3.02 | N |
| ATOM | 2979 | CE2 | TRP | C | 100 | 56.466 | −40.208 | −2.504 | 1.00 | 3.02 | C |
| ATOM | 2980 | CD2 | TRP | C | 100 | 57.069 | −40.869 | −1.413 | 1.00 | 3.02 | C |
| ATOM | 2981 | CE3 | TRP | C | 100 | 56.618 | −40.585 | −0.117 | 1.00 | 3.02 | C |
| ATOM | 2982 | CZ3 | TRP | C | 100 | 55.583 | −39.648 | 0.046 | 1.00 | 3.02 | C |
| ATOM | 2983 | CH2 | TRP | C | 100 | 55.001 | −39.008 | −1.064 | 1.00 | 3.02 | C |
| ATOM | 2984 | CZ2 | TRP | C | 100 | 55.428 | −39.273 | −2.342 | 1.00 | 3.02 | C |
| ATOM | 2985 | C | TRP | C | 100 | 60.667 | −42.806 | 0.583 | 1.00 | 3.02 | C |
| ATOM | 2986 | O | TRP | C | 100 | 60.305 | −42.462 | 1.703 | 1.00 | 3.02 | O |
| ATOM | 2987 | N | MET | C | 101 | 61.418 | −43.879 | 0.373 | 1.00 | 3.02 | N |
| ATOM | 2988 | CA | MET | C | 101 | 61.914 | −44.682 | 1.483 | 1.00 | 3.02 | C |
| ATOM | 2989 | CB | MET | C | 101 | 62.559 | −45.988 | 0.989 | 1.00 | 3.02 | C |
| ATOM | 2990 | CG | MET | C | 101 | 61.565 | −47.151 | 0.800 | 1.00 | 3.02 | C |
| ATOM | 2991 | SD | MET | C | 101 | 62.322 | −48.801 | 0.737 | 1.00 | 3.02 | S |
| ATOM | 2992 | CE | MET | C | 101 | 62.884 | −49.019 | 2.435 | 1.00 | 3.02 | C |
| ATOM | 2993 | C | MET | C | 101 | 62.912 | −43.856 | 2.272 | 1.00 | 3.02 | C |
| ATOM | 2994 | O | MET | C | 101 | 62.783 | −43.687 | 3.489 | 1.00 | 3.02 | O |
| ATOM | 2995 | N | HIS | C | 102 | 63.891 | −43.324 | 1.553 | 1.00 | 3.02 | N |
| ATOM | 2996 | CA | HIS | C | 102 | 64.930 | −42.513 | 2.145 | 1.00 | 3.02 | C |
| ATOM | 2997 | CB | HIS | C | 102 | 65.974 | −42.158 | 1.087 | 1.00 | 3.02 | C |
| ATOM | 2998 | CG | HIS | C | 102 | 66.834 | −43.319 | 0.667 | 1.00 | 3.02 | C |
| ATOM | 2999 | ND1 | HIS | C | 102 | 67.328 | −44.248 | 1.560 | 1.00 | 3.02 | N |
| ATOM | 3000 | CE1 | HIS | C | 102 | 68.058 | −45.135 | 0.909 | 1.00 | 3.02 | C |
| ATOM | 3001 | NE2 | HIS | C | 102 | 68.071 | −44.807 | −0.369 | 1.00 | 3.02 | N |
| ATOM | 3002 | CD2 | HIS | C | 102 | 67.315 | −43.676 | −0.547 | 1.00 | 3.02 | C |
| ATOM | 3003 | C | HIS | C | 102 | 64.393 | −41.282 | 2.901 | 1.00 | 3.02 | C |
| ATOM | 3004 | O | HIS | C | 102 | 64.787 | −41.056 | 4.045 | 1.00 | 3.02 | O |
| ATOM | 3005 | N | PHE | C | 103 | 63.478 | −40.522 | 2.300 | 1.00 | 3.02 | N |
| ATOM | 3006 | CA | PHE | C | 103 | 62.895 | −39.359 | 2.985 | 1.00 | 3.02 | C |
| ATOM | 3007 | CB | PHE | C | 103 | 62.158 | −38.435 | 2.000 | 1.00 | 3.02 | C |
| ATOM | 3008 | CG | PHE | C | 103 | 63.072 | −37.778 | 0.984 | 1.00 | 3.02 | C |
| ATOM | 3009 | CD1 | PHE | C | 103 | 63.337 | −38.382 | −0.234 | 1.00 | 3.02 | C |
| ATOM | 3010 | CE1 | PHE | C | 103 | 64.186 | −37.781 | −1.182 | 1.00 | 3.02 | C |
| ATOM | 3011 | CZ | PHE | C | 103 | 64.789 | −36.568 | −0.908 | 1.00 | 3.02 | C |
| ATOM | 3012 | CE2 | PHE | C | 103 | 64.548 | −35.953 | 0.310 | 1.00 | 3.02 | C |
| ATOM | 3013 | CD2 | PHE | C | 103 | 63.677 | −36.558 | 1.249 | 1.00 | 3.02 | C |
| ATOM | 3014 | C | PHE | C | 103 | 62.074 | −39.695 | 4.256 | 1.00 | 3.02 | C |
| ATOM | 3015 | O | PHE | C | 103 | 62.272 | −39.065 | 5.297 | 1.00 | 3.02 | O |
| ATOM | 3016 | N | LEU | C | 104 | 61.201 | −40.707 | 4.179 | 1.00 | 3.02 | N |
| ATOM | 3017 | CA | LEU | C | 104 | 60.383 | −41.174 | 5.333 | 1.00 | 3.02 | C |
| ATOM | 3018 | CB | LEU | C | 104 | 59.338 | −42.217 | 4.897 | 1.00 | 3.02 | C |
| ATOM | 3019 | CG | LEU | C | 104 | 57.937 | −41.807 | 4.432 | 1.00 | 3.02 | C |
| ATOM | 3020 | CD1 | LEU | C | 104 | 57.165 | −43.046 | 3.973 | 1.00 | 3.02 | C |
| ATOM | 3021 | CD2 | LEU | C | 104 | 57.160 | −41.059 | 5.523 | 1.00 | 3.02 | C |
| ATOM | 3022 | C | LEU | C | 104 | 61.163 | −41.741 | 6.542 | 1.00 | 3.02 | C |
| ATOM | 3023 | O | LEU | C | 104 | 61.081 | −41.184 | 7.646 | 1.00 | 3.02 | O |
| ATOM | 3024 | N | VAL | C | 105 | 61.884 | −42.849 | 6.331 | 1.00 | 3.02 | N |
| ATOM | 3025 | CA | VAL | C | 105 | 62.602 | −43.578 | 7.391 | 1.00 | 3.02 | C |
| ATOM | 3026 | CB | VAL | C | 105 | 63.355 | −44.791 | 6.804 | 1.00 | 3.02 | C |
| ATOM | 3027 | CG1 | VAL | C | 105 | 64.407 | −45.326 | 7.778 | 1.00 | 3.02 | C |
| ATOM | 3028 | CG2 | VAL | C | 105 | 62.382 | −45.888 | 6.407 | 1.00 | 3.02 | C |
| ATOM | 3029 | C | VAL | C | 105 | 63.573 | −42.714 | 8.210 | 1.00 | 3.02 | C |
| ATOM | 3030 | O | VAL | C | 105 | 63.616 | −42.823 | 9.438 | 1.00 | 3.02 | O |
| ATOM | 3031 | N | PHE | C | 106 | 64.352 | −41.881 | 7.508 | 1.00 | 3.02 | N |
| ATOM | 3032 | CA | PHE | C | 106 | 65.240 | −40.857 | 8.096 | 1.00 | 3.02 | C |
| ATOM | 3033 | CB | PHE | C | 106 | 65.714 | −39.895 | 7.009 | 1.00 | 3.02 | C |
| ATOM | 3034 | CG | PHE | C | 106 | 67.142 | −40.069 | 6.594 | 1.00 | 3.02 | C |
| ATOM | 3035 | CD1 | PHE | C | 106 | 67.492 | −40.995 | 5.621 | 1.00 | 3.02 | C |
| ATOM | 3036 | CE1 | PHE | C | 106 | 68.827 | −41.133 | 5.218 | 1.00 | 3.02 | C |
| ATOM | 3037 | CZ | PHE | C | 106 | 69.822 | −40.320 | 5.775 | 1.00 | 3.02 | C |
| ATOM | 3038 | CE2 | PHE | C | 106 | 69.481 | −39.373 | 6.727 | 1.00 | 3.02 | C |
| ATOM | 3039 | CD2 | PHE | C | 106 | 68.139 | −39.248 | 7.129 | 1.00 | 3.02 | C |
| ATOM | 3040 | C | PHE | C | 106 | 64.543 | −40.007 | 9.158 | 1.00 | 3.02 | C |
| ATOM | 3041 | O | PHE | C | 106 | 65.018 | −39.911 | 10.296 | 1.00 | 3.02 | O |
| ATOM | 3042 | N | LEU | C | 107 | 63.432 | −39.375 | 8.759 | 1.00 | 3.02 | N |
| ATOM | 3043 | CA | LEU | C | 107 | 62.687 | −38.451 | 9.611 | 1.00 | 3.02 | C |
| ATOM | 3044 | CB | LEU | C | 107 | 61.572 | −37.764 | 8.822 | 1.00 | 3.02 | C |
| ATOM | 3045 | CG | LEU | C | 107 | 61.896 | −36.982 | 7.557 | 1.00 | 3.02 | C |
| ATOM | 3046 | CD1 | LEU | C | 107 | 60.592 | −36.561 | 6.896 | 1.00 | 3.02 | C |
| ATOM | 3047 | CD2 | LEU | C | 107 | 62.708 | −35.757 | 7.925 | 1.00 | 3.02 | C |
| ATOM | 3048 | C | LEU | C | 107 | 62.073 | −39.153 | 10.811 | 1.00 | 3.02 | C |
| ATOM | 3049 | O | LEU | C | 107 | 62.221 | −38.693 | 11.944 | 1.00 | 3.02 | O |
| ATOM | 3050 | N | VAL | C | 108 | 61.384 | −40.263 | 10.555 | 1.00 | 3.02 | N |
| ATOM | 3051 | CA | VAL | C | 108 | 60.714 | −41.018 | 11.603 | 1.00 | 3.02 | C |
| ATOM | 3052 | CB | VAL | C | 108 | 59.648 | −41.997 | 11.002 | 1.00 | 3.02 | C |
| ATOM | 3053 | CG1 | VAL | C | 108 | 59.030 | −42.926 | 12.063 | 1.00 | 3.02 | C |
| ATOM | 3054 | CG2 | VAL | C | 108 | 58.541 | −41.211 | 10.271 | 1.00 | 3.02 | C |
| ATOM | 3055 | C | VAL | C | 108 | 61.774 | −41.718 | 12.461 | 1.00 | 3.02 | C |
| ATOM | 3056 | O | VAL | C | 108 | 61.500 | −42.104 | 13.611 | 1.00 | 3.02 | O |
| ATOM | 3057 | N | GLY | C | 109 | 62.981 | −41.848 | 11.900 | 1.00 | 3.02 | N |
| ATOM | 3058 | CA | GLY | C | 109 | 64.154 | −42.338 | 12.633 | 1.00 | 3.02 | C |
| ATOM | 3059 | C | GLY | C | 109 | 64.881 | −41.258 | 13.420 | 1.00 | 3.02 | C |
| ATOM | 3060 | O | GLY | C | 109 | 65.365 | −41.491 | 14.520 | 1.00 | 3.02 | O |
| ATOM | 3061 | N | ARG | C | 110 | 64.963 | −40.066 | 12.858 | 1.00 | 3.02 | N |
| ATOM | 3062 | CA | ARG | C | 110 | 65.587 | −38.953 | 13.561 | 1.00 | 3.02 | C |
| ATOM | 3063 | CB | ARG | C | 110 | 66.160 | −37.926 | 12.568 | 1.00 | 3.02 | C |
| ATOM | 3064 | CG | ARG | C | 110 | 66.614 | −36.607 | 13.207 | 1.00 | 3.02 | C |
| ATOM | 3065 | CD | ARG | C | 110 | 67.842 | −36.806 | 14.098 | 1.00 | 3.02 | C |
| ATOM | 3066 | NE | ARG | C | 110 | 69.071 | −37.002 | 13.331 | 1.00 | 3.02 | N |
| ATOM | 3067 | CZ | ARG | C | 110 | 70.269 | −36.556 | 13.697 | 1.00 | 3.02 | C |
| ATOM | 3068 | NH1 | ARG | C | 110 | 70.411 | −35.880 | 14.823 | 1.00 | 3.02 | N |
| ATOM | 3069 | NH2 | ARG | C | 110 | 71.326 | −36.783 | 12.931 | 1.00 | 3.02 | N |
| ATOM | 3070 | C | ARG | C | 110 | 64.667 | −38.249 | 14.586 | 1.00 | 3.02 | C |
| ATOM | 3071 | O | ARG | C | 110 | 65.171 | −37.665 | 15.563 | 1.00 | 3.02 | O |
| ATOM | 3072 | N | VAL | C | 111 | 63.346 | −38.274 | 14.357 | 1.00 | 3.02 | N |
| ATOM | 3073 | CA | VAL | C | 111 | 62.392 | −37.675 | 15.301 | 1.00 | 3.02 | C |
| ATOM | 3074 | CB | VAL | C | 111 | 60.958 | −37.503 | 14.722 | 1.00 | 3.02 | C |
| ATOM | 3075 | CG1 | VAL | C | 111 | 59.937 | −37.112 | 15.818 | 1.00 | 3.02 | C |
| ATOM | 3076 | CG2 | VAL | C | 111 | 60.954 | −36.443 | 13.636 | 1.00 | 3.02 | C |
| ATOM | 3077 | C | VAL | C | 111 | 62.373 | −38.462 | 16.602 | 1.00 | 3.02 | C |
| ATOM | 3078 | O | VAL | C | 111 | 62.445 | −37.883 | 17.681 | 1.00 | 3.02 | O |
| ATOM | 3079 | N | ALA | C | 112 | 62.294 | −39.779 | 16.507 | 1.00 | 3.02 | N |
| ATOM | 3080 | CA | ALA | C | 112 | 62.447 | −40.583 | 17.697 | 1.00 | 3.02 | C |
| ATOM | 3081 | CB | ALA | C | 112 | 62.210 | −42.054 | 17.386 | 1.00 | 3.02 | C |
| ATOM | 3082 | C | ALA | C | 112 | 63.837 | −40.352 | 18.329 | 1.00 | 3.02 | C |
| ATOM | 3083 | O | ALA | C | 112 | 63.984 | −40.412 | 19.544 | 1.00 | 3.02 | O |
| ATOM | 3084 | N | HIS | C | 113 | 64.846 | −40.057 | 17.515 | 1.00 | 3.02 | N |
| ATOM | 3085 | CA | HIS | C | 113 | 66.191 | −39.834 | 18.045 | 1.00 | 3.02 | C |
| ATOM | 3086 | CB | HIS | C | 113 | 67.253 | −39.906 | 16.944 | 1.00 | 3.02 | C |
| ATOM | 3087 | CG | HIS | C | 113 | 68.571 | −39.336 | 17.360 | 1.00 | 3.02 | C |
| ATOM | 3088 | ND1 | HIS | C | 113 | 69.605 | −40.110 | 17.840 | 1.00 | 3.02 | N |
| ATOM | 3089 | CE1 | HIS | C | 113 | 70.624 | −39.327 | 18.150 | 1.00 | 3.02 | C |
| ATOM | 3090 | NE2 | HIS | C | 113 | 70.280 | −38.076 | 17.904 | 1.00 | 3.02 | N |
| ATOM | 3091 | CD2 | HIS | C | 113 | 69.003 | −38.055 | 17.409 | 1.00 | 3.02 | C |
| ATOM | 3092 | C | HIS | C | 113 | 66.330 | −38.519 | 18.809 | 1.00 | 3.02 | C |
| ATOM | 3093 | O | HIS | C | 113 | 66.704 | −38.522 | 19.986 | 1.00 | 3.02 | O |
| ATOM | 3094 | N | THR | C | 114 | 66.070 | −37.408 | 18.117 | 1.00 | 3.02 | N |
| ATOM | 3095 | CA | THR | C | 114 | 66.078 | −36.059 | 18.694 | 1.00 | 3.02 | C |
| ATOM | 3096 | CB | THR | C | 114 | 65.477 | −35.055 | 17.684 | 1.00 | 3.02 | C |
| ATOM | 3097 | OG1 | THR | C | 114 | 65.918 | −35.369 | 16.350 | 1.00 | 3.02 | O |
| ATOM | 3098 | CG2 | THR | C | 114 | 65.836 | −33.616 | 18.053 | 1.00 | 3.02 | C |
| ATOM | 3099 | C | THR | C | 114 | 65.265 | −35.977 | 20.002 | 1.00 | 3.02 | C |
| ATOM | 3100 | O | THR | C | 114 | 65.794 | −35.614 | 21.055 | 1.00 | 3.02 | O |
| ATOM | 3101 | N | VAL | C | 115 | 63.981 | −36.333 | 19.897 | 1.00 | 3.02 | N |
| ATOM | 3102 | CA | VAL | C | 115 | 62.992 | −36.278 | 20.978 | 1.00 | 3.02 | C |
| ATOM | 3103 | CB | VAL | C | 115 | 61.600 | −36.768 | 20.460 | 1.00 | 3.02 | C |
| ATOM | 3104 | CG1 | VAL | C | 115 | 60.570 | −36.924 | 21.591 | 1.00 | 3.02 | C |
| ATOM | 3105 | CG2 | VAL | C | 115 | 61.072 | −35.825 | 19.389 | 1.00 | 3.02 | C |
| ATOM | 3106 | C | VAL | C | 115 | 63.417 | −37.067 | 22.216 | 1.00 | 3.02 | C |
| ATOM | 3107 | O | VAL | C | 115 | 63.282 | −36.579 | 23.347 | 1.00 | 3.02 | O |
| ATOM | 3108 | N | ALA | C | 116 | 63.918 | −38.286 | 21.978 | 1.00 | 3.02 | N |
| ATOM | 3109 | CA | ALA | C | 116 | 64.369 | −39.214 | 23.027 | 1.00 | 3.02 | C |
| ATOM | 3110 | CB | ALA | C | 116 | 64.979 | −40.458 | 22.411 | 1.00 | 3.02 | C |
| ATOM | 3111 | C | ALA | C | 116 | 65.384 | −38.542 | 23.917 | 1.00 | 3.02 | C |
| ATOM | 3112 | O | ALA | C | 116 | 65.123 | −38.304 | 25.099 | 1.00 | 3.02 | O |
| ATOM | 3113 | N | TYR | C | 117 | 66.541 | −38.249 | 23.326 | 1.00 | 3.02 | N |
| ATOM | 3114 | CA | TYR | C | 117 | 67.585 | −37.453 | 23.953 | 1.00 | 3.02 | C |
| ATOM | 3115 | CB | TYR | C | 117 | 68.620 | −37.042 | 22.906 | 1.00 | 3.02 | C |
| ATOM | 3116 | CG | TYR | C | 117 | 70.042 | −37.015 | 23.411 | 1.00 | 3.02 | C |
| ATOM | 3117 | CD1 | TYR | C | 117 | 71.073 | −37.541 | 22.647 | 1.00 | 3.02 | C |
| ATOM | 3118 | CE1 | TYR | C | 117 | 72.380 | −37.537 | 23.098 | 1.00 | 3.02 | C |
| ATOM | 3119 | CZ | TYR | C | 117 | 72.662 | −37.000 | 24.322 | 1.00 | 3.02 | C |
| ATOM | 3120 | OH | TYR | C | 117 | 73.962 | −36.988 | 24.762 | 1.00 | 3.02 | O |
| ATOM | 3121 | CE2 | TYR | C | 117 | 71.653 | −36.481 | 25.113 | 1.00 | 3.02 | C |
| ATOM | 3122 | CD2 | TYR | C | 117 | 70.355 | −36.486 | 24.653 | 1.00 | 3.02 | C |
| ATOM | 3123 | C | TYR | C | 117 | 66.986 | −36.205 | 24.596 | 1.00 | 3.02 | C |
| ATOM | 3124 | O | TYR | C | 117 | 67.190 | −35.970 | 25.787 | 1.00 | 3.02 | O |
| ATOM | 3125 | N | LEU | C | 118 | 66.230 | −35.435 | 23.804 | 1.00 | 3.02 | N |
| ATOM | 3126 | CA | LEU | C | 118 | 65.546 | −34.222 | 24.262 | 1.00 | 3.02 | C |
| ATOM | 3127 | CB | LEU | C | 118 | 64.488 | −33.778 | 23.244 | 1.00 | 3.02 | C |
| ATOM | 3128 | CG | LEU | C | 118 | 64.745 | −32.711 | 22.177 | 1.00 | 3.02 | C |
| ATOM | 3129 | CD1 | LEU | C | 118 | 63.649 | −32.742 | 21.110 | 1.00 | 3.02 | C |
| ATOM | 3130 | CD2 | LEU | C | 118 | 64.875 | −31.303 | 22.789 | 1.00 | 3.02 | C |
| ATOM | 3131 | C | LEU | C | 118 | 64.866 | −34.381 | 25.620 | 1.00 | 3.02 | C |
| ATOM | 3132 | O | LEU | C | 118 | 64.938 | −33.484 | 26.467 | 1.00 | 3.02 | O |
| ATOM | 3133 | N | GLY | C | 119 | 64.193 | −35.516 | 25.808 | 1.00 | 3.02 | N |
| ATOM | 3134 | CA | GLY | C | 119 | 63.383 | −35.747 | 27.002 | 1.00 | 3.02 | C |
| ATOM | 3135 | C | GLY | C | 119 | 64.190 | −36.069 | 28.250 | 1.00 | 3.02 | C |
| ATOM | 3136 | O | GLY | C | 119 | 64.722 | −35.156 | 28.927 | 1.00 | 3.02 | O |
| ATOM | 3137 | N | LYS | C | 120 | 64.279 | −37.356 | 28.565 | 1.00 | 3.02 | N |
| ATOM | 3138 | CA | LYS | C | 120 | 65.086 | −37.796 | 29.690 | 1.00 | 3.02 | C |
| ATOM | 3139 | CB | LYS | C | 120 | 64.973 | −39.321 | 29.889 | 1.00 | 3.02 | C |
| ATOM | 3140 | CG | LYS | C | 120 | 63.529 | −39.782 | 30.186 | 1.00 | 3.02 | C |
| ATOM | 3141 | CD | LYS | C | 120 | 63.449 | −41.106 | 30.901 | 1.00 | 3.02 | C |
| ATOM | 3142 | CE | LYS | C | 120 | 62.124 | −41.243 | 31.660 | 1.00 | 3.02 | C |
| ATOM | 3143 | NZ | LYS | C | 120 | 62.069 | −42.494 | 32.496 | 1.00 | 3.02 | N |
| ATOM | 3144 | C | LYS | C | 120 | 66.512 | −37.339 | 29.404 | 1.00 | 3.02 | C |
| ATOM | 3145 | O | LYS | C | 120 | 67.176 | −37.868 | 28.499 | 1.00 | 3.02 | O |
| ATOM | 3146 | N | LEU | C | 121 | 66.950 | −36.326 | 30.158 | 1.00 | 3.02 | N |
| ATOM | 3147 | CA | LEU | C | 121 | 68.227 | −35.621 | 29.927 | 1.00 | 3.02 | C |
| ATOM | 3148 | CB | LEU | C | 121 | 68.725 | −34.951 | 31.216 | 1.00 | 3.02 | C |
| ATOM | 3149 | CG | LEU | C | 121 | 67.733 | −34.058 | 31.980 | 1.00 | 3.02 | C |
| ATOM | 3150 | CD1 | LEU | C | 121 | 68.414 | −33.336 | 33.136 | 1.00 | 3.02 | C |
| ATOM | 3151 | CD2 | LEU | C | 121 | 66.983 | −33.054 | 31.072 | 1.00 | 3.02 | C |
| ATOM | 3152 | C | LEU | C | 121 | 69.322 | −36.506 | 29.343 | 1.00 | 3.02 | C |
| ATOM | 3153 | O | LEU | C | 121 | 70.125 | −36.063 | 28.533 | 1.00 | 3.02 | O |
| ATOM | 3154 | N | ARG | C | 122 | 69.314 | −37.770 | 29.745 | 1.00 | 3.02 | N |
| ATOM | 3155 | CA | ARG | C | 122 | 70.324 | −38.753 | 29.351 | 1.00 | 3.02 | C |
| ATOM | 3156 | CB | ARG | C | 122 | 70.110 | −40.077 | 30.110 | 1.00 | 3.02 | C |
| ATOM | 3157 | CG | ARG | C | 122 | 70.681 | −40.048 | 31.524 | 1.00 | 3.02 | C |
| ATOM | 3158 | CD | ARG | C | 122 | 70.382 | −41.316 | 32.304 | 1.00 | 3.02 | C |
| ATOM | 3159 | NE | ARG | C | 122 | 71.491 | −41.661 | 33.199 | 1.00 | 3.02 | N |
| ATOM | 3160 | CZ | ARG | C | 122 | 71.421 | −42.535 | 34.201 | 1.00 | 3.02 | C |
| ATOM | 3161 | NH1 | ARG | C | 122 | 70.276 | −43.166 | 34.460 | 1.00 | 3.02 | N |
| ATOM | 3162 | NH2 | ARG | C | 122 | 72.503 | −42.761 | 34.945 | 1.00 | 3.02 | N |
| ATOM | 3163 | C | ARG | C | 122 | 70.518 | −38.984 | 27.835 | 1.00 | 3.02 | C |
| ATOM | 3164 | O | ARG | C | 122 | 69.636 | −38.674 | 27.022 | 1.00 | 3.02 | O |
| ATOM | 3165 | N | ALA | C | 123 | 71.692 | −39.521 | 27.486 | 1.00 | 3.02 | N |
| ATOM | 3166 | CA | ALA | C | 123 | 72.103 | −39.768 | 26.106 | 1.00 | 3.02 | C |
| ATOM | 3167 | CB | ALA | C | 123 | 73.594 | −39.489 | 25.925 | 1.00 | 3.02 | C |
| ATOM | 3168 | C | ALA | C | 123 | 71.774 | −41.181 | 25.627 | 1.00 | 3.02 | C |
| ATOM | 3169 | O | ALA | C | 123 | 71.287 | −41.345 | 24.507 | 1.00 | 3.02 | O |
| ATOM | 3170 | N | PRO | C | 124 | 72.016 | −42.207 | 26.476 | 1.00 | 3.02 | N |
| ATOM | 3171 | CA | PRO | C | 124 | 71.824 | −43.612 | 26.063 | 1.00 | 3.02 | C |
| ATOM | 3172 | CB | PRO | C | 124 | 71.851 | −44.384 | 27.392 | 1.00 | 3.02 | C |
| ATOM | 3173 | CG | PRO | C | 124 | 71.737 | −43.311 | 28.474 | 1.00 | 3.02 | C |
| ATOM | 3174 | CD | PRO | C | 124 | 72.434 | −42.127 | 27.886 | 1.00 | 3.02 | C |
| ATOM | 3175 | C | PRO | C | 124 | 70.501 | −43.884 | 25.353 | 1.00 | 3.02 | C |
| ATOM | 3176 | O | PRO | C | 124 | 70.413 | −44.829 | 24.571 | 1.00 | 3.02 | O |
| ATOM | 3177 | N | ILE | C | 125 | 69.483 | −43.071 | 25.625 | 1.00 | 3.02 | N |
| ATOM | 3178 | CA | ILE | C | 125 | 68.207 | −43.212 | 24.928 | 1.00 | 3.02 | C |
| ATOM | 3179 | CB | ILE | C | 125 | 67.008 | −42.498 | 25.674 | 1.00 | 3.02 | C |
| ATOM | 3180 | CG1 | ILE | C | 125 | 65.664 | −42.863 | 25.021 | 1.00 | 3.02 | C |
| ATOM | 3181 | CD1 | ILE | C | 125 | 64.442 | −42.831 | 25.948 | 1.00 | 3.02 | C |
| ATOM | 3182 | CG2 | ILE | C | 125 | 67.193 | −40.987 | 25.751 | 1.00 | 3.02 | C |
| ATOM | 3183 | C | ILE | C | 125 | 68.336 | −42.841 | 23.432 | 1.00 | 3.02 | C |
| ATOM | 3184 | O | ILE | C | 125 | 67.495 | −43.225 | 22.616 | 1.00 | 3.02 | O |
| ATOM | 3185 | N | ARG | C | 126 | 69.402 | −42.125 | 23.075 | 1.00 | 3.02 | N |
| ATOM | 3186 | CA | ARG | C | 126 | 69.671 | −41.787 | 21.670 | 1.00 | 3.02 | C |
| ATOM | 3187 | CB | ARG | C | 126 | 70.530 | −40.517 | 21.565 | 1.00 | 3.02 | C |
| ATOM | 3188 | CG | ARG | C | 126 | 72.055 | −40.751 | 21.573 | 1.00 | 3.02 | C |
| ATOM | 3189 | CD | ARG | C | 126 | 72.608 | −41.192 | 20.202 | 1.00 | 3.02 | C |
| ATOM | 3190 | NE | ARG | C | 126 | 73.985 | −40.751 | 20.002 | 1.00 | 3.02 | N |
| ATOM | 3191 | CZ | ARG | C | 126 | 74.514 | −40.421 | 18.827 | 1.00 | 3.02 | C |
| ATOM | 3192 | NH1 | ARG | C | 126 | 73.779 | −40.456 | 17.735 | 1.00 | 3.02 | N |
| ATOM | 3193 | NH2 | ARG | C | 126 | 75.774 | −40.015 | 18.750 | 1.00 | 3.02 | N |
| ATOM | 3194 | C | ARG | C | 126 | 70.326 | −42.925 | 20.854 | 1.00 | 3.02 | C |
| ATOM | 3195 | O | ARG | C | 126 | 70.245 | −42.927 | 19.617 | 1.00 | 3.02 | O |
| ATOM | 3196 | N | SER | C | 127 | 70.986 | −43.866 | 21.538 | 1.00 | 3.02 | N |
| ATOM | 3197 | CA | SER | C | 127 | 71.730 | −44.942 | 20.879 | 1.00 | 3.02 | C |
| ATOM | 3198 | CB | SER | C | 127 | 72.291 | −45.913 | 21.919 | 1.00 | 3.02 | C |
| ATOM | 3199 | OG | SER | C | 127 | 71.277 | −46.379 | 22.788 | 1.00 | 3.02 | O |
| ATOM | 3200 | C | SER | C | 127 | 70.892 | −45.702 | 19.854 | 1.00 | 3.02 | C |
| ATOM | 3201 | O | SER | C | 127 | 71.404 | −46.173 | 18.837 | 1.00 | 3.02 | O |
| ATOM | 3202 | N | VAL | C | 128 | 69.593 | −45.790 | 20.125 | 1.00 | 3.02 | N |
| ATOM | 3203 | CA | VAL | C | 128 | 68.657 | −46.574 | 19.318 | 1.00 | 3.02 | C |
| ATOM | 3204 | CB | VAL | C | 128 | 67.330 | −46.848 | 20.115 | 1.00 | 3.02 | C |
| ATOM | 3205 | CG1 | VAL | C | 128 | 66.144 | −47.188 | 19.194 | 1.00 | 3.02 | C |
| ATOM | 3206 | CG2 | VAL | C | 128 | 67.565 | −47.980 | 21.127 | 1.00 | 3.02 | C |
| ATOM | 3207 | C | VAL | C | 128 | 68.441 | −46.039 | 17.883 | 1.00 | 3.02 | C |
| ATOM | 3208 | O | VAL | C | 128 | 68.991 | −46.604 | 16.927 | 1.00 | 3.02 | O |
| ATOM | 3209 | N | THR | C | 129 | 67.684 | −44.952 | 17.735 | 1.00 | 3.02 | N |
| ATOM | 3210 | CA | THR | C | 129 | 67.219 | −44.512 | 16.410 | 1.00 | 3.02 | C |
| ATOM | 3211 | CB | THR | C | 129 | 65.986 | −43.626 | 16.515 | 1.00 | 3.02 | C |
| ATOM | 3212 | OG1 | THR | C | 129 | 66.336 | −42.445 | 17.223 | 1.00 | 3.02 | O |
| ATOM | 3213 | CG2 | THR | C | 129 | 64.870 | −44.323 | 17.262 | 1.00 | 3.02 | C |
| ATOM | 3214 | C | THR | C | 129 | 68.251 | −43.827 | 15.497 | 1.00 | 3.02 | C |
| ATOM | 3215 | O | THR | C | 129 | 68.001 | −43.645 | 14.304 | 1.00 | 3.02 | O |
| ATOM | 3216 | N | TYR | C | 130 | 69.398 | −43.450 | 16.058 | 1.00 | 3.02 | N |
| ATOM | 3217 | CA | TYR | C | 130 | 70.489 | −42.827 | 15.293 | 1.00 | 3.02 | C |
| ATOM | 3218 | CB | TYR | C | 130 | 71.518 | −42.209 | 16.237 | 1.00 | 3.02 | C |
| ATOM | 3219 | CG | TYR | C | 130 | 72.720 | −43.086 | 16.560 | 1.00 | 3.02 | C |
| ATOM | 3220 | CD1 | TYR | C | 130 | 74.001 | −42.755 | 16.095 | 1.00 | 3.02 | C |
| ATOM | 3221 | CE1 | TYR | C | 130 | 75.106 | −43.531 | 16.403 | 1.00 | 3.02 | C |
| ATOM | 3222 | CZ | TYR | C | 130 | 74.941 | −44.662 | 17.173 | 1.00 | 3.02 | C |
| ATOM | 3223 | OH | TYR | C | 130 | 76.027 | −45.434 | 17.478 | 1.00 | 3.02 | O |
| ATOM | 3224 | CE2 | TYR | C | 130 | 73.687 | −45.024 | 17.644 | 1.00 | 3.02 | C |
| ATOM | 3225 | CD2 | TYR | C | 130 | 72.583 | −44.231 | 17.341 | 1.00 | 3.02 | C |
| ATOM | 3226 | C | TYR | C | 130 | 71.185 | −43.789 | 14.317 | 1.00 | 3.02 | C |
| ATOM | 3227 | O | TYR | C | 130 | 71.727 | −43.358 | 13.307 | 1.00 | 3.02 | O |
| ATOM | 3228 | N | THR | C | 131 | 71.200 | −45.082 | 14.654 | 1.00 | 3.02 | N |
| ATOM | 3229 | CA | THR | C | 131 | 71.556 | −46.158 | 13.705 | 1.00 | 3.02 | C |
| ATOM | 3230 | CB | THR | C | 131 | 71.876 | −47.510 | 14.446 | 1.00 | 3.02 | C |
| ATOM | 3231 | OG1 | THR | C | 131 | 72.984 | −47.332 | 15.341 | 1.00 | 3.02 | O |
| ATOM | 3232 | CG2 | THR | C | 131 | 72.232 | −48.617 | 13.468 | 1.00 | 3.02 | C |
| ATOM | 3233 | C | THR | C | 131 | 70.416 | −46.316 | 12.671 | 1.00 | 3.02 | C |
| ATOM | 3234 | O | THR | C | 131 | 70.655 | −46.465 | 11.470 | 1.00 | 3.02 | O |
| ATOM | 3235 | N | LEU | C | 132 | 69.175 | −46.242 | 13.145 | 1.00 | 3.02 | N |
| ATOM | 3236 | CA | LEU | C | 132 | 68.018 | −46.158 | 12.249 | 1.00 | 3.02 | C |
| ATOM | 3237 | CB | LEU | C | 132 | 66.697 | −46.411 | 13.009 | 1.00 | 3.02 | C |
| ATOM | 3238 | CG | LEU | C | 132 | 66.529 | −47.743 | 13.766 | 1.00 | 3.02 | C |
| ATOM | 3239 | CD1 | LEU | C | 132 | 65.348 | −47.669 | 14.735 | 1.00 | 3.02 | C |
| ATOM | 3240 | CD2 | LEU | C | 132 | 66.375 | −48.917 | 12.800 | 1.00 | 3.02 | C |
| ATOM | 3241 | C | LEU | C | 132 | 67.983 | −44.825 | 11.471 | 1.00 | 3.02 | C |
| ATOM | 3242 | O | LEU | C | 132 | 67.142 | −44.643 | 10.586 | 1.00 | 3.02 | O |
| ATOM | 3243 | N | ALA | C | 133 | 68.894 | −43.907 | 11.807 | 1.00 | 3.02 | N |
| ATOM | 3244 | CA | ALA | C | 133 | 69.133 | −42.696 | 11.010 | 1.00 | 3.02 | C |
| ATOM | 3245 | CB | ALA | C | 133 | 69.276 | −41.480 | 11.914 | 1.00 | 3.02 | C |
| ATOM | 3246 | C | ALA | C | 133 | 70.372 | −42.842 | 10.118 | 1.00 | 3.02 | C |
| ATOM | 3247 | O | ALA | C | 133 | 70.361 | −42.457 | 8.945 | 1.00 | 3.02 | O |
| ATOM | 3248 | N | GLN | C | 134 | 71.431 | −43.414 | 10.689 | 1.00 | 3.02 | N |
| ATOM | 3249 | CA | GLN | C | 134 | 72.718 | −43.568 | 10.021 | 1.00 | 3.02 | C |
| ATOM | 3250 | CB | GLN | C | 134 | 73.776 | −43.992 | 11.037 | 1.00 | 3.02 | C |
| ATOM | 3251 | CG | GLN | C | 134 | 74.179 | −42.947 | 12.051 | 1.00 | 3.02 | C |
| ATOM | 3252 | CD | GLN | C | 134 | 75.296 | −43.424 | 12.983 | 1.00 | 3.02 | C |
| ATOM | 3253 | OE1 | GLN | C | 134 | 75.597 | −44.625 | 13.073 | 1.00 | 3.02 | O |
| ATOM | 3254 | NE2 | GLN | C | 134 | 75.924 | −42.476 | 13.673 | 1.00 | 3.02 | N |
| ATOM | 3255 | C | GLN | C | 134 | 72.708 | −44.600 | 8.888 | 1.00 | 3.02 | C |
| ATOM | 3256 | O | GLN | C | 134 | 73.556 | −44.532 | 7.991 | 1.00 | 3.02 | O |
| ATOM | 3257 | N | LEU | C | 135 | 71.790 | −45.575 | 8.954 | 1.00 | 3.02 | N |
| ATOM | 3258 | CA | LEU | C | 135 | 71.693 | −46.646 | 7.929 | 1.00 | 3.02 | C |
| ATOM | 3259 | CB | LEU | C | 135 | 71.001 | −47.914 | 8.462 | 1.00 | 3.02 | C |
| ATOM | 3260 | CG | LEU | C | 135 | 71.891 | −49.013 | 9.073 | 1.00 | 3.02 | C |
| ATOM | 3261 | CD1 | LEU | C | 135 | 71.056 | −50.011 | 9.885 | 1.00 | 3.02 | C |
| ATOM | 3262 | CD2 | LEU | C | 135 | 72.721 | −49.736 | 8.003 | 1.00 | 3.02 | C |
| ATOM | 3263 | C | LEU | C | 135 | 71.046 | −46.177 | 6.626 | 1.00 | 3.02 | C |
| ATOM | 3264 | O | LEU | C | 135 | 71.528 | −46.510 | 5.547 | 1.00 | 3.02 | O |
| ATOM | 3265 | N | PRO | C | 136 | 69.931 | −45.432 | 6.717 | 1.00 | 3.02 | N |
| ATOM | 3266 | CA | PRO | C | 136 | 69.540 | −44.610 | 5.573 | 1.00 | 3.02 | C |
| ATOM | 3267 | CB | PRO | C | 136 | 68.401 | −43.742 | 6.122 | 1.00 | 3.02 | C |
| ATOM | 3268 | CG | PRO | C | 136 | 68.138 | −44.179 | 7.498 | 1.00 | 3.02 | C |
| ATOM | 3269 | CD | PRO | C | 136 | 68.933 | −45.389 | 7.802 | 1.00 | 3.02 | C |
| ATOM | 3270 | C | PRO | C | 136 | 70.688 | −43.751 | 4.967 | 1.00 | 3.02 | C |
| ATOM | 3271 | O | PRO | C | 136 | 70.799 | −43.685 | 3.742 | 1.00 | 3.02 | O |
| ATOM | 3272 | N | CYS | C | 137 | 71.521 | −43.107 | 5.795 | 1.00 | 3.02 | N |
| ATOM | 3273 | CA | CYS | C | 137 | 72.755 | −42.465 | 5.302 | 1.00 | 3.02 | C |
| ATOM | 3274 | CB | CYS | C | 137 | 73.677 | −42.055 | 6.450 | 1.00 | 3.02 | C |
| ATOM | 3275 | SG | CYS | C | 137 | 73.308 | −40.497 | 7.255 | 1.00 | 3.02 | S |
| ATOM | 3276 | C | CYS | C | 137 | 73.550 | −43.397 | 4.394 | 1.00 | 3.02 | C |
| ATOM | 3277 | O | CYS | C | 137 | 74.155 | −42.937 | 3.418 | 1.00 | 3.02 | O |
| ATOM | 3278 | N | ALA | C | 138 | 73.543 | −44.693 | 4.736 | 1.00 | 3.02 | N |
| ATOM | 3279 | CA | ALA | C | 138 | 74.399 | −45.696 | 4.119 | 1.00 | 3.02 | C |
| ATOM | 3280 | CB | ALA | C | 138 | 74.618 | −46.846 | 5.065 | 1.00 | 3.02 | C |
| ATOM | 3281 | C | ALA | C | 138 | 73.811 | −46.191 | 2.824 | 1.00 | 3.02 | C |
| ATOM | 3282 | O | ALA | C | 138 | 74.558 | −46.541 | 1.907 | 1.00 | 3.02 | O |
| ATOM | 3283 | N | SER | C | 139 | 72.478 | −46.216 | 2.757 | 1.00 | 3.02 | N |
| ATOM | 3284 | CA | SER | C | 139 | 71.763 | −46.696 | 1.570 | 1.00 | 3.02 | C |
| ATOM | 3285 | CB | SER | C | 139 | 70.489 | −47.472 | 1.942 | 1.00 | 3.02 | C |
| ATOM | 3286 | OG | SER | C | 139 | 69.539 | −46.673 | 2.634 | 1.00 | 3.02 | O |
| ATOM | 3287 | C | SER | C | 139 | 71.458 | −45.573 | 0.594 | 1.00 | 3.02 | C |
| ATOM | 3288 | O | SER | C | 139 | 71.382 | −45.817 | −0.614 | 1.00 | 3.02 | O |
| ATOM | 3289 | N | MET | C | 140 | 71.280 | −44.358 | 1.125 | 1.00 | 3.02 | N |
| ATOM | 3290 | CA | MET | C | 140 | 71.157 | −43.131 | 0.313 | 1.00 | 3.02 | C |
| ATOM | 3291 | CB | MET | C | 140 | 70.788 | −41.910 | 1.165 | 1.00 | 3.02 | C |
| ATOM | 3292 | CG | MET | C | 140 | 69.399 | −41.894 | 1.726 | 1.00 | 3.02 | C |
| ATOM | 3293 | SD | MET | C | 140 | 68.816 | −40.251 | 2.219 | 1.00 | 3.02 | S |
| ATOM | 3294 | CE | MET | C | 140 | 68.530 | −39.431 | 0.642 | 1.00 | 3.02 | C |
| ATOM | 3295 | C | MET | C | 140 | 72.463 | −42.795 | −0.399 | 1.00 | 3.02 | C |
| ATOM | 3296 | O | MET | C | 140 | 72.441 | −42.365 | −1.559 | 1.00 | 3.02 | O |
| ATOM | 3297 | N | ALA | C | 141 | 73.578 | −42.980 | 0.321 | 1.00 | 3.02 | N |
| ATOM | 3298 | CA | ALA | C | 141 | 74.921 | −42.641 | −0.141 | 1.00 | 3.02 | C |
| ATOM | 3299 | CB | ALA | C | 141 | 75.907 | −42.701 | 1.011 | 1.00 | 3.02 | C |
| ATOM | 3300 | C | ALA | C | 141 | 75.376 | −43.545 | −1.280 | 1.00 | 3.02 | C |
| ATOM | 3301 | O | ALA | C | 141 | 76.171 | −43.137 | −2.128 | 1.00 | 3.02 | O |
| ATOM | 3302 | N | LEU | C | 142 | 74.873 | −44.776 | −1.286 | 1.00 | 3.02 | N |
| ATOM | 3303 | CA | LEU | C | 142 | 75.111 | −45.709 | −2.385 | 1.00 | 3.02 | C |
| ATOM | 3304 | CB | LEU | C | 142 | 74.989 | −47.167 | −1.921 | 1.00 | 3.02 | C |
| ATOM | 3305 | CG | LEU | C | 142 | 76.223 | −47.742 | −1.204 | 1.00 | 3.02 | C |
| ATOM | 3306 | CD1 | LEU | C | 142 | 75.825 | −48.817 | −0.238 | 1.00 | 3.02 | C |
| ATOM | 3307 | CD2 | LEU | C | 142 | 77.316 | −48.254 | −2.162 | 1.00 | 3.02 | C |
| ATOM | 3308 | C | LEU | C | 142 | 74.191 | −45.434 | −3.561 | 1.00 | 3.02 | C |
| ATOM | 3309 | O | LEU | C | 142 | 74.651 | −45.366 | −4.704 | 1.00 | 3.02 | O |
| ATOM | 3310 | N | GLN | C | 143 | 72.896 | −45.272 | −3.279 | 1.00 | 3.02 | N |
| ATOM | 3311 | CA | GLN | C | 143 | 71.891 | −44.947 | −4.314 | 1.00 | 3.02 | C |
| ATOM | 3312 | CB | GLN | C | 143 | 70.505 | −44.767 | −3.709 | 1.00 | 3.02 | C |
| ATOM | 3313 | CG | GLN | C | 143 | 69.639 | −45.998 | −3.829 | 1.00 | 3.02 | C |
| ATOM | 3314 | CD | GLN | C | 143 | 68.229 | −45.758 | −3.319 | 1.00 | 3.02 | C |
| ATOM | 3315 | OE1 | GLN | C | 143 | 67.906 | −44.669 | −2.838 | 1.00 | 3.02 | O |
| ATOM | 3316 | NE2 | GLN | C | 143 | 67.378 | −46.783 | −3.415 | 1.00 | 3.02 | N |
| ATOM | 3317 | C | GLN | C | 143 | 72.252 | −43.742 | −5.178 | 1.00 | 3.02 | C |
| ATOM | 3318 | O | GLN | C | 143 | 71.890 | −43.686 | −6.352 | 1.00 | 3.02 | O |
| ATOM | 3319 | N | ILE | C | 144 | 72.957 | −42.783 | −4.591 | 1.00 | 3.02 | N |
| ATOM | 3320 | CA | ILE | C | 144 | 73.639 | −41.769 | −5.383 | 1.00 | 3.02 | C |
| ATOM | 3321 | CB | ILE | C | 144 | 74.485 | −40.804 | −4.509 | 1.00 | 3.02 | C |
| ATOM | 3322 | CG1 | ILE | C | 144 | 73.637 | −40.185 | −3.403 | 1.00 | 3.02 | C |
| ATOM | 3323 | CD1 | ILE | C | 144 | 74.392 | −39.975 | −2.146 | 1.00 | 3.02 | C |
| ATOM | 3324 | CG2 | ILE | C | 144 | 75.115 | −39.704 | −5.363 | 1.00 | 3.02 | C |
| ATOM | 3325 | C | ILE | C | 144 | 74.541 | −42.449 | −6.418 | 1.00 | 3.02 | C |
| ATOM | 3326 | O | ILE | C | 144 | 74.214 | −42.458 | −7.604 | 1.00 | 3.02 | O |
| ATOM | 3327 | N | LEU | C | 145 | 75.641 | −43.047 | −5.956 | 1.00 | 3.02 | N |
| ATOM | 3328 | CA | LEU | C | 145 | 76.727 | −43.533 | −6.831 | 1.00 | 3.02 | C |
| ATOM | 3329 | CB | LEU | C | 145 | 77.849 | −44.193 | −6.014 | 1.00 | 3.02 | C |
| ATOM | 3330 | CG | LEU | C | 145 | 78.171 | −43.603 | −4.648 | 1.00 | 3.02 | C |
| ATOM | 3331 | CD1 | LEU | C | 145 | 78.893 | −44.609 | −3.773 | 1.00 | 3.02 | C |
| ATOM | 3332 | CD2 | LEU | C | 145 | 78.943 | −42.306 | −4.811 | 1.00 | 3.02 | C |
| ATOM | 3333 | C | LEU | C | 145 | 76.265 | −44.512 | −7.898 | 1.00 | 3.02 | C |
| ATOM | 3334 | O | LEU | C | 145 | 76.728 | −44.453 | −9.043 | 1.00 | 3.02 | O |
| ATOM | 3335 | N | TRP | C | 146 | 75.365 | −45.414 | −7.513 | 1.00 | 3.02 | N |
| ATOM | 3336 | CA | TRP | C | 146 | 74.910 | −46.458 | −8.405 | 1.00 | 3.02 | C |
| ATOM | 3337 | CB | TRP | C | 146 | 74.073 | −47.486 | −7.648 | 1.00 | 3.02 | C |
| ATOM | 3338 | CG | TRP | C | 146 | 74.867 | −48.189 | −6.604 | 1.00 | 3.02 | C |
| ATOM | 3339 | CD1 | TRP | C | 146 | 74.755 | −48.041 | −5.258 | 1.00 | 3.02 | C |
| ATOM | 3340 | NE1 | TRP | C | 146 | 75.673 | −48.831 | −4.618 | 1.00 | 3.02 | N |
| ATOM | 3341 | CE2 | TRP | C | 146 | 76.411 | −49.511 | −5.553 | 1.00 | 3.02 | C |
| ATOM | 3342 | CD2 | TRP | C | 146 | 75.928 | −49.132 | −6.822 | 1.00 | 3.02 | C |
| ATOM | 3343 | CE3 | TRP | C | 146 | 76.517 | −49.693 | −7.966 | 1.00 | 3.02 | C |
| ATOM | 3344 | CZ3 | TRP | C | 146 | 77.557 | −50.607 | −7.803 | 1.00 | 3.02 | C |
| ATOM | 3345 | CH2 | TRP | C | 146 | 78.013 | −50.967 | −6.526 | 1.00 | 3.02 | C |
| ATOM | 3346 | CZ2 | TRP | C | 146 | 77.454 | −50.436 | −5.392 | 1.00 | 3.02 | C |
| ATOM | 3347 | C | TRP | C | 146 | 74.148 | −45.858 | −9.567 | 1.00 | 3.02 | C |
| ATOM | 3348 | O | TRP | C | 146 | 74.578 | −45.984 | −10.723 | 1.00 | 3.02 | O |
| ATOM | 3349 | N | GLU | C | 147 | 73.029 | −45.201 | −9.255 | 1.00 | 3.02 | N |
| ATOM | 3350 | CA | GLU | C | 147 | 72.263 | −44.448 | −10.248 | 1.00 | 3.02 | C |
| ATOM | 3351 | CB | GLU | C | 147 | 71.113 | −43.693 | −9.590 | 1.00 | 3.02 | C |
| ATOM | 3352 | CG | GLU | C | 147 | 69.753 | −43.914 | −10.253 | 1.00 | 3.02 | C |
| ATOM | 3353 | CD | GLU | C | 147 | 68.657 | −43.098 | −9.596 | 1.00 | 3.02 | C |
| ATOM | 3354 | OE1 | GLU | C | 147 | 67.868 | −43.693 | −8.820 | 1.00 | 3.02 | O |
| ATOM | 3355 | OE2 | GLU | C | 147 | 68.619 | −41.862 | −9.834 | 1.00 | 3.02 | O |
| ATOM | 3356 | C | GLU | C | 147 | 73.165 | −43.459 | −10.976 | 1.00 | 3.02 | C |
| ATOM | 3357 | O | GLU | C | 147 | 73.106 | −43.375 | −12.200 | 1.00 | 3.02 | O |
| ATOM | 3358 | N | ALA | C | 148 | 74.007 | −42.737 | −10.227 | 1.00 | 3.02 | N |
| ATOM | 3359 | CA | ALA | C | 148 | 74.888 | −41.709 | −10.794 | 1.00 | 3.02 | C |
| ATOM | 3360 | CB | ALA | C | 148 | 75.472 | −40.849 | −9.696 | 1.00 | 3.02 | C |
| ATOM | 3361 | C | ALA | C | 148 | 75.985 | −42.294 | −11.684 | 1.00 | 3.02 | C |
| ATOM | 3362 | O | ALA | C | 148 | 76.708 | −41.561 | −12.362 | 1.00 | 3.02 | O |
| ATOM | 3363 | N | ALA | C | 149 | 76.098 | −43.620 | −11.680 | 1.00 | 3.02 | N |
| ATOM | 3364 | CA | ALA | C | 149 | 76.964 | −44.332 | −12.618 | 1.00 | 3.02 | C |
| ATOM | 3365 | CB | ALA | C | 149 | 77.593 | −45.555 | −11.950 | 1.00 | 3.02 | C |
| ATOM | 3366 | C | ALA | C | 149 | 76.171 | −44.743 | −13.862 | 1.00 | 3.02 | C |
| ATOM | 3367 | O | ALA | C | 149 | 76.752 | −45.029 | −14.915 | 1.00 | 3.02 | O |
| ATOM | 3368 | N | ARG | C | 150 | 74.846 | −44.773 | −13.732 | 1.00 | 3.02 | N |
| ATOM | 3369 | CA | ARG | C | 150 | 73.969 | −45.122 | −14.847 | 1.00 | 3.02 | C |
| ATOM | 3370 | CB | ARG | C | 150 | 72.768 | −45.947 | −14.351 | 1.00 | 3.02 | C |
| ATOM | 3371 | CG | ARG | C | 150 | 72.090 | −46.779 | −15.433 | 1.00 | 3.02 | C |
| ATOM | 3372 | CD | ARG | C | 150 | 70.716 | −47.242 | −14.993 | 1.00 | 3.02 | C |
| ATOM | 3373 | NE | ARG | C | 150 | 69.689 | −46.965 | −16.004 | 1.00 | 3.02 | N |
| ATOM | 3374 | CZ | ARG | C | 150 | 68.370 | −47.078 | −15.807 | 1.00 | 3.02 | C |
| ATOM | 3375 | NH1 | ARG | C | 150 | 67.878 | −47.490 | −14.641 | 1.00 | 3.02 | N |
| ATOM | 3376 | NH2 | ARG | C | 150 | 67.537 | −46.792 | −16.790 | 1.00 | 3.02 | N |
| ATOM | 3377 | C | ARG | C | 150 | 73.528 | −43.881 | −15.662 | 1.00 | 3.02 | C |
| ATOM | 3378 | O | ARG | C | 150 | 72.792 | −43.993 | −16.659 | 1.00 | 3.02 | O |
| ATOM | 3379 | N | HIS | C | 151 | 73.974 | −42.697 | −15.252 | 1.00 | 3.02 | N |
| ATOM | 3380 | CA | HIS | C | 151 | 73.714 | −41.485 | −16.037 | 1.00 | 3.02 | C |
| ATOM | 3381 | CB | HIS | C | 151 | 73.211 | −40.355 | −15.134 | 1.00 | 3.02 | C |
| ATOM | 3382 | CG | HIS | C | 151 | 72.211 | −40.816 | −14.127 | 1.00 | 3.02 | C |
| ATOM | 3383 | ND1 | HIS | C | 151 | 71.005 | −41.374 | −14.484 | 1.00 | 3.02 | N |
| ATOM | 3384 | CE1 | HIS | C | 151 | 70.337 | −41.710 | −13.395 | 1.00 | 3.02 | C |
| ATOM | 3385 | NE2 | HIS | C | 151 | 71.076 | −41.406 | −12.345 | 1.00 | 3.02 | N |
| ATOM | 3386 | CD2 | HIS | C | 151 | 72.255 | −40.848 | −12.777 | 1.00 | 3.02 | C |
| ATOM | 3387 | C | HIS | C | 151 | 74.947 | −41.075 | −16.844 | 1.00 | 3.02 | C |
| ATOM | 3388 | O | HIS | C | 151 | 75.063 | −39.950 | −17.308 | 1.00 | 3.02 | O |
| ATOM | 3389 | N | LEU | C | 152 | 75.866 | −42.012 | −17.006 | 1.00 | 3.02 | N |
| ATOM | 3390 | CA | LEU | C | 152 | 77.106 | −41.775 | −17.714 | 1.00 | 3.02 | C |
| ATOM | 3391 | CB | LEU | C | 152 | 78.259 | −41.512 | −16.723 | 1.00 | 3.02 | C |
| ATOM | 3392 | CG | LEU | C | 152 | 78.161 | −40.726 | −15.394 | 1.00 | 3.02 | C |
| ATOM | 3393 | CD1 | LEU | C | 152 | 79.553 | −40.373 | −14.898 | 1.00 | 3.02 | C |
| ATOM | 3394 | CD2 | LEU | C | 152 | 77.328 | −39.449 | −15.468 | 1.00 | 3.02 | C |
| ATOM | 3395 | C | LEU | C | 152 | 77.400 | −43.025 | −18.540 | 1.00 | 3.02 | C |
| ATOM | 3396 | O | LEU | C | 152 | 78.359 | −43.087 | −19.308 | 1.00 | 3.02 | O |
| ATOM | 3397 | O31 | GTT | T | 153 | 75.170 | −38.077 | 20.264 | 1.00 | 3.02 | O |
| ATOM | 3398 | C3 | GTT | T | 153 | 73.996 | −37.789 | 19.935 | 1.00 | 3.02 | C |
| ATOM | 3399 | O32 | GTT | T | 153 | 73.098 | −38.198 | 20.685 | 1.00 | 3.02 | O |
| ATOM | 3400 | CA3 | GTT | T | 153 | 73.662 | −37.007 | 18.672 | 1.00 | 3.02 | C |
| ATOM | 3401 | N3 | GTT | T | 153 | 73.649 | −37.866 | 17.494 | 1.00 | 3.02 | N |
| ATOM | 3402 | C2 | GTT | T | 153 | 73.039 | −37.619 | 16.333 | 1.00 | 3.02 | C |
| ATOM | 3403 | O2 | GTT | T | 153 | 71.871 | −36.850 | 16.224 | 1.00 | 3.02 | O |
| ATOM | 3404 | CA2 | GTT | T | 153 | 73.621 | −38.236 | 15.100 | 1.00 | 3.02 | C |
| ATOM | 3405 | CB2 | GTT | T | 153 | 72.535 | −39.104 | 14.491 | 1.00 | 3.02 | C |
| ATOM | 3406 | SG2 | GTT | T | 153 | 73.197 | −40.717 | 13.996 | 1.00 | 3.02 | S |
| ATOM | 3407 | N2 | GTT | T | 153 | 74.776 | −39.069 | 15.388 | 1.00 | 3.02 | N |
| ATOM | 3408 | CD1 | GTT | T | 153 | 75.868 | −39.132 | 14.630 | 1.00 | 3.02 | C |
| ATOM | 3409 | OE1 | GTT | T | 153 | 75.943 | −38.670 | 13.298 | 1.00 | 3.02 | O |
| ATOM | 3410 | CG1 | GTT | T | 153 | 77.055 | −39.760 | 15.311 | 1.00 | 3.02 | C |
| ATOM | 3411 | CB1 | GTT | T | 153 | 78.027 | −38.669 | 15.719 | 1.00 | 3.02 | C |
| ATOM | 3412 | CA1 | GTT | T | 153 | 78.556 | −38.899 | 17.131 | 1.00 | 3.02 | C |
| ATOM | 3413 | N1 | GTT | T | 153 | 79.978 | −38.566 | 17.185 | 1.00 | 3.02 | N |
| ATOM | 3414 | C1 | GTT | T | 153 | 77.791 | −38.074 | 18.133 | 1.00 | 3.02 | C |
| ATOM | 3415 | O11 | GTT | T | 153 | 77.035 | −37.144 | 17.762 | 1.00 | 3.02 | O |
| ATOM | 3416 | O12 | GTT | T | 153 | 77.893 | −38.318 | 19.362 | 1.00 | 3.02 | O |
| ATOM | 3417 | O31 | GTT | T | 154 | 88.318 | −23.460 | 15.302 | 1.00 | 3.02 | O |
| ATOM | 3418 | C3 | GTT | T | 154 | 88.417 | −24.556 | 14.659 | 1.00 | 3.02 | C |
| ATOM | 3419 | O32 | GTT | T | 154 | 88.140 | −25.646 | 15.237 | 1.00 | 3.02 | O |
| ATOM | 3420 | CA3 | GTT | T | 154 | 88.864 | −24.632 | 13.204 | 1.00 | 3.02 | C |
| ATOM | 3421 | N3 | GTT | T | 154 | 88.182 | −23.765 | 12.236 | 1.00 | 3.02 | N |
| ATOM | 3422 | C2 | GTT | T | 154 | 87.853 | −24.265 | 11.040 | 1.00 | 3.02 | C |
| ATOM | 3423 | O2 | GTT | T | 154 | 88.117 | −25.619 | 10.723 | 1.00 | 3.02 | O |
| ATOM | 3424 | CA2 | GTT | T | 154 | 87.158 | −23.406 | 10.011 | 1.00 | 3.02 | C |
| ATOM | 3425 | CB2 | GTT | T | 154 | 87.882 | −23.470 | 8.676 | 1.00 | 3.02 | C |
| ATOM | 3426 | SG2 | GTT | T | 154 | 89.641 | −23.814 | 8.919 | 1.00 | 3.02 | S |
| ATOM | 3427 | N2 | GTT | T | 154 | 87.024 | −22.027 | 10.385 | 1.00 | 3.02 | N |
| ATOM | 3428 | CD1 | GTT | T | 154 | 85.850 | −21.426 | 10.230 | 1.00 | 3.02 | C |
| ATOM | 3429 | OE1 | GTT | T | 154 | 84.701 | −22.004 | 9.639 | 1.00 | 3.02 | O |
| ATOM | 3430 | CG1 | GTT | T | 154 | 85.812 | −20.025 | 10.764 | 1.00 | 3.02 | C |
| ATOM | 3431 | CB1 | GTT | T | 154 | 85.359 | −20.041 | 12.219 | 1.00 | 3.02 | C |
| ATOM | 3432 | CA1 | GTT | T | 154 | 86.539 | −20.098 | 13.198 | 1.00 | 3.02 | C |
| ATOM | 3433 | N1 | GTT | T | 154 | 86.611 | −18.796 | 13.857 | 1.00 | 3.02 | N |
| ATOM | 3434 | C1 | GTT | T | 154 | 86.427 | −21.245 | 14.204 | 1.00 | 3.02 | C |
| ATOM | 3435 | O11 | GTT | T | 154 | 86.262 | −22.454 | 13.857 | 1.00 | 3.02 | O |
| ATOM | 3436 | O12 | GTT | T | 154 | 86.508 | −21.058 | 15.457 | 1.00 | 3.02 | O |
| ATOM | 3437 | O31 | GTT | T | 155 | 70.213 | −18.252 | 20.585 | 1.00 | 3.02 | O |
| ATOM | 3438 | C3 | GTT | T | 155 | 70.466 | −17.146 | 20.050 | 1.00 | 3.02 | C |
| ATOM | 3439 | O32 | GTT | T | 155 | 70.396 | −16.130 | 20.767 | 1.00 | 3.02 | O |
| ATOM | 3440 | CA3 | GTT | T | 155 | 70.829 | −17.037 | 18.587 | 1.00 | 3.02 | C |
| ATOM | 3441 | N3 | GTT | T | 155 | 70.112 | −18.065 | 17.846 | 1.00 | 3.02 | N |
| ATOM | 3442 | C2 | GTT | T | 155 | 69.782 | −17.989 | 16.557 | 1.00 | 3.02 | C |
| ATOM | 3443 | O2 | GTT | T | 155 | 70.339 | −17.032 | 15.687 | 1.00 | 3.02 | O |
| ATOM | 3444 | CA2 | GTT | T | 155 | 68.786 | −19.014 | 16.073 | 1.00 | 3.02 | C |
| ATOM | 3445 | CB2 | GTT | T | 155 | 68.139 | −18.634 | 14.745 | 1.00 | 3.02 | C |
| ATOM | 3446 | SG2 | GTT | T | 155 | 67.378 | −16.996 | 14.819 | 1.00 | 3.02 | S |
| ATOM | 3447 | N2 | GTT | T | 155 | 67.765 | −19.288 | 17.067 | 1.00 | 3.02 | N |
| ATOM | 3448 | CD1 | GTT | T | 155 | 66.820 | −20.238 | 16.966 | 1.00 | 3.02 | C |
| ATOM | 3449 | OE1 | GTT | T | 155 | 66.681 | −21.197 | 15.928 | 1.00 | 3.02 | O |
| ATOM | 3450 | CG1 | GTT | T | 155 | 65.833 | −20.270 | 18.100 | 1.00 | 3.02 | C |
| ATOM | 3451 | CB1 | GTT | T | 155 | 66.243 | −21.414 | 19.022 | 1.00 | 3.02 | C |
| ATOM | 3452 | CA1 | GTT | T | 155 | 66.662 | −20.884 | 20.390 | 1.00 | 3.02 | C |
| ATOM | 3453 | N1 | GTT | T | 155 | 66.240 | −21.826 | 21.404 | 1.00 | 3.02 | N |
| ATOM | 3454 | C1 | GTT | T | 155 | 68.153 | −20.660 | 20.507 | 1.00 | 3.02 | C |
| ATOM | 3455 | O11 | GTT | T | 155 | 68.869 | −20.425 | 19.504 | 1.00 | 3.02 | O |
| ATOM | 3456 | O12 | GTT | T | 155 | 68.717 | −20.676 | 21.634 | 1.00 | 3.02 | O |

1-26. (canceled)

27. A method for identifying a drug candidate compound, comprising:
(a) identifying a protein molecule having at least 20% sequence identity with Microsomal Prostaglandin E Synthase 1 (MPGES1);
(b) utilizing the atomic coordinates of MPGES1 to obtain an atomic model of the identified protein molecule;
(c) using the atomic model of the identified protein molecule to determine whether a compound is a drug candidate;
(d) contacting the drug candidate compound with the identified protein molecule; and
(e) measuring for a change in the expression or activity of the identified protein molecule.

28. The method of claim 27, wherein the protein molecule is a Membrane Associated Protein in Eicosanoid and Glutathione Metabolism (MAPEG) protein molecule.

29. The method of claim 27, wherein the atomic model comprises a homology model.

30. The method of claim 29, wherein the homology model is obtained by a modelling software program.

31. The method of claim 27, wherein the atomic model comprises an experimental model.

32. The method of claim 31, wherein the experimental model is obtained with molecular replacement.

33. The method of claim 27, further comprising (f) analyzing the interaction of the drug candidate compound with the identified protein molecule.

34. The method of claim 33, wherein the interaction of the drug candidate compound with the active site of the identified protein molecule is analyzed with a docking-program.

35. The method of claim 33, wherein the structure of the interaction of the drug candidate compound with the identified protein molecule is obtained using molecular replacement.

36. The method of claim 27, wherein
a drug candidate compound that decreases the expression or activity of the identified protein molecule indicates that the drug candidate compound is an inhibitor of the identified protein molecule; and
a drug candidate compound that increases the expression or activity of the identified protein molecule indicates that the drug candidate compound is a promoter of the identified protein molecule.

37. The method of claim 32, wherein at least one catalytic position of the identified protein molecule is mutated prior to identifying a drug candidate compound that interacts with the identified protein molecule.

38. The method of claim 37, wherein the mutation comprises a substitution of at least one amino acid.

39. The method of claim 37, wherein the mutation comprises a deletion of at least one amino acid.

40. The method of claim 27, wherein the atomic coordinates of MPGES1 are as defined in Table 3.

41. A method for determining a drug candidate compound that interacts with Microsomal Prostaglandin E Synthase 1 (MPGES1), comprising:
(a) identifying a drug candidate compound that interacts with MPGES1 and
(b) analyzing the interaction of the drug candidate compound with MPGES1.

42. The method of claim 41, wherein the interaction of the drug candidate compound with the active site of the MPGES1 is analyzed with a docking-program.

43. The method of claim 41, wherein the structure of the interaction of the drug candidate compound with MPGES1 is obtained using molecular replacement.

44. The method of claim 41, wherein the drug candidate compound is identified by using the atomic structure of MPGES1 to design a drug candidate compound.

45. The method of claim 41, wherein the drug candidate compound is identified by
(a) contacting the drug candidate compound with MPGES1; and
(b) measuring for a change in the expression or activity of the protein molecule.

46. The method of claim 45, wherein
a drug candidate compound that decreases the expression or activity of MPGES1 indicates that the drug candidate compound is an inhibitor of MPGES1; and
a drug candidate compound that increases the expression or activity of MPGES1 indicates that the drug candidate compound is a promoter of MPGES1.

47. The method of claim 41, wherein at least one catalytic position of MPGES1 is mutated prior to identifying a drug candidate compound that interacts with MPGES1.

48. The method of claim 47, wherein the mutation comprises a substitution of at least one amino acid.

49. The method of claim 47, wherein the mutation comprises a deletion of at least one amino acid.

50. A crystalline form of MPGES1, wherein two-dimensional crystal parameters of the crystal comprise two-sided plane group p22₁2₁ and a unit cell having a=93.2 Å and b=84.6 Å.

51. The crystalline form according to claim 50, comprising atoms arranged in a spatial relationship represented by the coordinates of Table 3.


SEQUENCE LISTING (7 sequences)

SEQ ID NO: 1 - protein, 158 aa, Homo sapiens
HHHHHHMPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 2 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 3 - protein, 155 aa, Homo sapiens
MVDLTQVMDDEVFMAFASYATIILSKMMLMSTATAFYRLTRKVFANPEDCVAFGKGENAKKYLRTDDRVERVRRAHLNDLENIIPELGIGLLYSLSGPDPSTAILHFRLFVGARIYHTIAYLTPLPQPNRALSFFVGYGVTLSMAYRLLKSKLYL

SEQ ID NO: 4 - protein, 150 aa, Homo sapiens
MKDEVALLAAVTLLGVLLQAYFSLQVISARRAFRVSPPLTTGPPEFERVYRAQVNCSEYFPLFLATLQVAGIFFHEGAAALCGLVYLFARLRYFQGYARSAQLRLAPLYASARALWLLVALAALGLLAHFLPAALRAALLGRLRTLLPWA

SEQ ID NO: 5 - protein, 147 aa, Homo sapiens
MAGNSILLAAVSILSACQQSYFALQVGKARLKYKVTPPAVTGSPEFERVFRAQQNCVEFYPIFIITLWMAGWYFNQVFATCLGLVYIYGRHLYFWGYSEAAKKRITGFRLSLGILALLTLLGALGIANSFLDEYLDLNIAKKLRRQF

SEQ ID NO: 6 - protein, 161 aa, Homo sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 7 - protein, 152 aa, Homo sapiens
MAVLSKEYGFVLLTGAASFIMVAHLAINVSKARKKYKVEYPIMYSTDPENGHIFNCIQRAHQNTLEVYPPFLFFLAVGGVYHPRIASGLGLAWIVGRVLYAYGYYTGEPSKRSRGALGSIALLGLVGTTVCSAFQHLGWVKSGLGSGPKCCH